(19) United States
(12) Patent Application Publication
(10) Pub. No.: US 2005/0208500 A1
(43) Pub. Date: Sep. 22, 2005

(54) Signatures of ER status in breast cancer

(76) Inventors: Mark G. Erlander, Encinitas, CA (US); Xiao-Jun Ma, San Diego, CA (US); Wei Wang, San Marcos, CA (US); James L. Wittliff, Louisville, KY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/794,263

(22) Filed: Mar. 4, 2004

Related U.S. Application Data
(60) Provisional application No. 60/451,942, filed on Mar. 4, 2003.

(51) Int. Cl. C12Q 1/68; C12M 1/34

(52) U.S. Cl. 435/6; 435/287.2

(57) ABSTRACT

The invention relates to the identification and use of gene expression profiles, or patterns, suitable for identification of populations that are positive and negative for estrogen receptor expression. The gene expression profiles may be embodied in nucleic acid expression, protein expression, or other expression formats, and may be used in the study and/or diagnosis of cells and tissue in breast cancer as well as for the study and/or determination of prognosis of a patient, including breast cancer survival.

### RELATED APPLICATIONS

This application claims benefit of priority from U.S. Provisional Patent application 60/451,942, filed Mar. 4, 2003, which is hereby incorporated by reference in its entirety as if fully set forth.

### FIELD OF THE INVENTION

The invention relates to the identification and use of gene expression profiles, or patterns, involved in breast cancer survival. In particular, the invention provides the identities of genes that may be used to identify populations that are positive and negative for estrogen receptor expression. The gene expression profiles, whether embodied in nucleic acid expression, protein expression, or other expression formats, are used in the study and/or diagnosis of cells and tissue in breast cancer as well as for the study and/or determination of prognosis of a patient. The profiles may also be used in methods of diagnosis or prognosis.

### BACKGROUND OF THE INVENTION

Breast cancer is by far the most common cancer among women. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease (National Cancer Institute, Surveillance Epidemiology and End Results (NCI SEER) program, Cancer Statistics Review (CSR), www-seer.ims.nci.nih.gov/Publications/CSR1973 (1998)). Based on cancer rates from 1995 through 1997, a report from the National Cancer Institute (NCI) estimates that about 1 in 8 women in the United States (approximately 12.8 percent) will develop breast cancer during her lifetime (NCI's Surveillance, Epidemiology, and End Results Program (SEER) publication SEER Cancer Statistics Review 1973-1997). Breast cancer is the second most common form of cancer, after skin cancer, among women in the United States. An estimated 250,100 new cases of breast cancer are expected to be diagnosed in the United States in 2001. Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & Figures 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMD-92-12; 1991). Only 5 to 10% of breast cancers are linked to a family history of breast cancer (Henderson I C, Breast Cancer. In: Murphy G P, Lawrence W L, Lenhard R E (eds). Clinical Oncology. Atlanta, Ga.: American Cancer Society; 1995:198-219).

Each breast has 15 to 20 sections called lobes. Within each lobe are many smaller lobules. Lobules end in dozens of tiny bulbs that can produce milk. The lobes, lobules, and bulbs are all linked by thin tubes called ducts. These ducts lead to the nipple in the center of a dark area of skin called the areola. Fat surrounds the lobules and ducts. There are no muscles in the breast, but muscles lie under each breast and cover the ribs. Each breast also contains blood vessels and lymph vessels. The lymph vessels carry colorless fluid called lymph, and lead to the lymph nodes. Clusters of lymph nodes are found near the breast in the axilla (under the arm), above the collarbone, and in the chest.

Breast tumors can be either benign or malignant. Benign tumors are not cancerous, they do not spread to other parts of the body, and are not a threat to life. They can usually be removed, and in most cases, do not come back. Malignant tumors are cancerous, and can invade and damage nearby tissues and organs. Malignant tumor cells may metastasize, entering the bloodstream or lymphatic system. When breast cancer cells metastasize outside the breast, they are often found in the lymph nodes under the arm (axillary lymph nodes). If the cancer has reached these nodes, it means that cancer cells may have spread to other lymph nodes or other organs, such as bones, liver, or lungs.

Major and intensive research has been focused on early detection, treatment and prevention. This has included an emphasis on determining the presence of precancerous or cancerous ductal epithelial cells. These cells are analyzed, for example, for cell morphology, for protein markers, for nucleic acid markers, for chromosomal abnormalities, for biochemical markers, and for other characteristic changes that would signal the presence of cancerous or precancerous cells. This has led to various molecular alterations that have been reported in breast cancer, few of which have been well characterized in human clinical breast specimens. Molecular alterations include presence/absence of estrogen and progesterone steroid receptors, HER-2 expression/amplification (Mark H F, et al. HER-2/neu gene amplification in stages I-IV breast cancer detected by fluorescent in situ hybridization. Genet Med; 1(3):98-103 1999), Ki-67 (an antigen that is present in all stages of the cell cycle except G0 and used as a marker for tumor cell proliferation, and prognostic markers (including oncogenes, tumor suppressor genes, and angiogenesis markers) like p53, p27, Cathepsin D, pS2, multi-drug resistance (MDR) gene, and CD31.

Estrogen receptor (ER) status has been of particular interest because it has been correlated with prognosis and treatment regimens. Generally speaking, patients identified as having ER positive breast cancer biopsies have a better overall survival expectation while patients with ER negative biopsies are treated more aggressively, such as with immediate chemotherapy after surgical intervention, because of a poor prognosis.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The present invention relates to the identification and use of gene expression patterns (or profiles or “signatures”) which are correlated with (and thus able to discriminate between) cells that are positive or negative for estrogen receptor (ER) expression in breast cancer specimens. The patterns may thus serve as a supplement to assays for ER status in breast cancer samples or used as a substitute for known assays for ER status. The patterns may thus be used in diagnostic or prognostic methods or assays in the clinic to determine the course of treatment following identification of the presence of breast cancer or subsequent surgical removal thereof.

The patterns also provide the identity of genes that may be the focus of therapeutic efforts to identify agents and treatment methods to alleviate the severity of breast cancer, improve the chances for surviving breast cancer, and/or decrease the chances of breast cancer recurrence or metastases. Such agents and methods may be used to increase or decrease the expression of one or more genes of the patterns to restore cells to a less cancerous state or a state with a better prognosis for the patient. The patterns may also be used to identify cellular mechanisms or pathways, as well as the components of such mechanisms or pathways, to be altered or modulated in the treatment of breast cancer.

The present invention provides a non-subjective means for the identification of ER status in breast cancer samples by assaying for the expression patterns associated with ER status. Thus subjective interpretation is necessary and a more accurate assessment of ER status, and breast cancer status and prognosis, is provided. Furthermore, the expression patterns can also be used as a means to assay small, node negative tumors that are not readily assayed by other means.

The gene expression patterns comprise one or more than one gene capable of discriminating between breast cancer that is ER positive or ER negative with significant accuracy. The gene(s) are identified as correlated with ER expression status in breast cancer such that the levels of their expression are relevant to a determination of ER status in breast cancer of a cell. Thus in one aspect, the invention provides a method to determine the ER status of breast cancer of a subject afflicted with, or suspected of having, breast cancer by assaying a cell containing sample from said subject for expression of one or more than one gene disclosed herein as correlated with ER status in breast cancer.

Gene expression patterns of the invention are identified by analysis of gene expression in multiple samples of ER positive and ER negative samples from breast cancer biopsies. The overall gene expression profile of a sample is obtained through quantifying the expression levels of mRNA corresponding to multiple genes to identify genes that are positively, or negatively, correlated, with ER positive and ER negative sample.

A profile of genes that are highly correlated with ER status may be used to assay an sample from a subject afflicted with, or suspected of having, breast cancer to identify the ER status of breast cancer to which the sample belongs. This may be done in combination with, or separate from a direct assay for ER expression. Such assays may be used as part of a method to determine the therapeutic treatment for said subject based upon the ER status identified. The present invention also provides for the advantageous ability to determine ER status in combination with other information to provide more detailed information in diagnosing and treating breast cancer.

The correlated genes may be used singly with significant accuracy or in combination to increase the ability to accurately identify ER status. The present invention thus provides means for correlating a molecular expression phenotype with ER expression and thus a physiological (cellular) state. This correlation also provides a way to molecularly diagnose and/or monitor a cell's status. Additional uses of the correlated gene(s) are in the classification of cells and tissues; determination of diagnosis and/or prognosis; and determination and/or alteration of therapy.

An assay of the invention may utilize a means related to the expression level of the sequences disclosed herein as long as the assay reflects, quantitatively or qualitatively, expression of the sequence. Preferably, however, a quantitative assay means is preferred. The ability to discriminate is conferred by the identification of expression of the individual genes as relevant and not by the form of the assay used to determine the actual level of expression. An assay may utilize any identifying feature of an identified individual gene as disclosed herein as long as the assay reflects, quantitatively or qualitatively, expression of the gene. Identifying features include, but are not limited to, unique nucleic acid sequences used to encode (DNA), or express (RNA), said gene or epitopes specific to, or activities of, a protein encoded by said gene. Alternative means include detection of nucleic acid amplification as indicative of increased expression levels and nucleic acid inactivation, deletion, or methylation, as indicative of decreased expression levels. Stated differently, the invention may be practiced by assaying one or more aspect of the DNA template(s) underlying the expression of the disclosed sequence(s), of the RNA used as an intermediate to express the sequence(s), or of the proteinaceous product expressed by the sequence(s), as well as proteolytic fragments of such products. As such, the detection of the presence of, amount of, stability of, or degradation (including rate) of, such DNA, RNA and proteinaceous molecules may be used in the practice of the invention. As such, all that is required is the identity of the gene(s) necessary to discriminate between ER positive and negative samples and an appropriate cell containing sample for use in an expression assay.

In one aspect, the invention provides for the identification of the gene expression patterns by analyzing global, or near global, gene expression from single cells or homogenous cell populations which have been dissected away from, or otherwise isolated or purified from, contaminating cells beyond that possible by a simple biopsy. Because the expression of numerous genes fluctuate between cells from different patients as well as between cells from the same patient sample, the expression of multiple individual genes may be analyzed to for the best the ability to discriminate ER positive and negative samples.

In a further aspect, the gene(s) capable of discriminating between ER positive and negative samples may be used to identify ER status of an unknown sample of cell(s) from the breast. Preferably, the sample is isolated via non-invasive means. The expression of said gene(s) in said unknown sample may be determined and compared to the expression of said gene(s) in reference data of gene expression patterns from ER positive and/or negative samples. Alternatively, the expression level may be compared to expression levels in normal or non-cancerous cells, preferably from the same sample or subject. In embodiments of the invention utilizing quantitative PCR, the expression level may be compared to expression levels of reference genes in the same sample or a ratio of expression levels may be used. The invention provides for ratios of the expression level of a sequence that is under expressed to the expression level of a sequence that is over expressed as a indicator of ER positive or ER negative status. The use of a ratio can reduce comparisons with normal or non-cancerous cells.

One advantage provided by the present invention is that contaminating, non-breast cells (such as infiltrating lymphocytes or other immune system cells) are not present to possibly affect the genes identified or the subsequent analysis of gene expression to identify the status of suspected breast cancer cells. Such contamination is present where a biopsy is used to generate gene expression profiles.

While the present invention has been described mainly in the context of human breast cancer, it may be practiced in the context of breast cancer of any animal known to be potentially afflicted by breast cancer. Preferred animals for the application of the present invention are mammals, particularly those important to agricultural applications (such as, but not limited to, cattle, sheep, horses, and other “farm animals”) and for human companionship (such as, but not limited to, dogs and cats).

### DETAILED DESCRIPTION OF THE SPECIFIC EMBODIMENTS

Definitions of Terms as Used Herein:

A gene expression “pattern” or “profile” or “signature” refers to the relative expression of a gene between ER positive and negative cells which expression is correlated with being able to distinguish between ER positive and negative cells.

A “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature, as well as the ordered bases that precede or follow a “coding region”. Non-limiting examples of the latter include 5′ and 3′ untranslated regions of a gene. It is appreciated that more than one polynucleotide may be capable of encoding a discrete product. It is also appreciated that alleles and polymorphisms of the disclosed sequences may exist and may be used in the practice of the invention to identify the expression level(s) of the disclosed sequences or the allele or polymorphism. Identification of an allele or polymorphism depends in part upon chromosomal location and ability to recombine during mitosis.

The terms “correlate” or “correlation” or equivalents thereof refer to an association between expression of one or more genes and the ER status of a breast cancer cell and/or a breast cancer patient. A gene may be expressed at higher or lower levels and still be correlated with ER status and thus breast cancer survival or outcome. The invention provides for the correlation between increases, as well as decreases, in expression of gene sequences and ER positive or negative status. Increases and decreases may be readily expressed in the form of a ratio between expression in a non-normal cell and a normal cell such that a ratio of one (1) indicates no difference while ratios of two (2) and one-half indicate twice as much, and half as much, expression in the non-normal cell versus the normal cell, respectively. Expression levels can be readily determined by quantitative methods as described below.

For example, increases in gene expression can be indicated by ratios of or about 1.1, of or about 1.2, of or about 1.3, of or about 1.4, of or about 1.5, of or about 1.6, of or about 1.7, of or about 1.8, of or about 1.9, of or about 2, of or about 2.5, of or about 3, of or about 3.5, of or about 4, of or about 4.5, of or about 5, of or about 5.5, of or about 6, of or about 6.5, of or about 7, of or about 7.5, of or about 8, of or about 8.5, of or about 9, of or about 9.5, of or about 10, of or about 15, of or about 20, of or about 30, of or about 40, of or about 50, of or about 60, of or about 70, of or about 80, of or about 90, of or about 100, of or about 150, of or about 200, of or about 300, of or about 400, of or about 500, of or about 600, of or about 700, of or about 800, of or about 900, or of or about 1000. A ratio of 2 is a 100% (or a two-fold) increase in expression. Decreases in gene expression can be indicated by ratios of or about 0.9, of or about 0.8, of or about 0.7, of or about 0.6, of or about 0.5, of or about 0.4, of or about 0.3, of or about 0.2, of or about 0.1, of or about 0.05, of or about 0.01, of or about 0.005, of or about 0.001, of or about 0.0005, of or about 0.0001, of or about 0.00005, of or about 0.00001, of or about 0.000005, or of or about 0.000001.

For a given phenotype, a ratio of the expression of a gene sequence expressed at increased levels in correlation with an ER status to the expression of a gene sequence expressed at decreased levels in correlation with the ER status may also be used as an indicator of the phenotype. As a non-limiting example, the ER positive status may be correlated with increased expression of a gene sequence over expressed in ER positive cells as well as decreased expression of a gene sequence under expressed in ER positive cells. Therefore, a ratio of the expression levels of the under expressed sequence to the expression levels of the over expressed sequence may be used as an indicator of ER status. Ratios comprising gene sequences that are differentially expressed in ER negative cells may also be used.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence.

By corresponding is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17). Methods for amplifying mRNA are generally known in the art, and include reverse transcription PCR (RT-PCR) and those described in U.S. patent application Ser. No. 10/062,857 (filed on Oct. 25, 2001), as well as U.S. Provisional Patent Applications 60/298,847 (filed Jun. 15, 2001) and 60/257,801 (filed Dec. 22, 2000), all of which are hereby incorporated by reference in their entireties as if fully set forth. Another method which may be used is quantitative PCR (or Q-PCR). Alternatively, RNA may be directly labeled as the corresponding cDNA by methods known in the art.

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray.

Because the invention relies upon the identification of genes that are over- or under-expressed, one embodiment of the invention involves determining expression by hybridization of mRNA, or an amplified or cloned version thereof, of a sample cell to a polynucleotide that is unique to a particular gene sequence. Preferred polynucleotides of this type contain at least about 20, at least about 22, at least about 24, at least about 26, at least about 28, at least about 30, or at least about 32 consecutive base pairs of a gene sequence that is not found in other gene sequences. The term “about” as used in the previous sentence refers to an increase or decrease of 1 from the stated numerical value. Even more preferred are polynucleotides of at least or about 50, at least or about 100, at least about or 150, at least or about 200, at least or about 250, at least or about 300, at least or about 350, or at least or about 400 base pairs of a gene sequence that is not found in other gene sequences. The term “about” as used in the preceding sentence refers to an increase or decrease of 10% from the stated numerical value. Such polynucleotides may also be referred to as polynucleotide probes that are capable of hybridizing to sequences of the genes, or unique portions thereof, described herein. Preferably, the sequences are those of mRNA encoded by the genes, the corresponding cDNA to such mRNAs, and/or amplified versions of such sequences. In preferred embodiments of the invention, the polynucleotide probes are immobilized on an array, other devices, or in individual spots that localize the probes.

In another embodiment of the invention, all or part of a disclosed sequence may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a disclosed sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization. Additional methods to detect the expression of expressed nucleic acids include RNAse protection assays, including liquid phase hybridizations, and in situ hybridization of cells.

Alternatively, and in yet another embodiment of the invention, gene expression may be determined by analysis of expressed protein in a cell sample of interest by use of one or more antibodies specific for one or more epitopes of individual gene products (proteins), or proteolytic fragments thereof, in said cell sample or in a bodily fluid of a subject. The cell sample may be one of breast cancer epithelial cells enriched from the blood of a subject, such as by use of labeled antibodies against cell surface markers followed by fluorescence activated cell sorting (FACS). Such antibodies are preferably labeled to permit their easy detection after binding to the gene product. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “breast tissue sample” or “breast cell sample” refers to a sample of breast tissue or fluid isolated from an individual suspected of being afflicted with, or at risk of developing, breast cancer. Such samples are primary isolates (in contrast to cultured cells) and may be collected by any non-invasive means, including, but not limited to, ductal lavage, fine needle aspiration, needle biopsy, the devices and methods described in U.S. Pat. No. 6,328,709, or any other suitable means recognized in the art. Alternatively, the “sample” may be collected by an invasive method, including, but not limited to, surgical biopsy. A sample of the invention may also be one that has been formalin fixed and paraffin embedded (FFPE).

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in non-coding regions of genes as disclosed herein may also be assayed in the practice of the invention.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

Increases and decreases in expression of the disclosed sequences are defined in the following terms based upon percent or fold changes over expression in normal cells. Increases may be of 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 10, 20, 30, 40, 50, 55, 60, 65, 70, 75, 80, 82, 84, 86, 88, 90, 92, 94, 96, 98, 99 or 100% relative to expression levels in normal cells.

Unless defined otherwise all technical and scientific terms used herein have the same meaning as commonly understood to one of ordinary skill in the art to which this invention belongs.

### SPECIFIC EMBODIMENTS

The present invention relates to the identification and use of gene expression patterns (or profiles or “signatures”) which discriminate between (or are correlated with) breast cancer cells that are ER positive or negative, preferably ERα positive or negative. Because the overall gene expression profile differs from person to person, cancer to cancer, and cancer cell to cancer cell, correlations between genes expressed or under expressed in ER positive and negative cells are capable of identifying ER status. ER status has been used as a factor in determinations of therapeutic treatment of patients with breast cancer. For example, ER positive status has been used as an indicator of responsiveness to treatment with tamoxifen and other selective estrogen receptor modulators (SERMs).

The present invention may be practiced with any subset of the genes disclosed herein. Gene expression in cells of breast cancer biopsies were used to identify thousands of genes capable of discriminating between ER positive and negative breast cancer cells as described in the following Example. The identification may be made by using expression profiles of various homogenous breast cancer cell populations, which are optionally isolated by microdissection, such as, but not limited to, laser capture microdissection (LCM) of 100-1000 cells.

Genes that are identified as being expressed differently between ER positive and ER negative cells may be analyzed by standard statistical analysis, such as the t-test, to assign a value for the significance of the difference. Genes with a significance above a particular threshold may be included in a pattern that segregates breast cancer based on ER status.

The expression of other genes in a breast cancer cell would be relatively unable to provide information concerning, and thus assist in the discrimination of ER status in breast cancer.

To determine the (increased or decreased) expression levels of genes in the practice of the present invention, any method known in the art may be utilized. In one preferred embodiment of the invention, expression based on detection of RNA which hybridizes to the genes identified and disclosed herein is used. This is readily performed by any RNA detection or amplification+detection method known or recognized as equivalent in the art such as, but not limited to, reverse transcription-PCR, the methods disclosed in U.S. patent application Ser. No. 10/062,857 (filed on Oct. 25, 2001) as well as U.S. Provisional Patent Applications 60/298,847 (filed Jun. 15, 2001) and 60/257,801 (filed Dec. 22, 2000), and methods to detect the presence, or absence, of RNA stabilizing or destabilizing sequences.

Alternatively, expression based on detection of DNA status may be used. Detection of the DNA of an identified gene as methylated or deleted may be used for genes that have decreased expression in correlation with ER status. This may be readily performed by PCR based methods known in the art, including, but not limited to, quantitative PCR (Q-PCR). Conversely, detection of the DNA of an identified gene as amplified may be used for genes that have increased expression in correlation with ER status. This may be readily performed by PCR based, fluorescent in situ hybridization (FISH) and chromosome in situ hybridization (CISH) methods known in the art.

Expression based on detection of a presence, increase, or decrease in protein levels or activity may also be used. Detection may be performed by any immunohistochemistry (IHC) based, blood based (especially for secreted proteins), antibody (including autoantibodies against the protein) based, ex foliate cell (from the cancer) based, mass spectroscopy based, and image (including used of labeled ligand) based method known in the art and recognized as appropriate for the detection of the protein. Antibody and image based methods are additionally useful for the localization of tumors after determination of cancer by use of cells obtained by a non-invasive procedure (such as ductal lavage or fine needle aspiration), where the source of the cancerous cells is not known. A labeled antibody or ligand may be used to localize the carcinoma(s) within a patient.

A preferred embodiment using a nucleic acid based assay to determine expression is by immobilization of one or more sequences of the genes identified herein on a solid support, including, but not limited to, a solid substrate as an array or to beads or bead based technology as known in the art. Alternatively, solution based expression assays known in the art may also be used. The immobilized gene(s) may be in the form of polynucleotides that are unique or otherwise specific to the gene(s) such that the polynucleotide would be capable of hybridizing to a DNA or RNA corresponding to the gene(s). These polynucleotides may be the full length of the gene(s) or be short sequences of the genes (up to one nucleotide shorter than the full length sequence known in the art by deletion from the 5′ or 3′ end of the sequence) that are optionally minimally interrupted (such as by mismatches or inserted non-complementary base pairs) such that hybridization with a DNA or RNA corresponding to the gene(s) is not affected. Preferably, the polynucleotides used are from the 3′ end of the gene, such as within about 350, about 300, about 250, about 200, about 150, about 100, or about 50 nucleotides from the polyadenylation signal or polyadenylation site of a gene or expressed sequence. Polynucleotides containing mutations relative to the sequences of the disclosed genes may also be used so long as the presence of the mutations still allows hybridization to produce a detectable signal.

Alternatively, amplification of such sequences from the 3′ end of genes by methods such as quantitative PCR may be used to determine the expression levels of the sequences. The Ct values generated by such methods may be used to generate the ratios of expression levels as described herein.

The immobilized gene(s) may be used to determine the state of nucleic acid samples prepared from sample breast cell(s) for which the ER status is not known or for confirmation of a status that is already assigned to the sample breast cell(s). Without limiting the invention, such a cell may be from a patient suspected of being afflicted with, or at risk of developing, breast cancer. The immobilized polynucleotide(s) need only be sufficient to specifically hybridize, optionally under stringent conditions, to the corresponding nucleic acid molecules derived from the sample. While even a single correlated gene sequence may to able to provide adequate accuracy in discriminating between ER status, two or more, three or more, four or more, five or more, six or more, seven or more, eight or more, nine or more, ten or more, or eleven or more of the genes identified herein may be used as a subset capable of discriminating may be used in combination to increase the accuracy of the method. The invention specifically contemplates the selection of more than one, two or more, three or more, four or more, five or more, six or more, seven or more, eight or more, nine or more, ten or more, or eleven or more of the genes disclosed in the tables and figures herein for use as a subset in the identification of whether a breast cancer sample is ER positive or negative.

Of course 15 or more, 20 or more, 30 or more, 40 or more, 50 or more, 60 or more, 70 or more, 80 or more, 90 or more, 100 or more, 150 or more, 200 or more, 250 or more, 300 or more, 350 or more, 400 or more, 450 or more, 500 or more, 600 or more, 700 or more, 800 or more, 900 or more, 1000 or more, or all the genes provided in Table 1 or 2 below may be used. Additionally, genes that identify ER positive and ER negative may be used together to permit differential identification of a test sample as being ER positive or ER negative.

In embodiments where only one or a few genes are to be analyzed, the nucleic acid derived from the sample breast cancer cell(s) may be preferentially amplified by use of appropriate primers such that only the genes to be analyzed are amplified to reduce contaminating background signals from other genes expressed in the breast cell. Alternatively, and where multiple genes are to be analyzed or where very few cells (or one cell) is used, the nucleic acid from the sample may be globally amplified before hybridization to the immobilized polynucleotides. Of course RNA, or the cDNA counterpart thereof may be directly labeled and used, without amplification, by methods known in the art.

The invention is preferably practiced with unique sequences present within the gene sequences disclosed herein. The uniqueness of a disclosed gene sequence refers to the portions or entireties of the sequences which are found in each gene to the exclusion of other genes. Such unique sequences include those found at the 3′ untranslated portion of the genes. Preferred unique sequences for the practice of the invention are those which contribute to the consensus sequences for each gene such that the unique sequences will be useful in detecting expression in a variety of individuals rather than being specific for a polymorphism present in some individuals. Alternatively, sequences unique to an individual or a subpopulation may be used. The preferred unique sequences are preferably of the lengths of polynucleotides of the invention as discussed herein.

In particularly preferred embodiments of the invention, polynucleotides having sequences present in the 3′ untranslated and/or non-coding regions of the disclosed gene sequences are used to detect expression levels in breast cells. Such polynucleotides may optionally contain sequences found in the 3′ portions of the coding regions of the disclosed sequences. Polynucleotides containing a combination of sequences from the coding and 3′ non-coding regions preferably have the sequences arranged contiguously, with no intervening heterologous sequence(s).

Alternatively, the invention may be practiced with polynucleotides having sequences present in the 5′ untranslated and/or non-coding regions of gene sequences in breast cells to detect their levels of expression. Such polynucleotides may optionally contain sequences found in the 5′ portions of the coding regions. Polynucleotides containing a combination of sequences from the coding and 5′ non-coding regions preferably have the sequences arranged contiguously, with no intervening heterologous sequence(s). The invention may also be practiced with sequences present in the coding regions of disclosed sequences.

Preferred polynucleotides contain sequences from 3′ or 5′ untranslated and/or non-coding regions of at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, or at least about 46 consecutive nucleotides. The term “about” as used in the previous sentence refers to an increase or decrease of 1 from the stated numerical value. Even more preferred are polynucleotides containing sequences of at least or about 50, at least or about 100, at least about or 150, at least or about 200, at least or about 250, at least or about 300, at least or about 350, or at least or about 400 consecutive nucleotides. The term “about” as used in the preceding sentence refers to an increase or decrease of 10% from the stated numerical value.

Sequences from the 3′ or 5′ end of the above described coding regions as found in polynucleotides of the invention are of the same lengths as those described above, except that they would naturally be limited by the length of the coding region. The 3′ end of a coding region may include sequences up to the 3′ half of the coding region. Conversely, the 5′ end of a coding region may include sequences up the 5′ half of the coding region. Of course the above described sequences, or the coding regions and polynucleotides containing portions thereof, may be used in their entireties.

Polynucleotides combining the sequences from a 3′ untranslated and/or non-coding region and the associated 3′ end of the coding region are preferably at least or about 100, at least about or 150, at least or about 200, at least or about 250, at least or about 300, at least or about 350, or at least or about 400 consecutive nucleotides. Preferably, the polynucleotides used are from the 3′ end of the gene, such as within about 350, about 300, about 250, about 200, about 150, about 100, or about 50 nucleotides from the polyadenylation signal or polyadenylation site of a gene or expressed sequence. Polynucleotides containing mutations relative to the sequences of the disclosed genes may also be used so long as the presence of the mutations still allows hybridization to produce a detectable signal.

In another embodiment of the invention, polynucleotides containing deletions of nucleotides from the 5′ and/or 3′ end of the above disclosed sequences may be used. The deletions are preferably of 1-5, 5-10, 10-15, 15-20, 20-25, 25-30, 30-35, 35-40, 40-45, 45-50, 50-60, 60-70, 70-80, 80-90, 90-100, 100-125, 125-150, 150-175, or 175-200 nucleotides from the 5′ and/or 3′ end, although the extent of the deletions would naturally be limited by the length of the disclosed sequences and the need to be able to use the polynucleotides for the detection of expression levels.

Other polynucleotides of the invention from the 3′ end of the above disclosed sequences include those of primers and optional probes for quantitative PCR. Preferably, the primers and probes are those which amplify a region less than about 350, less than about 300, less than about 250, less than about 200, less than about 150, less than about 100, or less than about 50 nucleotides from the from the polyadenylation signal or polyadenylation site of a gene or expressed sequence.

In yet another embodiment of the invention, polynucleotides containing portions of the above disclosed sequences including the 3′ end may be used in the practice of the invention. Such polynucleotides would contain at least or about 50, at least or about 100, at least about or 150, at least or about 200, at least or about 250, at least or about 300, at least or about 350, or at least or about 400 consecutive nucleotides from the 3′ end of the disclosed sequences.

The above assay embodiments may be used in a number of different ways to identify or detect ER status in a breast cancer cell sample from a patient. In many cases, this may reflect a secondary screen for the patient, who may have already undergone mammography or physical exam as a primary screen. If positive, the subsequent needle biopsy, ductal lavage, fine needle aspiration, or other analogous methods may provide the sample for use in the above assay embodiments. The present invention is particularly useful in combination with non-invasive protocols, such as ductal lavage or fine needle aspiration, to prepare a breast cell sample.

The present invention provides an objective set of criteria, in the form of gene expression profiles of a discrete set of genes, to discriminate (or delineate) between ER positive and negative breast cancer cells.

In one embodiment of the invention, the isolation and analysis of a breast cancer cell sample may be performed as follows:
(1) Ductal lavage or other non-invasive procedure is performed on a patient to obtain a sample.
(2) Sample is prepared and coated onto a microscope slide. Note that ductal lavage results in clusters of cells that are cytologically examined as stated above.
(3) Pathologist or image analysis software scans the sample for the presence of non-normal and/or atypical cells.
(4) If non-normal and/or atypical cells are observed, those cells are harvested (e.g. by microdissection such as LCM).
(5) RNA is extracted from the harvested cells.
(6) RNA is purified, amplified, and labeled.
(7) Labeled nucleic acid is contacted with a microarray containing polynucleotides of the genes identified herein as correlated to discriminations between ER status under hybridization conditions to allow hybridization to occur, then processed and scanned to obtain a pattern of intensities of each spot (relative to a control for general gene expression in cells) which determine the level of expression of the gene(s) in the cells.
(8) The pattern of intensities is analyzed by comparison to the expression patterns of the genes in known samples of ER positive and negative breast cancer cells (relative to the same control).

A specific example of the above method would be performing ductal lavage following a primary screen, observing and collecting non-normal and/or atypical cells for analysis. The comparison to known expression patterns, such as that made possible by a model based on the disclosed genes and patterns, identifies the cells as being ER positive or negative.

Alternatively, the sample may permit the collection of both normal as well as non-normal and/or atypical cells for analysis. The gene expression patterns for each of these two samples will be compared to each other as well as the model and the normal versus individual abnormal comparisons therein based upon the reference data set. This approach can be significantly more powerful that the non-normal and/or atypical cells only approach because it utilizes significantly more information from the normal cells and the differences between normal and non-normal/atypical cells (in both the sample and reference data sets) to determine the status of the non-normal and/or atypical cells from the sample.

With use of the present invention, skilled physicians may prescribe treatments based on non-invasive samples that they would have prescribed for a patient which had previously received a diagnosis via a solid tissue biopsy.

The above discussion is also applicable where a palpable lesion is detected followed by fine needle aspiration or needle biopsy of cells from the breast. The cells are plated and reviewed by a pathologist or automated imaging system which selects cells for analysis as described above.

The present invention may also be used, however, with solid tissue biopsies. For example, a solid biopsy may be collected and prepared for visualization followed by determination of expression of one or more genes identified herein to determine ER status in breast cancer. One preferred means is by use of in situ hybridization with polynucleotide or protein identifying probe(s) for assaying expression of said gene(s).

In an alternative method, the solid tissue biopsy may be used to extract molecules followed by analysis for expression of one or more gene(s). This provides the ability to leave out the need for visualization and collection of only those cells suspected of being non-normal and/or atypical. This method may of course be modified such that only cells suspected of being non-normal and/or atypical are collected and used to extract molecules for analysis. This would require visualization and selection as an prerequisite to gene expression analysis.

In a further modification of the above, both normal cells and cells suspected of being non-normal and/or atypical are collected and used to extract molecules for analysis of gene expression. The approach, benefits and results are as described above using non-invasive sampling.

The genes identified herein may be used to generate a model capable of predicting the breast cancer ER statis of an unknown breast cell sample based on the expression of the identified genes in the sample.

The detection of gene expression from the samples may be by use of a single microarray able to assay gene expression of the genes disclosed herein, whether correlated with ER positive or negative status.

Other uses of the present invention include providing the ability to identify breast cancer cell samples as being those of ER positive or negative for further research or study. This provides a particular advantage in many contexts requiring the identification of breast cancer ER status based on objective genetic or molecular criteria.

The materials for use in the methods of the present invention are ideally suited for preparation of kits produced in accordance with well known procedures. The invention thus provides kits comprising agents for the detection of expression of the disclosed genes for identifying breast cancer ER status. Such kits optionally comprising the agent with an identifying description or label or instructions relating to their use in the methods of the present invention, is provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of a subset of the disclosed genes to the exclusion of material irrelevant to the identification of ER status.

The present invention also provides for the use of the gene product of one or more of the disclosed gene in the identification of agents that increase or decrease the expression of, or the activity of, said gene product. Methods of identifying such agents are preferably used to identify agents that will return the expression of, or the activity of, a gene product to a more normal level as opposed to the level seen in ER positive or negative cell. Most preferred is the return of an ER negative cell to normalcy in light of the poor prognosis for patients with ER negative status.

Such methods may be used to identify agents that decrease the expression of, or the activity of, a gene product encoded by a gene that is over expressed in ER positive or ER negative cells. Alternatively, such methods may be used to identify agents that increase the expression of, or the activity of, a gene product encoded by a gene that is under expressed in ER positive or ER negative cells.

The following tables set forth the genes of the invention. For example, Tables 1 and 3 include the ESR1 (estrogen receptor alpha) gene. “CloneID” as used in the context of the present invention refers to the IMAGE Consortium clone ID number of each gene, the sequences of which are hereby incorporated by reference in their entireties as they are available from the Consortium at image.llnl.gov as accessed on the filing date of the present application. “GeneID” as used in the context of the Tables herein as well as the present invention refers to the GenBank accession number of a sequence of each gene, the sequences of which are hereby incorporated by reference in their entireties as they are available from GenBank as accessed on the filing date of the present application.

P value refers to values assigned as described in the Example below. The indications of “E−xx” where “xx” is a two digit number refers to alternative notation for exponential figures where “E−xx” is “10^{-xx}”. Thus in combination with the numbers to the left of “E−xx”, the value being represented is the numbers to the left times 10^{×xx}. Chromosome Location refers to the human chromosome to which the gene has been assigned. Description provides a brief identifier of what the gene encodes/expresses.

Table 5 provides non-limiting examples of the corresponding GenBank accession number, clone ID number, and Unigene (cluster) ID numbers for exemplary sequences disclosed herein. The sequences of the invention may thus be identified by any of these identifiers. The identification of other corresponding numbers (GenBank accession number, clone ID number, and/or Unigene (cluster) ID numbers) for sequences disclosed herein can be made as a matter of routine from public information sources and without undue experimentation.

TABLE 1
Genes that are overexpressed in ER positive cells, and underexpressed in ER negative cells

| Clone_ID | P_value | Gene_Description |
|---|---|---|
| 2308409 | 0.001730688 | KIAA1077\|KIAA1077 protein |
| 1569878 | 0.016682884 | ESTs |
| 757220 | 6.59E−06 | FUS1\|lung cancer candidate |
| 1570414 | 0.001162673 | DKFZP434J037\|hypothetical protein DKFZp434J037 |
| 1459742 | 2.71E−09 | MAP-1\|modulator of apoptosis 1 |
| 455025 | 1.04E−05 | CGR19\|cell growth regulatory with ring finger domain |
| 767784 | 0.001891407 | JUND\|jun D proto-oncogene |
| 1637767 | 1.80E−12 | CS2\|calsyntenin-2 |
| 242778 | 2.42E−19 | CEGP1\|CEGP1 protein |
| 897446 | 3.60E−05 | ESTs, Weakly similar to I78885 serine/threonine-specific protein kinase [H. sapiens] |
| 50577 | 2.20E−05 | MGC16028\|MGC16028 similar to RIKEN cDNA 1700019E19 gene |
| 566106 | 6.95E−05 | Homo sapiens cDNA FLJ31200 fis, clone KIDNE2000574, weakly similar to Staphylococcus epidermidis |
| | | putative cell-surface adhesin SdrF gene |
| 785710 | 5.12E−05 | KIAA1025\|KIAA1025 protein |
| 866702 | 0.034946932 | PTPN13\|protein tyrosine phosphatase, non-receptor type 13 (APO-1/CD95 (Fas)-associated |
| | | phosphatase) |
| 431505 | 0.000107772 | HS1-2\|putative transmembrane protein |
| 809876 | 7.98E−06 | ATP5G2\|ATP synthase, H+ transporting, mitochondrial F0 complex, subunit c (subunit 9), isoform 2 |
| 2321113 | 1.80E−12 | AGR2\|anterior gradient 2 (Xenepus laevis) homolog |
| 1910426 | 0.016882187 | BM-009\|hypothetical protein |
| 49249 | 0.000289664 | Homo sapiens, clone MGC: 10039 IMAGE: 3889701, mRNA, complete cds |
| 241043 | 0.009801049 | Human clone 137308 mRNA, partial cds |
| 111389 | 3.62E−12 | ESTs |
| 1866068 | 0.006067385 | ESTs |
| 590310 | 1.45E−10 | Homo sapiens, clone MGC: 17393 IMAGE: 3914851, mRNA, complete cds |
| 366966 | 0.004146925 | Homo sapiens cDNA: FLJ21333 fis, clone COL02535 |
| 840506 | 0.021449552 | 3-Apr\|apoptosis related protein APR-3 |
| 1620396 | 0.04743837 | ESTs |
| 307740 | 0.001654163 | ESTs |
| 1492780 | 0.040280641 | ESTs, Weakly similar to I38022 hypothetical protein [H. sapiens] |
| 845453 | 0.016037507 | ARL3\|ADP-ribosylation factor-like 3 |
| 682817 | 0.01385764 | MDM2\|mouse double minute 2, human homolog of; p53-binding protein |
| 773286 | 1.40E−08 | SLC9A3R1\|solute carrier family 9 (sodium/hydrogen exchanger), isoform 3 regulatory factor 1 |
| 144029 | 0.007899024 | EST |
| 197775 | 3.62E−05 | ESTs |
| 796152 | 0.014513254 | Homo sapiens cDNA FLJ11685 fis, clone HEMBA1004934 |
| 809892 | 1.77E−12 | SEMA3B\|sema domain, immunoglobulin domain (Ig), short basic domain, secreted, (semaphorin) 3B |
| 740941 | 4.14E−08 | APM2\|adipose specific 2 |
| 84965 | 0.000389422 | C5orf3\|chromosome 5 open reading frame 3 |
| 785732 | 2.50E−05 | BM036\|uncharacterized bone marrow protein BM036 |
| 417884 | 0.007045683 | Homo sapiens cDNA FLJ12052 fis, clone HEMBB1002042, moderately similar to CYTOCHROME P450 |
| | | 4C1 (EC 1.14.14.1) |
| 67988 | 5.65E−05 | SNRPD2\|small nuclear ribonucleoprotein D2 polypeptide (16.5 kD) |
| 342378 | 9.49E−06 | DUSP5\|dual specificity phosphatase 5 |
| 138550 | 0.006400652 | FLJ11137\|hypothetical protein FLJ11137 |
| 162753 | 0.043575209 | DD5\|progestin induced protein |
| 1881469 | 9.79E−05 | OCLN\|occludin |
| 136954 | 1.57E−07 | ESTs, Weakly similar to YEX0_YEAST HYPOTHETICAL 64.8 KDA PROTEIN IN GDI1-COX15 |
| | | INTERGENIC REGION [S. cerevisiae] |
| 320456 | 0.00177597 | FLJ10300\|hypothetical protein FLJ10300 |
| 134270 | 0.02511167 | Human hbc647 mRNA sequence |
| 840524 | 0.011860244 | GOLGB1\|golgi autoantigen, golgin subfamily b, macrogolgin (with transmembrane signal), 1 |
| 1681520 | 0.005746627 | FLJ11068\|hypothetical protein FLJ11068 |
| 31225 | 0.01680194 | BCRP2\|Breakpoint cluster region protein, uterine leiomyoma, 2 |
| 1518845 | 7.98E−08 | ESTs |
| 590338 | 0.001677738 | LOC51065\|40S ribosomal protein S27 isoform |
| 491706 | 0.017583577 | Homo sapiens mRNA; cDNA DKFZp434F1622 (from clone DKFZp434F1622) |
| 812042 | 0.000429178 | TSC1\|tuberous sclerosis 1 |
| 296702 | 0.01747597 | DIO1\|deiodinase, iodothyronine, type I |
| 785745 | 1.15E−06 | DXS1283E\|GS2 gene |
| 279720 | 5.88E−07 | Homo sapiens, Similar to RIKEN cDNA 1700008D07 gene, clone MGC: 9830 IMAGE: 3863323, mRNA, |
| | | complete cds |
| 239877 | 5.74E−07 | HDAC3\|histone deacetylase 3 |
| 49273 | 0.030979591 | SLC27A4\|solute carrier family 27 (fatty acid transporter), member 4 |
| 758206 | 4.62E−06 | PMS2L2\|postmeiotic segregation increased 2-like 2 |
| 810446 | 0.011693351 | KIAA1522\|KIAA1522 protein |
| 135220 | 0.007130449 | MGC12981\|hypothetical protein MGC12981 |
| 284261 | 0.001705915 | MDS030\|uncharacterized hematopoietic stem/progenitor cells protein MDS030 |
| 594627 | 0.000265791 | FLJ11164\|hypothetical protein FLJ11164 |
| 487831 | 0.007143248 | Homo sapiens cDNA FLJ14059 fis, clone HEMBB1000573 |
| 127063 | 0.000920217 | ESTs |
| 1323448 | 3.15E−07 | CRIP1\|cysteine-rich protein 1 (intestinal) |
| 742565 | 0.000443835 | MYG1\|MYG1 protein |
| 2017756 | 0.005004126 | MOG1\|homolog of yeast MOG1 |
| 2171616 | 0.004755604 | ITGB5\|integrin, beta 5 |
| 490778 | 0.003255264 | QP-C\|low molecular mass ubiquinone-binding protein (9.5 kD) |
| 359781 | 0.004382798 | SPTB\|spectrin, beta, erythrocytic (includes spherocytosis, clinical type I) |
| 268234 | 2.81E−06 | DMXL1\|Dmx-like 1 |
| 1609616 | 7.68E−06 | ESTs |
| 135238 | 4.44E−05 | ESTs |
| 2016022 | 0.022592867 | p25\|brain-specific protein p25 alpha |
| 741919 | 0.001246921 | TJP3\|tight junction protein 3 (zona occludens 3) |
| 22428 | 0.03083658 | ESTs |
| 2017769 | 0.005529556 | EIF4EBP1\|eukaryotic translation initiation factor 4E binding protein 1 |
| 289505 | 1.89E−09 | ESTs, Weakly similar to 2004399A chromosomal protein [H. sapiens] |
| 731073 | 0.041342882 | LOC51126\|N-terminal acetyltransferase complex ard1subunit |
| 785766 | 0.004538178 | LOC51321\|hypothetical protein |
| 251936 | 0.02996845 | NSF\|N-ethylmaleimide-sensitive factor |
| 810463 | 0.003602021 | DKFZp566O084\|DKFZP566O084 protein |
| 1554430 | 7.94E−13 | Homo sapiens cDNA FLJ10561 fis, clone NT2RP2002672 |
| 251938 | 0.016335389 | CA14\|carbonic anhydrase XIV |
| 1032762 | 0.027468511 | Homo sapiens mRNA; cDNA DKFZp434G2127 (from clone DKFZp434G2127) |
| 705274 | 0.027821949 | DGKD\|diacylglycerol kinase, delta (130 kD) |
| 268240 | 0.019288575 | FXC1\|fracture callus 1 (rat) homolog |
| 50915 | 0.030739747 | MGC2752\|hypothetical protein MGC2752 |
| 1535851 | 0.001352198 | COX15\|COX15 (yeast) homolog, cytochrome c oxidase assembly protein |
| 429284 | 0.023974067 | ZNF226\|zinc finger protein 226 |
| 124824 | 1.26E−05 | RPL10A\|ribosomal protein L10a |
| 24032 | 0.001081701 | CRADD\|CASP2 and RIPK1 domain containing adaptor with death domain |
| 788185-2 | 4.38E−06 | TNFRSF10B\|tumor necrosis factor receptor superfamily, member 10b |
| 1517136 | 0.002531495 | RBM8A\|RNA binding motif protein 8A |
| 343731-2 | 0.012232736 | KLF2\|Kruppel-like factor 2 (lung) |
| 111721 | 0.020116819 | LOC51141\|insulin induced protein 2 |
| 71101 | 3.59E−07 | PROCR\|protein C receptor, endothelial (EPCR) |
| 530197 | 0.004873141 | Homo sapiens, clone MGC: 5522 IMAGE: 3454199, mRNA, complete cds |
| 261706 | 0.010699069 | Homo sapiens cDNA FLJ32056 fis, clone NTONG2001619 |
| 122147 | 0.040911534 | ELMO2\|engulfment and cell motility 2 (ced-12 homolog, C. elegans) |
| 773617 | 0.038655611 | UBE2D2\|ubiquitin-conjugating enzyme E2D 2 (homologous to yeast UBC4/5) |
| 279752 | 0.038399841 | DC8\|DKFZP566O1646 protein |
| 838285 | 7.61E−05 | LOC51092\|CGI-40 protein |
| 277074 | 0.042882678 | UNC5C\|unc5 (C. elegans homolog) c |
| 2492422 | 0.019925171 | SERPINI2\|serine (or cysteine) proteinase inhibitor, clade I (neuroserpin), member 2 |
| 2009904 | 0.015782601 | FLJ11230\|hypothetical protein FLJ11230 |
| 175103 | 7.33E−06 | CELSR2\|cadherin, EGF LAG seven-pass G-type receptor 2, flamingo (Drosophila) homolog |
| 795543 | 0.02996845 | PRDX4\|peroxiredoxin 4 |
| 130845-2 | 0.004607159 | PWP1\|nuclear phosphoprotein similar to S. cerevisiae PWP1 |
| 137940 | 3.89E−07 | GSTM3\|glutathione S-transferase M3 (brain) |
| 810486 | 0.014811257 | EST |
| 767069 | 0.012408476 | Homo sapiens cDNA: FLJ21930 fis, clone HEP04301, highly similar to HSU90916 Human clone 23815 |
| | | mRNA sequence |
| 143426 | 1.35E−08 | ARHB\|ras homolog gene family, member B |
| 826133 | 1.99E−08 | BTBD2\|BTB (POZ) domain containing 2 |
| 301122 | 0.016918654 | ECM1\|extracellular matrix protein 1 |
| 26736 | 0.000107772 | Homo sapiens cDNA FLJ30872 fis, clone FEBRA2004293 |
| 826138 | 3.22E−09 | GAMT\|guanidinoacetate N-methyltransferase |
| 1909163 | 1.63E−06 | B3GNT6\|UDP-GlcNAc: betaGal beta-1,3-N-acetylglucosaminyltransferase 6 |
| 788334 | 2.20E−06 | MRPL23\|mitochondrial ribosomal protein L23 |
| 1518890 | 4.42E−08 | MTL5\|metallothionein-like 5, testis-specific (tesmin) |
| 1703339 | 0.000289561 | STXBP2\|syntaxin binding protein 2 |
| 120561 | 0.025636692 | KIDINS220\|likely homolog of rat kinase D-interacting substance of 220 kDa |
| 785795 | 4.55E−15 | FLJ12910\|hypothetical protein FLJ12910 |
| 2012854 | 0.000522332 | Homo sapiens cDNA FLJ13137 fis, clone NT2RP3003150 |
| 321455 | 7.33E−07 | SARM\|sterile alpha and HEAT/Armadillo motif protein, ortholog of Drosophila |
| 179403 | 0.035275484 | CYP2E\|cytochrome P450, subfamily IIE (ethanol-inducible) |
| 126455 | 0.025654559 | RAB5B\|RAB5B, member RAS oncogene family |
| 44387 | 1.24E−05 | ELOVL2\|elongation of very long chain fatty acids (FEN1/Elo2, SUR4/Elo3, yeast)-like 2 |
| 590390 | 0.013880919 | MADP-1\|MADP-1 protein |
| 487882 | 0.01198186 | DKFZP761D0211\|hypothetical protein DKFZp761D0211 |
| 342720 | 0.00238904 | KIAA0096\|KIAA0096 protein |
| 75415 | 5.47E−09 | HINT\|histidine triad nucleotide-binding protein |
| 415550 | 7.94E−07 | Homo sapiens cDNA FLJ13598 fis, clone PLACE1009921 |
| 741954 | 0.016770876 | Homo sapiens cDNA FLJ14656 fis, clone NT2RP2002439 |
| 759200 | 0.000754507 | MGC4238\|hypothetical protein MGC4238 |
| 1657385 | 0.005619887 | EST |
| 626208 | 0.000852377 | Homo sapiens cDNA: FLJ21904 fis, clone HEP03585 |
| 1709233 | 0.006870641 | SLC1A4\|solute carrier family 1 (glutamate/neutral amino acid transporter), member 4 |
| 758266 | 0.01526361 | THBS4\|thrombospondin 4 |
| 753321 | 1.76E−05 | KIAA0232\|KIAA0232 gene product |
| 1455842 | 3.72E−06 | Homo sapiens cDNA: FLJ22448 fis, clone HRC09541 |
| 2028515 | 0.028548635 | SMARCA1\|SWI/SNF related, matrix associated, actin dependent regulator of chromatin, subfamily a, |
| | | member 1 |
| 1932725 | 0.00620659 | ZNF281\|zinc finger protein 281 |
| 1517171 | 1.50E−10 | IL2RA\|interleukin 2 receptor, alpha |
| 1910800 | 0.022735296 | ESTs, Weakly similar to 2004399A chromosomal protein [H. sapiens] |
| 2018758 | 0.033107716 | PEX7\|peroxisomal biogenesis factor 7 |
| 469172 | 0.001026742 | SEC22C\|vesicle trafficking protein |
| 814792 | 0.011398824 | USP10\|ubiquitin specific protease 10 |
| 415564 | 0.000103761 | Homo sapiens, clone MGC: 20208 IMAGE: 3936339, mRNA, complete cds |
| 31299 | 0.002342367 | Homo sapiens mRNA; cDNA DKFZp761I1912 (from clone DKFZp761I1912) |
| 1882481 | 6.16E−05 | ESTs |
| 1035359 | 0.012284709 | FLJ22593\|hypothetical protein FLJ22593 |
| 139573 | 0.024069066 | RNPEP\|arginyl aminopeptidase (aminopeptidase B) |
| 79712 | 0.024734713 | IGF2R\|insulin-like growth factor 2 receptor |
| 177827 | 0.005854823 | SYT7\|synaptotagmin VII |
| 121533 | 0.043542397 | WHSC1L1\|Wolf-Hirschhorn syndrome candidate 1-like 1 |
| 49630 | 8.03E−10 | CACNA1D\|calcium channel, voltage-dependent, L type, alpha 1D subunit |
| 741977 | 0.030962737 | BF\|B-factor, properdin |
| 1584577 | 0.019525111 | ESTs |
| 1566937 | 0.012408476 | Homo sapiens cDNA FLJ32325 fis, clone PROST2003922 |
| 726597 | 0.000877702 | Homo sapiens cDNA FLJ32642 fis, clone SYNOV2001144 |
| 277403 | 0.019916149 | Homo sapiens mRNA; cDNA DKFZp586D0918 (from clone DKFZp586D0918) |
| 42018 | 0.000140658 | KIAA1468\|KIAA1468 protein |
| 823912 | 0.005767924 | UBL3\|ubiquitin-like 3 |
| 79726 | 1.98E−06 | ESTs, Moderately similar to T46395 hypothetical protein DKFZp434I1120.1 [H. sapiens] |
| 971212 | 0.002192731 | HIVEP2\|human immunodeficiency virus type\|enhancer-binding protein 2 |
| 1507713 | 0.030416081 | CHAD\|chondroadherin |
| 418129 | 0.000216424 | IL18BP\|interleukin 18 binding protein |
| 299332 | 1.25E−10 | KIAA0575\|KIAA0575 gene product |
| 70500 | 1.01E−05 | TBX3\|T-box 3 (ulnar mammary syndrome) |
| 741988 | 0.000741391 | ACY1\|aminoacylase 1 |
| 769796 | 2.23E−08 | MGC17330\|hypothetical protein MGC17330 |
| 1731860 | 2.65E−05 | GADD45B\|growth arrest and DNA-damage-inducible, beta |
| 219929 | 0.000438842 | KIAA1415\|KIAA1415 protein |
| 773674 | 0.005209931 | RRAS2\|related RAS viral (r-ras) oncogene homolog 2 |
| 712848 | 0.018338006 | MADD\|MAP-kinase activating death domain |
| 782497 | 1.22E−05 | BC008967\|hypothetical gene BC008967 |
| 843163 | 8.73E−06 | LCP\|host cell factor homolog |
| 1707667 | 1.78E−08 | Homo sapiens cDNA FLJ31065 fis, clone HSYRA2001142 |
| 2325804 | 2.47E−05 | KIAA0661\|95 kDa retinoblastoma protein binding protein |
| 131318 | 0.000194501 | LOC55971\|insulin receptor tyrosine kinase substrate |
| 823928 | 0.009945291 | GSTT2\|glutathione S-transferase theta 2 |
| 824588 | 0.000600627 | Homo sapiens cDNA FLJ32203 fis, clone PLACE6003038, weakly similar to ZINC FINGER PROTEIN 84 |
| 2049813 | 0.042566514 | LOC51063\|hypothetical protein |
| 768068 | 1.40E−08 | DKFZP761E2110\|hypothetical protein DKFZp761E2110 |
| 726901 | 0.037164505 | Homo sapiens cDNA FLJ33095 fis, clone TRACH2000708 |
| 826194 | 1.45E−10 | SYTL2\|synaptotagmin-like 2 |
| 1690739 | 0.000152094 | HSPA4\|heat shock 70 kD protein 4 |
| 108864 | 0.000132671 | OASIS\|old astrocyte specifically induced substance |
| 23454 | 0.036615517 | HRLP5\|H-rev107-like protein 5 |
| 1884404 | 0.001613372 | KIAA0285\|KIAA0285 gene product |
| 200954 | 0.001076393 | EST |
| 1556401 | 0.023586705 | EST |
| 2029503 | 0.041795601 | ESTs |
| 2027900 | 4.84E−09 | Homo sapiens cDNA FLJ12187 fis, clone MAMMA1000831 |
| 278093 | 0.000144715 | BRAP\|BRCA1 associated protein |
| 823940 | 3.40E−05 | TOB1\|transducer of ERBB2, 1 |
| 809507 | 6.95E−05 | FLJ20568\|hypothetical protein FLJ20568 |
| 504763 | 0.019901982 | SDC4\|syndecan 4 (amphiglycan, ryudocan) |
| 84295 | 0.004226757 | IL1RN\|interleukin 1 receptor antagonist |
| 262739 | 9.25E−05 | P125\|Sec23-interacting protein p125 |
| 725978 | 0.016317028 | ESTs, Moderately similar to ALU7_HUMAN ALU SUBFAMILY SQ SEQUENCE CONTAMINATION |
| | | WARNING ENTRY [H. sapiens] |
| 714472 | 0.019175238 | KIAA0397\|KIAA0397 gene product |
| 854691 | 0.028565804 | Homo sapiens, clone MGC: 17299 IMAGE: 3845811, mRNA, complete cds |
| 288948 | 0.040000458 | GTF2H3\|general transcription factor IIH, polypeptide 3 (34 kD subunit) |
| 356835 | 0.010661105 | MGC10500\|hypothetical protein MGC10500 |
| 2016410 | 0.046542451 | HIVEP3\|human immunodeficiency virus type I enhancer-binding protein 3 |
| 343731 | 0.012195178 | KLF2\|Kruppel-like factor 2 (lung) |
| 815794 | 1.67E−05 | NUCB2\|nucleobindin 2 |
| 1575008 | 0.000123427 | WBP1\|WW domain binding protein 1 |
| 857243 | 4.97E−07 | RPS13\|ribosomal protein S13 |
| 296030 | 0.027904683 | Homo sapiens cDNA: FLJ20944 fis, clone ADSE01780 |
| 1882823 | 0.041142646 | ESTs |
| 139912 | 0.010208539 | ESTs |
| 202577 | 0.003099407 | HNMT\|histamine N-methyltransferase |
| 502096 | 0.000365845 | Homo sapiens mRNA; cDNA DKFZp761K2024 (from clone DKFZp761K2024) |
| 1404841 | 0.000764648 | ZNF175\|zinc finger protein 175 |
| 809521 | 0.003132437 | HMT-1\|beta-1,4 mannosyltransferase |
| 666128 | 2.10E−05 | DF\|D component of complement (adipsin) |
| 770377 | 0.0445364 | ATP6N1A\|ATPase, H+ transporting, lysosomal (vacuolar proton pump) non-catalytic accessory protein |
| | | 1A (110/116 kD) |
| 1860909 | 8.46E−08 | Homo sapiens cDNA FLJ31136 fis, clone IMR322001029 |
| 27769 | 0.001838545 | Homo sapiens cDNA FLJ32051 fis, clone NTONG2001428 |
| 257135 | 0.018649252 | SLC22A4\|solute carrier family 22 (organic cation transporter), member 4 |
| 83653 | 0.015480089 | HSPC128\|HSPC128 protein |
| 345348 | 0.024128019 | PRDM2\|PR domain containing 2, with ZNF domain |
| 1908593 | 0.01198186 | ESTs |
| 811803 | 0.001032481 | SHFM3\|split hand/foot malformation (ectrodactyly) type 3 |
| 813408 | 0.032712756 | PSK-1\|type I transmembrane receptor (seizure-related protein) |
| 1568597 | 0.000166213 | FLJ21939\|hypothetical protein FLJ21939 similar to 5-azacytidine induced gene 2 |
| 39136 | 0.008152393 | ESTs |
| 135640 | 1.11E−08 | STX3A\|syntaxin 3A |
| 277463 | 8.33E−05 | C18orf1\|chromosome 18 open reading frame 1 |
| 827171 | 1.92E−05 | LRRC2\|leucine-rich repeat-containing 2 |
| 303109 | 0.028556348 | P2Y5\|purinergic receptor (family A group 5) |
| 666138 | 0.002192122 | DKFZp761J1523\|hypothetical protein DKFZp761J1523 |
| 79782 | 0.002399976 | ZNF161\|zinc finger protein 161 |
| 824916 | 0.038531356 | FLJ14511\|hypothetical protein FLJ14511 |
| 788714 | 0.009823852 | E46L\|like mouse brain protein E46 |
| 124143 | 1.91E−05 | DKFZP761H1710\|hypothetical protein DKFZp761H1710 |
| 2028597 | 0.009457359 | Homo sapiens cDNA FLJ30306 fis, clone BRACE2003319 |
| 815017 | 2.20E−05 | Homo sapiens HSPC337 mRNA, partial cds |
| 491053 | 0.000490828 | ARIH2\|ariadne (Drosophila) homolog 2 |
| 796598 | 0.031676644 | LCMT\|leucine carboxyl methyltransferase |
| 810873 | 0.014436416 | SCNN1A\|sodium channel, nonvoltage-gated 1 alpha |
| 752752 | 0.005021691 | ESTs |
| 809541 | 0.026417563 | BCL2L2\|BCL2-like 2 |
| 33267 | 1.26E−05 | EST |
| 813424 | 0.000140667 | PPID\|peptidylprolyl isomerase D (cyclophilin D) |
| 80338 | 9.63E−06 | SELENBP1\|selenium binding protein 1 |
| 627248 | 0.000251776 | SBBI31\|SBBI31 protein |
| 755301 | 0.001147972 | PRKCD\|protein kinase C, delta |
| 52933 | 5.32E−10 | LIV-1\|LIV-1 protein, estrogen regulated |
| 269606 | 0.025359903 | MPG\|N-methylpurine-DNA glycosylase |
| 109863 | 2.63E−06 | EMP2\|epithelial membrane protein 2 |
| 1592021 | 1.16E−06 | HFL1\|H factor (complement)-like 1 |
| 263727 | 1.73E−05 | D5S346\|DNA segment, single copy probe LNS-CAI/LNS-CAII (deleted in polyposis |
| 295410 | 0.015532036 | PVT1\|pvt-1 (murine) oncogene homolog, MYC activator |
| 814095 | 0.015960541 | LTA4H\|leukotriene A4 hydrolase |
| 2014856 | 0.007182327 | HLALS\|major histocompatibility complex, class I-like sequence |
| 782851 | 0.039199758 | FLJ12799\|hypothetical protein FLJ12799 |
| 2011515 | 0.000180794 | DKFZP586B0923\|DKFZP586B0923 protein |
| 1751466 | 0.000262724 | Homo sapiens, Similar to C-terminal modulator protein, clone MGC: 29636 IMAGE: 4870462, mRNA, |
| | | complete cds |
| 278430 | 0.0220126 | KIF5C\|kinesin family member 5C |
| 767475 | 0.046030837 | PCANAP7\|prostate cancer associated protein 7 |
| 767477 | 2.38E−09 | ANKRA2\|ankyrin repeat, family A (RFXANK-like), 2 |
| 179804 | 1.77E−06 | PWP2H\|PWP2 (periodic tryptophan protein, yeast) homolog |
| 126851 | 7.99E−08 | FLJ11160\|hypothetical protein FLJ11160 |
| 754376 | 0.034945258 | ESTs, Weakly similar to Z195_HUMAN ZINC FINGER PROTEIN 195 [H. sapiens] |
| 1592039 | 0.001012294 | COL4A5\|collagen, type IV, alpha 5 (Alport syndrome) |
| 811843 | 8.82E−08 | SUOX\|sulfite oxidase |
| 143661-2 | 3.51E−07 | NTN4\|netrin 4 |
| 1947826 | 0.006003565 | ITGAL\|integrin, alpha L (antigen CD11A (p180), lymphocyte function-associated antigen 1; alpha |
| | | polypeptide) |
| 1492468 | 4.10E−08 | KIAA0452\|DEME-6 protein |
| 684890 | 0.000308434 | FLJ20274\|hypothetical protein FLJ20274 |
| 143846 | 0.000133999 | LRP2\|low density lipoprotein-related protein 2 |
| 135688 | 0.016682884 | GATA2\|GATA-binding protein 2 |
| 23819 | 7.73E−09 | ABCG1\|ATP-binding cassette, sub-family G (WHITE), member 1 |
| 415962 | 5.02E−06 | PACE4\|paired basic amino acid cleaving system 4 |
| 149735 | 2.37E−08 | CSNK1G3\|casein kinase 1, gamma 3 |
| 71545 | 3.96E−05 | MAP4K1\|mitogen-activated protein kinase kinase kinase kinase 1 |
| 1900362 | 0.005096825 | Homo sapiens chromosome 17 open reading frame 26 (C17orf26) mRNA, complete cds |
| 325079 | 0.002196708 | SRA1\|steroid receptor RNA activator 1 |
| 767495 | 2.38E−09 | GLI3\|GLI-Kruppel family member GLI3 (Greig cephalopolysyndactyly syndrome) |
| 897153 | 0.005755017 | PTD009\|PTD009 protein |
| 589479 | 0.009372382 | HPCL2\|2-hydroxyphytanoyl-CoA lyase |
| 42408 | 0.004066019 | MGC4604\|hypothetical protein MGC4604 |
| 744917 | 4.06E−06 | NINJ1\|ninjurin 1 |
| 2239290 | 0.047401567 | SDF1\|stromal cell-derived factor 1 |
| 2018084 | 0.000879242 | SPAK\|Ste-20 related kinase |
| 1700429 | 2.65E−09 | GFRA1\|GDNF family receptor alpha 1 |
| 810124 | 0.009454178 | PAFAH1B3\|platelet-activating factor acetylhydrolase, isoform, lb, gamma subunit (29 kD) |
| 470121 | 6.56E−06 | TCTA\|T-cell leukemia translocation altered gene |
| 155227 | 4.49E−06 | Homo sapiens clone 25194 mRNA sequence |
| 781145 | 0.029514943 | NT5M\|5′ nucleotidase, mitochondrial |
| 127821 | 0.018649252 | ACP5\|acid phosphatase 5, tartrate resistant |
| 2818432 | 0.004788915 | EST |
| 897164 | 0.013650395 | CTNNA1\|catenin (cadherin-associated protein), alpha 1 (102 kD) |
| 753743 | 0.000185264 | IL6ST\|interleukin 6 signal transducer (gp130, oncostatin M receptor) |
| 1630942 | 0.002517943 | CRN\|crooked neck protein (crn) |
| 1700436 | 1.41E−11 | ESTs |
| 1877281 | 0.028655076 | SGCG\|sarcoglycan, gamma (35 kD dystrophin-associated glycoprotein) |
| 810131 | 0.021198683 | KRT19\|keratin 19 |
| 22895 | 0.004091636 | INSM1\|insulinoma-associated 1 |
| 882467 | 1.44E−07 | EST |
| 768452 | 0.026097881 | Homo sapiens EST from clone 491476, full insert |
| 786096 | 0.048422373 | APC7\|anaphase-promoting complex subunit 7 |
| 768454 | 0.001618599 | KIAA1673\|KIAA1673 |
| 239568 | 4.44E−11 | ANXA9\|annexin A9 |
| 1325816 | 0.000370005 | POLR2L\|polymerase (RNA) \|\| (DNA directed) polypeptide L (7.6 kD) |
| 266312 | 8.82E−07 | ATP7B\|ATPase, Cu++ transporting, beta polypeptide (Wilson disease) |
| 128493 | 0.028655076 | MLH1\|mutL (E. coli) homolog 1 (colon cancer, nonpolyposis type 2) |
| 814423 | 0.003013488 | CNNM4\|cyclin M4 |
| 68636 | 0.036880506 | MGC2477\|hypothetical protein MGC2477 |
| 69293 | 0.004896866 | PSG6\|pregnancy specific beta-1-glycoprotein 6 |
| 2413337 | 0.00254811 | SORL1\|sortilin-related receptor, L(DLR class) A repeats-containing |
| 148022 | 0.000123978 | Homo sapiens clone Z′3-1 placenta expressed mRNA from chromosome X |
| 1159963 | 0.017851437 | IRF7\|interferon regulatory factor 7 |
| 46716 | 2.46E−07 | CBX4\|chromobox homolog 4 (Drosophila Pc class) |
| 470144 | 4.55E−06 | EST |
| 701751 | 0.046183854 | CUTL1\|cut (Drosophila)-like 1 (CCAAT displacement protein) |
| 43090 | 0.002358408 | H-L(3)MBT\|lethal (3) malignant brain tumor I(3)mbt protein (Drosophila) homolog |
| 1860990 | 0.003691973 | Homo sapiens cDNA FLJ32269 fis, clone PROST1000526 |
| 85624 | 0.025579525 | C4BPA\|complement component 4-binding protein, alpha |
| 592728 | 0.021741547 | LOC51205\|LPAP for lysophosphatidic acid phosphatase |
| 1569902 | 7.55E−08 | KIAA0556\|KIAA0556 protein |
| 744944 | 0.017120553 | MYO6\|myosin VI |
| 132381 | 0.028459057 | ENDOFIN\|endosome-associated FYVE-domain protein |
| 1658777 | 0.032434932 | MY038\|hypothetical brain protein my038 |
| 882484 | 0.028227375 | CCT7\|chaperonin containing TCP1, subunit 7 (eta) |
| 725284 | 0.044510834 | PHKG2\|phosphorylase kinase, gamma 2 (testis) |
| 1568967 | 0.000127809 | ESTs |
| 1571106 | 4.93E−06 | KIAA0876\|KIAA0876 protein |
| 767819 | 0.02727988 | KIAA0763\|KIAA0763 gene product |
| 73188 | 0.001353778 | FLJ20259\|hypothetical protein FLJ20259 |
| 2303214 | 0.017204034 | NPPB\|natriuretic peptide precursor B |
| 145491 | 0.021270804 | PCDH1\|protocadherin 1 (cadherin-like 1) |
| 283315 | 0.016037507 | PGAM2\|phosphoglycerate mutase 2 (muscle) |
| 842925 | 0.006759973 | MGC13064\|hypothetical protein MGC13064 |
| 809904 | 0.014055609 | EST |
| 2975668 | 4.56E−07 | RAG2\|recombination activating gene 2 |
| 1848168 | 0.003247172 | ZAK\|sterile-alpha motif and leucine zipper containing kinase AZK |
| 460395 | 0.000805296 | FLJ22625\|hypothetical protein FLJ22625 |
| 1086070 | 0.013113828 | RPH3AL\|rabphilin 3A-like (without C2 domains) |
| 773308 | 0.032700759 | Homo sapiens, clone MGC: 20276 IMAGE: 3844806, mRNA, complete cds |
| 842933 | 0.046030837 | LOC51143\|dynein light chain-A |
| 898147 | 1.07E−07 | FLJ20417\|hypothetical protein FLJ20417 |
| 50615 | 1.84E−06 | HSPA1L\|heat shock 70 kD protein-like 1 |
| 1456160 | 0.039135747 | AZGP1\|alpha-2-glycoprotein 1, zinc |
| 1929165 | 0.043830461 | ESTs, Highly similar to HXB3_HUMAN HOMEOBOX PROTEIN HOX-B3 [H. sapiens] |
| 277848 | 6.68E−06 | Homo sapiens cDNA FLJ13900 fis, clone THYRO1001746 |
| 743224 | 0.011593805 | Homo sapiens cDNA FLJ11344 fis, clone PLACE1010870, moderately similar to ZINC FINGER PROTEIN |
| | | 91 |
| 302549 | 0.000135733 | BTEB1\|basic transcription element binding protein 1 |
| 1603446 | 0.002762432 | ESTs, Weakly similar to unknown [H. sapiens] |
| 2307514 | 0.00207763 | MLC1\|KIAA0027 protein |
| 415229 | 5.28E−05 | VAV3\|vav 3 oncogene |
| 75886 | 0.00958954 | ESTs, Weakly similar to E54024 protein kinase [H. sapiens] |
| 1601845 | 0.016882187 | CAPRI\|Ca2+-promoted Ras inactivator |
| 308437 | 7.30E−08 | FUCA1\|fucosidase, alpha-L-1, tissue |
| 186301 | 7.86E−05 | Homo sapiens cDNA FLJ12924 fis, clone NT2RP2004709 |
| 2524445 | 0.030973781 | NPAS1\|neuronal PAS domain protein 1 |
| 703383 | 3.54E−05 | ESTs |
| 129478 | 0.010814891 | ESTs |
| 230385 | 0.006373505 | PIP5K2B\|phosphatidylinositol-4-phosphate 5-kinase, type II, beta |
| 1915416 | 0.002066945 | CDK7\|cyclin-dependent kinase 7 (homolog of Xenopus MO15 cdk-activating kinase) |
| 489169 | 0.000415287 | ESTs, Moderately similar to S65657 alpha-1C-adrenergic receptor splice form 2 [H. sapiens] |
| 796986 | 1.44E−06 | SIG11\|putative secreted protein |
| 811121 | 3.47E−05 | Homo sapiens mRNA; cDNA DKFZp586B1922 (from clone DKFZp586B1922) |
| 782147 | 0.011517902 | KIAA0544\|KIAA0544 protein |
| 1637829 | 1.21E−12 | ESTs |
| 898162 | 0.047678928 | C9orf5\|chromosome 9 open reading frame 5 |
| 1500542 | 6.03E−08 | RGS11\|regulator of G-protein signalling 11 |
| 760224 | 0.004693782 | XRCC1\|X-ray repair complementing defective repair in Chinese hamster cells 1 |
| 770785 | 0.004763371 | HMIC\|1,2-alpha-mannosidase IC |
| 488516 | 0.002192122 | Homo sapiens, clone IMAGE: 3875012, mRNA |
| 290567 | 0.002138612 | KBRAS1\|I-kappa-B-interacting Ras-like protein 1 |
| 813813 | 0.017612 | Homo sapiens cDNA FLJ31638 fis, clone NT2RI2003556 |
| 82322 | 6.98E−08 | RBSK\|ribokinase |
| 809939 | 0.001725113 | MAPK3\|mitogen-activated protein kinase 3 |
| 430623 | 2.33E−05 | ESTs |
| 49303 | 0.033476578 | PPP2R2B\|protein phosphatase 2 (formerly 2A), regulatory subunit B (PR 52), beta isoform |
| 796996 | 1.40E−06 | IGBP1\|immunoglobulin (CD79A) binding protein 1 |
| 431286 | 4.10E−08 | PTD002\|PTD002 protein |
| 773332 | 0.00185329 | ITGAE\|integrin, alpha E (antigen CD103, human mucosal lymphocyte antigen 1; alpha polypeptide) |
| 866488 | 4.10E−08 | ESTs, Weakly similar to 2004399A chromosomal protein [H. sapiens] |
| 2048524 | 0.005767924 | JAK2\|Janus kinase 2 (a protein tyrosine kinase) |
| 61061 | 0.018883013 | FNBP3\|formin-binding protein 3 |
| 357120 | 0.000238959 | Homo sapiens, clone IMAGE: 3538007, mRNA, partial cds |
| 1560723 | 0.029910221 | HOXB3\|homeo box B3 |
| 1412344 | 0.007631302 | GP2\|glycoprotein 2 (zymogen granule membrane) |
| 809944 | 0.00289345 | KIAA0310\|KIAA0310 gene product |
| 1607755 | 7.06E−05 | HSPC141\|HSPC141 protein |
| 1460075 | 0.042644552 | PIN1\|protein (peptidyl-prolyl cis/trans isomerase) NIMA-interacting 1 |
| 120271 | 0.004728263 | MGC4692\|hypothetical protein MGC4692 |
| 1637843 | 0.001725113 | HSPA5\|heat shock 70 kD protein 5 (glucose-regulated protein, 78 kD) |
| 501868 | 0.000425258 | RAI2\|retinoic acid induced 2 |
| 308466 | 6.75E−05 | LOC51128\|GTP-binding protein Sara |
| 362755 | 0.005875967 | PRKAG1\|protein kinase, AMP-activated, gamma 1 non-catalytic subunit |
| 362756 | 0.020883314 | KIAA1164\|hypothetical protein KIAA1164 |
| 1604423 | 0.013359346 | ESTs |
| 243800 | 0.017125244 | RYBP\|RING1 and YY1 binding protein |
| 731104 | 3.25E−07 | ESTs |
| 266389 | 8.87E−09 | SLC16A6\|solute carrier family 16 (monocarboxylic acid transporters), member 6 |
| 502818 | 2.33E−06 | ARHA\|ras homolog gene family, member A |
| 1636111 | 0.019413865 | HNRPU\|heterogeneous nuclear ribonucleoprotein U (scaffold attachment factor A) |
| 1570502 | 0.017495868 | Homo sapiens cDNA FLJ12936 fis, clone NT2RP2005018 |
| 251682 | 1.50E−06 | TINF2\|TERF1 (TRF1)-interacting nuclear factor 2 |
| 773353 | 0.026514552 | Homo sapiens cDNA FLJ31753 fis, clone NT2RI2007468 |
| 111469 | 5.71E−05 | Homo sapiens clone IMAGE: 111469 mRNA sequence |
| 40100 | 0.029910221 | Homo sapiens, clone MGC: 18084 IMAGE: 4150952, mRNA, complete cds |
| 454173 | 0.003570072 | WDR7\|WD repeat domain 7 |
| 842980 | 1.87E−06 | DRG1\|developmentally regulated GTP-binding protein 1 |
| 454175 | 0.009321073 | HIT-17\|PKCI-1-related HIT protein |
| 1609372 | 0.008647658 | RIPK3\|receptor-interacting serine-threonine kinase 3 |
| 302591 | 3.97E−10 | ARHH\|ras homolog gene family, member H |
| 66317 | 0.029073646 | H1F2\|H1 histone family, member 2 |
| 201282 | 0.030274401 | DKFZP434N126\|DKFZP434N126 protein |
| 472103 | 0.005357267 | SHOC2\|soc-2 (suppressor of clear, C. elegans) homolog |
| 795288 | 0.017376841 | USP4\|ubiquitin specific protease 4 (proto-oncogene) |
| 2016869 | 0.006544656 | KIAA1131\|KIAA1131 protein |
| 811162 | 0.024982615 | FMOD\|fibromodulin |
| 1910516 | 0.000122585 | ESTs |
| 491486 | 0.000923428 | LOC51578\|adrenal gland protein AD-004 |
| 1558473 | 0.020974589 | ESTs |
| 1947144 | 0.028543112 | ESTs |
| 757327 | 0.01394813 | LOC51249 hypothetical protein |
| 429322 | 0.008351255 | Homo sapiens, clone IMAGE: 3447394, mRNA, partial cds |
| 868400 | 0.013032007 | QARS\|glutaminyl-tRNA synthetase |
| 50675 | 2.78E−08 | KIAA0423\|KIAA0423 protein |
| 143169 | 4.17E−06 | Homo sapiens, clone IMAGE: 3881549, mRNA |
| 823615 | 0.014557615 | Homo sapiens cDNA: FLJ21245 fis, clone COL01184 |
| 1917063 | 0.00188129 | Homo sapiens, clone MGC: 15400 IMAGE: 4040570, mRNA, complete cds |
| 1455566 | 0.000327751 | ADORA3\|adenosine A3 receptor |
| 813854 | 0.028548635 | PURA\|purine-rich element binding protein A |
| 795296 | 6.64E−07 | CCNH\|cyclin H |
| 725649 | 3.06E−05 | NFATC4\|nuclear factor of activated T-cells, cytoplasmic, calcineurin-dependent 4 |
| 49344 | 0.000860578 | KIAA0350\|KIAA0350 protein |
| 415287 | 0.023418315 | ESTs |
| 149058 | 0.042155684 | Homo sapiens cDNA FLJ10174 fis, clone HEMBA1003959 |
| 2306919 | 4.44E−05 | SLC35A3\|solute carrier family 35 (UDP-N-acetylglucosamine (UDP-GIcNAc) transporter), member 3 |
| 2072768 | 0.044848397 | NCOA3\|nuclear receptor coactivator 3 |
| 757337 | 6.19E−07 | ESTs |
| 1686547 | 0.039985007 | ESTs |
| 74119-2 | 0.002954849 | SNRPN\|small nuclear ribonucleoprotein polypeptide N |
| 489509 | 0.031251603 | Homo sapiens mRNA; cDNA DKFZp564L0822 (from clone DKFZp564L0822) |
| 1870049 | 0.005305054 | DDO\|D-aspartate oxidase |
| 683569 | 0.000182323 | ITPR1\|inositol 1,4,5-triphosphate receptor, type 1 |
| 50685 | 0.048777689 | KIAA1414\|KIAA1414 protein |
| 611239 | 0.000438876 | Homo sapiens cDNA: FLJ22361 fis, clone HRC06524, highly similar to HSU15426 Human anonymous |
| | | mRNA sequence with CCA repeat region |
| 261472 | 0.017638942 | ORF1-FL49\|putative nuclear protein ORF1-FL49 |
| 279800 | 5.50E−05 | SLMAP\|sarcolemma associated protein |
| 49351 | 0.004160342 | HSSEXGENE\|SEX gene |
| 49354 | 1.84E−06 | ANK2\|ankyrin 2, neuronal |
| 773381 | 0.000877702 | NAPA\|N-ethylmaleimide-sensitive factor attachment protein, alpha |
| 714159 | 0.030468197 | Homo sapiens cDNA FLJ32185 fis, clone PLACE6001925 |
| 754002 | 0.040830931 | DKK3\|dickkopf (Xenopus laevis) homolog 3 |
| 51631 | 6.17E−07 | DKFZP586B1621\|DKFZP586B1621 protein |
| 289287 | 0.017659789 | Homo sapiens mRNA; cDNA DKFZpS86J101 (from clone DKFZpS86J101) |
| 301963 | 0.001486067 | SNX11\|sorting nexin 11 |
| 742641 | 0.017924982 | MGC5540\|hypothetical protein MGC5540 |
| 823634 | 0.000159834 | ESTs |
| 564621 | 0.011672024 | SERPINI1\|serine (or cysteine) proteinase inhibitor, clade I (neuroserpin), member 1 |
| 814815 | 0.00048204 | EST |
| 796255 | 6.94E−06 | MRPS14\|mitochondrial ribosomal protein 514 |
| 344073 | 7.94E−08 | Homo sapiens cDNA FLJ31763 fis, clone NT2RI2007827 |
| 1557841 | 0.001309802 | ESTs |
| 2020772 | 0.000172997 | TM7SF2\|transmembrane 7 superfamily member 2 |
| 1637893 | 1.84E−06 | Homo sapiens, clone MGC: 9575 IMAGE: 3879368, mRNA, complete cds |
| 753071 | 0.005767924 | Homo sapiens cDNA: FLJ22528 fis, clone HRC12825 |
| 42827 | 0.003388822 | Homo sapiens cDNA FLJ31604 fis, clone NT2RI2002699 |
| 22500 | 0.000613835 | ESTs |
| 841287 | 0.001159126 | GNPAT\|glyceronephosphate O-acyltransferase |
| 301976 | 0.000553787 | PPP3CC\|protein phosphatase 3 (formerly 2B), catalytic subunit, gamma isoform (calcineurin A gamma) |
| 785840 | 0.028503208 | SEC24D\|SEC24 (S. cerevisiae) related gene family, member D |
| 814826 | 0.000212133 | ESTs |
| 725672 | 0.030333035 | Homo sapiens, Similar to transducin (beta)-like 3, clone MGC: 8613 IMAGE: 2961321, mRNA, complete |
| | | cds |
| 784105 | 0.03689826 | ESTs |
| 812143 | 0.009962681 | FLRT3\|fibronectin leucine rich transmembrane protein 3 |
| 1948122 | 0.0285188 | FLJ10450\|hypothetical protein FLJ10450 |
| 1877697 | 0.000466212 | ESTs |
| 1901735 | 0.004586404 | RPP14\|ribonuclease P (14 kD) |
| 454503 | 1.11E−05 | Homo sapiens, clone IMAGE: 3346451, mRNA, partial cds |
| 80798 | 0.002033157 | ITM2B\|integral membrane protein 2B |
| 594724 | 0.001075123 | YR-29\|hypothetical protein |
| 681992 | 0.038338214 | Homo sapiens cDNA FLJ13384 fis, clone PLACE1001062, highly similar to Homo sapiens mRNA for |
| | | lysine-ketoglutarate reductase/saccharopine dehydrogenase |
| 487932 | 5.59E−09 | SYTL2\|synaptotagmin-like 2 |
| 868441 | 0.003083769 | FLJ20727\|hypothetical protein FLJ20727 |
| 729964 | 0.004902138 | SMPD1\|sphingomyelin phosphodiesterase 1, acid lysosomal (acid sphingomyelinase) |
| 345034 | 0.017833089 | SCYB14\|small inducible cytokine subfamily B (Cys-X-Cys), member 14 (BRAK) |
| 884480 | 0.022202501 | COX7C\|cytochrome c oxidase subunit VIIc |
| 823655 | 3.25E−08 | AD036\|AD036 protein |
| 823659 | 1.46E−06 | FYCO1\|FYVE and coiled-coil domain containing 1 |
| 298417 | 1.30E−14 | TFF3\|trefoil factor 3 (intestinal) |
| 2063982 | 2.33E−07 | KCNK6\|potassium channel, subfamily K, member 6 (TWIK-2) |
| 44443 | 0.042068695 | SCYE1\|small inducible cytokine subfamily E, member 1 (endothelial monocyte-activating) |
| 838366 | 1.25E−05 | HMGCL\|3-hydroxymethyl-3-methylglutaryl-Coenzyme A lyase (hydroxymethylglutaricaciduria) |
| 754033 | 7.67E−06 | LZTFL1\|leucine zipper transcription factor-like 1 |
| 754034 | 0.001228195 | THPO\|thrombopoietin (myeloproliferative leukemia virus oncogene ligand, megakaryocyte growth and |
| | | development factor) |
| 897593 | 1.41E−06 | KIAA1548\|KIAA1548 protein |
| 770074 | 0.00038924 | PRRG2\|proline-rich Gla (G-carboxyglutamic acid) polypeptide 2 |
| 753092 | 1.98E−06 | EST |
| 1631238 | 0.007429314 | KIAA1483\|KIAA1483 protein |
| 415610 | 0.000498483 | FLJ22402\|hypothetical protein FLJ22402 |
| 2018808 | 0.038787441 | PRCP\|prolylcarboxypeptidase (angiotensinase C) |
| 823661 | 0.000706071 | Homo sapiens cDNA FLJ31768 fis, clone NT2RI2007891, moderately similar to DMR-N9 PROTEIN |
| 486208 | 0.000218364 | TGFB3\|transforming growth factor, beta 3 |
| 487948 | 0.000529233 | Homo sapiens cDNA: FLJ22566 fis, clone HSI01980 |
| 67318 | 0.012702024 | Homo sapiens, Similar to RIKEN cDNA 0610039G24 gene, clone MGC: 15403 IMAGE: 4126342, mRNA, |
| | | complete cds |
| 83358 | 1.39E−11 | ESTs |
| 1583673 | 2.86E−06 | ESTs, Weakly similar to KIAA0351 [H. sapiens] |
| 784126 | 0.009372382 | TST\|thiosulfate sulfurtransferase (rhodanese) |
| 366315 | 0.024091716 | Homo sapiens, clone MGC: 20500 IMAGE: 4053084, mRNA, complete cds |
| 2366057 | 0.000958877 | CDH12\|cadherin 12, type 2 (N-cadherin 2) |
| 47795 | 0.01192016 | ZNF161 zinc finger protein 161 |
| 51672 | 9.79E−05 | ARRB1\|arrestin, beta 1 |
| 124922 | 0.000642521 | SZF1\|KRAB-zinc finger protein SZF1-1 |
| 193916 | 0.030979591 | RHBG\|Rhesus blood group, B glycoprotein |
| 1925280 | 5.88E−07 | NPR2L\|homologous to yeast nitrogen permease (candidate tumor suppressor) |
| 266747 | 0.034115322 | Homo sapiens, Similar to RIKEN cDNA 2010001O09 gene, clone MGC: 21387 IMAGE: 4471592, mRNA, |
| | | complete cds |
| 429387 | 0.001030569 | CHN2\|chimerin (chimaerin) 2 |
| 345055 | 9.76E−05 | ESTs, Highly similar to LB4D_HUMAN NADP-DEPENDENT LEUKOTRIENE B4 12- |
| | | HYDROXYDEHYDROGENASE [H. sapiens] |
| 345056 | 0.000129022 | KIAA1404\|KIAA1404 protein |
| 258589 | 0.000383745 | REL\|v-rel avian reticuloendotheliosis viral oncogene homolog |
| 190295 | 2.00E−05 | FLJ23590\|hypothetical protein FLJ23590 |
| 782537 | 0.045949613 | Homo sapiens cDNA FLJ30428 fis, clone BRACE2008941 |
| 1554549 | 1.10E−06 | HAGH\|hydroxyacyl glutathione hydrolase |
| 143519 | 0.023557456 | FKBP2\|FK506-binding protein 2 (13 kD) |
| 838389 | 1.40E−08 | UNC119\|unc119 (C. elegans) homolog |
| 184022 | 0.010544493 | APBB1\|amyloid beta (A4) precursor protein-binding, family B, member 1 (Fe65) |
| 840664 | 0.000657157 | EST |
| 203240 | 3.98E−06 | DAB2\|disabled (Drosophila) homolog 2 (mitogen-responsive phosphoprotein) |
| 300271 | 0.003251584 | SAS10\|disrupter of silencing 10 |
| 131070 | 0.00841697 | KIAA0670\|KIAA0670 protein/acinus |
| 196189 | 1.68E−07 | CYB5\|cytochrome b-5 |
| 453599 | 0.001069723 | DKFZP564F1123\|DKFZP564F1123 protein |
| 823682 | 0.026331677 | KIAA0710\|KIAA0710 gene product |
| 784140 | 0.003891255 | WDR11\|WD40 repeat domain 11 protein |
| 31366 | 0.011252089 | MYT1\|myelin transcription factor 1 |
| 1558832 | 0.003860708 | MAT2B\|methionine adenosyltransferase II, beta |
| 742695 | 3.48E−07 | Homo sapiens cDNA FLJ31534 fis, clone NT2RI2000671 |
| 726658 | 8.52E−08 | NME3\|non-metastatic cells 3, protein expressed in |
| 823688 | 0.045171023 | MAN1A1\|mannosidase, alpha, class 1A, member 1 |
| 2322223 | 2.50E−05 | SNRPA\|small nuclear ribonucleoprotein polypeptide A |
| 866874 | 8.19E−08 | HNRPH2\|heterogeneous nuclear ribonucleoprotein H2 (H') |
| 250313 | 0.000892123 | ESTs |
| 685516 | 2.07E−10 | GPCR150\|putative G protein-coupled receptor |
| 288683 | 0.00367697 | ESTs |
| 461761 | 1.58E−05 | ANG\|angiogenin, ribonuclease, RNase A family, 5 |
| 758347 | 6.94E−05 | SLC2A10\|solute carrier family 2 (facilitated glucose transporter), member 10 |
| 1650927 | 0.004105721 | DKFZp547E052\|hypothetical protein DKFZp547E052 |
| 128143 | 2.88E−06 | PON1\|paraoxonase 1 |
| 753404 | 0.037494688 | KIAA0887\|KIAA0887 protein |
| 214751 | 0.000487816 | Homo sapiens, clone MGC: 12617 IMAGE: 2964706, mRNA, complete cds |
| 1694775 | 0.000354216 | EST |
| 277185 | 0.000614717 | PRO0461\|PRO0461 protein |
| 2432360 | 0.006342148 | BSN\|bassoon (presynaptic cytomatrix protein) |
| 1609748 | 2.16E−06 | MGC10882\|hypothetical protein MGC10882 |
| 1915824 | 0.003398986 | ESTs, Weakly similar to I38022 hypothetical protein [H. sapiens] |
| 2067500 | 4.36E−11 | LDB3\|LIM domain binding 3 |
| 823691 | 4.27E−08 | CCNG2\|cyclin G2 |
| 1895664 | 0.026431801 | PRO2198\|hypothetical protein PRO2198 |
| 243100 | 1.01E−06 | ACADSB\|acyl-Coenzyme A dehydrogenase, short/branched chain |
| 784150 | 3.38E−10 | RAB31\|RAB31, member RAS oncogene family |
| 785890 | 0.032434657 | Homo sapiens cDNA FLJ14030 fis, clone HEMBA1004086 |
| 784154 | 0.014124729 | ESTs, Highly similar to unnamed protein product [H. sapiens] |
| 767170 | 0.026562375 | LOC51606\|CGI-11 protein |
| 767171 | 5.14E−05 | FLJ20015\|hypothetical protein |
| 812196 | 0.000269329 | UGCG\|UDP-glucose ceramide glucosyltransferase |
| 145132 | 0.017780719 | MPDU1\|mannose-P-dolichol utilization defect 1 |
| 767176 | 0.003072281 | TNFSF13\|tumor necrosis factor (ligand) superfamily, member 13 |
| 855385 | 0.008682257 | CLN2\|ceroid-lipofuscinosis, neuronal 2, late infantile (Jansky-Bielschowsky disease) |
| 843224 | 1.13E−05 | DSCR2\|Down syndrome critical region gene 2 |
| 283751 | 0.032429761 | CORT\|cortistatin |
| 233349 | 0.000116838 | FLJ10761\|hypothetical protein FLJ10761 |
| 1536925 | 6.56E−06 | PDPK1\|3-phosphoinositide dependent protein kinase-1 |
| 771058 | 1.40E−08 | CISH\|cytokine inducible SH2-containing protein |
| 1323591 | 1.62E−07 | SERPINA5\|serine (or cysteine) proteinase inhibitor, clade A (alpha-1 antiproteinase, antitrypsin), |
| | | member 5 |
| 826245 | 1.66E−05 | L0C54505\|hypothetical protein |
| 1895672 | 9.67E−05 | ESTs, Weakly similar to S57447 HPBRII-7 protein [H. sapiens] |
| 840687 | 0.004816698 | MUC1\|mucin 1, transmembrane |
| 80050 | 0.036628745 | FLJ23153\|likely ortholog of mouse tumor necrosis-alpha-induced adipose-related protein |
| 82738 | 2.39E−09 | DNASE1L3\|deoxyribonuclease I-like 3 |
| 503866 | 0.011391526 | SPA17\|sperm autoantigenic protein 17 |
| 1027283 | 0.009413465 | ESTs |
| 147826 | 0.003162135 | ESTs |
| 38213 | 0.000167261 | FLJ22174\|hypothetical protein FLJ22174 |
| 1502566 | 1.61E−05 | ESTs, Weakly similar to T21437 hypothetical protein F26H9.4 - Caenorhabditis elegans [C. elegans] |
| 754080 | 0.012652328 | ICAM3\|intercellular adhesion molecule 3 |
| 267725 | 5.65E−05 | BC-2\|putative breast adenocarcinoma marker (32 kD) |
| 1703455 | 0.004124332 | EDG2\|endothelial differentiation, lysophosphatidic acid G-protein-coupled receptor, 2 |
| 344432 | 0.001735764 | MGST2\|microsomal glutathione S-transferase 2 |
| 2015517 | 6.88E−09 | FLJ22237\|hypothetical protein FLJ22237 |
| 813154 | 0.046806631 | NFIA\|nuclear factor I/A |
| 322511 | 4.76E−08 | Homo sapiens mRNA; cDNA DKFZp564D1462 (from clone DKFZp564D1462) |
| 814899 | 0.001468215 | BNIP3L\|BCL2/adenovirus E1B 19 kD-interacting protein 3-like |
| 160532 | 0.004538178 | ESTs, Weakly similar to JX0331 laurate omega-hydroxylase [H. sapiens] |
| 784178 | 1.98E−06 | Homo sapiens mRNA; cDNA DKFZp586M0723 (from clone DKFZp586M0723) |
| 1536941 | 0.000449552 | ESTs, Moderately similar to ALU2_HUMAN ALU SUBFAMILY SB SEQUENCE CONTAMINATION |
| | | WARNING ENTRY [H. sapiens] |
| 841641 | 4.35E−06 | CCND1\|cyclin D1 (PRAD1: parathyroid adenomatosis 1) |
| 843248 | 0.001755052 | VAMP3\|vesicle-associated membrane protein 3 (cellubrevin) |
| 127519 | 2.29E−08 | POH1\|26S proteasome-associated pad1 homolog |
| 43849 | 0.001286711 | ESTs |
| 815847 | 0.038913997 | C11orf5\|chromosome 11 open reading frame 5 |
| 261852 | 0.001941321 | ESTs |
| 2306221 | 1.79E−12 | WNT10B\|wingless-type MMTV integration site family, member 10B |
| 813165 | 0.003482886 | PGRMC2\|progesterone receptor membrane component 2 |
| 811565 | 1.51E−05 | KIAA1694\|KIAA1694 protein |
| 726699 | 4.73E−07 | Homo sapiens, clone MGC: 9889 IMAGE: 3868330, mRNA, complete cds |
| 1555536 | 3.04E−06 | LGP1\|likely ortholog of mouse D11Igp1 |
| 771084 | 0.000459658 | BECN1\|beclin 1 (coiled-coil, myosin-like BCL2-interacting protein) |
| 526757 | 1.84E−05 | CCND1\|cyclin D1 (PRAD1: parathyroid adenomatosis 1) |
| 42118 | 8.31E−05 | P2RX4\|purinergic receptor P2X, ligand-gated ion channel, 4 |
| 28475 | 0.001063475 | CRYZ\|crystallin, zeta (quinone reductase) |
| 841655 | 1.97E−05 | RBBP2\|retinoblastoma-binding protein 2 |
| 1632247 | 0.001096449 | FLJ23436\|hypothetical protein FLJ23436 |
| 753447 | 0.014351752 | DDB2\|damage-specific DNA binding protein 2 (48 kD) |
| 280444 | 0.000352001 | KIAA0608\|KIAA0608 protein |
| 745289 | 0.029910221 | PNAS-127\|PNAS-127 protein |
| 502153 | 0.005704405 | Homo sapiens, clone MGC: 16377 IMAGE: 3936171, mRNA, complete cds |
| 378824 | 0.000177644 | HSPC009\|HSPC009 protein |
| 784190 | 0.000123726 | Homo sapiens, Similar to RIKEN cDNA 5530601I19 gene, clone MGC: 9743 IMAGE: 3854028, mRNA, |
| | | complete cds |
| 70606 | 0.022570057 | ESTs |
| 854763 | 9.74E−07 | MGC20702\|hypothetical protein MGC20702 |
| 2284473 | 0.01376248 | ZK1\|Kruppel-type zinc finger (C2H2) |
| 1588973 | 0.000173284 | IMAGE3451454\|hypothetical protein IMAGE3451454 |
| 26883 | 1.17E−07 | PKIB\|protein kinase (cAMP-dependent, catalytic) inhibitor beta |
| 796643 | 0.012443564 | NEBL\|nebulette |
| 877636 | 2.16E−05 | DCTN4\|dynactin 4 (p62) |
| 2017144 | 0.002872765 | LOC51093\|CGI-41 protein |
| 1048586 | 0.009471574 | BMI1\|murine leukemia viral (bmi-1) oncogene homolog |
| 813183 | 0.01280152 | AZU1\|azurocidin 1 (cationic antimicrobial protein 37) |
| 486288 | 0.012126378 | Homo sapiens clone CDABP0028 mRNA sequence |
| 811582 | 1.35E−07 | GOLPH2\|golgi phosphoprotein 2 |
| 838732 | 1.49E−05 | LOC51596\|divalent cation tolerant protein CUTA |
| 813189 | 2.96E−05 | EST |
| 811585 | 0.000116498 | HD\|huntingtin (Huntington disease) |
| 1558895 | 0.041918884 | PGAM2\|phosphoglycerate mutase 2 (muscle) |
| 208531 | 5.51E−06 | FLJ20551\|hypothetical protein FLJ20551 |
| 150003 | 1.31E−07 | FLJ13187\|phafin 2 |
| 293569 | 9.71E−05 | C1orf21\|chromosome 1 open reading frame 21 |
| 25154 | 0.004517557 | PLAT\|plasminogen activator, tissue |
| 824692 | 4.04E−06 | SEL1L\|sel-1 (suppressor of lin-12, C. elegans)-like |
| 841679 | 0.00456714 | SIP2-28\|calcium and integrin binding protein (DNA-dependent protein kinase interacting protein) |
| 1908666 | 0.018949143 | ZNF79\|zinc finger protein 79 (pT7) |
| 1558108 | 0.000693345 | ABCC8\|ATP-binding cassette, sub-family C (CFTR/MRP), member 8 |
| 810937 | 0.001309095 | TPST2\|tyrosylprotein sulfotransferase 2 |
| 1473914 | 0.000225716 | C21orf15\|chromosome 21 open reading frame 15 |
| 897971 | 0.001037841 | COPB\|coatomer protein complex, subunit beta |
| 489922 | 0.006100942 | AKAP10\|A kinase (PRKA) anchor protein 10 |
| 754417 | 0.038568449 | Homo sapiens mRNA; cDNA DKFZp586H021 (from clone DKFZp586H021) |
| 1456937 | 0.000129703 | OVGP1\|oviductal glycoprotein 1, 120 kD (mucin 9, oviductin) |
| 841685 | 0.03461124 | SID6-306\|inorganic pyrophosphatase |
| 796663 | 0.002337587 | DRIP78\|dopamine receptor interacting protein |
| 267778 | 0.004321341 | ESTs, Weakly similar to I78885 serine/threonine-specific protein kinase [H. sapiens] |
| 1896676 | 1.24E−06 | Homo sapiens cDNA: FLJ22113 fis, clone HEP18418 |
| 814145 | 2.06E−05 | JTB\|jumping translocation breakpoint |
| 1637504 | 0.017405243 | EST |
| 296123 | 0.009311435 | Homo sapiens PRO1851 mRNA, complete cds |
| 48033 | 9.67E−07 | ESTs |
| 810947 | 0.006557959 | NUDE1\|LIS1-interacting protein NUDE1, rat homolog |
| 810948 | 0.001616352 | TRAP240\|thyroid hormone receptor-associated protein, 240 kDa subunit |
| 454908 | 0.000286005 | CGA\|glycoprotein hormones, alpha polypeptide |
| 283023 | 0.010085994 | CX3CR1\|chemokine (C—X3—C) receptor 1 |
| 897982 | 0.009962681 | EIF3S8\|eukaryotic translation initiation factor 3, subunit 8 (110 kD) |
| 380057 | 0.000839497 | APPBP2\|amyloid beta precursor protein (cytoplasmic tail)-binding protein 2 |
| 179572 | 3.91E−10 | Homo sapiens cDNA FLJ14227 fis, clone NT2RP3004095 |
| 841695 | 0.008164837 | CG018\|hypothetical gene CG018 |
| 30793 | 0.002706225 | PEX11B\|peroxisomal biogenesis factor 11B |
| 23579 | 0.010974699 | Homo sapiens clone 23579 mRNA sequence |
| 1914168 | 0.045350687 | FLJ20558\|hypothetical protein FLJ20558 |
| 1637516 | 0.009609456 | GS2NA\|nuclear autoantigen |
| 1635913 | 0.041470052 | LNX\|multi-PDZ-domain-containing protein |
| 502198 | 0.00087773 | PPP1R3C\|protein phosphatase 1, regulatory (inhibitor) subunit 3C |
| 1520559 | 0.031251603 | ESTs |
| 294537 | 2.82E−08 | RAB17\|RAB17, member RAS oncogene family |
| 1899230 | 0.047956858 | H. sapiens mRNA for metallothionein isoform 1R |
| 488202 | 6.46E−07 | Homo sapiens cDNA FLJ31235 fis, clone KIDNE2004681, moderately similar to Mus musculus |
| | | peroxisomal long chain acyl-CoA thioesterase lb (Pte1b) gene |
| 271670 | 0.012020011 | TNFSF12\|tumor necrosis factor (ligand) superfamily, member 12 |
| 430318 | 0.00244659 | PVALB\|parvalbumin |
| 2284803 | 0.002768702 | SCAMP1\|secretory carrier membrane protein 1 |
| 470964 | 1.58E−07 | GALNT6\|UDP-N-acetyl-alpha-D-galactosamine: polypeptide N-acetylgalactosaminyltransferase 6 |
| | | (GalNAc-T6) |
| 196570 | 0.000194501 | MGC2747\|hypothetical protein MGC2747 |
| 754443 | 0.023018366 | Homo sapiens cDNA FLJ13771 fis, clone PLACE4000270 |
| 1536215 | 0.008360311 | GALNAC4S-6ST\|B cell RAG associated protein |
| 700836 | 0.001618877 | ZNF19\|zinc finger protein 19 (KOX 12) |
| 1553996 | 2.38E−05 | ESTs, Weakly similar to ALU1_HUMAN ALU SUBFAMILY J SEQUENCE CONTAMINATION WARNING |
| | | ENTRY [H. sapiens] |
| 50339 | 1.99E−07 | ESTs, Moderately similar to hypothetical protein [H. sapiens] |
| 2103000 | 0.019869834 | ESTs |
| 25194 | 0.000197727 | Homo sapiens clone 25194 mRNA sequence |
| 1523116 | 0.000863455 | NESH\|NESH protein |
| 1593849 | 0.013132955 | PDE4A\|phosphodiesterase 4A, cAMP-specific (dunce (Drosophila)-homolog phosphodiesterase E2) |
| 813518 | 0.001929727 | ESTs |
| 2138030 | 0.0247432 | KCNS3\|potassium voltage-gated channel, delayed-rectifier, subfamily S, member 3 |
| 66787 | 0.037494688 | TRIP3\|thyroid hormone receptor interactor 3 |
| 215000 | 1.30E−06 | VIPR1\|vasoactive intestinal peptide receptor 1 |
| 197525 | 0.009040685 | FMO5\|flavin containing monooxygenase 5 |
| 826622 | 1.28E−06 | KIAA0430\|KIAA0430 gene product |
| 291216 | 0.011893765 | CETN2\|centrin, EF-hand protein, 2 |
| 2460159 | 0.040000458 | TNK1\|tyrosine kinase, non-receptor, 1 |
| 1410444 | 0.002729944 | AREG\|amphiregulin (schwannoma-derived growth factor) |
| 882511 | 0.000981881 | M17S2\|membrane component, chromosome 17, surface marker 2 (ovarian carcinoma antigen CA125) |
| 214068 | 4.60E−25 | GATA3\|GATA-binding protein 3 |
| 417637 | 0.02117967 | KIAA1276\|KIAA1276 protein |
| 111150 | 0.008720987 | LNX\|multi-PDZ-domain-containing protein |
| 1558151 | 3.27E−06 | BATF\|basic leucine zipper transcription factor, ATF-like |
| 2052113 | 6.56E−06 | FLJ10903\|hypothetical protein FLJ10903 |
| 321945 | 0.000909807 | ESTs |
| 810987 | 1.62E−06 | MBC3205\|hypothetical protein MBC3205 |
| 26152 | 0.011314426 | KIAA1557\|KIAA1557 protein |
| 1534633 | 0.019925171 | MGC2479\|hypothetical protein MGC2479 |
| 788832 | 9.78E−06 | PLAB\|prostate differentiation factor |
| 725321 | 1.53E−22 | ESR1\|estrogen receptor 1 |
| 85384 | 0.000225058 | ATP11\|hypothetical protein FLJ22351 |
| 2018158 | 0.019146124 | ESTs |
| 814191 | 0.04385949 | KIAA0295\|KIAA0295 protein |
| 487297 | 0.001907323 | CAP2\|adenylyl cyclase-associated protein 2 |
| 730942 | 0.002682409 | DECR2\|2,4-dienoyl CoA reductase 2, peroxisomal |
| 502518 | 0.000152832 | LAMB2\|laminin, beta 2 (laminin S) |
| 811937 | 0.015875339 | SNX9\|sorting nexin 9 |
| 278531 | 7.09E−07 | COX6C\|cytochrome c oxidase subunit VIc |
| 1558166 | 1.96E−07 | ESTs, Moderately similar to AF072733 1 putative secreted protein [H. sapiens] |
| 137370 | 0.007518112 | ESTs |
| 704062 | 5.44E−05 | SIRT3\|sirtuin (silent mating type information regulation 2, S. cerevisiae, homolog) 3 |
| 593431 | 8.35E−13 | Homo sapiens mRNA full length insert cDNA clone EUROIMAGE 1287006 |
| 595037 | 0.008509083 | RAI3\|retinoic acid induced 3 |
| 240966 | 0.012397017 | PCBP2\|poly(rC)-binding protein 2 |
| 754474 | 0.016470796 | ESTs |
| 344820 | 0.021127692 | KIF3A\|kinesin family member 3A |
| 486641 | 0.000140204 | MOCS2\|molybdenum cofactor synthesis 2 |
| 1898619 | 7.34E−10 | MGC15737\|hypothetical protein MGC15737 |
| 450673 | 7.31E−05 | SPATA2\|spermatogenesis associated 2 |
| 1917837 | 0.034946932 | FLJ11838\|hypothetical protein FLJ11838 |
| 85394 | 0.01312499 | PPAP2B\|phosphatidic acid phosphatase type 2B |
| 345487 | 0.035143688 | Homo sapiens, clone MGC: 23280 IMAGE: 4637504, mRNA, complete cds |
| 811944 | 0.045490722 | ESTs |
| 786169 | 0.003709448 | FLJ11088\|hypothetical protein FLJ11088 |
| 376516 | 7.09E−07 | LRBA\|vesicle trafficking, beach and anchor containing |
| 70692 | 0.025359903 | SERPINB2\|serine (or cysteine) proteinase inhibitor, clade B (ovalbumin), member 2 |
| 147147 | 0.005871121 | ESTs |
| 1563792 | 0.001655476 | LOC51333\|mesenchymal stem cell protein DSC43 |
| 1687976 | 0.005209931 | SIL1\|endoplasmic reticulum chaperone SIL1, homolog of yeast |
| 1898625 | 0.00480123 | ANKTM1\|ankyrin-like with transmembrane domains 1 |
| 130845 | 0.016285335 | PWP1\|nuclear phosphoprotein similar to S. cerevisiae PWP1 |
| 1947933 | 0.032222366 | FLJ12800\|hypothetical protein FLJ12800 |
| 296190 | 0.000356685 | KIAA0321\|KIAA0321 protein |
| 588915 | 0.001292252 | IFI27\|interferon, alpha-inducible protein 27 |
| 50383 | 0.019695801 | KIAA1598\|KIAA1598 protein |
| 2346188 | 0.037722909 | PNKP\|polynucleotide kinase 3′-phosphatase |
| 41569 | 2.04E−09 | FLJ12650\|hypothetical protein FLJ12650 |
| 868119 | 0.000560528 | LOC57117\|hypothetical nuclear factor SBBI22 |
| 815167 | 0.001112368 | PLEKHA3\|pleckstrin homology domain-containing, family A (phosphoinositide binding specific) member 3 |
| 342164 | 0.037052367 | FLJ14486\|hypothetical protein FLJ14486 |
| 377193 | 0.024734713 | LOC54505\|hypothetical protein |
| 1492587 | 0.01198186 | Homo sapiens, Similar to RAB37, member of RAS oncogene family, clone MGC: 21391 IMAGE: 4520191, |
| | | mRNA, complete cds |
| 276962 | 0.000184101 | LOC57149\|hypothetical protein A-211C6.1 |
| 143966 | 0.044999609 | 2-Sep\|septin 6 |
| 1707078 | 0.023623028 | LOC51646\|CGI-127 protein |
| 108265 | 0.027824136 | Homo sapiens, clone MGC: 21518 IMAGE: 3900532, mRNA, complete cds |
| 785535 | 0.029849385 | DDXBP1\|DEAD/H (Asp-Glu-Ala-Asp/His) box binding protein 1 |
| 1415720 | 0.032290985 | EST |
| 243549 | 1.35E−05 | MYB\|v-myb avian myeloblastosis viral oncogene homolog |
| 1031592 | 9.21E−07 | KIF9\|kinesin family member 9 |
| 1460110 | 0.026915103 | PSMB5\|proteasome (prosome, macropain) subunit, beta type, 5 |
| 285798 | 1.41E−06 | ESTs, Weakly similar to S65824 reverse transcriptase homolog [H. sapiens] |
| 176606 | 0.032429761 | NELL2\|nel (chicken)-like 2 |
| 272694 | 0.037494688 | C1orf9\|chromosome 1 open reading frame 9 |
| 814528 | 4.69E−05 | p53DINP1\|p53-inducible p53DINP1 |
| 342181 | 1.41E−11 | BCL2\|B-cell CLL/lymphoma 2 |
| 813584 | 0.028503208 | PA26\|p53 regulated PA26 nuclear protein |
| 813585 | 0.004222762 | XAP4\|HBV associated factor |
| 2011660 | 0.005969978 | DKFZp434K1210\|hypothetical protein DKFZp434K1210 |
| 248499 | 0.010004815 | AF5Q31\|ALL 1 fused gene from 5q31 |
| 898221 | 5.65E−05 | NR4A2\|nuclear receptor subfamily 4, group A, member 2 |
| 898222 | 0.000898114 | ATP12\|homolog of yeast ATP12 |
| 770840 | 0.012755509 | AVIL\|advillin |
| 1640821 | 3.60E−08 | Homo sapiens, clone IMAGE: 3899073, mRNA, partial cds |
| 470249 | 0.000754806 | ESTs, Weakly similar to 2004399A chromosomal protein [H. sapiens] |
| 491486-2 | 0.002434509 | LOC51578\|adrenal gland protein AD-004 |
| 143989 | 0.020286058 | KIAA1340\|KIAA1340 protein |
| 665658 | 0.000136335 | MSX2\|msh (Drosophila) homeo box homolog 2 |
| 1762326 | 4.41E−05 | ESTs |
| 74223 | 0.00021037 | HTPAP\|HTPAP protein |
| 344988-2 | 2.10E−05 | EST |
| 768570 | 3.26E−12 | FLJ11280\|hypothetical protein FLJ11280 |
| 858387 | 0.000198568 | MGC4767\|hypothetical protein MGC4767 |
| 1030953 | 0.007749864 | LOC80298\|transcription termination factor-like protein |
| 365060 | 0.000392359 | RAB11A\|RAB11A, member RAS oncogene family |
| 283413 | 0.004226861 | LRDD\|leucine-rich and death domain containing |
| 1709786 | 0.026592724 | TRPS1\|trichorhinophalangeal syndrome\| |
| 143995 | 0.025539645 | Homo sapiens mRNA from chromosome 5q21-22, clone: FBR89 |
| 1526826 | 4.26E−06 | HOXB2\|homeo box B2 |
| 140655 | 0.004076409 | dJ453C12.4.1\|similar to RIKEN cDNA 2610042O14 gene (M. musculus) |
| 322160-2 | 0.023425543 | PTEN\|phosphatase and tensin homolog (mutated in multiple advanced cancers 1) |
| 814546 | 0.019909138 | ESTs, Highly similar to PCD1_HUMAN CYTOHESIN 1 [H. sapiens] |
| 130895 | 8.00E−10 | Homo sapiens cDNA: FLJ22930 fis, clone KAT07255 |
| 725394 | 0.005095011 | CLPX\|ClpX (caseinolytic protease X, E. coli) homolog |
| 504187 | 4.31E−05 | ESTs, Moderately similar to ALU8_HUMAN ALU SUBFAMILY SX SEQUENCE CONTAMINATION |
| | | WARNING ENTRY [H. sapiens] |
| 725395 | 0.037816121 | UBE2L6\|ubiquitin-conjugating enzyme E2L 6 |
| 502585 | 0.001007942 | RAB18\|RAB18, member RAS oncogene family |
| 782385-2 | 0.011252089 | DKFZP566D193\|DKFZP566D193 protein |
| 132381-2 | 1.73E−05 | ENDOFIN\|endosome-associated FYVE-domain protein |
| 154707 | 0.02165445 | MPV17\|MpV17 transgene, murine homolog, glomerulosclerosis |
| 163528 | 0.000751027 | PALM\|paralemmin |
| 306924 | 6.86E−07 | KIAA0222\|KIAA0222 gene product |
| 296529 | 0.027905446 | KIAA1504\|KIAA1504 protein |
| 470261 | 7.82E−05 | SMA5\|SMA5 |
| 1709791 | 9.70E−08 | BAIAP1\|BAl1-associated protein 1 |
| 110578 | 0.000133284 | ESTs |
| 1457205 | 4.94E−05 | Homo sapiens cDNA FLJ20525 fis, clone KAT10610 |
| 1708055 | 0.026818494 | GOV\|glioblastoma overexpressed |
| 358936 | 4.04E−06 | ESTs, Weakly similar to T2D3_HUMAN TRANSCRIPTION INITIATION FACTOR TEIID 135 KDA |
| | | SUBUNIT [H. sapiens] |
| 1626409 | 0.005619887 | AP2A1\|adaptor-related protein complex 2, alpha 1 subunit |
| 770866 | 0.042071414 | Homo sapiens, clone MGC: 9709 IMAGE: 3850147, mRNA, complete cds |
| 347434 | 0.000306027 | SC65\|nucleolar autoantigen (55 kD) similar to rat synaptonemal complex protein |
| 795330 | 0.043246672 | NR1D1\|nuclear receptor subfamily 1, group D, member 1 |
| 347436 | 0.000531118 | GSTTLp28\|glutathione-S-transferase like; glutathione transferase omega |
| 357996 | 0.024330122 | FLJ20156\|hypothetical protein FLJ20156 |
| 770869 | 0.002110844 | MGC2592\|hypothetical protein MGC2592 |
| 126229 | 0.007870589 | MGC12760\|hypothetical protein MGC12760 |
| 130104 | 0.025309814 | Homo sapiens, clone IMAGE: 3352566, mRNA, partial cds |
| 269788 | 0.001755224 | SEDLP\|spondyloepiphyseal dysplasia, late, pseudogene |
| 785571 | 0.000352444 | DNAJL1\|hypothetical protein similar to mouse Dnajl1 |
| 325515 | 2.23E−08 | FLJ10980\|hypothetical protein FLJ10980 |
| 868168 | 6.52E−05 | SPRR2A\|small praline-rich protein 2A |
| 785574 | 0.000174826 | SNX1\|sorting nexin 1 |
| 199505 | 0.000661388 | SENP7\|sentrin/SUMO-specific protease |
| 46843 | 0.012102208 | SLI\|neuronal Shc adaptor homolog |
| 898251 | 0.020270563 | FLJ20727\|hypothetical protein FLJ20727 |
| 1950130 | 0.009056557 | Homo sapiens cDNA FLJ14115 fis, clone MAMMA1001760 |
| 739450 | 0.002041876 | LASS2\|longevity assurance (LAG1, S. cerevisiae) homolog 2 |
| 781295 | 0.004349202 | SCAD-SRL\|peroxisomal short-chain alcohol dehydrogenase |
| 127970 | 0.029073646 | Homo sapiens cDNA FLJ13557 fis, clone PLACE1007737 |
| 772477 | 0.008407492 | Homo sapiens, clone MGC: 19570 IMAGE: 3942960, mRNA, complete cds |
| 241988 | 7.09E−06 | WDR6\|WD repeat domain 6 |
| 795342 | 0.026143501 | ESTs |
| 753897 | 3.10E−07 | AMER\|autocrine motility factor receptor |
| 786525 | 0.020146808 | KIAA1917\|KIAA1917 protein |
| 812968 | 0.00014351 | PTPRN2\|protein tyrosine phosphatase, receptor type, N polypeptide 2 |
| 1559198 | 0.000278805 | Homo sapiens cDNA FLJ14923 fis, clone PLACE1008244, weakly similar to VEGETATIBLE |
| | | INCOMPATIBILITY PROTEIN HET-E-1 |
| 141623 | 0.037935139 | LOC51056\|leucine aminopeptidase |
| 366039 | 2.23E−05 | ESTs, Weakly similar to ABP2_HUMAN ENDOTHELIAL ACTIN-BINDING PROTEIN [H. sapiens] |
| 2054896 | 0.007810967 | FLJ21669\|hypothetical protein FLJ21669 |
| 969495 | 0.001699546 | TIGA1\|TIGA1 |
| 786530 | 0.001147277 | FLJ23027\|hypothetical protein FLJ23027 |
| 82421 | 0.010334304 | CD44\|CD44 antigen (homing function and Indian blood group system) |
| 1892025 | 0.019936342 | MGC13170\|hypothetical protein MGC13170 |
| 343174 | 1.92E−05 | Homo sapiens cDNA FLJ31204 fis, clone KIDNE2003305, weakly similar to PUTATIVE AMIDASE |
| | | AF1954 (EC 3.5.1.4) |
| 769552 | 0.001219419 | LENG4 leukocyte receptor cluster (LRC) member 4 |
| 1409509 | 0.007517038 | TNNT1\|troponin T1, skeletal, slow |
| 785595 | 0.033081925 | STE\|sulfotransferase, estrogen-preferring |
| 139354 | 8.04E−09 | HSPC195\|hypothetical protein |
| 1493596 | 0.018150022 | VLGR1\|very large G protein-coupled receptor 1 |
| 898276 | 0.000107772 | HSMNP1\|uncharacterized hypothalamus protein HSMNP1 |
| 788141 | 0.000137025 | XPA\|xeroderma pigmentosum, complementation group A |
| 3208314 | 0.017495868 | GPR27\|G protein-coupled receptor 27 |
| 2164248 | 0.041777908 | HMG20B\|high-mobility group 20B |
| 502909 | 0.000155359 | KIAA0326\|KIAA0326 protein |
| 812989 | 0.039337098 | DJ1141E15.2\|hypothetical protein dJ1141E15.2 |
| 1556957 | 0.013797398 | Homo sapiens cDNA FLJ32647 fis, clone SYNOV2001374, moderately similar to ZINC FINGER |
| | | PROTEIN 134 |
| 897625 | 0.024870477 | KIAA0532\|KIAA0532 protein |
| 701112 | 0.000600597 | XPC\|xeroderma pigmentosum, complementation group C |
| 179212 | 4.11E−11 | ESTs, Moderately similar to T12539 hypothetical protein DKFZp434J154.1 [H. sapiens] |
| 1539977 | 0.001651449 | SLC27A1\|solute carrier family 27 (fatty acid transporter), member 1 |
| 824354 | 0.004173726 | GRLF1\|glucocorticoid receptor DNA binding factor 1 |
| 327150 | 0.033503623 | DOCK1\|dedicator of cyto-kinesis 1 |
| 487697 | 0.001175473 | CROT\|carnitine O-octanoyltransferase |
| 1603583 | 3.02E−05 | SH3BGRL\|SH3 domain binding glutamic acid-rich protein like |
| 769571 | 0.000117954 | SREBF1\|sterol regulatory element binding transcription factor 1 |
| 510794 | 0.00135873 | MYCBP\|c-myc binding protein |
| 712622 | 0.000806331 | GTF2H2\|general transcription factor IIH, polypeptide 2 (44 kD subunit) |
| 1573946 | 7.33E−06 | MRPS30\|mitochondrial ribosomal protein S30 |
| 51700 | 8.17E−09 | ESTs |
| 854444 | 9.48E−13 | HLA-DQB1\|major histocompatibility complex, class II, DQ beta 1 |
| 712624 | 0.030232568 | Homo sapiens mRNA; cDNA DKFZp564C1216 (from clone DKFZp564C1216) |
| 2054122 | 6.11E−07 | SLC11A3\|solute carrier family 11 (proton-coupled divalent metal ion transporters), member 3 |
| 1698784 | 0.038568449 | Human glucose transporter pseudogene |
| 163579 | 0.006154605 | RAB3A\|RAB3A, member RAS oncogene family |
| 1641837 | 0.002757371 | Homo sapiens putative ion channel protein CATSPER2 (CATSPER2), mRNA |
| 1472638 | 0.010481096 | FLJ14117\|hypothetical protein FLJ14117 |
| 1592530 | 0.010585978 | IP6K2\|mammalian inositol hexakisphosphate kinase 2 |
| 364469 | 0.007213754 | NDUFA7\|NADH dehydrogenase (ubiquinone) 1 alpha subcomplex, 7 (14.5 kD, B14.5a) |
| 591157 | 0.01978301 | NET-7\|transmembrane 4 superfamily member (tetraspan NET-7) |
| 67070 | 0.00152487 | SLU7\|step II splicing factor SLU7 |
| 795382 | 0.000206162 | EPAC\|Rap1 guanine-nucleotide-exchange factor directly activated by cAMP |
| 786562 | 0.009803386 | ZNF43\|zinc finger protein 43 (HTF6) |
| 180561 | 0.001401571 | GSTM1\|glutathione S-transferase M1 |
| 263159 | 4.66E−06 | PL6\|PL6 protein |
| 73638 | 1.57E−05 | PTP4A2\|protein tyrosine phosphatase type IVA, member 2 |
| 156363 | 0.009457359 | FLJ12934\|likely ortholog of mouse actin-related protein 8 homolog (S. cerevisiae) |
| 810017-2 | 0.001130048 | PLAUR\|plasminogen activator, urokinase receptor |
| 1697055 | 0.022652474 | EST |
| 1031963 | 1.29E−08 | Homo sapiens mRNA; cDNA DKFZp586A0618 (from clone DKFZp586A0618) |
| 306982 | 0.006503031 | ESTs, Weakly similar to ALU8_HUMAN ALU SUBFAMILY SX SEQUENCE CONTAMINATION |
| | | WARNING ENTRY [H. sapiens] |
| 1952866 | 0.004498241 | Homo sapiens chromosome 9, P1 clone 11659 |
| 46896 | 1.52E−05 | PRO1331\|hypothetical protein PRO1331 |
| 27516 | 0.012811838 | CAMLG\|calcium modulating ligand |
| 796330 | 0.005722733 | FLJ14888\|hypothetical protein FLJ14888 |
| 32050 | 1.89E−09 | Homo sapiens mRNA; cDNA DKFZp586P1124 (from clone DKFZp586P1124) |
| 2017917 | 0.016701054 | Homo sapiens cDNA: FLJ23371 fis, clone HEP16068, highly similar to HSTFIISH Homo sapiens mRNA |
| | | for transcription elongation factor TFIIS |
| 823719 | 0.000166213 | ESTs |
| 1585327 | 0.007304469 | AXIN2\|axin 2 (conductin, axil) |
| 84068 | 0.000831262 | CL25084\|hypothetical protein |
| 1902462 | 0.048261732 | AF038169\|hypothetical protein |
| 1733262 | 1.95E−11 | BLu\|BLu protein |
| 950836 | 2.86E−08 | DDB1\|damage-specific DNA binding protein 1 (127 kD) |
| 839081 | 3.95E−05 | HELO1\|homolog of yeast long chain polyunsaturated fatty acid elongation enzyme 2 |
| 757435 | 0.014952072 | NKX3A\|NK homeobox (Drosophila), family 3, A |
| 161993 | 0.022731389 | CEBPB\|CCAAT/enhancer binding protein (C/EBP), beta |
| 756490 | 5.64E−05 | BCAT2\|branched chain aminotransferase 2, mitochondrial |
| 250069 | 0.001412816 | DUSP8\|dual specificity phosphatase 8 |
| 809939-2 | 0.000350689 | MAPK3\|mitogen-activated protein kinase 3 |
| 825323 | 0.004670589 | CKAP1\|cytoskeleton-associated protein 1 |
| 2018581 | 1.22E−05 | IL6ST\|interleukin 6 signal transducer (gp130, oncostatin M receptor) |
| 788180 | 0.011295686 | AMACR\|alpha-methylacyl-CoA racemase |
| 741790 | 0.000210007 | FLJ20080\|hypothetical protein FLJ20080 |
| 193736-2 | 0.035288396 | BRCA2\|breast cancer 2, early onset |
| 344168 | 0.003024674 | POLL\|polymerase (DNA directed), lambda |
| 812227 | 0.017120553 | SLC9A1\|solute carrier family 9 (sodium/hydrogen exchanger), isoform 1 (antiporter, Na+/H+, amiloride |
| | | sensitive) |
| 741795 | 6.69E−06 | RALGPS1A\|Ral guanine nucleotide exchange factor RalGPS1A |
| 246194 | 0.000395219 | ESTs |
| 565379 | 0.001699546 | GRB10\|growth factor receptor-bound protein 10 |
| 325583 | 3.88E−05 | EST |
| 73659 | 0.022122876 | Homo sapiens, clone IMAGE: 3463399, mRNA, partial cds |
| 47853 | 0.037258896 | ALDH4A1\|aldehyde dehydrogenase 4 family, member A1 |
| 198917 | 0.001513766 | Homo sapiens cDNA FLJ32847 fis, clone TESTI2003376 |
| 358217 | 0.027049095 | GPC4\|glypican 4 |
| 450301 | 0.00677521 | MLH3\|mutL (E. coli) homolog 3 |
| 127243 | 0.009413465 | ESTs |
| 194638 | 0.011615484 | ESTs, Weakly similar to alternatively spliced product using exon 13A [H. sapiens] |
| 726703 | 4.06E−10 | Homo sapiens clone 23736 mRNA sequence |
| 814913 | 2.00E−07 | C11orf15\|chromosome 11 open reading frame 15 |
| 1569306 | 0.019052814 | FLJ14225\|hypothetical protein FLJ14225 |
| 112541 | 5.78E−07 | Homo sapiens cDNA FLJ30580 fis, clone BRAWH2006996 |
| 108667 | 0.001139656 | SF3A1\|splicing factor 3a, subunit 1, 120 kD |
| 813973 | 0.044951328 | VPS11\|vacuolar protein sorting 11 (yeast homolog) |
| 340974 | 3.20E−05 | Homo sapiens cDNA: FLJ23270 fis, clone COL10309, highly similar to HSU33271 Human normal |
| | | keratinocyte mRNA |
| 1901824 | 0.001796004 | HSPC207\|hypothetical protein |
| 1636251 | 0.001241866 | BAG1\|BCL2-associated athanogene |
| 700500 | 0.000197697 | PCTK2\|PCTAIRE protein kinase 2 |
| 51741 | 0.000159834 | RHO6\|GTP-binding protein |
| 789147 | 0.007583738 | ENO2\|enolase 2, (gamma, neuronal) |
| 265874 | 0.030801508 | NFIC\|nuclear factor I/C (CCAAT-binding transcription factor) |
| 769901 | 0.006350535 | CDIPT\|CDP-diacylglycerol-inositol 3-phosphatidyltransferase (phosphatidylinositol synthase) |
| 785941 | 0.00543149 | ZNF278\|zinc finger protein 278 |
| 172721 | 0.006694279 | Homo sapiens cDNA FLJ31795 fis, clone NT2RI2008812 |
| 1908378 | 0.002044263 | MPP3\|membrane protein, palmitoylated 3 (MAGUK p55 subfamily member 3) |
| 70332 | 0.036594925 | ACP2\|acid phosphatase 2, lysosomal |
| 731257 | 8.33E−05 | UNC13\|UNC13 (C. elegans)-like |
| 825350 | 0.034288184 | KIAA1040\|KIAA1040 protein |
| 841393 | 0.003180363 | PRO0149\|PRO0149 protein |
| 360518 | 0.019741126 | GNGT1\|guanine nucleotide binding protein (G protein), gamma transducing activity polypeptide 1 |
| 784212 | 4.52E−09 | FLJ20152\|hypothetical protein |
| 49481 | 1.04E−06 | FLJ14800\|hypothetical protein FLJ14800 |
| 49482 | 0.000459287 | ESTs |
| 767236 | 0.008346481 | CGI-51\|CGI-51 protein |
| 1570663 | 1.29E−05 | FKBP4\|FK506-binding protein 4 (59 kD) |
| 1871127 | 0.005023894 | ESTs |
| 745314 | 0.008936352 | HIRIP5\|HIRA-interacting protein 5 |
| 611373 | 0.043542397 | PROSC\|proline synthetase co-transcribed (bacterial homolog) |
| 744374 | 0.032359098 | DKFZP564D166\|putative ankyrin-repeat containing protein |
| 23282 | 0.027026711 | IK\|IK cytokine, down-regulator of HLA II |
| 1738537 | 0.027971153 | ESTs, Weakly similar to I78885 serine/threonine-specific protein kinase [H. sapiens] |
| 1605539 | 4.91E−07 | IDUA\|iduronidase, alpha-L- |
| 416374 | 0.007441554 | NET-6\|tetraspan NET-6 protein |
| 884599 | 3.77E−06 | FLJ10579\|hypothetical protein FLJ10579 |
| 687990 | 0.026614804 | ARHGEF6\|Rac/Cdc42 guanine exchange factor (GEF) 6 |
| 2013685 | 0.025604814 | PTDSS2\|phosphatidylserine synthase 2 |
| 502988 | 1.97E−05 | ZNF20\|zinc finger protein 20 (KOX 13) |
| 51774 | 0.005356389 | ESTs |
| 277266 | 0.000518693 | ESTs, Highly similar to T46303 hypothetical protein DKFZp434N1710.1 [H. sapiens] |
| 138788 | 0.000130376 | PRLR\|prolactin receptor |
| 265103 | 0.000987968 | MMEL2\|membrane metallo-endopeptidase-like 2 |
| 489657 | 0.001447974 | WRB\|tryptophan rich basic protein |
| 34795 | 0.003683559 | DAZAP2\|DAZ associated protein 2 |
| 183170 | 0.003453647 | Homo sapiens cDNA: FLJ23199 fis, clone REC01091 |
| 511012 | 1.21E−07 | AGPS\|alkylglycerone phosphate synthase |
| 814951 | 0.023784403 | Homo sapiens, RIKEN cDNA 2310005G07 gene, clone MGC: 10049 IMAGE: 3890955, mRNA, complete |
| | | cds |
| 788518 | 0.026818494 | PXMP3\|peroxisomal membrane protein 3 (35 kD, Zellweger syndrome) |
| 360539 | 0.014513254 | PPP3CB\|protein phosphatase 3 (formerly 2B), catalytic subunit, beta isoform (calcineurin A beta) |
| 795738 | 0.013722183 | GNG10\|guanine nucleotide binding protein 10 |
| 810671 | 1.19E−06 | FLJ22269\|hypothetical protein FLJ22269 |
| 589232 | 8.03E−05 | FLJ11506\|hypothetical protein FLJ11506 |
| 309591 | 0.049912405 | EP300\|E1A binding protein p300 |
| 198960 | 2.65E−05 | LOC51604\|CGI-06 protein |
| 713647 | 0.040280641 | TSPAN-3\|tetraspan 3 |
| 2273013 | 0.001241866 | TESK2\|testis-specific kinase 2 |
| 1554646 | 0.034592388 | CPT1A\|carnitine palmitoyltransferase I, liver |
| 1609836 | 0.000260109 | GLUL\|glutamate-ammonia ligase (glutamine synthase) |
| 79592 | 7.09E−05 | AKR7A2\|aldo-keto reductase family 7, member A2 (aflatoxin aldehyde reductase) |
| 745339 | 1.79E−05 | KIAA0419\|KIAA0419 gene product |
| 1499864 | 0.009095192 | ESTs |
| 76252 | 0.000627204 | HSPC134\|HSPC134 protein |
| 840768 | 0.007517578 | ATP6L\|ATPase, H+ transporting, lysosomal (vacuolar proton pump) 16 kD |
| 1916575 | 0.011828437 | BIK\|BCL2-interacting killer (apoptosis-inducing) |
| 1582050 | 0.000266243 | IMPDH2\|IMP (inosine monophosphate) dehydrogenase 2 |
| 730633 | 0.019761169 | OA48-18\|acid-inducible phosphoprotein |
| 2011308 | 0.030221619 | DKFZP564O0463\|DKFZP564O0463 protein |
| 278222 | 0.010334304 | Homo sapiens, clone MGC: 10083 IMAGE: 3897118, mRNA, complete cds |
| 1559596 | 1.29E−05 | ESTs, Highly similar to AF175283 1 zinc metalloendopeptidase [H. sapiens] |
| 767268 | 2.03E−06 | DPP7\|dipeptidylpeptidase 7 |
| 1556259 | 4.63E−09 | ALAD\|aminolevulinate, delta-, dehydratase |
| 1635649 | 0.000560777 | CDS2\|CDP-diacylglycerol synthase (phosphatidate cytidylyltransferase) 2 |
| 772888 | 0.011513725 | KIAA1012\|KIAA1012 protein |
| 1551317 | 0.045171023 | EST |
| 203350 | 0.01354434 | KIAA0561\|KIAA0561 protein |
| 814971 | 0.001470365 | Homo sapiens cDNA FLJ10046 fis, clone HEMBA1001133 |
| 859761 | 0.000282387 | PVRL2\|poliovirus receptor-related 2 (herpesvirus entry mediator B) |
| 1584746 | 0.00604339 | FLJ22283\|hypothetical protein FLJ22283 |
| 162211 | 1.71E−05 | ABCD3\|ATP-binding cassette, sub-family D (ALD), member 3 |
| 1733625 | 0.023369612 | ESTs |
| 279970 | 0.038137895 | ADORA2A\|adenosine A2a receptor |
| 685626 | 0.007916953 | ATP2B1\|ATPase, Ca++ transporting, plasma membrane 1 |
| 625458 | 6.02E−05 | MGC3234\|hypothetical protein MGC3234 |
| 855487 | 4.99E−06 | ASAH\|N-acylsphingosine amidohydrolase (acid ceramidase) |
| 1900149 | 0.000390804 | SBB103\|hypothetical SBBI03 protein |
| 1470333 | 2.37E−05 | APBB2\|amyloid beta (A4) precursor protein-binding, family B, member 2 (Fe65-like) |
| 192306 | 8.94E−05 | MPP2\|membrane protein, palmitoylated 2 (MAGUK p55 subfamily member 2) |
| 86035 | 1.21E−05 | LOC51015\|CGI-111 protein |
| 1573251 | 2.47E−05 | ZAP128\|peroxisomal long-chain acyl-coA thioesterase |
| 2021882 | 0.000110404 | SCNN1D\|sodium channel, nonvoltage-gated 1, delta |
| 376214 | 6.82E−06 | MRPL43\|mitochondrial ribosomal protein L43 |
| 74677 | 4.70E−06 | Homo sapiens, Similar to RIKEN cDNA A430107J06 gene, clone MGC: 21416 IMAGE: 4452699, mRNA, |
| | | complete cds |
| 726779 | 0.021235222 | CNN1\|calponin 1, basic, smooth muscle |
| 1870512 | 0.000932203 | TNFRSF14\|tumor necrosis factor receptor superfamily, member 14 (herpesvirus entry mediator) |
| 1504268 | 0.002697689 | ESTs, Highly similar to dJ25J6.2 [H. sapiens] |
| 43933 | 0.007274143 | MAOA\|monoamine oxidase A |
| 1941742 | 0.015997677 | GSA7\|ubiquitin activating enzyme E1-like protein |
| 700571 | 0.02055268 | CDC14A\|CDC14 (cell division cycle 14, S. cerevisiae) homolog A |
| 745363 | 0.006742484 | ESTs |
| 380898 | 0.000625106 | AWP1\|protein associated with PRK1 |
| 1895781 | 0.000806373 | ESTs, Highly similar to AC004836 1 similar to cadherin and Drosophila Fat protein [H. sapiens] |
| 2018958 | 0.015480089 | Homo sapiens cDNA FLJ31025 fis, clone HLUNG2000501 |
| 505575 | 0.000134468 | FLJ10116\|hypothetical protein FLJ10116 |
| 647397 | 2.31E−09 | MAPT\|microtubule-associated protein tau |
| 149539 | 0.007870589 | MKP-7\|MAPK phosphatase-7 |
| 47142 | 0.000743442 | PEX12\|peroxisomal biogenesis factor 12 |
| 838807 | 0.019798041 | MGC11308\|hypothetical protein MGC11308 |
| 1492276 | 1.60E−06 | EST |
| 240680 | 6.30E−05 | BIG1\|brefeldin A-inhibited guanine nucleotide-exchange protein 1 |
| 2314197 | 0.006926366 | FLJ12671\|hypothetical protein FLJ12671 |
| 487301 | 5.30E−08 | FBXL5\|f-box and leucine-rich repeat protein 5 |
| 360778-2 | 1.36E−11 | ATM\|ataxia telangiectasia mutated (includes complementation groups A, C and D) |
| 364865 | 1.95E−05 | FLJ21062\|hypothetical protein FLJ21062 |
| 813261 | 0.021270804 | Homo sapiens clone 23645 mRNA sequence |
| 814209 | 0.000165303 | ESTs |
| 1883630 | 2.71E−11 | KIAA1547\|KIAA1547 protein |
| 324225 | 1.89E−05 | RARRES3\|retinoic acid receptor responder (tazarotene induced) 3 |
| 229596 | 0.001770222 | TIC\|SEC7 homolog |
| 1694113 | 0.030227648 | FLJ21031\|hypothetical protein FLJ21031 |
| 1635681 | 3.06E−05 | NDUFA2\|NADH dehydrogenase (ubiquinone) 1 alpha subcomplex, 2 (8 kD, B8) |
| 782688 | 4.97E−07 | P28\|dynein, axonemal, light intermediate polypeptide |
| 143661 | 1.59E−05 | NTN4\|netrin 4 |
| 825715 | 0.019909138 | IFI41\|interferon-induced protein 41, 30 kD |
| 271721 | 0.011868489 | EST |
| 30850 | 0.001756144 | MMP17\|matrix metalloproteinase 17 (membrane-inserted) |
| 814214 | 0.001725113 | D8S2298E\|reproduction 8 |
| 796739 | 1.69E−06 | MGC10924\|hypothetical protein MGC10924 similar to Nedd4 WW-binding protein 5 |
| 486374 | 0.00241838 | MGC4645\|hypothetical protein MGC4645 |
| 855800 | 0.000428278 | PREP\|prolyl endopeptidase |
| 120006 | 0.000302823 | ESTs, Weakly similar to I38022 hypothetical protein [H. sapiens] |
| 490251 | 0.014632388 | PPP1R2\|protein phosphatase 1, regulatory (inhibitor) subunit 2 |
| 270786 | 1.17E−05 | Homo sapiens cDNA FLJ30436 fis, clone BRACE2009037 |
| 813279 | 4.41E−05 | APEH\|N-acylaminoacyl-peptide hydrolase |
| 160656 | 3.25E−08 | LU\|Lutheran blood group (Auberger b antigen included) |
| 1850224 | 0.000270348 | ESTs |
| 788580 | 0.008853088 | CCNDBP1\|cyclin D-type binding-protein 1 |
| 82871 | 0.014746648 | SHBG\|sex hormone-binding globulin |
| 813281 | 5.45E−13 | WWP1\|WW domain-containing protein 1 |
| 547058 | 0.014177292 | CCNG1 cyclin G1 |
| 1416092 | 0.016682884 | CHDH\|choline dehydrogenase |
| 854874 | 0.000592334 | KIAA0212\|KIAA0212 gene product |
| 1631701 | 0.000187841 | JFC1\|NADPH oxidase-related, C2 domain-containing protein |
| 854879 | 0.001309802 | SPHK2\|sphingosine kinase type 2 isoform |
| 752903 | 1.78E−08 | KIAA0303\|KIAA0303 protein |
| 45578 | 1.91E−05 | MAP2K6\|mitogen-activated protein kinase kinase 6 |
| 51056 | 0.010636427 | CNIL\|cornichon-like |
| 43977 | 0.014632388 | KIAA0182\|KIAA0182 protein |
| 301380 | 0.002812591 | UGTREL7\|UDP-glucuronic acid/UDP-N-acetylgalactosamine dual transporter |
| 814232 | 0.043182465 | C21orf59\|chromosome 21 open reading frame 59 |
| 796754 | 0.00150122 | DREV1\|CGI-81 protein |
| 824792 | 0.001312689 | BM045\|uncharacterized bone marrow protein BM045 |
| 814234 | 3.88E−05 | SUCLG2\|succinate-CoA ligase, GDP-forming, beta subunit |
| 1915982 | 0.00065014 | Homo sapiens cDNA FLJ32488 fis, clone SKNSH1000086 |
| 229901 | 0.049474849 | CTSO\|cathepsin O |
| 503214 | 9.28E−09 | KIAA1243\|KIAA1243 protein |
| 796757 | 0.037697334 | AP3S1\|adaptor-related protein complex 3, sigma 1 subunit |
| 1917588 | 0.048777689 | DNASE2\|deoxyribonuclease II, lysosomal |
| 271748 | 6.83E−06 | RBMS1\|RNA binding motif, single stranded interacting protein 1 |
| 49860 | 9.02E−05 | PDK2\|pyruvate dehydrogenase kinase, isoenzyme 2 |
| 504959 | 7.12E−10 | Homo sapiens mRNA; cDNA DKFZp586G0321 (from clone DKFZp586G0321) |
| 1602284 | 1.87E−07 | EST |
| 687289 | 0.004049154 | Homo sapiens, clone MGC: 3245 IMAGE: 3505639, mRNA, complete cds |
| 150118 | 0.017865847 | DKFZp434F054\|hypothetical protein DKFZp434F054 |
| 140354 | 0.013770597 | Homo sapiens, Similar to secretory carrier membrane protein 4, clone MGC: 19661 IMAGE: 3161979, |
| | | mRNA, complete cds |
| 786202 | 0.002899676 | Homo sapiens cDNA FLJ13553 fis, clone PLACE1007454 |
| 361565 | 6.41E−05 | GLUD1\|glutamate dehydrogenase 1 |
| 512045 | 0.042758148 | JM1\|JM1 protein |
| 591597 | 0.011216747 | TNNI3\|troponin I, cardiac |
| 1574252 | 0.03289313 | DKFZP586D0623\|DKFZP586D0623 protein |
| 740476 | 0.002102096 | IRF1\|interferon regulatory factor 1 |
| 951125 | 1.70E−05 | PECI\|peroxisomal D3,D2-enoyl-CoA isomerase |
| 1636669 | 0.001613372 | FLJ21939\|hypothetical protein FLJ21939 similar to 5-azacytidine induced gene 2 |
| 283124 | 4.09E−07 | Homo sapiens, clone IMAGE: 3917549, mRNA, partial cds |
| 79960 | 0.006425318 | RRP40\|exosome component Rrp40 |
| 25274 | 2.51E−09 | LOC51161\|g20 protein |
| 261204 | 2.66E−16 | ESTs, Highly similar to JC5931 high mobility group I HMGI chromosomal protein isoform C-alpha |
| | | [H. sapiens] |
| 949938 | 2.51E−07 | CST3\|cystatin C (amyloid angiopathy and cerebral hemorrhage) |
| 49883 | 0.016175391 | SYT13\|synaptotagmin XIII |
| 155072 | 8.66E−16 | ESTs |
| 502297 | 0.000223246 | POLI\|polymerase (DNA directed) iota |
| 150137 | 0.040830931 | DKFZP564O123\|DKFZP564O123 protein |
| 85450 | 8.06E−09 | ACOX2\|acyl-Coenzyme A oxidase 2, branched chain |
| 212542 | 0.000127809 | Homo sapiens mRNA; cDNA DKFZp586J2118 (from clone DKFZp586J2118) |
| 813603 | 5.67E−06 | KIAA1075\|KIAA1075 protein |
| 361587 | 4.46E−05 | KIAA1789\|KIAA1789 protein |
| 742094 | 0.003398986 | FLJ20950\|hypothetical protein FLJ20950 |
| 156023 | 0.019869669 | MGC3123\|hypothetical protein MGC3123 |
| 1558233 | 8.16E−12 | ESTs |
| 229937 | 5.99E−05 | DDX30\|DEAD/H (Asp-Glu-Ala-Asp/His) box polypeptide 30 |
| 1469084 | 0.019869669 | KIAA0804\|KIAA0804 protein |
| 362532 | 0.002943504 | C12orf22\|chromosome 12 open reading frame 22 |
| 51093 | 0.003245464 | ZNFN2A1\|zinc finger protein, subfamily 2A (FYVE domain containing), 1 |
| 1631746 | 5.44E−05 | POLM\|polymerase (DNA directed), mu |
| 51097 | 1.98E−05 | BAIAP3\|BAI1-associated protein 3 |
| 2108048 | 8.16E−07 | DNB5\|DNB5 |
| 342181-2 | 8.94E−21 | BCL2\|B-cell CLL/lymphoma 2 |
| 785293 | 3.42E−07 | HREV107\|similar to rat HREV107 |
| 1741884 | 0.001594038 | ESTs, Highly similar to IRX3_HUMAN IROQUOIS-CLASS HOMEODOMAIN PROTEIN IRX-3 [H. sapiens] |
| 48167 | 0.001837947 | FLJ10483\|hypothetical protein FLJ10483 |
| 155094 | 0.010881395 | DNAJB12\|DnaJ (Hsp40) homolog, subfamily B, member 12 |
| 842762 | 0.014914359 | FLJ20363\|hypothetical protein FLJ20363 |
| 1685363 | 0.003881514 | GLRB\|glycine receptor, beta |
| 1840803 | 0.032260939 | FLJ20686\|hypothetical protein FLJ20686 |
| 825785 | 1.01E−05 | KIAA0435\|KIAA0435 gene product |
| 2016644 | 0.009775018 | LIMD1\|LIM domains containing 1 |
| 255754 | 2.24E−06 | LOC51297\|LUNX protein; PLUNC (palate lung and nasal epithelium clone); tracheal epithelium enriched |
| | | protein |
| 159380 | 0.000970624 | FLJ23462\|duodenal cytochrome b |
| 159381 | 1.10E−06 | RARA\|retinoic acid receptor, alpha |
| 1517749 | 0.012504399 | ESTs |
| 712314 | 3.40E−05 | MIR\|myosin regulatory light chain interacting protein |
| 2284924 | 1.10E−14 | KIAA0882\|KIAA0882 protein |
| 1475662 | 0.000802641 | AXUD1\|AXIN1 up-regulated |
| 1637647 | 7.42E−05 | ESTs |
| 878744 | 0.012204566 | TSG101\|tumor susceptibility gene 101 |
| 824052 | 0.031302263 | C6orf1\|chromosome 6 open reading frame 1 |
| 40695 | 0.044959539 | NAPB\|N-ethylmaleimide-sensitive factor attachment protein, beta |
| 773152 | 0.001310908 | ESTs |
| 289057 | 0.004505214 | HT021\|HT021 |
| 120271-2 | 0.015988127 | MGC4692\|hypothetical protein MGC4692 |
| 744010 | 7.00E−05 | Homo sapiens mRNA; cDNA DKFZp586K1318 (from clone DKFZp586K1318) |
| 79216 | 0.008448199 | AHNAK\|AHNAK nucleoprotein (desmoyokin) |
| 842785 | 0.011581406 | LRPAP1\|low density lipoprotein-related protein-associated protein 1 (alpha-2-macroglobulin receptor- |
| | | associated protein 1) |
| 41647 | 1.11E−06 | PTPRT\|protein tyrosine phosphatase, receptor type, T |
| 549016 | 0.019299231 | BTBD1\|BTB (POZ) domain containing 1 |
| 705110 | 0.001790158 | CASP9\|caspase 9, apoptosis-related cysteine protease |
| 2313921 | 0.001677464 | NDUFB3\|NADH dehydrogenase (ubiquinone) 1 beta subcomplex, 3 (12 kD, B12) |
| 810305 | 0.002373538 | Homo sapiens cDNA FLJ30463 fis, clone BRACE2009517 |
| 289060 | 0.000990298 | HSPA4\|heat shock 70 kD protein 4 |
| 262996 | 0.02908529 | CHD1\|chromodomain helicase DNA binding protein 1 |
| 687638 | 0.006365847 | ESTs |
| 1670688 | 0.006338445 | BACH2\|BTB and CNC homology 1, basic leucine zipper transcription factor 2 |
| 154466 | 5.44E−05 | STUB1\|STIP1 homology and U-Box containing protein 1 |
| 52071 | 0.026442939 | ESTs, Highly similar to AC004836 1 similar to cadherin and Drosophila Fat protein [H. sapiens] |
| 301740 | 0.018221151 | Homo sapiens cDNA FLJ30298 fis, clone BRACE2003172 |
| 52076 | 0.000531118 | OLFM1\|olfactomedin 1 |
| 34439 | 0.000548494 | FUS2\|putative tumor suppressor |
| 428184 | 8.50E−10 | Homo sapiens, clone MGC: 18216 IMAGE: 4156235, mRNA, complete cds |
| 743081 | 0.048000809 | NDUFA3\|NADH dehydrogenase (ubiquinone) 1 alpha subcomplex, 3 (9 kD, B9) |
| 565062 | 0.023268805 | Homo sapiens clone 24812 mRNA sequence |
| 31893 | 0.010529568 | LOC84548\|hypothetical protein from Xq28 |
| 646057 | 0.010403382 | Homo sapiens cDNA: FLJ21897 fis, clone HEP03447, highly similar to AF052178 Homo sapiens clone |
| | | 24523 mRNA sequence |
| 809779 | 8.13E−08 | KIAA0239\|KIAA0239 protein |
| 1575263 | 0.005751783 | DKFZP586N0721\|DKFZP586N0721 protein |
| 773170 | 2.17E−06 | TPBG\|trophoblast glycoprotein |
| 853506 | 0.002203842 | PISD\|phosphatidylserine decarboxylase |
| 757135 | 2.60E−05 | DKFZP564J0123\|DKFZP564J0123 protein |
| 381166 | 3.78E−06 | MORF\|histone acetyltransferase |
| 361922 | 0.040228151 | ZMPSTE24\|zinc metalloproteinase, STE24 (yeast, homolog) |
| 50484 | 2.41E−05 | CCRK\|cell cycle related kinase |
| 281590 | 0.026213959 | FLJ10656\|hypothetical protein FLJ10656 |
| 1700621 | 0.001782567 | EGFL4\|EGF-like-domain, multiple 4 |
| 26289 | 0.041064782 | FLJ12879\|hypothetical protein FLJ12879 |
| 565075 | 0.000170815 | STC1\|stanniocalcin 1 |
| 1568056 | 0.005442251 | ESTs, Moderately similar to 178885 serine/threonine-specific protein kinase [H. sapiens] |
| 252453 | 0.00590112 | LOC51635\|CGI-86 protein |
| 810325 | 2.48E−05 | IVD\|isovaleryl Coenzyme A dehydrogenase |
| 1469425 | 0.00010497 | SOX22\|SRY (sex determining region Y)-box 22 |
| 781342 | 2.74E−05 | MGC11115\|hypothetical protein MGC11115 |
| 154482 | 1.29E−05 | SERPINB1\|serine (or cysteine) proteinase inhibitor, clade B (ovalbumin), member 1 |
| 845352 | 0.000586706 | NUDT9\|nudix (nucleoside diphosphate linked moiety X)-type motif 9 |
| 266500 | 2.03E−08 | ESTs |
| 773185 | 0.008673574 | ESTs |
| 136845 | 0.024067407 | Homo sapiens, clone IMAGE: 3915000, mRNA |
| 1870938 | 0.007988734 | CDS1\|CDP-diacylglycerol synthase (phosphatidate cytidylyltransferase) 1 |
| 310584 | 7.42E−05 | ARL1\|ADP-ribosylation factor-like 1 |
| 1585040 | 1.45E−08 | TCEB1L\|transcription elongation factor B (SIII), polypeptide 1-like |
| 1916225 | 0.005767924 | ESTs, Weakly similar to A43932 mucin 2 precursor, intestinal [H. sapiens] |
| 26294 | 2.07E−09 | RNB6\|RNB6 |
| 416042 | 0.000488136 | Homo sapiens cDNA FLJ31053 fis, clone HSYRA2000640, highly similar to Homo sapiens FRG1 mRNA |
| 248583 | 0.036283521 | NFX1\|nuclear transcription factor, X-box binding 1 |
| 344959 | 2.38E−09 | HSA250839\|gene for serine/threonine protein kinase |
| 2010012 | 0.004201172 | P37NB\|37 kDa leucine-rich repeat (LRR) protein |
| 71763 | 0.001262603 | SIP\|Siah-interacting protein |
| 154493 | 0.015871 | IFI41\|interferon-induced protein 41, 30 kD |
| 2054945 | 0.000649145 | SLC23A2\|solute carrier family 23 (nucleobase transporters), member 2 |
| 797000 | 0.011811432 | TERE1\|transitional epithelia response protein |
| 594517 | 0.036288061 | SFRS6\|splicing factor, arginine/serine-rich 6 |
| 206341 | 0.011625517 | ESTs, Weakly similar to A49364 59 protein, brain [H. sapiens] |
| 280950 | 4.51E−05 | SRI\|sorcin |
| 109316 | 0.006324487 | SERPINA3\|serine (or cysteine) proteinase inhibitor, clade A (alpha-1 antiproteinase, antitrypsin), |
| | | member 3 |
| 203003 | 0.002285045 | NME4\|non-metastatic cells 4, protein expressed in |
| 503602 | 0.000403283 | PRO1489\|hypothetical protein PRO1489 |
| 769600 | 0.013770597 | UNG2\|uracil-DNA glycosylase 2 |
| 1968246 | 1.12E−05 | FUT8\|fucosyltransferase 8 (alpha (1,6) fucosyltransferase) |
| 432093 | 2.37E−06 | IL18BP\|interleukin 18 binding protein |
| 1879094 | 0.002741302 | FLJ23392\|hypothetical protein FLJ23392 |
| 782306 | 3.08E−06 | FLJ13110\|hypothetical protein FLJ13110 |
| 1635307 | 4.84E−05 | Homo sapiens, clone IMAGE: 3833472, mRNA |
| 1420370 | 0.036288061 | BLVRB\|biliverdin reductase B (flavin reductase (NADPH)) |
| 284160 | 3.04E−08 | CACNA2D2\|calcium channel, voltage-dependent, alpha 2/delta subunit 2 |
| 595181 | 0.000601881 | MIR\|myosin regulatory light chain interacting protein |
| 470348 | 0.001309802 | ESTs |
| 1926007 | 0.00216623 | EST |
| 33817 | 7.74E−06 | ESTs |
| 1456348 | 0.020321818 | SAS\|N-acetylneuraminic acid phosphate synthase; sialic acid synthase |
| 1056172 | 0.002436429 | NMES1\|normal mucosa of esophagus specific 1 |
| 84880 | 0.000162089 | LOC51004\|CGI-10 protein |
| 491612 | 0.000816756 | Homo sapiens cDNA FLJ11752 fis, clone HEMBA1005582, weakly similar to TROPOMYOSIN 1, NON- |
| | | MUSCLE ISOFORM |
| 344970 | 0.009457359 | ITGA4\|integrin, alpha 4 (antigen CD49D, alpha 4 subunit of VLA-4 receptor) |
| 796079 | 0.001202428 | MGC4171\|hypothetical protein MGC4171 |
| 504279 | 0.016946636 | TBL1\|transducin (beta)-like 1 |
| 898333 | 0.04010901 | ESTs |
| 950574 | 0.013952573 | H3F3B\|H3 histone, family 3B (H3.3B) |
| 250883 | 0.010544493 | UBE1L\|ubiquitin-activating enzyme E1-like |
| 230560 | 0.003417271 | ESTs |
| 666707 | 0.002506551 | ESTs, Weakly similar to I38022 hypothetical protein [H. sapiens] |
| 825060 | 0.012784217 | DUT\|dUTP pyrophosphatase |
| 1690262 | 0.032703827 | Homo sapiens cDNA FLJ20763 fis, clone COL09911 |
| 788208 | 7.66E−07 | FLJ10659\|hypothetical protein FLJ10659 |
| 265592 | 0.019761169 | Homo sapiens, activated RNA polymerase II transcription cofactor 4, clone MGC: 21503 IMAGE: 3881373, |
| | | mRNA, complete cds |
| 291399 | 0.000155359 | HMG2\|high-mobility group (nonhistone chromosomal) protein 2 |
| 262251 | 0.000333214 | CLCN7\|chloride channel 7 |
| 786607 | 0.000772083 | TCEAL1\|transcription elongation factor A (SII)-like 1 |
| 108395 | 1.56E−05 | DLG5\|discs, large (Drosophila) homolog 5 |
| 245273 | 0.019521612 | LOC57020\|hypothetical protein |
| 626861 | 0.009420842 | EIF4G2\|eukaryotic translation initiation factor 4 gamma, 2 |
| 1323361 | 0.008588081 | NR2F6\|nuclear receptor subfamily 2, group F, member 6 |
| 489351 | 0.00010205 | DKFZP566J2046\|hypothetical protein DKFZp566J2046 |
| 2054977 | 0.000148287 | KIAA0373\|KIAA0373 gene product |
| 26617 | 0.000316752 | ALCAM\|activated leucocyte cell adhesion molecule |
| 268148 | 0.000875061 | TRIM4\|tripartite motif-containing 4 |
| 665774 | 0.049423242 | EIF4E\|eukaryotic translation initiation factor 4E |
| 797038 | 0.011982092 | Homo sapiens mRNA full length insert cDNA clone EUROIMAGE 1502032 |
| 1652310 | 0.017604138 | Homo sapiens cDNA FLJ30306 fis, clone BRACE2003319 |
| 562867 | 0.044978437 | RRS1\|homolog of yeast ribosome biogenesis regulatory protein RRS1 |
| 1880743 | 0.008335378 | ESTs |
| 38781 | 0.03544066 | ATP9B\|ATPase, Class II, type 9B |
| 1609538 | 3.46E−05 | KIAA1324\|KIAA1324 protein |
| 487761 | 0.034130662 | TAF2S\|TATA box binding protein (TBP)-associated factor, RNA polymerase II, S, 150 kD |
| 36491-2 | 0.00247482 | ESTs |
| 755599 | 0.023780533 | IFITM1\|interferon induced transmembrane protein 1 (9-27) |
| 2017684 | 0.000107787 | LOC51644\|CGI-120 protein |
| 244637 | 0.000498127 | CRI1\|CREBBP/EP300 inhibitory protein 1 |
| 156437-2 | 0.001309095 | ATP1A2\|ATPase, Na+/K+ transporting, alpha 2 (+) polypeptide |
| 2222453 | 0.028657358 | EHD2\|EH-domain containing 2 |
| 53092 | 0.008880977 | KIAA0436\|putative L-type neutral amino acid transporter |
| 841415 | 4.94E−05 | EPLIN\|epithelial protein lost in neoplasm beta |
| 770983 | 0.010403382 | FLJ11730\|hypothetical protein FLJ11730 |
| 363590 | 0.00042623 | ARNT2\|aryl hydrocarbon receptor nuclear translocator 2 |
| 1586043 | 0.018119429 | ESTs |
| 2709505 | 4.45E−08 | LPHB\|lipophilin B (uteroglobin family member), prostatein-like |
| 204790 | 2.03E−09 | FLJ20273\|hypothetical protein |
| 1897065 | 0.017531828 | SMUG1\|single-strand selective monofunctional uracil DNA glycosylase |
| 487777 | 0.02848343 | RB1\|retinoblastoma 1 (including osteosarcoma) |
| 263229 | 0.001149746 | LAF4\|lymphoid nuclear protein related to AF4 |
| 1911343 | 3.74E−12 | RAB26\|RAB26, member RAS oncogene family |
| 1558642 | 1.95E−11 | MLPH\|melanophilin |
| 60602 | 3.22E−06 | CSH1\|chorionic somatomammotropin hormone 1 (placental lactogen) |
| 156437 | 0.036072406 | ATP1A2\|ATPase, Na+/K+ transporting, alpha 2 (+) polypeptide |
| 730346 | 0.003570072 | HEBP1\|heme binding protein 1 |
| 143332 | 0.005649342 | NPY1R\|neuropeptide Y receptor Y1 |
| 32122 | 2.31E−09 | FLJ10210\|hypothetical protein FLJ10210 |
| 195346 | 7.13E−06 | FLJ00005\|FLJ00005 protein |
| 786640 | 0.005990788 | VEZATIN\|transmembrane protein vezatin |
| 109379 | 0.003007609 | HMGCR\|3-hydroxy-3-methylglutaryl-Coenzyme A reductase |
| 590298 | 0.023157899 | MRPL45\|mitochondrial ribosomal protein L45 |
| 645485 | 0.005375632 | TERF1\|telomeric repeat binding factor (NIMA-interacting) 1 |
| 134523 | 5.65E−05 | EMAP-2\|microtubule-associated protein like echinoderm EMAP |
| 134525 | 0.013501077 | CUL3\|cullin 3 |
| 44292 | 0.027304029 | Homo sapiens mRNA; cDNA DKFZp434C107 (from clone DKFZp434C107) |
| 140008 | 0.018500323 | EBNA1BP2\|EBNA1-binding protein 2 |
| 511831 | 3.46E−05 | MGC12936\|hypothetical protein MGC12936 |
| 2014373 | 0.0330879 | HNK-1ST\|HNK-1 sulfotransferase |
| 928315 | 0.014172516 | CARKL\|carbohydrate kinase-like |
| 172495 | 0.002069329 | KIAA0028\|leucyl-tRNA synthetase, mitochondrial |
| 724888 | 4.45E−05 | CYP4B1\|cytochrome P450, subfamily IVB, polypeptide 1 |
| 296679 | 0.0002926 | Homo sapiens clone TCCCTA00151 mRNA sequence |
| 357344 | 0.035245897 | KCNQ1\|potassium voltage-gated channel, KQT-like subfamily, member 1 |
| 843046 | 0.009533011 | GPRK7\|G protein-coupled receptor kinase 7 |
| 789204 | 0.001843366 | TLOC1\|translocation protein 1 |
| 183200 | 0.012787936 | FAH\|fumarylacetoacetate hydrolase (fumarylacetoacetase) |
| 744417 | 0.006558294 | CRAT\|carnitine acetyltransferase |
| 812300 | 0.003557075 | FLJ20265\|hypothetical protein FLJ20265 |
| 418019 | 0.001030569 | KIAA0542\|KIAA0542 gene product |
| 1569096 | 0.022893459 | ESTs |
| 79629-2 | 0.014086588 | CXCR4\|chemokine (C—X—C motif), receptor 4 (fusin) |
| 782385 | 0.031219133 | DKFZP566D193\|DKFZP566D193 protein |
| 897745 | 0.002311826 | FLJ13868\|hypothetical protein FLJ13868 |
| 843054 | 0.004353571 | KIAA1533\|KIAA1533 protein |
| 826070 | 0.017970183 | ZFP91\|zinc finger protein homologous to Zfp91 in mouse |
| 1608928 | 0.028565804 | CYP4F11\|cytochrome P450, subfamily IVF, polypeptide 11 |
| 564801 | 0.006100942 | Homo sapiens cDNA: FLJ21409 fis, clone COL03924 |
| 826077 | 0.000373482 | PDHB\|pyruvate dehydrogenase (lipoamide) beta |
| 177737 | 0.014789221 | PURA\|purine-rich element binding protein A |
| 823819 | 0.001641999 | EST |
| 83508 | 0.026147968 | B9\|B9 protein |
| 795498 | 0.001139656 | HS1-2\|putative transmembrane protein |
| 505299 | 0.000389422 | BBP\|beta-amyloid binding protein precursor |
| 49546 | 0.02081588 | P29\|GCIP-interacting protein p29 |
| 2028440 | 0.001074094 | ESTs |
| 346942 | 0.0003267 | PIGQ\|phosphatidylinositol glycan, class Q |
| 34849 | 0.017962259 | EEF2\|eukaryotic translation elongation factor 2 |
| 247230 | 0.011626312 | Homo sapiens cDNA FLJ30532 fis, clone BRAWH2001129, weakly similar to OCCLUDIN |
| 50888 | 0.000228763 | PRDX3\|peroxiredoxin 3 |
| 754998 | 0.039096645 | SRP19\|signal recognition particle 19 kD |
| 2168667 | 0.002032038 | POLR2H\|polymerase (RNA) II (DNA directed) polypeptide H |
| 724112 | 0.001610749 | ESTs, Weakly similar to B34087 hypothetical protein [H. sapiens] |
| 741891 | 2.17E−05 | RAB2L\|RAB2, member RAS oncogene family-like |
| 130276 | 0.027971153 | Homo sapiens mRNA; cDNA DKFZp586H0324 (from clone DKFZp586H0324) |
| 66535 | 0.000646325 | OAZ2\|ornithine decarboxylase antizyme 2 |
| 1500000 | 0.000577003 | H2BFB\|H2B histone family, member B |
| 1049330 | 0.000116235 | SSPN\|sarcospan (Kras oncogene-associated gene) |
| 1683411 | 0.000739744 | ESTs |
| 611443 | 0.000106149 | MB\|myoglobin |
| 591281 | 0.021583844 | MVP\|major vault protein |
| 504657 | 0.013710873 | KIAA0438\|KIAA0438 gene product |
| 810734 | 6.48E−09 | POLD4\|polymerase (DNA-directed), delta 4 |
| 2297394 | 0.022064191 | FLJ13612\|hypothetical protein FLJ13612 |
| 1901924 | 0.014853485 | ESTs, Weakly similar to T21364 hypothetical protein F25H5.6 - Caenorhabditis elegans [C. elegans] |
| 1553766 | 0.000795582 | FLJ10283\|hypothetical protein FLJ10283 |
| 267571 | 2.41E−06 | SNAPC2\|small nuclear RNA activating complex, polypeptide 2, 45 kD |
| 1588791 | 0.02123298 | MGMT\|O-6-methylguanine-DNA methyltransferase |
| 1049346 | 1.26E−05 | FOXP1\|forkhead box P1 |
| 121470 | 0.030626 | BCCIP\|BRCA2 and CDKN1A-interacting protein |
| 298603 | 0.005015434 | KIAA1474\|KIAA1474 protein |
| 1569418 | 2.89E−07 | Homo sapiens cDNA FLJ11385 fis, clone HEMBA1000520 |
| 782700 | 0.000804231 | CLASP2\|CLIP-associating protein 2 |
| 884677 | 3.02E−05 | Homo sapiens, clone IMAGE: 3611719, mRNA, partial cds |
| 782702 | 8.52E−08 | LOC56270\|hypothetical protein 628 |
| 108425-2 | 0.006209446 | ESTs, Weakly similar to JC5314 CDC28/cdc2-like kinase associating arginine-serine cyclophilin |
| | | [H. sapiens] |
| 154103 | 0.000352444 | SCN1B\|sodium channel, voltage-gated, type I, beta polypeptide |
| 759163 | 2.95E−13 | MFAP4\|microfibrillar-associated protein 4 |
| 1566877 | 0.020116819 | C11orf2\|chromosome 11 open reading frame2 |
| 1635705 | 0.016769168 | ESTs, Highly similar to T46422 hypothetical protein DKFZp434M2023.1 [H. sapiens] |
| 35812 | 0.022973459 | PCLO\|piccolo (presynaptic cytomatrix protein) |
| 51851 | 0.004091636 | ESTs, Weakly similar to I78885 serine/threonine-specific protein kinase [H. sapiens] |
| 1839935 | 2.36E−06 | ESTs |
| 2169632 | 0.022764168 | Homo sapiens mRNA full length insert cDNA clone EUROIMAGE 42138 |
| 669359 | 0.00497373 | Homo sapiens clone 24405 mRNA sequence |
| 1625945 | 0.004424523 | NDRG3\|N-myc downstream-regulated gene 3 |
| 682058 | 0.00167005 | SPOP\|speckle-type POZ protein |
| 752625 | 0.00014539 | Homo sapiens mRNA; cDNA DKFZp667D095 (from clone DKFZp667D095) |
| 843094 | 0.046423899 | UBL1\|ubiquitin-like 1 (sentrin) |
| 298610 | 0.007478622 | Homo sapiens cDNA FLJ13927 fis, clone Y79AA1000627, moderately similar to Homo sapiens zinc finger |
| | | protein (ZF5128) mRNA |
| 299274 | 0.033282881 | Homo sapiens cDNA FLJ32430 fis, clone SKMUS2001129, weakly similar to NAD-DEPENDENT |
| | | METHANOL DEHYDROGENASE (EC 1.1.1.244) |
| 41302 | 0.002068339 | KIAA0643\|KIAA0643 protein |
| 682063 | 0.001692383 | ZNF189\|zinc finger protein 189 |
| 824802 | 1.92E−05 | HTGN29\|HTGN29 protein |
| 35828 | 0.024474794 | DTR\|diphtheria toxin receptor (heparin-binding epidermal growth factor-like growth factor) |
| 283919 | 0.046763683 | H2AFL\|H2A histone family, member L |
| 868652 | 4.71E−08 | C4B\|complement component 4B |
| 1668067 | 7.31E−05 | DKFZP434D146\|DKFZP434D146 protein |
| 345247 | 0.007295582 | ESTs, Moderately similar to T03094 A-kinase anchor protein DAKAP550 - fruit fly [D. melanogaster] |
| 823867 | 0.005767924 | ESTs |
| 2455403 | 0.000513257 | ZNF91\|zinc finger protein 91 (HPF7, HTF10) |
| 366511 | 0.003874401 | ACTA1\|actin, alpha 1, skeletal muscle |
| 767346 | 0.017970183 | C14orf4\|chromosome 14 open reading frame 4 |
| 241474-2 | 0.000122034 | BRCA1\|breast cancer 1, early onset |
| 772962 | 0.00014351 | Homo sapiens cDNA FLJ31149 fis, clone IMR322001491, moderately similar to Rattus norvegicus |
| | | tricarboxylate carrier-like protein mRNA |
| 855559 | 2.45E−10 | SAS\|sarcoma amplified sequence |
| 36491 | 0.01049982 | ESTs |
| 669379 | 0.031219133 | Homo sapiens, clone IMAGE: 3463399, mRNA, partial cds |
| 36493 | 0.015997677 | NAGA\|N-acetylgalactosaminidase, alpha- |
| 282980 | 0.001343272 | ESTs |
| 752643 | 1.91E−05 | PLA2G12\|group XII secreted phospholipase A2 |
| 69124 | 0.000606588 | CIRBP\|cold inducible RNA-binding protein |
| 566466 | 0.01931096 | HSU93850\|elongation factor-2 kinase |
| 510170 | 0.000277083 | PAIP2\|PABP-interacting protein 2 |
| 855563 | 5.61E−05 | ERBB3\|v-erb-b2 avian erythroblastic leukemia viral oncogene homolog 3 |
| 139835 | 7.14E−08 | UGDH\|UDP-glucose dehydrogenase |
| 154138 | 0.024566797 | Homo sapiens cDNA FLJ32174 fis, clone PLACE6001064 |
| 2284619 | 0.011581406 | ZNF132\|zinc finger protein 132 (clone pHZ-12) |
| 460218 | 0.018807962 | EST |
| 488025 | 0.000960715 | ZNF226\|zinc finger protein 226 |
| 745437 | 0.003016551 | ESTs |
| 208387 | 0.000636782 | KIAA1407\|KIAA1407 protein |
| 1843843 | 0.025604134 | SRGAP1\|KIAA1304 protein |
| 258790 | 0.006397727 | CCK\|cholecystokinin |
| 205049 | 0.000551716 | H11\|protein kinase H11 |
| 490306 | 0.000487266 | TM9SF1\|transmembrane 9 superfamily member 1 |
| 773922 | 0.004876463 | BZAP45\|basic leucine-zipper protein BZAP45 |
| 66599 | 1.39E−11 | NAT1\|N-acetyltransferase 1 (arylamine N-acetyltransferase) |
| 280602 | 0.000362169 | ESTs |
| 488033 | 0.002117729 | DNAJB9\|DnaJ (Hsp40) homolog, subfamily B, member 9 |
| 1860836 | 0.007295582 | Homo sapiens cDNA FLJ30927 fis, clone FEBRA2006736 |
| 726860 | 0.011916108 | C11orf14\|chromosome 11 open reading frame 14 |
| 840878 | 0.000600849 | DHCR24\|24-dehydrocholesterol reductase |
| 1702695 | 0.003491873 | MRPL53\|mitochondrial ribosomal protein L53 |
| 289830 | 0.024964587 | KIAA0329\|KIAA0329 gene product |
| 1569465 | 0.027305675 | IGHMBP2\|immunoglobulin mu binding protein 2 |
| 2367226 | 0.001193191 | HRC\|histidine-rich calcium-binding protein |
| 2048040 | 0.002874419 | Homo sapiens cDNA FLJ32130 fis, clone PEBLM2000248, weakly similar to ZINC FINGER PROTEIN |
| | | 157 |
| 810791 | 0.006604078 | MNAT1\|menage a trois 1 (CAK assembly factor) |
| 810795 | 1.05E−05 | FLJ21940\|hypothetical protein FLJ21940 |
| 1561981 | 7.62E−05 | EST |
| 28643 | 5.46E−05 | DKFZP564D1378\|hypothetical protein DKFZp564D1378 |
| 665405 | 4.33E−05 | MYO5C\|myosin 5C |
| 1518402 | 1.68E−09 | KIAA1361\|KIAA1361 protein |
| 283956 | 6.52E−05 | Homo sapiens cDNA FLJ13155 fis, clone NT2RP3003433 |
| 840882 | 1.48E−06 | NBP\|nucleotide binding protein |
| 742115 | 1.20E−06 | DCI\|dodecenoyl-Coenzyme A delta isomerase (3,2 trans-enoyl-Coenzyme A isomerase) |
| 809464 | 0.015509308 | FGFR2\|fibroblast growth factor receptor 2 (bacteria-expressed kinase, keratinocyte growth factor |
| | | receptor, craniofacial dysostosis 1, Crouzon syndrome, Pfeiffer syndrome, Jackson-Weiss syndrome) |
| 85195 | 0.00013313 | GADD45G\|growth arrest and DNA-damage-inducible, gamma |
| 1521341 | 8.51E−05 | HIRIP3\|HIRA-interacting protein 3 |
| 768324 | 0.010220908 | KIAA0107\|KIAA0107 gene product |
| 210688 | 1.39E−12 | ADCY9\|adenylate cyclase 9 |
| 1557318 | 0.028091838 | ESTs |
| 1636707 | 0.010283446 | EIF3S3\|eukaryotic translation initiation factor 3, subunit 3 (gamma, 40 kD) |
| 837953 | 1.10E−05 | NEDD4L\|neural precursor cell expressed, developmentally down-regulated 4-like |
| 429906 | 0.009533011 | PIGN\|phosphatidylinositol glycan, class N |
| 530814 | 0.000234182 | SEPP1\|selenoprotein P, plasma, 1 |
| 258026 | 0.000719645 | EST |
| 41356 | 0.035107908 | PPP2R5A\|protein phosphatase 2, regulatory subunit B (B56), alpha isoform |
| 1631487 | 0.001988084 | ESTs |
| 752688 | 1.73E−05 | ESTs |
| 565849 | 0.000900648 | C3IP1\|kelch-like protein C3IP1 |
| 1699142 | 3.36E−05 | AP1G2\|adaptor-related protein complex 1, gamma 2 subunit |
| 253884 | 0.043335326 | Homo sapiens, clone IMAGE: 3625286, mRNA, partial cds |
| 810017 | 0.000594167 | PLAUR\|plasminogen activator, urokinase receptor |
| 1422723 | 0.02727988 | IFI35\|interferon-induced protein 35 |
| 1868349 | 0.023383495 | PLA2G4B\|phospholipase A2, group IVB (cytosolic) |
| 730768 | 9.62E−05 | Homo sapiens mRNA; cDNA DKFZp434M245 (from clone DKFZp434M245) |
| 1537001 | 0.012963093 | ESTs |
| 470261-2 | 0.000165604 | SMA5\|SMA5 |
| 712049 | 0.01747597 | IL24\|interleukin 24 |
| 526945 | 0.013261832 | FABGL\|FabG (beta-ketoacyl-[acyl-carrier-protein] reductase, E coli) like |
| 814306 | 0.020168182 | TPD52\|tumor protein D52 |
| 726890 | 0.00099919 | MGC4643\|hypothetical protein MGC4643 |
| 1572710 | 0.008888435 | FLJ21213\|hypothetical protein FLJ21213 |
| 809488 | 0.007951698 | RAI17\|retinoic acid induced 17 |
| 502349 | 2.77E−06 | CAB56184\|hypothetical protein CAB56184 |
| 781046 | 0.000438842 | ERBB2IP\|erbb2-interacting protein ERBIN |
| 782787 | 2.14E−05 | FLJ21347\|hypothetical protein FLJ21347 |
| 365919 | 6.59E−07 | STAU\|staufen (Drosophila, RNA-binding protein) |
| 1898442 | 0.001557478 | ESTs, Weakly similar to A43932 mucin 2 precursor, intestinal [H. sapiens] |
| 1908840 | 1.16E−05 | ZNF174\|zinc finger protein 174 |
| 2298710 | 0.012030871 | TRIM16\|tripartite motif-containing 16 |
| 1560279 | 0.02187025 | Homo sapiens clone CDABP0155 mRNA sequence |
| 743880 | 0.014144736 | KIAA0263\|KIAA0263 gene product |
| 824879 | 5.97E−08 | MGC11275\|hypothetical protein MGC11275 |
| 270883 | 0.001399452 | CNNM3\|cyclin M3 |
| 341316 | 2.88E−07 | HTATSF1\|HIV TAT specific factor 1 |
| 1910040 | 2.37E−06 | LOC51701\|nemo-like kinase |
| 81229 | 0.00100873 | FTL\|ferritin, light polypeptide |
| 811774 | 5.01E−08 | LOC51097\|CGI-49 protein |
| 1521375 | 8.48E−06 | Homo sapiens cDNA FLJ13542 fis, clone PLACE1006779 |
| 839583 | 0.01394813 | ESTs, Moderately similar to T46386 hypothetical protein DKFZp434P011.1 [H. sapiens] |
| 214205 | 9.59E−12 | Homo sapiens, clone MGC: 17687 IMAGE: 3865868, mRNA, complete cds |
| 825822 | 0.000681333 | DJ167A19.1\|hypothetical protein |
| 1762111 | 0.000300738 | NPR3\|natriuretic peptide receptor C/guanylate cyclase C (atrionatriuretic peptide receptor C) |
| 487425 | 0.001309802 | CETN3\|centrin, EF-hand protein, 3 (CDC31 yeast homolog) |
| 195786 | 0.000395849 | EST |
| 2298723 | 0.014679158 | KIAA0708\|KIAA0708 protein |
| 66931 | 0.023818015 | FLJ20307\|hypothetical protein FLJ20307 |
| 490360 | 0.018119429 | MGC5560\|hypothetical protein MGC5560 |
| 767706 | 3.86E−05 | ESTs |
| 1732033 | 0.00024242 | FLJ14427\|hypothetical protein FLJ14427 |
| 234537 | 0.039367559 | Homo sapiens, clone IMAGE: 3869590, mRNA, partial cds |
| 1631807 | 0.013415846 | GMPPB\|GDP-mannose pyrophosphorylase B |
| 2017352 | 0.003753263 | B4GALT7\|xylosylprotein beta1,4-galactosyltransferase, polypeptide 7 (galactosyltransferase I) |
| 361659 | 0.023660933 | DKFZP434D146\|DKFZP434D146 protein |
| 66946 | 0.017962259 | U2AF1RS2\|U2 small nuclear ribonucleoprotein auxiliary factor, small subunit 2 |
| 1610546 | 2.73E−11 | HNF3A\|hepatocyte nuclear factor 3, alpha |
| 811790 | 0.024330122 | DKFZP564G0222\|DKFZP564G0222 protein |
| 951216 | 0.001063475 | NDUFB10\|NADH dehydrogenase (ubiquinone) 1 beta subcomplex, 10 (22 kD, PDSW) |
| 811792 | 0.00199207 | GSS\|glutathione synthetase |
| 813396 | 0.006131911 | KIAA1204\|KIAA1204 protein |
| 242706 | 0.033985397 | HSPC274\|HSPC274 protein |
| 173878 | 0.005539042 | MGC14327\|hypothetical protein MGC14327 |
| 52103 | 3.48E−05 | FLJ23045\|hypothetical protein FLJ23045 |
| 450810 | 0.031458387 | FLJ12154\|hypothetical protein FLJ12154 |
| 592630 | 0.002077599 | MINPP1\|multiple inositol polyphosphate histidine phosphatase, 1 |
| 1156538 | 4.81E−16 | ESTs |
| 487444 | 0.015004486 | ARPP-19\|cyclic AMP phosphoprotein, 19 kD |
| 273454 | 0.016029967 | ESTs |
| 2296063 | 0.010400431 | KIAA0528\|KIAA0528 gene product |
| 120138 | 0.000909767 | JDP1\|J domain containing protein 1 |
| 810063 | 0.016542848 | GFER\|growth factor, erv1 (S. cerevisiae)-like (augmenter of liver regeneration) |
| 1635022 | 0.007124761 | DDAH2\|dimethylarginine dimethylaminohydrolase 2 |
| 144740 | 6.16E−11 | SDCCAG28\|serologically defined colon cancer antigen 28 |
| 470061 | 8.03E−07 | SIAH2\|seven in absentia (Drosophila) homolog 2 |
| 1631820 | 0.038581914 | ESTs |
| 1456060 | 0.004330191 | ESTs, Weakly similar to A46010 X-linked retinopathy protein [H. sapiens] |
| 230271 | 0.000257296 | AD-017\|glycosyltransferase AD-017 |
| 138189 | 4.90E−09 | WFS1\|Wolfram syndrome 1 (wolframin) |
| 842838 | 0.02024356 | TADA3L\|transcriptional adaptor 3 (ADA3, yeast homolog)-like (PCAF histone acetylase complex) |
| 2028876 | 7.96E−05 | SFRS5\|splicing factor, arginine/serine-rich 5 |
| 1632487 | 0.02848343 | WISP2\|WNT1 inducible signaling pathway protein 2 |
| 530875 | 0.001185188 | TKT\|transketolase (Wernicke-Korsakoff syndrome) |
| 814350 | 0.010880033 | IDE\|insulin-degrading enzyme |
| 432108 | 0.001273848 | I-1\|imidazoline receptor candidate |
| 814353 | 0.012831119 | PMAIP1\|phorbol-12-myristate-13-acetate-induced protein 1 |
| 726147 | 0.024330122 | MAP2K4\|mitogen-activated protein kinase kinase 4 |
| 23776 | 3.95E−07 | QDPR\|quinoid dihydropteridine reductase |
| 503338 | 0.000584849 | WT1\|Wilms tumor 1 |
| 156117 | 0.033291452 | Homo sapiens, clone IMAGE: 3884331, mRNA, partial cds |
| 768395 | 0.013459172 | ESTs |
| 898058 | 0.001034397 | ESTs |
| 1854648 | 0.000117862 | HPX\|hemopexin |
| 35147 | 2.97E−07 | ESTs, Weakly similar to UOG1_HUMAN UOG-1 PROTEIN [H. sapiens] |
| 2388571 | 0.001899953 | AKAP8\|A kinase (PRKA) anchor protein 8 |
| 809828 | 1.10E−13 | E2F5\|E2F transcription factor 5, p130-binding |
| 811024 | 7.03E−05 | BST2\|bone marrow stromal cell antigen 2 |
| 625923 | 0.015565633 | PCK2\|phosphoenolpyruvate carboxykinase 2 (mitochondrial) |
| 1882051 | 0.014745604 | FLJ20080\|hypothetical protein FLJ20080 |
| 121898 | 2.79E−06 | Homo sapiens mRNA; cDNA DKFZp586J0720 (from clone DKFZp586J0720) |
| 1583198 | 1.28E−06 | ESTs, Weakly similar to S65824 reverse transcriptase homolog [H. sapiens] |
| 1031727 | 0.012784217 | SIRT2\|sirtuin (silent mating type information regulation 2, S. cerevisiae, homolog) 2 |
| 810088 | 0.025874177 | FLJ20258\|hypothetical protein FLJ20258 |
| 241798 | 0.03154152 | Homo sapiens cDNA FLJ30407 fis, clone BRACE2008553 |
| 85561 | 4.23E−05 | CYP2A7\|cytochrome P450, subfamily IIA (phenobarbital-inducible), polypeptide 7 |
| 809838 | 0.032429761 | EST |
| 743146 | 7.92E−05 | FLJ23403\|hypothetical protein FLJ23403 |
| 489079 | 0.003602021 | IL2RB\|interleukin 2 receptor, beta |
| 361698 | 0.002131744 | LIPE\|lipase, hormone-sensitive |
| 49204 | 0.00050472 | Homo sapiens mRNA; cDNA DKFZp566A1046 (from clone DKFZp566A1046) |
| 811038 | 0.016882187 | LOC51238\|hypothetical protein |
| 2012454 | 0.045950427 | Homo sapiens, clone IMAGE: 4391536, mRNA |
| 155197 | 3.41E−06 | ESTs |
| 470092 | 0.001366354 | LARGE\|like-glycosyltransferase |
| 1412245 | 0.016770876 | CPA2\|carboxypeptidase A2 (pancreatic) |
| 488422 | 0.028019793 | HMG20B\|high-mobility group 20B |
| 1635059 | 2.23E−09 | Homo sapiens, clone MGC: 16638 IMAGE: 4121964, mRNA, complete cds |
| 345663 | 0.002436388 | RAMP3\|receptor (calcitonin) activity modifying protein 3 |
| 42389 | 0.002362537 | ESTs, Weakly similar to Z195_HUMAN ZINC FINGER PROTEIN 195 [H. sapiens] |
| 203846 | 0.037582255 | FLJ22060\|hypothetical protein FLJ22060 |
| 1894519 | 0.000251134 | FLJ12085\|hypothetical protein FLJ12085 |
| 74070 | 0.000230079 | ENSA\|endosulfine alpha |
| 627542 | 6.23E−06 | BLVRA\|biliverdin reductase A |
| 773240 | 0.02515722 | ALG5\|Alg5, S. cerevisiae, homolog of |
| 1562231 | 0.007588458 | SETBP1\|SET binding protein 1 |
| 1635062 | 9.01E−06 | DKFZP586A011\|DKFZP586A011 protein |
| 773248 | 0.000803586 | Homo sapiens cDNA FLJ31373 fis, clone NB9N42000342 |
| 898083 | 0.041184366 | HBOA\|histone acetyltransferase |
| 291426 | 0.001816883 | KIAA1846\|similar to acetyl-coenzyme A synthetase |
| 123802 | 1.92E−06 | MFAP1\|microfibrillar-associated protein 1 |
| 177419 | 9.51E−05 | SH3GLB2\|SH3-domain, GRB2-like, endophilin B2 |
| 309316 | 0.040000458 | LOC58498\|myosin light chain 2a |
| 627555 | 0.001343272 | MGC3020\|hypothetical protein MGC3020 |
| 1637751 | 0.001227916 | OXA1L\|oxidase (cytochrome c) assembly 1-like |
| 49229 | 0.000209018 | GPHN\|gephyrin |
| 781418 | 0.011924329 | NPIP\|nuclear pore complex interacting protein |
| 767775 | 0.011293731 | LOC51242\|hypothetical protein |
| 48285 | 0.000352444 | PIG11\|p53-induced protein |
| 785701 | 0.011615484 | RAB31\|RAB31, member RAS oncogene family |
| 809869 | 0.020168182 | ESTs |
| 810402 | 0.002154308 | LOC51248\|hypothetical protein |
| 785708 | 0.044838359 | ESTs, Weakly similar to O4HUD1 debrisoquine 4-hydroxylase [H. sapiens] |
| 2306804 | 1.41E−11 | PSMD13\|proteasome (prosome, macropain) 26S subunit, non-ATPase, 13 |

TABLE 2
Genes that are overexpressed in ER negative cells, and underexpressed in ER positive cells

| Clone_ID | P_value | Gene_Description |
|---|---|---|
| 1557714 | 0.000249393 | ADAM19\|a disintegrin and metalloproteinase domain 19 (meltrin beta) |
| 340745 | 0.000173284 | Homo sapiens cDNA FLJ31683 fis, clone NT2RI2005353 |
| 325365 | 0.00025078 | HRB2\|HIV-1 rev binding protein 2 |
| 1467905 | 0.032429761 | NSG-X\|brain and nasopharyngeal carcinoma susceptibility protein |
| 757225 | 0.002362784 | SNX3\|sorting nexin 3 |
| 140806 | 0.002900139 | PAM\|peptidylglycine alpha-amidating monooxygenase |
| 796134 | 0.000177795 | MAP3K7\|mitogen-activated protein kinase kinase kinase 7 |
| 842896 | 0.018473624 | DKFZp762L0311\|hypothetical protein DKFZp762L0311 |
| 1550894 | 3.48E−08 | ESTs, Highly similar to A46297 beta-1,6-N-acetylglucosaminyltransferase [H. sapiens] |
| 813751 | 0.028826444 | SIAT4C\|sialyltransferase 4C (beta-galactosidase alpha-2,3-sialytransferase) |
| 740925 | 0.012998471 | INDO\|indoleamine-pyrrole 2,3 dioxygenase |
| 795191 | 1.39E−05 | XPNPEP1\|X-prolyl aminopeptidase (aminopeptidase P) 1, soluble |
| 824179 | 0.016037507 | KIAA0655\|huntingtin interacting protein-1-related |
| 731023 | 0.020902723 | WDR5\|WD repeat domain 5 |
| 2016775 | 4.63E−09 | GPRC5B\|G protein-coupled receptor, family C, group 5, member B |
| 324712 | 0.023557456 | RPS6KB2\|ribosomal protein S6 kinase, 70 kD, polypeptide 2 |
| 2326057 | 0.000136732 | MLN51\|MLN51 protein |
| 795197 | 0.000998067 | UBE2E2\|ubiquitin-conjugating enzyme E2E 2 (homologous to yeast UBC4/5) |
| 1570427 | 0.000231261 | MGC4309\|hypothetical protein MGC4309 |
| 199367 | 2.20E−05 | TNRC3\|trinucleotide repeat containing 3 |
| 40021 | 0.002599711 | HR\|hairless (mouse) homolog |
| 884355 | 0.000576316 | WASF3\|WAS protein family, member 3 |
| 1257131 | 2.20E−05 | ESTs, Weakly similar to SYN1 MOUSE SYNAPSIN I [M. musculus] |
| 731031 | 5.77E−06 | LOC51622\|CGI-43 protein |
| 415191 | 1.80E−12 | KIAA0161\|KIAA0161 gene product |
| 1637781 | 0.00021037 | EST |
| 1858992 | 0.004823705 | KIAA0615\|KIAA0615 gene product |
| 712454 | 2.82E−06 | Homo sapiens mRNA; cDNA DKFZp434O1317 (from clone DKFZp434O1317) |
| 1911099 | 0.02187025 | SCYA8\|small inducible cytokine subfamily A (Cys-Cys), member 8 (monocyte chemotactic protein 2) |
| 785733 | 1.41E−05 | FLJ12892\|hypothetical protein FLJ12892 |
| 2009574 | 0.000167648 | T\|T brachyury (mouse) homolog |
| 490753 | 0.001096449 | FLJ20420\|hypothetical protein FLJ20420 |
| 502753 | 0.003728424 | ANGPT2\|angiopoietin 2 |
| 172517 | 0.004544404 | HPCAL1\|hippocalcin-like 1 |
| 731044 | 0.006043174 | GLRX2\|glutaredoxin 2 |
| 155532 | 2.88E−07 | ESTs, Highly similar to bK989H11.1 [H. sapiens] |
| 731047 | 5.13E−08 | ESTs |
| 49266 | 0.001856861 | LOC51667\|NEDD8 ultimate buster-1 |
| 1723604 | 0.000868338 | MSP\|mosaic serine protease |
| 1637791 | 0.001388783 | MGC5350\|hypothetical protein MGC5350 |
| 1759290 | 9.79E−05 | FLJ11264\|hypothetical protein FLJ11264 |
| 221828 | 0.003359378 | ATSV\|axonal transport of synaptic vesicles |
| 291478 | 1.72E−06 | RUNX3\|runt-related transcription factor 3 |
| 141495 | 0.009886773 | SULT2B1\|sulfotransferase family, cytosolic, 2B, member 1 |
| 1534888 | 1.05E−06 | TNFRSF21\|tumor necrosis factor receptor superfamily, member 21 |
| 784005 | 0.003109725 | CARD12\|caspase recruitment domain protein 12 |
| 120516 | 0.004325708 | ESTs |
| 812048 | 5.76E−07 | PRNP\|prion protein (p27-30) (Creutzfeld-Jakob disease, Gerstmann-Strausler-Scheinker syndrome, fatal |
| | | familial insomnia) |
| 563598 | 1.56E−09 | GABRP\|gamma-aminobutyric acid (GABA) A receptor, pi |
| 759948 | 3.06E−06 | S100B\|S100 calcium-binding protein, beta (neural) |
| 757265 | 0.000137487 | MGC4399\|hypothetical protein MGC4399 |
| 1933324 | 0.017405243 | MPZ\|myelin protein zero (Charcot-Marie-Tooth neuropathy 1B) |
| 2027515 | 0.000231471 | SFN\|stratifin |
| 1614140 | 0.018506461 | LOC51285\|Ris |
| 526297 | 0.020076393 | NTSR1\|neurotensin receptor 1 (high affinity) |
| 113300 | 0.000965053 | TRIM14\|tripartite motif-containing 14 |
| 1587847 | 0.000486268 | MCM6\|minichromosome maintenance deficient (mis5, S. pombe) 6 |
| 40056 | 0.000446292 | CSPG4\|chondroitin sulfate proteoglycan 4 (melanoma-associated) |
| 270505 | 0.019185378 | MMP14\|matrix metalloproteinase 14 (membrane-inserted) |
| 2017754 | 0.015486907 | DGSI\|DiGeorge syndrome critical region gene DGSI; likely ortholog of mouse expressed sequence 2 |
| | | embryonic lethal |
| 1522734 | 0.039850127 | Homo sapiens cDNA FLJ31090 fis, clone IMR321000102 |
| 784016 | 0.000332608 | FLJ20624\|hypothetical protein FLJ20624 |
| 884388 | 0.017638942 | FLJ21103\|hypothetical protein FLJ21103 |
| 490772 | 0.000274015 | SNRPA1\|small nuclear ribonucleoprotein polypeptide A' |
| 810454 | 1.82E−05 | BACE2\|beta-site APP-cleaving enzyme 2 |
| 490777 | 0.001468215 | EST |
| 49284 | 2.69E−08 | FGF11\|fibroblast growth factor 11 |
| 1500815 | 0.003016551 | Homo sapiens cDNA: FLJ22130 fis, clone HEP19632 |
| 126413 | 0.013564319 | ITIH2\|inter-alpha (globulin) inhibitor, H2 polypeptide |
| 1631132 | 2.58E−05 | PHT2\|peptide transporter 3 |
| 593690 | 0.000827726 | TNFSF13B\|tumor necrosis factor (ligand) superfamily, member 13b |
| 133637 | 0.007572041 | PRKDC\|protein kinase, DNA-activated, catalytic polypeptide |
| 221846 | 0.006921653 | CHES1\|checkpoint suppressor 1 |
| 25763 | 0.000129642 | ANKH\|ankylosis, progressive (mouse) homolog |
| 68950 | 3.39E−09 | CCNE1\|cyclin E1 |
| 788309 | 0.035143688 | RMP\|RPB5-mediating protein |
| 730410 | 0.001453479 | LCK\|lymphocyte-specific protein tyrosine kinase |
| 503725 | 0.010812215 | ESTs |
| 346688 | 0.001091951 | MIA\|melanoma inhibitory activity |
| 2306860 | 0.000346451 | MAD2L2\|MAD2 (mitotic arrest deficient, yeast, homolog)-like 2 |
| 782428 | 0.044484074 | C1orf16\|chromosome 1 open reading frame 16 |
| 242037 | 6.76E−06 | FLJ20746\|hypothetical protein |
| 358052 | 0.005746627 | ESTs |
| 1946448 | 2.88E−05 | CAV2\|caveolin 2 |
| 2046118 | 2.22E−06 | RAD54L\|RAD54 (S. cerevisiae)-like |
| 1471841 | 0.000169912 | ATP1A1\|ATPase, Na+/K+ transporting, alpha 1 polypeptide |
| 824510 | 0.00041118 | LOC51647\|CGI-128 protein |
| 141806 | 0.002003247 | TNFAIP3\|tumor necrosis factor, alpha-induced protein 3 |
| 486110 | 0.004730919 | PFN2\|profilin 2 |
| 1586124 | 3.20E−05 | FLJ11413\|hypothetical protein FLJ11413 |
| 840556 | 0.00087773 | EIF4EL3\|eukaryotic translation initiation factor 4E-like 3 |
| 1589468 | 0.001850652 | EMP1\|epithelial membrane protein 1 |
| 2019376 | 0.03681114 | MGC2718\|hypothetical protein MGC2718 |
| 346696 | 0.021270804 | TEAD4\|TEA domain family member 4 |
| 796197 | 6.83E−06 | DMD\|dystrophin (muscular dystrophy, Duchenne and Becker types), includes DXS142, DXS164, |
| | | DXS206, DXS230, DXS239, DXS268, DXS269, DXS270, DXS272 |
| 796198 | 0.004000613 | EFNB2\|ephrin-B2 |
| 731080 | 2.98E−05 | FLJ12661\|hypothetical protein FLJ12661 |
| 324772 | 0.042068695 | LCCP\|Leman coiled-coil protein |
| 950690 | 0.035127941 | CCNA2\|cyclin A2 |
| 841501 | 2.51E−07 | KIAA0102\|KIAA0102 gene product |
| 1466237 | 0.002257726 | TES\|testis derived transcript (3 LIM domains) |
| 1457416 | 0.004076409 | KIAA0728\|KIAA0728 protein |
| 153977 | 0.00188129 | POP4\|POP4 (processing of precursor, S. cerevisiae) homolog |
| 840567 | 0.000523423 | TM4SF1\|transmembrane 4 superfamily member 1 |
| 768000 | 0.000909544 | RNASEH1\|ribonuclease H1 |
| 341763 | 0.044284858 | CASP5\|caspase 5, apoptosis-related cysteine protease |
| 768007 | 1.46E−07 | CL683\|weakly similar to glutathione peroxidase 2 |
| 768008 | 0.000389422 | BAG2\|BCL2-associated athanogene 2 |
| 39833 | 9.83E−05 | CPVL\|carboxypeptidase, vitellogenic-like |
| 843110 | 0.0039532 | GDI1\|GDP dissociation inhibitor 1 |
| 155583 | 0.034919482 | TRAF1\|TNF receptor-associated factor 1 |
| 810485 | 0.000238475 | ID1\|inhibitor of DNA binding 1, dominant negative helix-loop-helix protein |
| 767068 | 4.46E−13 | DKFZP586G1517\|DKFZP586G1517 protein |
| 108801 | 3.53E−05 | FLJ20392\|hypothetical protein FLJ20392 |
| 290866 | 1.21E−06 | RAF1\|v-raf-1 murine leukemia viral oncogene homolog 1 |
| 1604703 | 0.009410539 | HLA-F\|major histocompatibility complex, class I, F |
| 1584540 | 0.000352001 | Homo sapiens mRNA; cDNA DKFZp586M2022 (from clone DKFZp586M2022) |
| 1590021 | 3.88E−07 | ETV6\|ets variant gene 6 (TEL oncogene) |
| 204897 | 6.91E−07 | PLCG2\|phospholipase C, gamma 2 (phosphatidylinositol-specific) |
| 769751 | 2.75E−07 | KIAA0095\|KIAA0095 gene product |
| 430928 | 0.005339331 | BARD1\|BRCA1 associated RING domain 1 |
| 823598 | 0.029081002 | PSMD12\|proteasome (prosome, macropain) 26S subunit, non-ATPase, 12 |
| 843121 | 0.000124807 | CLIC1\|chloride intracellular channel 1 |
| 139558 | 0.008816 | Homo sapiens gastric cancer-related protein GCYS-20 (gcys-20) mRNA, complete cds |
| 843126 | 0.010510953 | SIPL\|SIPL protein |
| 753313 | 0.002864496 | LAPTM5\|Lysosomal-associated multispanning membrane protein-5 |
| 175123 | 0.037853387 | MAPK7\|mitogen-activated protein kinase 7 |
| 824547 | 0.000238959 | MHC2TA\|MHC class II transactivator |
| 1592715 | 1.98E−05 | HOMER-3\|Homer, neuronal immediate early gene, 3 |
| 272155 | 0.006067385 | SRGAP2\|KIAA0456 protein |
| 1845169 | 0.006064365 | RAB35\|RAB35, member RAS oncogene family |
| 489489 | 2.07E−07 | LBR\|lamin B receptor |
| 629542 | 0.002987142 | MYH1\|myosin, heavy polypeptide 1, skeletal muscle, adult |
| 490023 | 1.39E−05 | WNT5B\|wingless-type MMTV integration site family, member 5B |
| 2430676 | 0.039914681 | EZFIT\|endothelial zinc finger protein induced by tumor necrosis factor alpha |
| 868396 | 1.13E−05 | LOC64148\|17 kD fetal brain protein |
| 782460 | 0.009363602 | Homo sapiens cDNA FLJ10500 fis, clone NT2RP2000369 |
| 1948085 | 0.000313027 | CBR3\|carbonyl reductase 3 |
| 235938 | 0.009238649 | BAK1\|BCL2-antagonist/killer 1 |
| 897822 | 0.003765884 | SYK\|spleen tyrosine kinase |
| 586803 | 7.34E−08 | PGF\|placental growth factor, vascular endothelial growth factor-related protein |
| 746105 | 4.10E−05 | LTRPC7\|homolog of mouse transient receptor potential-phospholipase C-interacting kinase CHaK; |
| | | hypothetical protein FLJ20117 |
| 2271240 | 0.001624493 | RPS12\|ribosomal protein S12 |
| 32231 | 0.000744224 | FLJ12442\|hypothetical protein FLJ12442 |
| 1394099 | 0.000187841 | MGC5528\|hypothetical protein MGC5528 |
| 202514 | 0.000114404 | DNMT3A\|DNA (cytosine-5-)-methyltransferase 3 alpha |
| 788355 | 0.001475302 | STAG3\|stromal antigen 3 |
| 815737 | 3.71E−09 | ATP5A1\|ATP synthase, H+ transporting, mitochondrial F1 complex, alpha subunit, isoform 1, cardiac |
| | | muscle |
| 726582 | 1.36E−05 | ARP3BETA\|actin-related protein 3-beta |
| 244764 | 0.001161294 | B7-H3\|B7 homolog 3 |
| 814798 | 2.03E−08 | ALDH1A3\|aldehyde dehydrogenase 1 family, memberA3 |
| 854644 | 0.011182578 | NPC2\|Niemann-Pick disease, type C2 gene |
| 2011138 | 0.001310947 | KIAA1036\|KIAA1036 protein |
| 194804 | 0.0121963 | PITPN\|phosphotidylinositol transfer protein |
| 209066 | 0.001333458 | STK15\|serine/threonine kinase 15 |
| 121530 | 0.023976232 | MGC10966\|hypothetical protein MGC10966 |
| 430954 | 9.12E−06 | FLJ22341\|hypothetical protein FLJ22341 |
| 687054 | 0.016868879 | RELB\|v-rel avian reticuloendotheliosis viral oncogene homolog B (nuclear factor of kappa light |
| | | polypeptide gene enhancer in B-cells 3) |
| 624627 | 0.004546557 | RRM2\|ribonucleotide reductase M2 polypeptide |
| 357450 | 0.030931622 | MTVR\|Mouse Mammary Turmor Virus Receptor homolog |
| 25030 | 0.006951821 | PCMF\|potassium channel modulatory factor |
| 756687 | 0.004407309 | CD36L1\|CD36 antigen (collagen type I receptor, thrombospondin receptor)-like 1 |
| 666879 | 4.67E−06 | ANXA8\|annexin A8 |
| 68225 | 0.036628745 | Homo sapiens pTM5 mariner-like transposon mRNA, partial sequence |
| 486175 | 0.0005382 | SLC16A1\|solute carrier family 16 (monocarboxylic acid transporters), member 1 |
| 796539 | 0.000602202 | TRIM28\|tripartite motif-containing 28 |
| 745189 | 9.02E−05 | DAZAP1\|DAZ associated protein 1 |
| 810813 | 0.001864702 | S100A2\|S100 calcium-binding protein A2 |
| 486179 | 0.000858947 | NFAT5\|nuclear factor of activated T-cells 5, tonicity-responsive |
| 149355 | 0.011544468 | TRAM\|translocating chain-associating membrane protein |
| 1476053 | 0.03289313 | RAD51\|RAD51 (S. cerevisiae) homolog (E coli RecA homolog) |
| 2062507 | 2.18E−07 | KLF5\|Kruppel-like factor 5 (intestinal) |
| 1634832 | 0.026304968 | WSX-1\|class I cytokine receptor |
| 1420842 | 0.01376248 | MALT1\|mucosa associated lymphoid tissue lymphoma translocation gene 1 |
| 489805 | 0.028082851 | HSF4\|heat shock transcription factor 4 |
| 611532 | 7.05E−06 | TNNI2\|troponin I, skeletal, fast |
| 796542 | 8.87E−05 | ETV5\|ets variant gene 5 (ets-related molecule) |
| 727551 | 1.40E−08 | IRF2\|interferon regulatory factor 2 |
| 50987 | 0.016625494 | PABPC4\|poly(A)-binding protein, cytoplasmic 4 (inducible form) |
| 823925 | 0.001754502 | LOC51177\|CK2 interacting protein 1; HQ0024c protein |
| 121551 | 1.86E−08 | LAD1\|ladinin 1 |
| 869375 | 0.003859746 | IDH2\|isocitrate dehydrogenase 2 (NADP+), mitochondrial |
| 73852 | 0.017664041 | Homo sapiens cDNA FLJ14388 fis, clone HEMBA1002716 |
| 252953 | 0.019402013 | GS3786\|predicted osteoblast protein |
| 2321529 | 0.026304832 | KCNG1\|potassium voltage-gated channel, subfamily G, member 1 |
| 1476065 | 0.000528763 | STMN1\|stathmin 1/oncoprotein 18 |
| 854678 | 0.015997677 | DKFZP566J091\|hypothetical protein DKFZp566J091 |
| 196435 | 0.00388413 | ESTs, Weakly similar to KIAA0738 protein [H. sapiens] |
| 843174 | 0.001025358 | PGM1\|phosphoglucomutase 1 |
| 52594 | 0.036156197 | PCDH8\|protocadherin 8 |
| 20115 | 0.021509161 | SLC9A6\|solute carrier family 9 (sodium/hydrogen exchanger), isoform 6 |
| 669443 | 0.003570072 | HSF2\|heat shock transcription factor 2 |
| 770355 | 0.014696243 | LSS\|lanosterol synthase (2,3-oxidosqualene-lanosterol cyclase) |
| 51947 | 0.025874177 | Homo sapiens, clone MGC: 20842 IMAGE: 4542449, mRNA, complete cds |
| 809503 | 0.001947255 | ESTs, Weakly similar to AC004858 3 U1 small ribonucleoprotein 1SNRP homolog [H. sapiens] |
| 193106 | 0.003498139 | ATP5G3\|ATP synthase, H+ transporting, mitochondrial F0 complex, subunit c (subunit 9) isoform 3 |
| 1702742 | 2.07E−07 | SLC7A5\|solute carrier family 7 (cationic amino acid transporter, y+ system), member 5 |
| 815781 | 0.046183854 | HSP105B\|heat shock 105 kD |
| 782800 | 0.046687885 | UQCRFS1\|ubiquinol-cytochrome c reductase, Rieske iron-sulfur polypeptide 1 |
| 68259 | 0.000734236 | RENT2\|regulator of nonsense transcripts 2 |
| 121577 | 1.83E−06 | FLJ22393\|hypothetical protein FLJ22393 |
| 1912458 | 1.43E−08 | IL12A\|interleukin 12A (natural killer cell stimulatory factor 1, cytotoxic lymphocyte maturation factor 1, |
| | | p35) |
| 70533 | 0.00154863 | HPS\|Hermansky-Pudlak syndrome |
| 1660666 | 0.017638942 | CA5B\|carbonic anhydrase VB, mitochondrial |
| 51950 | 0.002759641 | AQP1\|aquaporin 1 (channel-forming integral protein, 28 kD) |
| 291880 | 2.95E−05 | MFAP2\|microfibrillar-associated protein 2 |
| 1555478 | 0.031251603 | ST2\|syntenin-2 protein |
| 51955 | 1.54E−08 | FLJ22282\|hypothetical protein FLJ22282 |
| 79761 | 0.038130306 | TMPO\|thymopoietin |
| 843195 | 0.005767924 | PSPH\|phosphoserine phosphatase |
| 292388-2 | 0.000484852 | LOC55977\|hypothetical protein 24636 |
| 246704 | 0.013447399 | RAI\|RelA-associated inhibitor |
| 782811 | 6.86E−07 | HMGIY\|high-mobility group (nonhistone chromosomal) protein isoforms I and Y |
| 271568 | 0.000866786 | ACTR3\|ARP3 (actin-related protein 3, yeast) homolog |
| 2307119 | 2.37E−06 | INPP4A\|inositol polyphosphate-4-phosphatase, type I, 107 kD |
| 838662 | 0.010070202 | HCNGP\|transcriptional regulator protein |
| 810859 | 0.047573891 | NK4\|natural killer cell transcript 4 |
| 241481 | 0.014557615 | CASP10\|caspase 10, apoptosis-related cysteine protease |
| 2094232 | 5.18E−06 | EGLN1\|EGL nine (C. elegans) homolog 1 |
| 267691 | 0.007853541 | FLJ20360\|hypothetical protein FLJ20360 |
| 788705 | 0.018859374 | USF1\|upstream transcription factor 1 |
| 592359 | 0.037494688 | HKE4\|HLA class II region expressed gene KE4 |
| 2159983 | 3.25E−08 | PORIMIN\|pro-oncosis receptor inducing membrane injury gene |
| 2016426 | 0.009898035 | KIAA0664\|KIAA0664 protein |
| 270917 | 3.65E−08 | SFRP1\|secreted frizzled-related protein 1 |
| 625623 | 0.001515188 | Homo sapiens mRNA; cDNA DKFZp434F152 (from clone DKFZp434F152) |
| 1492426 | 0.000486718 | C19orf3\|chromosome 19 open reading frame 3 |
| 1556433 | 0.013873827 | GRO3\|GRO3 oncogene |
| 2010543 | 0.000219946 | DDX28\|DEAD/H (Asp-Glu-Ala-Asp/His) box polypeptide 28 |
| 1466621 | 0.023035247 | ATP2C1\|ATPase, Ca++ transporting, type 2C, member 1 |
| 299388 | 1.27E−06 | PP15\|nuclear transport factor 2 (placental protein 15) |
| 1469966 | 0.002597189 | DLL3\|delta-like 3 (Drosophila) |
| 771323 | 6.28E−10 | PLOD\|procollagen-lysine, 2-oxoglutarate 5-dioxygenase (lysine hydroxylase, Ehlers-Danlos syndrome |
| | | type VI) |
| 306444 | 0.011573645 | TAF11\|TAF11 RNA polymerase II, TATA box binding protein (TBP)-associated factor, 28 kD |
| 382654 | 0.005845554 | PCSK2\|proprotein convertase subtilisin/kexin type 2 |
| 544639 | 1.14E−07 | ESTs |
| 306446 | 0.001825487 | C11orf24\|chromosome 11 open reading frame 24 |
| 838676 | 0.017828051 | CAPN6\|calpain 6 |
| 42070 | 0.000619438 | NT5\|5′ nucleotidase (CD73) |
| 809530 | 0.009640123 | MCM2\|minichromosome maintenance deficient (S. cerevisiae) 2 (mitotin) |
| 754346 | 2.60E−05 | ESTs, Weakly similar to B49647 GTP-binding protein rab8 [H. sapiens] |
| 882402 | 0.003800629 | KIAA0692\|KIAA0692 protein |
| 770388 | 0.004124332 | CLDN4\|claudin 4 |
| 815015 | 0.000124184 | TERA\|TERA protein |
| 1604005 | 1.58E−05 | SH3KBP1\|SH3-domain kinase binding protein 1 |
| 789376 | 0.02515722 | TXNRD1\|thioredoxin reductase 1 |
| 814072 | 0.000473 | Homo sapiens, clone MGC: 19517 IMAGE: 4335816, mRNA, complete cds |
| 504791 | 0.004943728 | GSTA4\|glutathione S-transferase A4 |
| 3220641 | 0.018173943 | OR7E24P\|olfactory receptor, family 7, subfamily E, member 24 pseudogene |
| 325033 | 0.016712654 | ESTs |
| 814076 | 0.01354434 | NSAP1\|NS1-associated protein 1 |
| 811817 | 0.009457359 | FLJ21032\|hypothetical protein FLJ21032 |
| 510273 | 0.030089117 | PLEC1\|plectin 1, intermediate filament binding protein, 500 kD |
| 377048 | 0.000173284 | MYO1B\|myosin IB |
| 1034776 | 0.000124401 | AD037\|AD037 protein |
| 322494 | 1.89E−05 | CBFB\|core-binding factor, beta subunit |
| 46367 | 0.001719417 | CHK\|choline kinase |
| 1519143 | 0.000508533 | RISC\|likely homolog of rat and mouse retinoid-inducible serine carboxypeptidase |
| 789382 | 0.029514943 | NOTCH4\|Notch (Drosophila) homolog 4 |
| 263716 | 0.003686053 | COL6A1\|collagen, type VI, alpha 1 |
| 823982 | 0.00726442 | TRIM26\|tripartite motif-containing 26 |
| 140197 | 0.004538178 | LOC51299\|neuritin |
| 786048 | 0.000316752 | E2F4\|E2F transcription factor 4, p107/p130-binding |
| 1947804 | 0.014717852 | TREX1\|three prime repair exonuclease 1 |
| 414999 | 0.00278887 | ETV4\|ets variant gene 4 (E1A enhancer-binding protein, E1AF) |
| 279085 | 0.012204566 | MYO9B\|myosin IXB |
| 259842 | 2.42E−05 | PMPCB\|peptidase (mitochondrial processing) beta |
| 232697 | 0.003201006 | TIMM10\|translocase of inner mitochondrial membrane 10 (yeast) homolog |
| 809557 | 0.006003565 | MCM3\|minichromosome maintenance deficient (S. cerevisiae) 3 |
| 725223 | 1.33E−05 | KIAA0077\|KIAA0077 protein |
| 840978 | 6.56E−06 | CD81\|CD81 antigen (target of antiproliferative antibody 1) |
| 490414 | 2.13E−08 | C2orf2\|chromosome 2 open reading frame 2 |
| 786053 | 0.038091532 | Homo sapiens cDNA FLJ30898 fis, clone FEBRA2005572 |
| 564981 | 2.34E−05 | Homo sapiens, Similar to RIKEN cDNA 2810433K01 gene, clone MGC: 10200 IMAGE: 3909951, mRNA, |
| | | complete cds |
| 810899 | 9.04E−06 | ESTs |
| 288999 | 0.000887435 | SPEC1\|small protein effector 1 of Cdc42 |
| 1473876 | 4.29E−05 | KIAA0187\|KIAA0187 gene product |
| 796904 | 1.58E−05 | PLAGL1\|pleiomorphic adenoma gene-like 1 |
| 344720 | 0.001678075 | GYPC\|glycophorin C (Gerbich blood group) |
| 1534544 | 0.004653836 | PCCX2\|protein containing CXXC domain 2 |
| 126858 | 0.038264032 | SOAT1\|sterol O-acyltransferase (acyl-Coenzyme A: cholesterol acyltransferase) 1 |
| 202904 | 0.00238904 | SFRS7\|splicing factor, arginine/serine-rich 7 (35 kD) |
| 840984 | 0.004457062 | CAV2\|caveolin 2 |
| 138672-2 | 5.39E−06 | ESTs |
| 882439 | 0.017638691 | CPR2\|cell cycle progression 2 protein |
| 2721773 | 0.027704333 | NOG\|noggin |
| 786067 | 8.88E−05 | CDC25B\|cell division cycle 25B |
| 1575059 | 0.028270162 | TBCC\|tubulin-specific chaperone c |
| 1947827 | 0.01489065 | MSTP028\|MSTP028 protein |
| 2390593 | 4.77E−06 | PTPN14\|protein tyrosine phosphatase, non-receptor type 14 |
| 767487 | 5.80E−05 | ARIH1\|ariadne (Drosophila) homolog, ubiquitin-conjugating enzyme E2-binding protein, 1 |
| 161484 | 4.46E−05 | DGAT2\|diacylglycerol O-acyltransferase homolog 2 (mouse) |
| 2052032 | 1.96E−07 | MYO10\|myosin X |
| 712139 | 2.31E−09 | ARL7\|ADP-ribosylation factor-like 7 |
| 770709 | 0.000831262 | KIAA1089\|KIAA1089 protein |
| 2017415 | 1.21E−05 | CENPA\|centromere protein A (17 kD) |
| 740620 | 2.38E−05 | TPM2\|tropomyosin 2 (beta) |
| 184354 | 6.99E−07 | EST |
| 182750 | 0.012645421 | MSI1\|Musashi (Drosophila) homolog 1 |
| 76481 | 0.024566797 | PTK9L\|protein tyrosine kinase 9-like (A6-related protein) |
| 785417 | 0.011581406 | NCUBE1\|non-canonical ubquitin conjugating enzyme 1 |
| 2313732 | 0.002401111 | PLAA\|phospholipase A2-activating protein |
| 1911864 | 0.005343697 | GNG4\|guanine nucleotide binding protein 4 |
| 503096 | 1.97E−05 | ESTs |
| 810119 | 3.53E−05 | SNAI1\|snail 1 (drosophila homolog), zinc finger protein |
| 713886 | 0.018673346 | CNN2\|calponin 2 |
| 281392 | 0.005737829 | Homo sapiens mRNA; cDNA DKFZp761G02121 (from clone DKFZp761G02121); partial cds |
| 431214 | 0.005427358 | FLJ12484\|hypothetical protein FLJ12484 |
| 205185 | 0.010013706 | THBD\|thrombomodulin |
| 813460 | 0.005587928 | PCSK7\|proprotein convertase subtilisin/kexin type 7 |
| 809588 | 0.022949968 | GGH\|gamma-glutamyl hydrolase (conjugase, folylpolygammaglutamyl hydrolase) |
| 1901310 | 0.001063475 | KIAA1209\|KIAA1209 protein |
| 257197 | 0.005064867 | NRBF-2\|nuclear receptor binding factor-2 |
| 502446 | 0.004574652 | DKFZP564A2416\|DKFZP564A2416 protein |
| 156962 | 0.016124526 | ESTs |
| 882459 | 0.007855578 | PPIC\|peptidylprolyl isomerase C (cyclophilin C) |
| 588822 | 0.000420841 | COVA1\|cytosolic ovarian carcinoma antigen 1 |
| 625683 | 4.54E−05 | NOC4\|neighbor of COX4 |
| 470128 | 2.92E−08 | MYO1E\|myosin IE |
| 233679 | 2.38E−05 | FLJ22362\|hypothetical protein FLJ22362 |
| 683177 | 0.016770876 | MAP4K5\|mitogen-activated protein kinase kinase kinase kinase 5 |
| 700792 | 0.026756579 | CDKN3\|cyclin-dependent kinase inhibitor 3 (CDK2-associated dual specificity phosphatase) |
| 882461 | 0.001070887 | ASS\|argininosuccinate synthetase |
| 1518538 | 0.000295474 | MGC4607\|hypothetical protein MGC4607 |
| 1517595 | 2.27E−06 | KIAA0175\|likely ortholog of maternal embryonic leucine zipper kinase |
| 950355 | 0.000138935 | ESTs, Weakly similar to TRHY_HUMAN TRICHOHYALI [H. sapiens] |
| 839682 | 4.07E−06 | UBE2N\|ubiquitin-conjugating enzyme E2N (homologous to yeast UBC13) |
| 625693 | 0.001165816 | MGC10911\|hypothetical protein MGC10911 |
| 289978 | 0.00024508 | UBL4\|ubiquitin-like 4 |
| 25440 | 0.003949353 | STAU2\|staufen (Drosophila, RNA-binding protein) homolog 2 |
| 346360 | 0.039034227 | LAT\|linker for activation of T cells |
| 1161155 | 0.000197727 | CDKN2A\|cyclin-dependent kinase inhibitor 2A (melanoma, p16, inhibits CDK4) |
| 2028949 | 0.034919482 | PRO1855\|hypothetical protein PRO1855 |
| 1925939 | 0.025350497 | PLAGL2\|pleiomorphic adenoma gene-like 2 |
| 32664 | 0.04577709 | GNAO1\|guanine nucleotide binding protein (G protein), alpha activating activity polypeptide O |
| 1542749 | 3.27E−08 | AMD1\|S-adenosylmethionine decarboxylase 1 |
| 2316305 | 0.000257732 | DKFZP566D213\|DKFZP566D213 protein |
| 724615 | 0.00452876 | CHC1\|chromosome condensation 1 |
| 951304 | 0.011615484 | KIAA0573\|KIAA0573 protein |
| 75859 | 4.68E−06 | NDRG2\|N-myc downstream-regulated gene 2 |
| 950361 | 0.002582165 | EST |
| 898122 | 0.003101713 | C7\|complement component 7 |
| 898123 | 1.31E−05 | GART\|phosphoribosylglycinamide formyltransferase, phosphoribosylglycinamide synthetase, |
| | | phosphoribosylaminoimidazole synthetase |
| 1881171 | 0.00258728 | ESTs |
| 594322 | 0.014086588 | PAPSS1\|3′-phosphoadenosine 5′-phosphosulfate synthase 1 |
| 530958 | 0.000152832 | SMOH\|smoothened (Drosophila) homolog |
| 813490 | 7.09E−07 | CORO1C\|coronin, actin-binding protein, 1C |
| 2308153 | 0.003570072 | ELL\|ELL gene (11-19 lysine-rich leukemia gene) |
| 785459 | 0.001112368 | SMTN\|smoothelin |
| 767817 | 7.55E−07 | POLR2F\|polymerase (RNA) II (DNA directed) polypeptide F |
| 153650 | 0.001255544 | KIAA1140\|KIAA1140 protein |
| 810156 | 0.007406628 | DTYMK\|deoxythymidylate kinase (thymidylate kinase) |
| 1035796 | 0.00021094 | ESTs, Weakly similar to T33068 hypothetical protein C35E7.9 - Caenorhabditis elegans [C. elegans] |
| 811897 | 0.002192122 | MKL1\|megakaryoblastic leukemia (translocation) 1 |
| 824203 | 0.018221151 | DNCLI2\|dynein, cytoplasmic, light intermediate polypeptide 2 |
| 744952 | 0.024481266 | ESTs, Moderately similar to UQHUR7 ubiquitin/ribosomal protein S27a, cytosolic [H. sapiens] |
| 85634 | 0.000676725 | C1S\|complement component 1, s subcomponent |
| 132395 | 0.008012849 | SRRM1\|serine/arginine repetitive matrix 1 |
| 2012523 | 6.10E−07 | FABP5\|fatty acid binding protein 5 (psoriasis-associated) |
| 773301 | 3.74E−12 | CDH3\|cadherin 3, type 1, P-cadherin (placental) |
| 811108 | 0.000222819 | TRIP6\|thyroid hormone receptor interactor 6 |
| 416959 | 1.42E−08 | NFIB\|nuclear factor I/B |
| 250699 | 0.019002987 | Homo sapiens, clone IMAGE: 3888869, mRNA, partial cds |
| 144849 | 3.25E−08 | CLP\|coactosin-like protein |
| 743220 | 0.000600627 | FLJ12517\|hypothetical protein FLJ12517 |
| 283328 | 0.000987968 | EST |
| 842939 | 0.022592867 | ADARB1\|adenosine deaminase, RNA-specific, B1 (homolog of rat RED1) |
| 417905 | 0.00632472 | Homo sapiens, Similar to RIKEN cDNA 5730528L13 gene, clone MGC: 17337 IMAGE: 4213591, mRNA, |
| | | complete cds |
| 809918 | 0.000355143 | FLJ21047\|hypothetical protein FLJ21047 |
| 811116 | 0.027129738 | Homo sapiens, clone IMAGE: 3632168, mRNA |
| 2014138 | 0.003381521 | FACL2\|fatty-acid-Coenzyme A ligase, long-chain 2 |
| 309092 | 3.07E−05 | APG5L\|APG5 (autophagy 5, S. cerevisiae)-like |
| 128811 | 0.000579172 | MGC2452\|hypothetical protein MGC2452 |
| 768496 | 0.011680975 | EBI3\|Epstein-Barr virus induced gene 3 |
| 772373 | 0.001284654 | Homo sapiens cDNA FLJ30865 fis, clone FEBRA2004099 |
| 140574 | 4.08E−13 | SCYD1\|small inducible cytokine subfamily D (Cys-X3-Cys), member 1 (fractalkine, neurotactin) |
| 263013 | 0.000365845 | PLOD2\|procollagen-lysine, 2-oxoglutarate 5-dioxygenase (lysine hydroxylase) 2 |
| 2161427-2 | 0.000159834 | PGR\|progesterone receptor |
| 1606790 | 0.002826291 | MGC12217\|hypothetical protein MGC12217 |
| 1509761 | 1.92E−06 | KRTHB6\|keratin, hair, basic, 6 (monilethrix) |
| 469924 | 0.00156327 | PCTP\|phosphatidylcholine transfer protein |
| 724652 | 0.000324169 | CD8B1\|CD8 antigen, beta polypeptide 1 (p37) |
| 773322 | 0.001724267 | SHOX2\|short stature homeobox 2 |
| 773324 | 0.00075407 | FLJ10439\|hypothetical protein FLJ10439 |
| 711552 | 0.004197328 | CBR1\|carbonyl reductase 1 |
| 773329 | 0.003063069 | KIAA1204\|KIAA1204 protein |
| 1422894 | 0.01526361 | NOTCH2\|Notch (Drosophila) homolog 2 |
| 2010949 | 0.000264549 | ME2\|malic enzyme 2, NAD(+)-dependent, mitochondrial |
| 897509 | 0.000195032 | ESTs, Weakly similar to S23650 retrovirus-related hypothetical protein II [H. sapiens] |
| 223176 | 0.004691656 | MAD\|MAX dimerization protein |
| 744980 | 0.029325674 | KIAA0304\|KIAA0304 gene product |
| 1518591 | 0.000137744 | EST |
| 784830 | 0.0001858 | D123\|D123 gene product |
| 431280 | 0.000580358 | EST |
| 246041 | 0.00099919 | MRPS35\|mitochondrial ribosomal protein S35 |
| 25499 | 0.031951505 | DKFZp564A176\|hypothetical protein DKFZp564A176 |
| 505059 | 0.017104301 | HSD11B1\|hydroxysteroid (11-beta) dehydrogenase 1 |
| 377441 | 0.000151691 | S100A3\|S100 calcium-binding protein A3 |
| 80727 | 3.81E−05 | ROR1\|receptor tyrosine kinase-like orphan receptor 1 |
| 814478 | 0.009751136 | BCL2A1\|BCL2-related protein A1 |
| 714106 | 0.003765884 | PLAU\|plasminogen activator, urokinase |
| 773330 | 0.000805296 | GPNMB\|glycoprotein (transmembrane) nmb |
| 343079 | 0.044429786 | Homo sapiens mRNA; cDNA DKFZp761P0114 (from clone DKFZp761P0114) |
| 773331 | 1.90E−05 | ESTs, Weakly similar to YK61_YEAST HYPOTHETICAL 39.6 KDA PROTEIN IN MTD1-NUP133 |
| | | INTERGENIC REGION [S. cerevisiae] |
| 324492 | 0.029371795 | MMP3\|matrix metalloproteinase 3 (stromelysin 1, progelatinase) |
| 132711 | 5.39E−08 | KLF5\|Kruppel-like factor 5 (intestinal) |
| 841226 | 0.005177763 | NS1-BP\|NS1-binding protein |
| 842968 | 0.000543163 | BUB1B\|budding uninhibited by benzimidazoles 1 (yeast homolog), beta |
| 795263 | 0.004420295 | FLJ22638\|hypothetical protein FLJ22638 |
| 2309141 | 0.007213754 | CD97\|CD97 antigen |
| 950700 | 0.002257726 | PP\|pyrophosphatase (inorganic) |
| 725618 | 0.007582436 | Homo sapiens mRNA containing (CAG)4 repeat, clone CZ-CAG-7 |
| 1655480 | 0.00653861 | ESTs |
| 377452 | 0.037494688 | LOC55818\|putative zinc finger protein |
| 845513 | 0.000814278 | AP47\|clathrin-associated protein AP47 |
| 811149 | 0.002784531 | C9orf3\|chromosome 9 open reading frame 3 |
| 160126 | 0.009121318 | COX10\|COX10 (yeast) homolog, cytochrome c oxidase assembly protein (heme A: farnesyltransferase) |
| 144880 | 8.33E−05 | LOC56932\|hypothetical protein from EUROIMAGE 1759349 |
| 144881 | 0.000416861 | CALU\|calumenin |
| 845519 | 0.002618542 | ATP5C1\|ATP synthase, H+ transporting, mitochondrial F1 complex, gamma polypeptide 1 |
| 884425 | 0.01173607 | CCT5\|chaperonin containing TCP1, subunit 5 (epsilon) |
| 950710 | 3.51E−06 | PCCA\|propionyl Coenzyme A carboxylase, alpha polypeptide |
| 2013515 | 6.64E−07 | SGK\|serum/glucocorticoid regulated kinase |
| 1869486 | 0.001399452 | PRKX\|protein kinase, X-linked |
| 897531 | 4.76E−06 | MCAM\|melanoma cell adhesion molecule |
| 782176 | 0.001737318 | LOC136442\|similar to MRJ gene for a member of the DNAJ protein family (H. sapiens) |
| 703707 | 8.41E−06 | ASPH\|aspartate beta-hydroxylase |
| 154657 | 0.030945971 | Homo sapiens cDNA: FLJ21286 fis, clone COL01915 |
| 753034 | 0.006100942 | NFIX\|nuclear factor I/X (CCAAT-binding transcription factor) |
| 753038 | 3.93E−06 | KIFC3\|kinesin family member C3 |
| 795280 | 0.017638942 | ESTs |
| 628602 | 0.003953106 | MYL3\|myosin, light polypeptide 3, alkali; ventricular, skeletal, slow |
| 813841 | 0.00266223 | PLAT\|plasminogen activator, tissue |
| 785804 | 0.000661605 | CECR5\|cat eye syndrome chromosome region, candidate 5 |
| 73531 | 2.64E−07 | NIFU\|nitrogen fixation cluster-like |
| 271219 | 0.010004815 | Homo sapiens cDNA FLJ12566 fis, clone NT2RM4000852 |
| 1879255 | 0.021427151 | DPYSL4\|dihydropyrimidinase-like 4 |
| 1913855 | 0.005854823 | MRPS17\|mitochondrial ribosomal protein S17 |
| 345787 | 1.23E−05 | HEC\|highly expressed in cancer, rich in leucine heptad repeats |
| 810504 | 0.020902723 | PLP2\|proteolipid protein 2 (colonic epithelium-enriched) |
| 811161 | 0.000316188 | ABCF1\|ATP-binding cassette, sub-family F (GCN20), member 1 |
| 1556872 | 0.012702024 | FLJ12628\|hypothetical protein FLJ12628 |
| 897542 | 9.81E−05 | DKFZP564F0522\|DKFZP564F0522 protein |
| 282720 | 0.02278302 | OIP2\|Opa-interacting protein 2 |
| 897544 | 0.02996845 | LMNA\|lamin A/C |
| 757328 | 0.002130274 | FLJ22678\|hypothetical protein FLJ22678 |
| 589869 | 2.56E−07 | TAZ\|transcriptional co-activator with PDZ-binding motif (TAZ) |
| 1474149 | 0.042782235 | PVRL1\|poliovirus receptor-related 1 (herpesvirus entry mediator C; nectin) |
| 789012 | 0.000225033 | FBLN2\|fibulin 2 |
| 842994 | 0.002759641 | CTSZ\|cathepsin Z |
| 249705 | 0.037596341 | DSS1\|Deleted in split-hand/split-foot 1 region |
| 2028238 | 0.000866786 | KIAA0300\|KIAA0300 protein |
| 530237 | 0.012438932 | Homo sapiens cDNA FLJ14208 fis, clone NT2RP3003264 |
| 664975 | 0.008779706 | CLDN1\|claudin 1 |
| 70201 | 3.09E−06 | LOC51312\|mitochondrial solute carrier |
| 340850 | 0.0041899 | MGC12992\|hypothetical protein MGC12992 |
| 191978 | 5.46E−05 | ATP5J2\|ATP synthase, H+ transporting, mitochondrial F0 complex, subunit f, isoform 2 |
| 782193 | 0.002728134 | TXN\|thioredoxin |
| 485220 | 7.62E−05 | HSA011916\|hypothetical protein |
| 2019426 | 3.13E−05 | PTPNS1\|protein tyrosine phosphatase, non-receptor type substrate 1 |
| 825229 | 0.001223619 | MRPL11\|mitochondrial ribosomal protein L11 |
| 809981 | 7.94E−07 | GPX4\|glutathione peroxidase 4 (phospholipid hydroperoxidase) |
| 179163-2 | 0.029938789 | GRIN2C\|glutamate receptor, ionotropic, N-methyl D-aspartate 2C |
| 41869 | 0.034236898 | FLJ11017\|hypothetical protein FLJ11017 |
| 563673 | 0.008164837 | ALDH7A1\|aldehyde dehydrogenase 7 family, member A1 |
| 2714973 | 8.88E−05 | GALNS\|galactosamine (N-acetyl)-6-sulfate sulfatase (Morquio syndrome, mucopolysaccharidosis type |
| | | IVA) |
| 1638827 | 0.015500819 | RFPL3S\|ret finger protein-like 3 antisense |
| 295843 | 0.042020398 | CYP27A1\|cytochrome P450, subfamily XXVIIA (steroid 27-hydroxylase, cerebrotendinous |
| | | xanthomatosis), polypeptide 1 |
| 156283 | 0.041270668 | ESTs |
| 307843 | 5.77E−07 | PPP2CA\|protein phosphatase 2 (formerly 2A), catalytic subunit, alpha isoform |
| 1474164 | 0.010013706 | FLJ12886\|hypothetical protein FLJ12886 |
| 289288 | 0.003697618 | ATP2C1\|ATPase, Ca++ transporting, type 2C, member 1 |
| 50693 | 0.000289561 | FLJ20232\|hypothetical protein |
| 884462 | 0.000194201 | DSCR1\|Down syndrome critical region gene 1 |
| 338599 | 1.85E−05 | NRBP\|nuclear receptor binding protein |
| 279810 | 0.039210986 | Homo sapiens mRNA; cDNA DKFZp564O1016 (from clone DKFZp564O1016) |
| 1948119 | 0.001273848 | GRAF\|GTPase regulator associated with the focal adhesion kinase pp125 |
| 292512 | 0.001680803 | MLL3\|myeloid/lymphoid or mixed-lineage leukemia 3 |
| 685182 | 0.00023067 | CCT6A\|chaperonin containing TCP1, subunit 6A (zeta 1) |
| 1636156 | 0.005363775 | FLJ21709\|hypothetical protein FLJ21709 |
| 1553560 | 2.41E−06 | ESTs, Weakly similar to hyperpolarization-activated cyclic nucleotide-gated channel hHCN2 [H. sapiens] |
| 471196 | 2.95E−13 | ITM3\|integral membrane protein 3 |
| 754018 | 0.000287497 | SVIL\|supervillin |
| 179163 | 0.019936342 | GRIN2C\|glutamate receptor, ionotropic, N-methyl D-aspartate 2C |
| 770059 | 0.002506551 | HSPG2\|heparan sulfate proteoglycan 2 (perlecan) |
| 504461 | 0.00094691 | OPN3\|opsin 3 (encephalopsin, panopsin) |
| 760298 | 0.018009132 | PRSC1\|protease, cysteine, 1 (legumain) |
| 796268 | 0.010529568 | PPP1R1A\|protein phosphatase 1, regulatory (inhibitor) subunit 1A |
| 782501 | 0.000195601 | PP1665\|hypothetical protein PP1665 |
| 782503 | 0.015719214 | FADS1\|fatty acid desaturase 1 |
| 263097 | 0.000642521 | RPIA\|ribose 5-phosphate isomerase A (ribose 5-phosphate epimerase) |
| 687875 | 0.027149103 | CTSS\|cathepsin S |
| 1733191 | 0.009227097 | ESTs |
| 771004 | 0.026126878 | Homo sapiens, clone IMAGE: 3880654, mRNA |
| 1708335 | 0.005854823 | GDF5\|growth differentiation factor 5 (cartilage-derived morphogenetic protein-1) |
| 756708 | 1.43E−08 | KCNN4\|potassium intermediate/small conductance calcium-activated channel, subfamily N, member 4 |
| 1636166 | 0.000160656 | KIAA0668\|KIAA0668 protein |
| 684539 | 0.011902794 | Homo sapiens mRNA; cDNA DKFZp761E212 (from clone DKFZp761E212) |
| 712577 | 0.000236843 | HCCS\|holocytochrome c synthase (cytochrome c heme-lyase) |
| 754026 | 1.64E−09 | SMURF2\|E3 ubiquitin ligase SMURF2 |
| 1161564 | 9.84E−11 | DMN\|desmuslin |
| 429368 | 0.018119429 | HOX11\|homeo box 11 (T-cell lymphoma 3-associated breakpoint) |
| 41898 | 0.005343697 | PTGDS\|prostaglandin D2 synthase (21 kD, brain) |
| 810551 | 0.000677873 | LRP1\|low density lipoprotein-related protein 1 (alpha-2-macroglobulin receptor) |
| 812159 | 8.87E−05 | FLJ20337\|hypothetical protein FLJ20337 |
| 70245 | 0.011187375 | Homo sapiens mRNA full length insert cDNA clone EUROIMAGE 50374 |
| 138672 | 7.63E−08 | ESTs |
| 1874367 | 0.019549691 | SCYA20\|small inducible cytokine subfamily A (Cys-Cys), member 20 |
| 49389 | 0.000722653 | STX1A\|syntaxin 1A (brain) |
| 897590 | 0.036633129 | NESP55\|neuroendocrine secretory protein 55 |
| 824602 | 0.014186983 | IFI16\|interferon, gamma-inducible protein 16 |
| 140951 | 4.83E−06 | ACTN4\|actinin, alpha 4 |
| 41108 | 0.039873583 | Homo sapiens clone 25023 mRNA sequence |
| 40164 | 0.008273187 | Homo sapiens clone 23836 mRNA sequence |
| 415613 | 0.042602065 | LOC51304\|DHHC1 protein |
| 180803 | 0.01441978 | INPP1\|inositol polyphosphate-1-phosphatase |
| 823665 | 0.000516552 | PBEF\|pre-B-cell colony-enhancing factor |
| 122238 | 0.013397185 | KIAA0669\|KIAA0669 gene product |
| 502880 | 0.003571882 | MGC11352\|hypothetical protein MGC11352 |
| 812164 | 0.006694394 | 384D8-2\|hypothetical protein 384D8_6 |
| 784129 | 0.003286809 | YWHAG\|tyrosine 3-monooxygenase/tryptophan 5-monooxygenase activation protein, gamma |
| | | polypeptide |
| 812167 | 0.045013394 | GGA3\|ADP-ribosylation factor-binding protein GGA3 |
| 810567 | 0.007408045 | ARHGEF2\|rho/rac guanine nucleotide exchange factor (GEF) 2 |
| 593001 | 4.44E−05 | WHSC1\|Wolf-Hirschhorn syndrome candidate 1 |
| 285323 | 0.000245162 | ESTs, Weakly similar to A28996 proline-rich protein M14 precursor - mouse [M. musculus] |
| 754040 | 6.62E−06 | Homo sapiens cDNA FLJ31626 fis, clone NT2RI2003317 |
| 364716 | 0.000137407 | MSH6\|mutS (E. coli) homolog 6 |
| 193913 | 4.65E−09 | LYN\|v-yes-1 Yamaguchi sarcoma viral related oncogene homolog |
| 205913 | 4.76E−05 | FLJ10955\|hypothetical protein FLJ10955 |
| 489553 | 0.014887446 | ESRRA\|estrogen-related receptor alpha |
| 122241 | 0.000328148 | PSMB2\|proteasome (prosome, macropain) subunit, beta type, 2 |
| 360436 | 0.002697689 | COPEB\|core promoter element binding protein |
| 1584623 | 0.000523143 | CCNC\|cyclin C |
| 1914863 | 3.34E−10 | DYSF\|dysferlin, limb girdle muscular dystrophy 2B (autosomal recessive) |
| 2244561 | 0.026955181 | CROC4\|transcriptional activator of the c-fos promoter |
| 742685 | 0.000245162 | DAB2\|disabled (Drosophila) homolog 2 (mitogen-responsive phosphoprotein) |
| 289615 | 0.008351255 | LAMP2\|lysosomal-associated membrane protein 2 |
| 1576468 | 0.001426413 | HSD17B2\|hydroxysteroid (17-beta) dehydrogenase 2 |
| 145112 | 0.009363602 | ICAM1\|intercellular adhesion molecule 1 (CD54), human rhinovirus receptor |
| 756731 | 0.022873245 | MAP3K3\|mitogen-activated protein kinase kinase kinase 3 |
| 624390 | 0.000380341 | DC13\|DC13 protein |
| 35642 | 0.007213754 | FLJ10826\|hypothetical protein FLJ10826 |
| 310034 | 5.32E−10 | PRKY\|protein kinase, Y-linked |
| 1846982 | 0.016285335 | INHBC\|inhibin, beta C |
| 825284 | 0.028295772 | MGC5508\|hypothetical protein MGC5508 |
| 868472 | 4.10E−08 | CLIC4\|chloride intracellular channel 4 |
| 327425 | 0.019017794 | ESTs |
| 2237353 | 0.021840957 | GAL\|galanin |
| 1911531 | 0.000758456 | SH3GLB1\|SH3-domain, GRB2-like, endophilin B1 |
| 345069 | 3.99E−06 | NFE2L3\|nuclear factor (erythroid-derived 2)-like 3 |
| 767164 | 2.48E−07 | Homo sapiens cDNA FLJ32401 fis, clone SKMUS2000339 |
| 812187 | 0.029639799 | MGC3279\|hypothetical protein MGC3279 similar to collectins |
| 758343 | 0.000196981 | PPIF\|peptidylprolyl isomerase F (cyclophilin F) |
| 780947 | 0.02676531 | POLD1\|polymerase (DNA directed), delta 1, catalytic subunit (125 kD) |
| 593023 | 0.008498512 | DTNB\|dystrobrevin, beta |
| 209066-2 | 0.000805479 | STK15\|serine/threonine kinase 15 |
| 209137 | 3.88E−07 | GABRE\|gamma-aminobutyric acid (GABA) A receptor, epsilon |
| 1588924 | 0.001432315 | HHGP\|HHGP protein |
| 292567 | 0.000125692 | MCPR\|anaphase-promoting complex 1; meiotic checkpoint regulator |
| 44477 | 0.008204839 | VCAM1\|vascular cell adhesion molecule 1 |
| 1721076 | 3.74E−05 | ZNF136\|zinc finger protein 136 (clone pHZ-20) |
| 825293 | 0.031216746 | KIAA0082\|KIAA0082 protein |
| 840677 | 0.004604961 | IGKC\|immunoglobulin kappa constant |
| 768111 | 0.008346481 | Homo sapiens mRNA; cDNA DKFZp586L0120 (from clone DKFZp586L0120) |
| 769857 | 0.002264658 | CBS\|cystathionine-beta-synthase |
| 841620 | 0.000244327 | DPYSL2\|dihydropyrimidinase-like 2 |
| 855389 | 0.04294858 | TACC1\|transforming, acidic coiled-coil containing protein 1 |
| 28444 | 0.045698004 | CRSP2\|cofactor required for Sp1 transcriptional activation, subunit 2 (150 kD) |
| 43828 | 0.002262746 | Homo sapiens, clone MGC: 5564, mRNA, complete cds |
| 42880 | 2.84E−07 | CDK8\|cyclin-dependent kinase 8 |
| 896978 | 0.00231445 | FLJ12810\|hypothetical protein FLJ12810 |
| 767180 | 0.000175172 | C1orf6\|chromosome 1 open reading frame 6 |
| 43833 | 0.001478693 | DGKG\|diacylglycerol kinase, gamma (90 kD) |
| 207550 | 0.004415317 | KIAA0057\|TRAM-like protein |
| 188335 | 0.016029967 | EMR2\|egf-like module containing, mucin-like, hormone receptor-like sequence 2 |
| 276547 | 0.01022325 | DNMT1\|DNA (cytosine-5-)-methyltransferase 1 |
| 744605 | 0.000194318 | FLJ10297\|hypothetical protein FLJ10297 |
| 756769 | 0.000566667 | CHAF1B\|chromatin assembly factor 1, subunit B (p60) |
| 753428 | 0.016749826 | Homo sapiens, Similar to RIKEN cDNA 1110014B07 gene, clone MGC: 20766 IMAGE: 4586039, mRNA, |
| | | complete cds |
| 344430 | 0.008499295 | BMP7\|bone morphogenetic protein 7 (osteogenic protein 1) |
| 345090 | 0.006154605 | GL004\|GL004 protein |
| 1622465 | 0.026837772 | ESTs, Weakly similar to ALU1_HUMAN ALU SUBFAMILY J SEQUENCE CONTAMINATION WARNING |
| | | ENTRY [H. sapiens] |
| 1917449 | 0.000329467 | SAA4\|serum amyloid A4, constitutive |
| 76182 | 0.000472307 | DKFZP761F241\|hypothetical protein DKFZp761F241 |
| 254625 | 0.010898804 | KIAA0229\|KIAA0229 protein |
| 840697 | 0.040193056 | FKBP9\|FK506-binding protein 9 (63 kD) |
| 255285 | 5.16E−09 | ESTs |
| 813157 | 7.60E−08 | DKFZp547A023\|hypothetical protein DKFZp547A023 |
| 259842-2 | 1.51E−06 | PMPCB\|peptidase (mitochondrial processing) beta |
| 782575 | 0.000103262 | HSJ001348\|cDNA for differentially expressed CO16 gene |
| 612274 | 0.000642521 | TUBA1\|tubulin, alpha 1 (testis specific) |
| 28469 | 0.015871 | OXCT\|3-oxoacid CoA transferase |
| 1630639 | 0.043830461 | ATP1A3\|ATPase, Na+/K+transporting, alpha 3 polypeptide |
| 302996 | 0.022945273 | CLIC3\|chloride intracellular channel 3 |
| 378813 | 5.00E−10 | SLPI\|secretory leukocyte protease inhibitor (antileukoproteinase) |
| 782581 | 1.03E−05 | KIAA0996\|KIAA0996 protein |
| 471568 | 8.73E−06 | HN1\|hematological and neurological expressed 1 |
| 877621 | 0.013066696 | AF9Q34\|nGAP-like protein |
| 746229 | 1.17E−13 | MAP4K4\|mitogen-activated protein kinase kinase kinase kinase 4 |
| 265060 | 0.000287204 | KIT\|v-kit Hardy-Zuckerman 4 feline sarcoma viral oncogene homolog |
| 1632248 | 0.019761169 | Homo sapiens cDNA FLJ14181 fis, clone NT2RP2004300 |
| 488956 | 0.002056402 | CUGBP2\|CUG triplet repeat, RNA-binding protein 2 |
| 591465 | 0.032429761 | Homo sapiens, clone MGC: 2908 IMAGE: 3029644, mRNA, complete cds |
| 2163910 | 0.021936941 | CSPG3\|chondroitin sulfate proteoglycan 3 (neurocan) |
| 814117 | 0.01403347 | DDX21\|DEAD/H (Asp-Glu-Ala-Asp/His) box polypeptide 21 |
| 769890 | 0.000279148 | NP\|nucleoside phosphorylase |
| 234325 | 0.006737438 | CUL4A\|cullin 4A |
| 45464 | 6.10E−07 | AK2\|adenylate kinase 2 |
| 284734 | 0.044838359 | WASF1\|WAS protein family, member 1 |
| 843263 | 0.005565298 | MRPL37\|mitochondrial ribosomal protein L37 |
| 897956 | 0.000566151 | PRAME\|preferentially expressed antigen in melanoma |
| 282051 | 0.007261049 | ESTs, Highly similar to I38945 melanoma ubiquitous mutated protein [H. sapiens] |
| 744632 | 5.13E−08 | EST |
| 43865 | 1.57E−05 | DNCI1\|dynein, cytoplasmic, intermediate polypeptide 1 |
| 841663 | 3.62E−05 | NARF\|nuclear prelamin A recognition factor |
| 1525461 | 0.001442019 | RIPK2\|receptor-interacting serine-threonine kinase 2 |
| 826286 | 0.002032859 | IMP13\|importin 13 |
| 1470278 | 0.000152832 | FLJ21841\|hypothetical protein FLJ21841 |
| 796646 | 6.57E−07 | ODC1\|ornithine decarboxylase 1 |
| 502161 | 0.001298549 | APPBP1\|amyloid beta precursor protein-binding protein 1, 59 kD |
| 1610338 | 0.021681051 | FLJ22324\|hypothetical protein FLJ22324 |
| 146123 | 0.003881514 | PTPRK\|protein tyrosine phosphatase, receptor type, K |
| 153340 | 0.009064405 | GRO2\|GRO2 oncogene |
| 243155 | 0.048299748 | FLJ12549\|hypothetical protein FLJ12549 |
| 897963 | 6.59E−06 | PPAP2A\|phosphatidic acid phosphatase type 2A |
| 304908 | 1.10E−05 | E2F3\|E2F transcription factor 3 |
| 2701197 | 0.019362001 | C11orf9\|chromosome 11 open reading frame 9 |
| 119384 | 0.000965202 | CHIT1\|chitinase 1 (chitotriosidase) |
| 877644 | 0.001738247 | PRCC\|papillary renal cell carcinoma (translocation-associated) |
| 220293 | 0.010938828 | Homo sapiens cDNA: FLJ21800 fis, clone HEP00618 |
| 1732922 | 0.001365668 | Homo sapiens mRNA; cDNA DKFZp762H106 (from clone DKFZp762H106) |
| 470930 | 2.82E−05 | DDX11\|DEAD/H (Asp-Glu-Ala-Asp/His) box polypeptide 11 (S. cerevisiae CHL1-like helicase) |
| 701460 | 0.001790158 | CHFR\|checkpoint with forkhead and ring finger domains |
| 269374 | 0.000195253 | KIAA0301\|KIAA0301 protein |
| 825641 | 3.34E−06 | B4GALT5\|UDP-Gal: betaGlcNAc beta 1,4-galactosyltransferase, polypeptide 5 |
| 531608 | 0.002759613 | HMGIC\|high-mobility group (nonhistone chromosomal) protein isoform I-C |
| 300632 | 0.042347323 | FLJ21044\|hypothetical protein FLJ21044 similar to Rbig1 |
| 266037 | 0.00106935 | MAFG\|v-maf musculoaponeurotic fibrosarcoma (avian) oncogene family, protein G |
| 1585650 | 0.01494333 | FLJ10781\|hypothetical protein FLJ10781 |
| 841689 | 0.018506257 | ATP6S1\|ATPase, H+ transporting, lysosomal (vacuolar proton pump), subunit 1 |
| 1461477 | 1.32E−05 | Homo sapiens mRNA; cDNA DKFZp586I0324 (from clone DKFZp586I0324) |
| 784504 | 0.000195601 | MBTPS1\|membrane-bound transcription factor protease, site 1 |
| 1493160 | 0.001295674 | SCYB10\|small inducible cytokine subfamily B (Cys-X-Cys), member 10 |
| 47096 | 0.014632388 | FUSIP2\|FUS-interacting protein (serine-arginine rich) 2 |
| 770462 | 0.000758456 | CPZ\|carboxypeptidase Z |
| 745606 | 0.014811257 | PP591\|hypothetical protein PP591 |
| 268727 | 0.001267014 | MUTYH\|mutY (E. coli) homolog |
| 787851 | 0.042347323 | Homo sapiens PRO2893 mRNA, complete cds |
| 825659 | 0.006087414 | NDRG1\|N-myc downstream regulated |
| 22917 | 0.013187912 | Homo sapiens mRNA; cDNA DKFZp761M0111 (from clone DKFZp761M0111) |
| 1593829 | 0.044484074 | TIA1\|TIA1 cytotoxic granule-associated RNA-binding protein |
| 111120 | 7.01E−06 | KIAA0063\|KIAA0063 gene product |
| 810958 | 0.004710677 | CPNE1\|copine I |
| 950096 | 0.004536135 | ARCN1\|archain 1 |
| 853066 | 0.003225295 | CNAP1\|chromosome condensation-related SMC-associated protein 1 |
| 292936 | 4.65E−06 | FLJ10468\|hypothetical protein FLJ10468 |
| 701481 | 0.003482886 | MX2\|myxovirus (influenza) resistance 2, homolog of murine |
| 897997 | 0.04142324 | SMC1L1\|SMC1 (structural maintenance of chromosomes 1, yeast)-like 1 |
| 136399 | 3.96E−05 | DKFZP586F2423\|hypothetical protein DKFZp586F2423 |
| 752837 | 4.76E−08 | Homo sapiens mRNA for FLJ00074 protein, partial cds |
| 278650-2 | 0.015052222 | PPP1R8\|protein phosphatase 1, regulatory (inhibitor) subunit 8 |
| 809627 | 0.000137744 | NRIP1\|nuclear receptor interacting protein 1 |
| 787860 | 2.92E−08 | ESTs |
| 183468 | 7.58E−08 | EGFR\|epidermal growth factor receptor (avian erythroblastic leukemia viral (v-erb-b) oncogene homolog) |
| 416676 | 2.51E−09 | PELI1\|pellino (Drosophila) homolog 1 |
| 323522 | 8.66E−06 | NRBP\|nuclear receptor binding protein |
| 49794 | 0.005649538 | LOC51700\|cytochrome b5 reductase b5R.2 |
| 278504 | 0.036283521 | KIAA0607\|neurochondrin |
| 951048 | 0.009825328 | DKFZP434K046\|hypothetical protein DKFZp434K046 |
| 460403 | 0.006497654 | LAMC2\|laminin, gamma 2 (nicein (100 kD), kalinin (105 kD), BM600 (100 kD), Herlitz junctional |
| | | epidermolysis bullosa)) |
| 124246 | 0.044429786 | ESTs, Weakly similar to ALUC_HUMAN !!!! ALU CLASS C WARNING ENTRY !!! [H. sapiens] |
| 505836 | 0.00219195 | ESTs, Moderately similar to reduced expression in cancer [H. sapiens] |
| 796694 | 0.00244749 | BIRC5\|baculoviral IAP repeat-containing 5 (survivin) |
| 811918 | 0.002709516 | KIAA0952\|KIAA0952 protein |
| 1556545 | 0.012132745 | Homo sapiens, Similar to RIKEN cDNA 2600001B17 gene, clone IMAGE: 2822298, mRNA, partial cds |
| 704045 | 4.14E−09 | MGC4126\|hypothetical protein MGC4126 |
| 1856063 | 0.013677014 | TTYH1\|tweety (Drosophila) homolog 1 |
| 276915 | 1.29E−05 | DNMT3B\|DNA (cytosine-5-)-methyltransferase 3 beta |
| 211800 | 0.005621271 | KIAA0833\|KIAA0833 protein |
| 232789 | 0.021224486 | POLR2J\|polymerase (RNA) II (DNA directed) polypeptide J (13.3 kD) |
| 277579 | 0.008167816 | ESTs |
| 813520 | 0.000287377 | EPHB3\|EphB3 |
| 486626 | 0.0454965 | Homo sapiens, clone IMAGE: 4332938, mRNA |
| 882515 | 0.001000384 | EIF3S9\|eukaryotic translation initiation factor 3, subunit 9 (eta, 116 kD) |
| 429799 | 0.031044349 | FLJ21939\|hypothetical protein FLJ21939 similar to 5-azacytidine induced gene 2 |
| 1569658 | 0.006181327 | ESTs |
| 1474900 | 0.043509438 | KRT15\|keratin 15 |
| 810983 | 0.001630467 | DKFZP434H132\|DKFZP434H132 protein |
| 308163 | 0.018779849 | ESTs, Weakly similar to TRHY_HUMAN TRICHOHYALI [H. sapiens] |
| 839736 | 7.03E−08 | CRYAB\|crystallin, alpha B |
| 1899263 | 0.04697029 | FLJ20113\|hypothetical protein FLJ20113 |
| 1536236 | 1.53E−14 | FLJ13154\|hypothetical protein FLJ13154 |
| 124261 | 0.010467742 | SNRP70\|small nuclear ribonucleoprotein 70 kD polypeptide (RNP antigen) |
| 2018154 | 4.91E−07 | ETFB\|electron-transfer-flavoprotein, beta polypeptide |
| 67741 | 0.000180391 | PP2135\|PP2135 protein |
| 266085 | 0.0003394 | KHSRP\|KH-type splicing regulatory protein (FUSE binding protein 2) |
| 825695 | 0.004394757 | HSPC111\|hypothetical protein |
| 809657 | 0.013236102 | Homo sapiens cDNA FLJ31373 fis, clone NB9N42000342 |
| 110507 | 0.000223108 | ESTs, Weakly similar to T46471 hypothetical protein DKFZp434L0130.1 [H. sapiens] |
| 724387 | 0.012976841 | SNRPC\|small nuclear ribonucleoprotein polypeptide C |
| 855786 | 0.002192122 | WARS\|tryptophanyl-tRNA synthetase |
| 259950 | 1.91E−05 | CML66\|chronic myelogenous leukemia tumor antigen 66 |
| 1899274 | 0.00024151 | EGLN2\|EGL nine (C. elegans) homolog 2 |
| 23903 | 0.020116819 | Homo sapiens clone 23903 mRNA sequence |
| 302292 | 0.005746627 | EXT2\|exostoses (multiple) 2 |
| 815142 | 0.000812059 | SACS\|spastic ataxia of Charlevoix-Saguenay (sacsin) |
| 505864 | 0.000602202 | RGL\|RaIGDS-like gene |
| 325160 | 1.02E−11 | NP25\|neuronal protein |
| 1868534 | 3.65E−05 | MGC2408\|hypothetical protein MGC2408 |
| 376515 | 0.00998974 | FLJ10416\|similar to constitutive photomorphogenic protein 1 (Arabidopsis) |
| 136722 | 1.06E−05 | ATP1B3\|ATPase, Na+/K+ transporting, beta 3 polypeptide |
| 284022 | 0.001908121 | ARHGEF10\|Rho guanine nucleotide exchange factor (GEF) 10 |
| 2010684 | 0.009238649 | KIAA0640\|SWAP-70 protein |
| 1739821 | 0.022220862 | LOC83690\|CocoaCrisp |
| 1618987 | 0.008755709 | RLBP1\|retinaldehyde-binding protein 1 |
| 282428 | 0.003858041 | Homo sapiens, Similar to RIKEN cDNA 9030409E16 gene, clone MGC: 26939 IMAGE: 4796761, mRNA, |
| | | complete cds |
| 1607482 | 5.44E−05 | CEBPG\|CCAAT/enhancer binding protein (C/EBP), gamma |
| 123627 | 0.012829341 | P5\|protein disulfide isomerase-related protein |
| 1606829 | 0.005336709 | HDAC4\|histone deacetylase 4 |
| 81409 | 0.016588172 | GABARAPL1\|GABA(A) receptor-associated protein like 1 |
| 340558 | 0.000227672 | ARPC5\|actin related protein 2/3 complex, subunit 5 (16 kD) |
| 1635970 | 1.87E−07 | MEHAS1\|malignant fibrous histiocytoma amplified sequence 1 |
| 454970 | 7.09E−05 | HAIK1\|type\|intermediate filament cytokeratin |
| 198509 | 0.034037336 | NUDEL\|LIS1-interacting protein NUDEL; endooligopeptidase A |
| 51328 | 0.000752302 | CDC34\|cell division cycle 34 |
| 41565 | 0.004757679 | MYCN\|v-myc avian myelocytomatosis viral related oncogene, neuroblastoma derived |
| 1691868 | 0.000858947 | PTX3\|pentaxin-related gene, rapidly induced by IL-1 beta |
| 505881 | 0.006498381 | ADA\|adenosine deaminase |
| 813560 | 0.002137013 | TRN-SR\|transportin-SR |
| 505882 | 0.023035247 | FLJ20550\|hypothetical protein FLJ20550 |
| 469686 | 5.61E−06 | RIT\|Ric (Drosophila)-like, expressed in many tissues |
| 1493527 | 2.33E−05 | ASNS\|asparagine synthetase |
| 325182 | 0.027251157 | CDH2\|cadherin 2, type 1, N-cadherin (neuronal) |
| 784589 | 0.008673574 | MMP15\|matrix metalloproteinase 15 (membrane-inserted) |
| 2579848 | 0.016470796 | CHRAC1\|chromatin accessibility complex 1 |
| 854079 | 0.024580007 | ACTN1\|actinin, alpha 1 |
| 344854 | 2.86E−06 | ANKRD3\|ankyrin repeat domain 3 |
| 809696 | 0.0324664 | DDX38\|DEAD/H (Asp-Glu-Ala-Asp/His) box polypeptide 38 |
| 725365 | 0.015956866 | GAS1\|growth arrest-specific 1 |
| 713202 | 0.00119998 | HSU79252\|hypothetical protein |
| 1723400 | 0.000511825 | ESTs |
| 950451 | 0.015225688 | MGC10818\|hypothetical protein MGC10818 |
| 844601 | 0.00263797 | CCKBR\|cholecystokinin B receptor |
| 502558 | 0.010898808 | Homo sapiens, Similar to RIKEN cDNA 1110060O18 gene, clone MGC: 17236 IMAGE: 3864137, mRNA, |
| | | complete cds |
| 811976 | 0.002743023 | Homo sapiens cDNA FLJ10071 fis, clone HEMBA1001702 |
| 587992 | 0.000692211 | MGC5350\|hypothetical protein MGC5350 |
| 795309 | 1.99E−05 | SOD3\|superoxide dismutase 3, extracellular |
| 486683 | 0.001570786 | Homo sapiens mRNA; cDNA DKFZp564J0323 (from clone DKFZp564J0323) |
| 768561 | 0.006003565 | SCYA2\|small inducible cytokine A2 (monocyte chemotactic protein 1, homologous to mouse Sig-je) |
| 756401 | 0.008336499 | RHEB2\|Ras homolog enriched in brain 2 |
| 756405 | 0.014632388 | ID3\|inhibitor of DNA binding 3, dominant negative helix-loop-helix protein |
| 144924 | 0.001209009 | C6orf5\|chromosome 6 open reading frame 5 |
| 768569 | 0.038661233 | Homo sapiens mRNA full length insert cDNA clone EUROIMAGE 1517766 |
| 1688953 | 1.02E−07 | ESTs |
| 144926 | 0.012004594 | MGC3133\|hypothetical protein MGC3133 |
| 594428 | 0.038751321 | SEC3\|Sec3-like |
| 770848 | 0.00619877 | ESTs, Weakly similar to ALU1_HUMAN ALU SUBFAMILY J SEQUENCE CONTAMINATION WARNING |
| | | ENTRY [H. sapiens] |
| 429065 | 0.00025476 | ANKH\|ankylosis, progressive (mouse) homolog |
| 1933127 | 4.99E−05 | CX46.6\|connexin46.6 |
| 1551675 | 0.002246831 | Homo sapiens cDNA FLJ13903 fis, clone THYRO1001854 |
| 30093 | 3.59E−07 | RANBP1\|RAN binding protein 1 |
| 768571 | 2.06E−05 | SOX8\|SRY (sex determining region Y)-box 8 |
| 252382 | 0.004755604 | TM4SF6\|transmembrane 4 superfamily member 6 |
| 1493557 | 0.014557615 | PRP18\|pre-mRNA processing factor 18 |
| 144932 | 6.88E−09 | CDK2AP1\|CDK2-associated protein 1 |
| 705064 | 0.028746951 | TACC3\|transforming, acidic coiled-coil containing protein 3 |
| 391949 | 7.81E−05 | SH2D2A\|SH2 domain protein 2A |
| 1688965 | 0.016029967 | Homo sapiens mRNA for putative NSE1 protein |
| 361861 | 0.000352001 | PCBP4\|poly(rC)-binding protein 4 |
| 142259 | 0.001096449 | FIP2\|tumor necrosis factor alpha-inducible cellular protein containing leucine zipper domains; Huntingtin |
| | | interacting protein L; transcrption factor IIIA-interacting protein |
| 131839 | 0.000190493 | FOLR1\|folate receptor 1 (adult) |
| 130892 | 0.000109361 | EHD3\|EH-domain containing 3 |
| 2252417 | 0.004138974 | RPS10\|ribosomal protein S10 |
| 298122 | 0.003448915 | FZD7\|frizzled (Drosophila) homolog 7 |
| 725390 | 2.24E−09 | GSTP1\|glutathione S-transferase pi |
| 2308263 | 0.015071436 | FDXR\|ferredoxin reductase |
| 153760 | 4.24E−05 | EPHB1\|EphB1 |
| 713238 | 0.000107772 | MONDOA\|KIAA0867 protein |
| 250797 | 0.034063029 | FLJ20038\|hypothetical protein FLJ20038 |
| 126221 | 0.001359734 | TPD52L2\|tumor protein D52-like 2 |
| 898249 | 0.001409555 | ESTs |
| 129569 | 0.000115962 | HSPC049\|HSPC049 protein |
| 2115545 | 7.00E−05 | ADCY7\|adenylate cyclase 7 |
| 269787 | 0.027049095 | L1CAM\|L1 cell adhesion molecule (hydrocephalus, stenosis of aqueduct of Sylvius 1, MASA (mental |
| | | retardation, aphasia, shuffling gait and adducted thumbs) syndrome, spastic paraplegia 1) |
| 246120 | 0.008472265 | HRMT1L2\|HMT1 (hnRNP methyltransferase, S. cerevisiae)-like 2 |
| 415326 | 0.023366836 | ESTs |
| 868169 | 0.000993361 | LPL\|lipoprotein lipase |
| 1903066 | 7.49E−07 | KRTHB1\|keratin, hair, basic, 1 |
| 306933 | 0.036825 | Homo sapiens clone 25012 mRNA sequence |
| 773419 | 0.002212104 | ESTs |
| 758037 | 0.001366354 | HIVEP1\|human immunodeficiency virus type I enhancer-binding protein 1 |
| 308539 | 0.001531987 | Homo sapiens cDNA FLJ12777 fis, clone NT2RP2001720 |
| 1469377 | 9.20E−06 | LHFPL2\|lipoma HMGIC fusion partner-like 2 |
| 898258 | 0.014789221 | CHN1\|chimerin (chimaerin) 1 |
| 841308 | 2.88E−07 | MYLK\|myosin, light polypeptide kinase |
| 815501 | 2.47E−08 | MGC2721\|hypothetical protein MGC2721 |
| 208969 | 0.000183624 | EST |
| 814562 | 0.003359378 | ABT1\|TATA-binding protein-binding protein |
| 204686 | 0.015480089 | FXYD1\|FXYD domain-containing ion transport regulator 1 (phospholemman) |
| 491545 | 0.001014724 | KIAA0965\|KIAA0965 protein |
| 261518 | 2.50E−06 | FLJ20287\|hypothetical protein FLJ20287 |
| 154720 | 0.04010901 | ARD1\|N-acetyltransferase, homolog of S. cerevisiae ARD1 |
| 859359 | 5.91E−05 | PIG3\|quinone oxidoreductase homolog |
| 2306682 | 0.028235912 | AKAP9\|A kinase (PRKA) anchor protein (yotiao) 9 |
| 756442 | 0.000637212 | POR\|P450 (cytochrome) oxidoreductase |
| 1388395 | 0.013736819 | ELK1\|ELK1, member of ETS oncogene family |
| 770880 | 0.000953714 | PRIM2A\|primase, polypeptide 2A (58 kD) |
| 770884 | 0.00070891 | TIP-1\|Tax interaction protein 1 |
| 788136 | 0.026617009 | PDE4B\|phosphodiesterase 4B, cAMP-specific (dunce (Drosophila)-homolog phosphodiesterase E4) |
| 1639531 | 0.000121707 | RAB27A\|RAB27A, member RAS oncogene family |
| 248829 | 0.000127033 | LOX\|lysyl oxidase |
| 28140 | 0.009335762 | Homo sapiens mRNA; cDNA; DKFZp586F2224 (from clone DKFZp586F2224) |
| 50743 | 0.038854573 | TXNL2\|thioredoxin-like 2 |
| 884511 | 0.024982068 | COX7B\|cytochrome c oxidase subunit VIIb |
| 41929 | 0.015950143 | PICALM\|phosphatidylinositol binding clathrin assembly protein |
| 82434 | 0.021226526 | EEF1G\|eukaryotic translation elongation factor 1 gamma |
| 1574594 | 0.000255461 | MDK\|midkine (neurite growth-promoting factor 2) |
| 1031940 | 0.001333654 | CAMP\|cathelicidin antimicrobial peptide |
| 898281 | 8.51E−05 | FLNA\|filamin A, alpha (actin-binding protein-280) |
| 287749 | 9.83E−05 | CDC7L1\|CDC7 (cell division cycle 7, S. cerevisiae, homolog)-like 1 |
| 701115 | 1.56E−08 | PRO2013\|hypothetical protein PRO2013 |
| 283461 | 0.009028782 | FLJ13910\|hypothetical protein FLJ13910 |
| 188036 | 0.000166323 | BPAG1\|bullous pemphigoid antigen 1 (230/240 kD) |
| 196992 | 0.00144083 | AKR1C1\|aldo-keto reductase family 1, member C1 (dihydrodiol dehydrogenase 1; 20-alpha (3-alpha)- |
| | | hydroxysteroid dehydrogenase) |
| 150466 | 0.043662375 | Homo sapiens cDNA FLJ14885 fis, clone PLACE1003711 |
| 760344 | 0.000228463 | UMPS\|uridine monophosphate synthetase (orotate phosphoribosyl transferase and orotidine-5′- |
| | | decarboxylase) |
| 841338 | 0.000233667 | PRNPIP\|prion protein interacting protein |
| 509458 | 0.029325674 | LOC57228\|hypothetical protein from clone 643 |
| 132828 | 0.004269143 | DSCR1L2\|Down syndrome critical region gene 1-like 2 |
| 2015212 | 0.007053992 | MRPS25\|mitochondrial ribosomal protein S25 |
| 742707 | 1.36E−05 | ESTs, Weakly similar to MUC2_HUMAN MUCIN 2 PRECURSOR [H. sapiens] |
| 815534 | 0.000247752 | ICAP-1A\|integrin cytoplasmic domain-associated protein 1 |
| 344134 | 0.00261847 | IGLL1\|immunoglobulin lambda-like polypeptide 1 |
| 795371 | 0.004029813 | LSM5\|U6 snRNA-associated Sm-like protein |
| 261541 | 2.09E−09 | PRKCN\|protein kinase C, nu |
| 24958 | 1.98E−06 | Homo sapiens mRNA; cDNA DKFZp434C2016 (from clone DKFZp434C2016) |
| 66406 | 4.34E−09 | ESTs, Highly similar to T47163 hypothetical protein DKFZp762E1312.1 [H. sapiens] |
| 2316471 | 1.76E−05 | DEDD\|death effector domain-containing |
| 22278 | 3.17E−05 | FLJ20511\|hypothetical protein FLJ20511 |
| 844680 | 0.01394813 | TRD@\|T cell receptor delta locus |
| 1716265 | 0.001534143 | WDR3\|WD repeat domain 3 |
| 278938 | 0.009614286 | LOC51144\|steroid dehydrogenase homolog |
| 177621 | 0.04563828 | RXRB\|retinoid X receptor, beta |
| 1642496 | 0.00087773 | MGC11266\|hypothetical protein MGC11266 |
| 796323 | 0.00106935 | ADD3\|adducin 3 (gamma) |
| 50768 | 6.93E−06 | DKFZp667O2416\|hypothetical protein DKFZp667O2416 |
| 26566 | 1.26E−06 | POMT1\|protein-O-mannosyltransferase 1 |
| 1461138 | 0.029910221 | H4FG\|H4 histone family, member G |
| 130153 | 5.61E−06 | SUPT5H\|suppressor of Ty (S. cerevisiae) 5 homolog |
| 190325 | 0.000121317 | Homo sapiens mRNA; cDNA DKFZp434E235 (from clone DKFZp434E235) |
| 2149968 | 8.36E−07 | FZD9\|frizzled (Drosophila) homolog 9 |
| 767982 | 9.30E−07 | Homo sapiens, clone IMAGE: 4134852, mRNA, partial cds |
| 51710 | 5.76E−07 | Homo sapiens, clone IMAGE: 3605655, mRNA |
| 153025 | 0.00010788 | LIF\|leukemia inhibitory factor (cholinergic differentiation factor) |
| 773469 | 0.037388104 | PTGFRN\|prostaglandin F2 receptor negative regulator |
| 758088 | 8.07E−05 | CALD1\|caldesmon 1 |
| 823715 | 0.035143688 | TRIP-Br2\|transcriptional regulator interacting with the PHS-bromodomain 2 |
| 26578 | 0.012675854 | PES1\|pescadillo (zebrafish) homolog 1, containing BRCT domain |
| 815556 | 0.003550049 | FLJ10430\|hypothetical protein FLJ10430 |
| 810612 | 0.013015797 | S100A11\|S100 calcium-binding protein A11 (calgizzarin) |
| 795395 | 0.037638089 | MGC5306\|hypothetical protein MGC5306 |
| 182177 | 0.031563977 | ADAM17\|a disintegrin and metalloproteinase domain 17 (tumor necrosis factor, alpha, converting |
| | | enzyme) |
| 155717 | 0.002588609 | CD79B\|CD79B antigen (immunoglobulin-associated beta) |
| 897650 | 0.000154041 | PUS1\|pseudouridylate synthase 1 |
| 825320 | 0.031634717 | Homo sapiens, clone IMAGE: 3355383, mRNA, partial cds |
| 1705861 | 0.003557075 | KLK1\|kallikrein 1, renal/pancreas/salivary |
| 2055888 | 0.040566492 | CENTG3\|centaurin, gamma 3 |
| 161998 | 0.017117327 | FLJ23138\|hypothetical protein FLJ23138 |
| 265853 | 0.001192546 | TEM8\|tumor endothelial marker 8 |
| 2325609 | 0.001089545 | MCM7\|minichromosome maintenance deficient (S. cerevisiae) 7 |
| 774420 | 0.011363913 | LMAN1\|lectin, mannose-binding, 1 |
| 324927 | 0.00185329 | KIAA0375\|KIAA0375 gene product |
| 151449 | 9.30E−05 | PTPN21\|protein tyrosine phosphatase, non-receptor type 21 |
| 753162 | 0.005108161 | KIAA0603\|KIAA0603 gene product |
| 897669 | 0.025225569 | PRKCSH\|protein kinase C substrate 80K-H |
| 2017930 | 0.020336469 | KIP2\|DNA-dependent protein kinase catalytic subunit-interacting protein 2 |
| 50794 | 0.011154216 | ZNF133\|zinc finger protein 133 (clone pHZ-13) |
| 530359 | 0.013769878 | FNTA\|farnesyltransferase, CAAX box, alpha |
| 785933 | 0.003257805 | SRPX\|sushi-repeat-containing protein, X chromosome |
| 731241 | 0.01978529 | MPST\|mercaptopyruvate sulfurtransferase |
| 61500 | 1.32E−08 | HLF\|hepatic leukemia factor |
| 248886 | 0.020116819 | RAB3-GAP150\|rab3 GTPase-activating protein, non-catalytic subunit (150 kD) |
| 1416782 | 0.015739275 | CKB\|creatine kinase, brain |
| 154790 | 0.038568449 | NGFR\|nerve growth factor receptor (TNFR superfamily, member 16) |
| 969843 | 2.51E−09 | ESTs, Weakly similar to LKHU proteoglycan link protein precursor [H. sapiens] |
| 2062329 | 1.54E−08 | TTK\|TTK protein kinase |
| 1323636 | 0.001297059 | MGAT1\|mannosyl (alpha-1,3-)-glycoprotein beta-1,2-N-acetylglucosaminyltransferase |
| 27544 | 4.45E−11 | PROML1\|prominin (mouse)-like 1 |
| 825343 | 0.015128158 | Homo sapiens, clone MGC: 15887 IMAGE: 3530481, mRNA, complete cds |
| 41987 | 3.24E−06 | Homo sapiens mRNA full length insert cDNA clone EUROIMAGE 966164 |
| 812244 | 0.00582367 | CKLF1\|chemokine-like factor 1 |
| 2321341 | 5.72E−06 | PGC\|progastricsin (pepsinogen C) |
| 1698036 | 0.030089117 | UBE2V1\|ubiquitin-conjugating enzyme E2 variant 1 |
| 855438 | 0.013975654 | ATP6S14\|ATPase, vacuolar, 14 kD |
| 774446 | 0.000137487 | ADM\|adrenomedullin |
| 626068 | 0.025636692 | XPOT\|exportin, tRNA (nuclear export receptor for tRNAs) |
| 36374 | 0.008011127 | CCNB1\|cyclin B1 |
| 69002 | 0.000262283 | ANGPTL4\|angiopoietin-like 4 |
| 531319 | 6.30E−08 | STK12\|serine/threonine kinase 12 |
| 1704155 | 0.046542451 | IER5\|immediate early response 5 |
| 753184 | 0.003943783 | SOX9\|SRY (sex determining region Y)-box 9 (campomelic dysplasia, autosomal sex-reversal) |
| 2032251 | 0.000446292 | ESTs |
| 34778 | 0.001089665 | VEGF\|vascular endothelial growth factor |
| 264146 | 0.008936352 | FLJ13194\|hypothetical protein FLJ13194 |
| 812256 | 0.01030915 | Homo sapiens cDNA: FLJ21693 fis, clone COL09609 |
| 509800 | 0.008621514 | CASK\|calcium/calmodulin-dependent serine protein kinase (MAGUK family) |
| 712683 | 0.000116468 | NCK1\|NCK adaptor protein 1 |
| 2020898 | 0.012093702 | PLOD3\|procollagen-lysine, 2-oxoglutarate 5-dioxygenase 3 |
| 1597813 | 0.001549458 | PP1044\|hypothetical protein PP1044 |
| 359982 | 0.008864958 | BNIP3\|BCL2/adenovirus E1B 19 kD-interacting protein 3 |
| 1674521 | 0.026617009 | EST |
| 41208 | 3.23E−05 | BMP1\|bone morphogenetic protein 1 |
| 486304 | 5.01E−05 | RASAL2\|RAS protein activator like 2 |
| 2017960 | 0.001121595 | PP1201\|PP1201 protein |
| 788507 | 0.046423899 | ESTs, Weakly similar to LIN1_HUMAN LINE-1 REVERSE TRANSCRIPTASE HOMOLOG [H. sapiens] |
| 327506 | 0.000554961 | Homo sapiens mRNA full length insert cDNA clone EUROIMAGE 327506 |
| 753198 | 4.39E−09 | JPO1\|c-Myc target JPO1 |
| 769921 | 6.30E−05 | UBE2C\|ubiquitin-conjugating enzyme E2C |
| 201727 | 0.000831262 | BCL6\|B-cell CLL/lymphoma 6 (zinc finger protein 51) |
| 2262296 | 0.012689251 | MUC6\|mucin 6, gastric |
| 1568391 | 4.48E−10 | PLS3\|plastin 3 (T isoform) |
| 785963 | 0.00019309 | KIAA0191\|KIAA0191 protein |
| 431759 | 0.043544579 | TEAD3\|TEA domain family member 3 |
| 785967 | 0.001744131 | EPB41L2\|erythrocyte membrane protein band 4.1-like 2 |
| 731279 | 0.015827676 | HRB\|HIV-1 Rev binding protein |
| 28510 | 0.000923428 | CNTN2\|contactin 2 (axonal) |
| 838478 | 0.000657157 | NCALD\|neurocalcin delta |
| 36393 | 0.003553426 | ACAT2\|acetyl-Coenzyme A acetyltransferase 2 (acetoacetyl Coenzyme A thiolase) |
| 788511 | 0.022786019 | RPS6KA1\|ribosomal protein S6 kinase, 90 kD, polypeptide 1 |
| 259627 | 4.41E−05 | KIAA1126\|KIAA1126 protein |
| 840753 | 0.00460254 | SCYA5\|small inducible cytokine A5 (RANTES) |
| 795730 | 0.000182825 | EFS2\|signal transduction protein (SH3 containing) |
| 281145 | 0.006338467 | AOC2\|amine oxidase, copper containing 2 (retina-specific) |
| 1894005 | 0.044484074 | FLJ12615\|hypothetical protein FLJ12615 similar to membrane protein, palmitoylated 3 (MAGUK p55 |
| | | subfamily member 5) |
| 823775 | 0.000549733 | GNAI3\|guanine nucleotide binding protein (G protein), alpha inhibiting activity polypeptide 3 |
| 774471 | 0.000851455 | LAMB1\|laminin, beta 1 |
| 454632 | 7.14E−08 | KIAA0481\|KIAA0481 gene product |
| 323238 | 3.88E−05 | GRO1\|GRO1 oncogene (melanoma growth stimulating activity, alpha) |
| 950897 | 0.001851412 | Homo sapiens mRNA; cDNA DKFZp586G1922 (from clone DKFZp586G1922) |
| 878330 | 0.037388104 | Homo sapiens cDNA: FLJ22044 fis, clone HEP09141 |
| 755891 | 0.002132318 | KIAA0317\|KIAA0317 gene product |
| 3172883 | 0.002068426 | ESTs, Weakly similar to S24195 dopamine receptor D4 [H. sapiens] |
| 358267 | 4.15E−05 | ESTs, Moderately similar to AF119917 63 PRO2831 [H. sapiens] |
| 230882 | 1.51E−06 | PAX6\|paired box gene 6 (aniridia, keratitis) |
| 754157 | 3.44E−05 | Homo sapiens mRNA; cDNA DKFZp434K2172 (from clone DKFZp434K2172) |
| 1915913 | 0.006354505 | CLIC2\|chloride intracellular channel 2 |
| 788524 | 0.040583119 | HSPC156\|HSPC156 protein |
| 282895 | 0.025901698 | FLJ20048\|hypothetical protein FLJ20048 |
| 795746 | 0.036395976 | Homo sapiens cDNA FLJ33167 fis, clone UTERU2000569 |
| 1608898 | 1.20E−08 | JRKL\|jerky (mouse) homolog-like |
| 71312 | 0.000227672 | UACA\|uveal autoantigen with coiled-coil domains and ankyrin repeats |
| 162208 | 0.004091636 | ARPC2\|actin related protein 2/3 complex, subunit 2 (34 kD) |
| 160609 | 0.000448621 | ESTs, Weakly similar to ALUA_HUMAN !!!! ALU CLASS A WARNING ENTRY !!! [H. sapiens] |
| 136409 | 0.000809442 | DKFZp547A023\|hypothetical protein DKFZp547A023 |
| 1572298 | 0.046169524 | CD3Z\|CD3Z antigen, zeta polypeptide (TiT3 complex) |
| 460126 | 8.06E−07 | KIAA0819\|KIAA0819 protein |
| 1049291 | 0.004674543 | OR7E47P\|olfactory receptor, family 7, subfamily E, member 47 pseudogene |
| 489677 | 0.000218116 | UP\|uridine phosphorylase |
| 814970 | 0.000351004 | ELOVL1\|elongation of very long chain fatty acids (FEN1/Elo2, SUR4/Elo3, yeast)-like 1 |
| 795757 | 6.07E−05 | FLJ11896\|hypothetical protein FLJ11896 |
| 795758 | 0.001312689 | DKFZP434B044\|hypothetical protein DKFZp434B044 |
| 1486770 | 0.001073614 | FLJ10569\|hypothetical protein FLJ10569 |
| 812294 | 0.001477658 | ESTs |
| 784257 | 0.001547684 | KIF3C\|kinesin family member 3C |
| 769959 | 2.17E−06 | COL4A2\|collagen, type IV, alpha 2 |
| 843321 | 2.31E−05 | KRT7\|keratin 7 |
| 1350439 | 1.82E−11 | KIAA0015\|KIAA0015 gene product |
| 767277 | 1.69E−05 | PPIH\|peptidyl prolyl isomerase H (cyclophilin H) |
| 2018941 | 2.34E−06 | D21S2056E\|DNA segment on chromosome 21 (unique) 2056 expressed sequence |
| 1755555 | 0.001985435 | MAP3K6\|mitogen-activated protein kinase kinase kinase 6 |
| 840788 | 0.008900276 | TMSB10\|thymosin, beta 10 |
| 814989 | 0.001107206 | PPM1G\|protein phosphatase 1G (formerly 2C), magnesium-dependent, gamma isoform |
| 172785 | 0.001708977 | LOC51754\|NAG-5 protein |
| 1608120 | 0.0041899 | AIB3\|nuclear receptor coactivator RAP250; peroxisome proliferator-activated receptor interacting protein; |
| | | thyroid hormone receptor binding protein |
| 767289 | 0.038751321 | KIAA1001\|KIAA1001 protein |
| 1926364 | 0.000305567 | RBM9\|RNA binding motif protein 9 |
| 346134 | 0.024280742 | CRHSP-24\|calcium-regulated heat-stable protein (24 kD) |
| 824753 | 0.000389422 | FLJ22624\|hypothetical protein FLJ22624 |
| 212198 | 3.41E−06 | TP53BP2\|tumor protein p53-binding protein, 2 |
| 813256 | 5.48E−07 | ABCB1\|ATP-binding cassette, sub-family B (MDR/TAP), member 1 |
| 1902841 | 0.001271788 | SLC16A7\|solute carrier family 16 (monocarboxylic acid transporters), member 7 |
| 415766 | 8.23E−06 | KIAA1357\|KIAA1357 protein |
| 562409 | 0.022876597 | NDUFS5\|NADH dehydrogenase (ubiquinone) Fe-S protein 5 (15 kD) (NADH-coenzyme Q reductase) |
| 48886 | 0.037983017 | MRC1\|mannose receptor, C type 1 |
| 713685 | 1.49E−06 | PRSS2\|protease, serine, 2 (trypsin 2) |
| 233464 | 2.63E−06 | EVA1\|epithelial V-like antigen 1 |
| 2010393 | 0.001127212 | FTSJ1\|FtsJ homolog 1 (E. coli) |
| 826363 | 3.96E−05 | LYPLA2\|lysophospholipase II |
| 726791 | 0.016588172 | EST00098\|hypothetical protein EST00098 |
| 788568 | 0.003344334 | ARHGEF7\|Rho guanine nucleotide exchange factor (GEF) 7 |
| 813265 | 4.16E−05 | Homo sapiens mRNA; cDNA DKFZp564H1916 (from clone DKFZp564H1916) |
| 289760 | 0.000134622 | ESTs, Highly similar to T00391 hypothetical protein KIAA0612 [H. sapiens] |
| 768246 | 0.000180794 | Homo sapiens cDNA FLJ30869 fis, clone FEBRA2004224 |
| 783629 | 0.002812591 | HUMAUANTIG\|nucleolar GTPase |
| 2322367 | 2.80E−05 | RTN4\|reticulon 4 |
| 837870 | 0.001262114 | PRELP\|proline arginine-rich end leucine-rich repeat protein |
| 1690915 | 0.005767924 | ESTs |
| 220372 | 0.000449552 | HS3ST1\|heparan sulfate (glucosamine) 3-O-sulfotransferase 1 |
| 140328 | 0.000370802 | ESTs |
| 361539 | 0.002866566 | Homo sapiens sortilin 1 (SORT1), mRNA |
| 84464 | 0.00852718 | FLJ12806\|hypothetical protein FLJ12806 |
| 2018976 | 0.000159096 | PTTG1\|pituitary tumor-transforming 1 |
| 235080 | 0.000566403 | PDGFRA\|platelet-derived growth factor receptor, alpha polypeptide |
| 307255 | 1.71E−06 | ICB-1\|basement membrane-induced gene |
| 154093 | 8.41E−06 | CIR\|CBF1 interacting corepressor |
| 138059 | 0.017914037 | FLJ20374\|hypothetical protein FLJ20374 |
| 363146 | 0.002345896 | PPP3R1\|protein phosphatase 3 (formerly 2B), regulatory subunit B (19 kD), alpha isoform (calcineurin B, |
| | | type I) |
| 1704527 | 4.34E−05 | BCRP2\|Breakpoint cluster region protein, uterine leiomyoma, 2 |
| 150897 | 7.05E−06 | B3GNT3\|UDP-GlcNAc: betaGal beta-1,3-N-acetylglucosaminyltransferase 3 |
| 504940 | 0.00048204 | ESTs |
| 267859 | 0.000661605 | LOC51256\|hypothetical protein |
| 813280 | 0.000241441 | ADSL\|adenylosuccinate lyase |
| 1649746 | 0.039806156 | ESTs |
| 725076 | 0.002237924 | NT5B\|5′-nucleotidase (purine), cytosolic type B |
| 236034 | 0.036288061 | UCP2\|uncoupling protein 2 (mitochondrial, proton carrier) |
| 754509 | 0.000164308 | MET\|met proto-oncogene (hepatocyte growth factor receptor) |
| 31818 | 0.003705111 | Homo sapiens clone 23700 mRNA sequence |
| 220395 | 3.49E−09 | FLJ23293\|likely ortholog of mouse ADP-ribosylation-like factor 6 interacting protein 2 |
| 796759 | 0.03681114 | VDAC3\|voltage-dependent anion channel 3 |
| 768271 | 7.84E−07 | Homo sapiens cDNA FLJ11436 fis, clone HEMBA1001213 |
| 951117 | 0.000316188 | SHMT2\|serine hydroxymethyltransferase 2 (mitochondrial) |
| 731648 | 0.019893343 | NFYA\|nuclear transcription factor Y, alpha |
| 135811 | 2.09E−09 | DEPP\|decidual protein induced by progesterone |
| 1631711 | 0.000920277 | EST |
| 51063 | 0.002903498 | ESTs |
| 824001 | 0.003653268 | FLJ10342\|hypothetical protein FLJ10342 |
| 825740 | 0.043917654 | DKFZP434J1813\|DKFZp434J1813 protein |
| 530761 | 0.007489303 | ADSS\|adenylosuccinate synthase |
| 266135 | 0.016029967 | ESTs, Highly similar to S21424 nestin [H. sapiens] |
| 1509540 | 0.003570072 | HRH1\|histamine receptor H1 |
| 796767 | 0.00038577 | DKFZP564D0764\|DKFZP564D0764 protein |
| 344588 | 0.004877897 | KLK5\|kallikrein 5 |
| 155064 | 0.015807797 | USP13\|ubiquitin specific protease 13 (isopeptidase T-3) |
| 592540 | 0.001362977 | KRT5\|keratin 5 (epidermolysis bullosa simplex, Dowling-Meara/Kobner/Weber-Cockayne types) |
| 624867 | 0.000152832 | FLJ20186\|hypothetical protein FLJ20186 |
| 214131 | 0.00084937 | NIT2\|Nit protein 2 |
| 1854539 | 5.38E−06 | SAM68\|GAP-associated tyrosine phosphoprotein p62 (Sam68) |
| 51078 | 8.56E−06 | ESTs, Weakly similar to JC5314 CDC28/cdc2-like kinase associating arginine-serine cyclophilin |
| | | [H. sapiens] |
| 1659533 | 6.94E−09 | MATN4\|matrilin 4 |
| 42258 | 0.008593378 | ESTs, Weakly similar to CA13_HUMAN COLLAGEN ALPHA 1(III) CHAIN PRECURSOR [H. sapiens] |
| 843398 | 0.042747042 | BMP2\|bone morphogenetic protein 2 |
| 32493 | 2.00E−05 | ITGA6\|integrin, alpha 6 |
| 1671299 | 0.000270513 | HRK\|harakiri, BCL2-interacting protein (contains only BH3 domain) |
| 757873 | 5.86E−06 | CDK5R1\|cyclin-dependent kinase 5, regulatory subunit 1 (p35) |
| 770579 | 0.034946932 | CLDN3\|claudin 3 |
| 665384 | 1.40E−09 | KIAA1609\|KIAA1609 protein |
| 877772 | 7.08E−10 | MRAS\|muscle RAS oncogene homolog |
| 813609 | 1.83E−05 | MP1\|metalloprotease 1 (pitrilysin family) |
| 122739 | 0.005245158 | FLJ21918\|hypothetical protein FLJ21918 |
| 783681 | 0.024373058 | UREB1\|upstream regulatory element binding protein 1 |
| 289027 | 0.029539479 | Homo sapiens cDNA FLJ31413 fis, clone NT2NE2000259, moderately similar to OOCYTE ZINC FINGER |
| | | PROTEIN XLCOF6.1 |
| 52031 | 0.005871121 | ESTs |
| 243295 | 0.000174047 | DKFZP434J154\|DKFZP434J154 protein |
| 376298 | 0.008888435 | CGI-203\|CGI-203 protein |
| 770588 | 0.047831695 | Homo sapiens TTF-I interacting peptide 20 mRNA, partial cds |
| 815214 | 7.86E−05 | CHI3L2\|chitinase 3-like 2 |
| 2016632 | 0.028295772 | ESTs, Moderately similar to T00359 hypothetical protein KIAA0680 [H. sapiens] |
| 131563 | 4.01E−05 | Homo sapiens cDNA FLJ13443 fis, clone PLACE1002853 |
| 254004 | 0.047241769 | LOC51639\|CGI-110 protein |
| 814270 | 0.000160656 | PMSCL1\|polymyositis/scleroderma autoantigen 1 (75 kD) |
| 1468431 | 4.32E−05 | LOC131601\|seven transmembrane domain orphan receptor |
| 783698 | 3.71E−07 | LPIN1\|lipin 1 |
| 504201 | 0.014811257 | Homo sapiens, clone IMAGE: 3677194, mRNA, partial cds |
| 248531 | 4.67E−07 | GMPS\|guanine monphosphate synthetase |
| 108316 | 0.004877897 | RAB3IL1\|RAB3A interacting protein (rabin3)-like 1 |
| 200402 | 0.001715387 | DJ616B8.3\|hypothetical protein dJ616B8.3 |
| 1610490 | 1.12E−05 | CSNK1D\|casein kinase 1, delta |
| 813629 | 0.001013874 | YME1L1\|YME1 (S. cerevisiae)-like 1 |
| 377252 | 5.60E−06 | ADORA2B\|adenosine A2b receptor |
| 49117 | 0.010535254 | KIAA0215\|KIAA0215 gene product |
| 377253 | 0.003016551 | FLJ14728\|hypothetical protein FLJ14728 |
| 366830 | 0.006482896 | ESTs |
| 855872 | 0.035143688 | NRD1\|nardilysin (N-arginine dibasic convertase) |
| 366834 | 0.021925399 | EVPL\|envoplakin |
| 1686314 | 3.96E−09 | Homo sapiens cDNA FLJ31627 fis, clone NT2RI2003338 |
| 755506 | 0.020241721 | ANXA4\|annexin A4 |
| 756163 | 0.003928565 | BCR\|breakpoint cluster region |
| 1642186 | 0.009231307 | DPH2L2\|diptheria toxin resistance protein required for diphthamide biosynthesis (Saccharomyces)-like 2 |
| 1552374 | 0.000107772 | PLSCR3\|phospholipid scramblase 3 |
| 428163 | 0.04294858 | ESTs, Weakly similar to NAH6_HUMAN SODIUM/HYDROGEN EXCHANGER 6 [H. sapiens] |
| 1604220 | 0.000124487 | LRPPRC\|leucine-rich PPR-motif containing |
| 280907 | 0.001755052 | LOC57209\|Kruppel-type zinc finger protein |
| 815235 | 0.000153521 | RCD-8\|autoantigen |
| 1541958 | 0.001831779 | POU2AF1\|POU domain, class 2, associating factor 1 |
| 239724 | 0.000451254 | Homo sapiens mRNA; cDNA DKFZp667B0711 (from clone DKFZp667B0711) |
| 272748 | 0.001611957 | D1S155E\|NRAS-related gene |
| 1868626 | 0.033272185 | PFKL\|phosphofructokinase, liver |
| 814297 | 0.002489422 | PEPD peptidase D |
| 768619 | 0.016678558 | Homo sapiens cDNA FLJ32643 fis, clone SYNOV2001212 |
| 950516 | 0.007644628 | CNNM4\|cyclin M4 |
| 80549 | 0.02497657 | PBX2\|pre-B-cell leukemia transcription factor 2 |
| 51408 | 0.00712188 | DSCR1L1\|Down syndrome critical region gene 1-like 1 |
| 366848 | 3.47E−08 | ESTs |
| 825005 | 1.29E−05 | IPLA2(GAMMA)\|intracellular membrane-associated calcium-independent phospholipase A2 gamma |
| 739193 | 0.002561152 | CRABP1\|cellular retinoic acid-binding protein 1 |
| 301735 | 0.039966603 | TGM3\|transglutaminase 3 (E polypeptide, protein-glutamine-gamma-glutamyltransferase) |
| 358689 | 0.039496567 | EPHB2\|EphB2 |
| 2030301 | 0.017481981 | SLIT3\|slit (Drosophila) homolog 3 |
| 1550783 | 0.009173647 | CHORDC1\|cysteine and histidine-rich domain (CHORD)-containing, zinc-binding protein 1 |
| 824068 | 0.047848128 | COX5A\|cytochrome c oxidase subunit Va |
| 345586 | 0.003770645 | TNFRSF12\|tumor necrosis factor receptor superfamily, member 12 (translocating chain-association |
| | | membrane protein) |
| 813645 | 0.000248914 | FLJ20500\|hypothetical protein |
| 741474 | 4.08E−06 | GPI\|glucose phosphate isomerase |
| 502625 | 0.000483976 | FLJ21212\|hypothetical protein FLJ21212 |
| 180263 | 0.005375632 | KIAA0537\|KIAA0537 gene product |
| 377275 | 5.69E−15 | TRIM29\|tripartite motif-containing 29 |
| 127510-2 | 7.79E−05 | CDC25A\|cell division cycle 25A |
| 41650 | 0.044529607 | HGF\|hepatocyte growth factor (hepapoietin A; scatter factor) |
| 2019214 | 0.00105118 | CAD\|carbamoyl-phosphate synthetase 2, aspartate transcarbamylase, and dihydroorotase |
| 281580 | 0.035143688 | ESTs |
| 842794 | 2.57E−05 | KIAA1668\|KIAA1668 protein |
| 841057 | 1.17E−05 | MAGE-E1\|MAGE-E1 protein |
| 530036 | 0.007079545 | FLJ13078\|hypothetical protein FLJ13078 |
| 813651 | 0.000139851 | ALAS1\|aminolevulinate, delta-, synthase 1 |
| 2308314 | 0.016063781 | KIAA1055\|KIAA1055 protein |
| 785616 | 0.005985046 | SSR1\|signal sequence receptor, alpha (translocon-associated protein alpha) |
| 2604635 | 0.001601276 | YAP1\|Yes-associated protein 1, 65 kDa |
| 50483 | 0.001534143 | FBLN5\|fibulin 5 |
| 1509904 | 0.020270563 | PTGS2\|prostaglandin-endoperoxide synthase 2 (prostaglandin G/H synthase and cyclooxygenase) |
| 809784 | 1.53E−05 | KLK6\|kallikrein 6 (neurosin, zyme) |
| 725454 | 0.010208539 | CKS2\|CDC28 protein kinase 2 |
| 490649 | 3.45E−05 | MCCC1\|methylcrotonoyl-Coenzyme A carboxylase 1 (alpha) |
| 146605 | 0.012998471 | FPRL1\|formyl peptide receptor-like 1 |
| 773183 | 9.02E−05 | AF093680\|similar to mouse Glt3 or D. malanogaster transcription factor IIB |
| 845355 | 0.038531356 | CTSC\|cathepsin C |
| 1570339 | 1.02E−06 | Homo sapiens, clone IMAGE: 3948909, mRNA, partial cds |
| 83120 | 0.000644819 | RBM10\|RNA binding motif protein 10 |
| 753946 | 0.001034397 | HKE2\|HLA class II region expressed gene KE2 |
| 744047 | 0.000853947 | PLK\|polo (Drosophia)-like kinase |
| 814617 | 0.001106068 | MEF2A\|MADS box transcription enhancer factor 2, polypeptide A (myocyte enhancer factor 2A) |
| 813671 | 7.06E−05 | ESTs, Weakly similar to T31636 hypothetical protein Y57A10A.n - Caenorhabditis elegans [C. elegans] |
| 815276 | 0.038667897 | NUP62\|nucleoporin 62 kD |
| 813673 | 0.011252089 | HDGF\|hepatoma-derived growth factor (high-mobility group protein 1-like) |
| 2018297 | 0.038531356 | CTL2\|CTL2 gene |
| 2009477 | 0.006324566 | CD6\|CD6 antigen |
| 2713047 | 0.000148737 | PVR\|poliovirus receptor |
| 844703 | 7.67E−08 | T-STAR\|Sam68-like phosphotyrosine protein, T-STAR |
| 703541 | 1.21E−05 | KIAA1858\|KIAA1858 protein |
| 2062028 | 0.001614105 | ZNF259\|zinc finger protein 259 |
| 770935 | 0.000827726 | 7h3\|hypothetical protein FLJ13511 |
| 29927 | 0.012880496 | FLJ10737\|hypothetical protein FLJ10737 |
| 42627 | 0.035383143 | COCH\|coagulation factor C (Limulus polyphemus homolog); cochlin |
| 51448 | 0.00048184 | ATF3\|activating transcription factor 3 |
| 133518 | 0.000238959 | MAPRE2\|microtubule-associated protein, RP/EB family, member 2 |
| 1455394 | 2.92E−06 | HCS\|cytochrome c |
| 1898758 | 0.011460543 | BCE-1\|BCE-1 protein |
| 36393-2 | 0.005878639 | ACAT2\|acetyl-Coenzyme A acetyltransferase 2 (acetoacetyl Coenzyme A thiolase) |
| 815285 | 0.012980026 | ERPROT213-21\|protein with polyglutamine repeat; calcium (ca2+) homeostasis endoplasmic reticulum |
| | | protein |
| 486787 | 0.001271197 | CNN3\|calponin 3, acidic |
| 626841 | 0.012861341 | MET\|met proto-oncogene (hepatocyte growth factor receptor) |
| 2308346 | 0.027141267 | CDK2\|cyclin-dependent kinase 2 |
| 756502 | 0.000554935 | NUDT1\|nudix (nucleoside diphosphate linked moiety X)-type motif 1 |
| 502669 | 9.98E−06 | HDAC2\|histone deacetylase 2 |
| 129644 | 0.003508671 | SSH3BP1\|spectrin SH3 domain binding protein 1 |
| 898328 | 6.78E−07 | EDR2\|early development regulator 2 (homolog of polyhomeotic 2) |
| 756509 | 0.041594075 | H105E3\|NAD(P) dependent steroid dehydrogenase-like; H105e3 |
| 745003 | 0.002269285 | ZNF76\|zinc finger protein 76 (expressed in testis) |
| 590242 | 0.000669517 | ESTs |
| 841091 | 0.000803586 | FAF1\|Fas (TNFRSF6) associated factor 1 |
| 841093 | 0.000742995 | CUL1\|cullin 1 |
| 345916 | 0.000621801 | Homo sapiens mRNA; cDNA DKFZp564N1063 (from clone DKFZp564N1063) |
| 1460224 | 0.006815479 | ESTs, Weakly similar to I38133 protein kinase [H. sapiens] |
| 379200 | 0.00041118 | IRAK1\|interleukin-1 receptor-associated kinase 1 |
| 2009491 | 0.005321091 | LOC51191\|cyclin-E binding protein 1 |
| 41698 | 0.00083009 | PGRMC1\|progesterone receptor membrane component 1 |
| 2306752 | 0.000328235 | STMN2\|stathmin-like 2 |
| 813698 | 0.009151254 | SPRY2\|sprouty (Drosophila) homolog 2 |
| 898332 | 0.001879562 | NPR1\|natriuretic peptide receptor A/guanylate cyclase A (atrionatriuretic peptide receptor A) |
| 856887 | 0.02806084 | ESTs |
| 1570364 | 0.005587928 | FLJ21324\|hypothetical protein FLJ21324 |
| 950578 | 0.000159834 | NDUFA5\|NADH dehydrogenase (ubiquinone) 1 alpha subcomplex, 5 (13 kD, B13) |
| 745011 | 3.47E−05 | KIAA1750\|KIAA1750 protein |
| 178324 | 0.04004725 | ESTs |
| 280970 | 0.00148396 | NOL1\|nucleolar protein 1 (120 kD) |
| 309161-2 | 0.001831364 | TBX2\|T-box 2 |
| 788205 | 0.001756144 | SOX4\|SRY (sex determining region Y)-box 4 |
| 755578 | 7.43E−08 | SLC7A5\|solute carrier family 7 (cationic amino acid transporter, y+system), member 5 |
| 786605 | 8.78E−05 | APG-1\|heat shock protein (hsp110 family) |
| 123774 | 0.003147591 | ESTs |
| 83156 | 0.003569567 | C6orf34\|chromosome 6 open reading frame 34 |
| 416075 | 0.003099407 | TCF19\|transcription factor 19 (SC1) |
| 773502 | 0.018979013 | ESTs, Weakly similar to S65824 reverse transcriptase homolog [H. sapiens] |
| 502682 | 0.037642103 | ENIGMA\|enigma (LIM domain protein) |
| 502689 | 0.029859423 | Homo sapiens clone HH409 unknown mRNA |
| 309288 | 0.005363273 | RFC4\|replication factor C (activator 1) 4 (37 kD) |
| 307687 | 0.000599469 | PRSS16\|protease, serine, 16 (thymus) |
| 44255 | 6.27E−06 | MRPL3\|mitochondrial ribosomal protein L3 |
| 1926024 | 0.004189378 | BCL2L12\|BCL2-like 12 (proline rich) |
| 1160723 | 2.20E−05 | LIMK2\|LIM domain kinase 2 |
| 85840 | 0.000112138 | NNMT\|nicotinamide N-methyltransferase |
| 130201 | 0.000950305 | ICAM2\|intercellular adhesion molecule 2 |
| 33837 | 0.004299371 | KIAA1500\|KIAA1500 protein |
| 530093 | 0.000479617 | MPZL1\|myelin protein zero-like 1 |
| 1358393 | 0.0011328 | MAP2K3\|mitogen-activated protein kinase kinase 3 |
| 282587 | 0.000754806 | CA11\|carbonic anhydrase XI |
| 502690 | 0.00352586 | RPN1\|ribophorin I |
| 626874 | 9.10E−08 | FLJ23399\|hypothetical protein FLJ23399 |
| 853562 | 0.023644346 | TRIM28\|tripartite motif-containing 28 |
| 782339 | 8.33E−05 | PRKAB1\|protein kinase, AMP-activated, beta 1 non-catalytic subunit |
| 162308-2 | 0.000128308 | CYYR1\|cysteine and tyrosine-rich protein 1 |
| 366126 | 2.91E−05 | KIAA0354\|KIAA0354 gene product |
| 703581 | 0.034266941 | PRG1\|proteoglycan 1, secretory granule |
| 741831 | 0.02005317 | PLTP\|phospholipid transfer protein |
| 825085 | 3.88E−07 | ST14\|suppression of tumorigenicity 14 (colon carcinoma, matriptase, epithin) |
| 814662 | 0.016952234 | AP4B1\|adaptor-related protein complex 4, beta 1 subunit |
| 1468820 | 0.004338248 | NOS3\|nitric oxide synthase 3 (endothelial cell) |
| 852913 | 0.033456546 | SNRPF\|small nuclear ribonucleoprotein polypeptide F |
| 704532 | 0.000420604 | NMI\|N-myc (and STAT) interactor |
| 245296 | 0.049442498 | RAD52\|RAD52 (S. cerevisiae) homolog |
| 1880757 | 0.006991988 | MAP3K5\|mitogen-activated protein kinase kinase kinase 5 |
| 788234 | 4.76E−08 | ID4\|inhibitor of DNA binding 4, dominant negative helix-loop-helix protein |
| 741841 | 0.003707205 | DDX11\|DEAD/H (Asp-Glu-Ala-Asp/His) box polypeptide 11 (S. cerevisiae CHL1-like helicase) |
| 211216 | 0.024810654 | KLF1\|Kruppel-like factor 1 (erythroid) |
| 360254 | 0.000831262 | CYR61\|cysteine-rich, angiogenic inducer, 61 |
| 741842 | 0.000446292 | LOC51690\|U6 snRNA-associated Sm-like protein LSm7 |
| 363597 | 0.000180473 | C11orf8\|chromosome 11 open reading frame 8 |
| 726464 | 0.001854106 | SPAG9\|sperm associated antigen 9 |
| 49502 | 0.0009273 | CYLN2\|cytoplasmic linker 2 |
| 47900 | 0.006683198 | KIAA1382\|amino acid transporter 2 |
| 529861 | 1.29E−05 | PSMB6 proteasome (prosome, macropain) subunit, beta type, 6 |
| 810391 | 0.016029967 | HYAL1\|hyaluronoglucosaminidase 1 |
| 2322038 | 5.74E−07 | COL4A6\|collagen, type IV, alpha 6 |
| 2012757 | 0.000801869 | D2S448\|Melanoma associated gene |
| 1632011 | 0.0039532 | NPR2\|natriuretic peptide receptor B/guanylate cyclase B (atrionatriuretic peptide receptor B) |
| 845709 | 1.35E−05 | ESTs |
| 470393 | 1.08E−05 | MMP7\|matrix metalloproteinase 7 (matrilysin, uterine) |
| 132911 | 0.001603821 | PPP1CB\|protein phosphatase 1, catalytic subunit, beta isoform |
| 756556 | 0.008247752 | SERPING1\|serine (or cysteine) proteinase inhibitor, clade G (C1 inhibitor), member 1 |
| 1635359 | 0.031634717 | RASD1\|RAS, dexamethasone-induced 1 |
| 753215 | 4.53E−06 | GNAI1\|guanine nucleotide binding protein (G protein), alpha inhibiting activity polypeptide 1 |
| 738916 | 0.014632388 | REV1L\|REV1 (yeast homolog)- like |
| 361204 | 1.64E−06 | COL9A3\|collagen, type IX, alpha 3 |
| 1536727 | 0.022064191 | MGC11324\|hypothetical protein MGC11324 |
| 2096458 | 4.36E−08 | CALU\|calumenin |
| 869234 | 0.005357267 | ESTs |
| 739578 | 0.013280693 | C4.4A\|GPI-anchored metastasis-associated protein homolog |
| 788247 | 0.020732026 | CUL2\|cullin 2 |
| 430834 | 0.027276602 | Homo sapiens, Similar to RIKEN cDNA 2810405F18 gene, clone MGC: 22960 IMAGE: 4865283, mRNA, |
| | | complete cds |
| 1639640 | 0.001388783 | EIF5A\|eukaryotic translation initiation factor 5A |
| 564492 | 2.90E−05 | MTCH2\|mitochondrial carrier homolog 2 |
| 244654 | 0.001763838 | ELAC1\|elaC (E. coli) homolog 1 |
| 1574693 | 0.000601881 | SLB\|selective LIM binding factor, rat homolog |
| 860013 | 0.024373058 | MGC1136\|hypothetical protein MGC1136 |
| 897720 | 0.000629574 | TRO\|trophinin |
| 137862 | 0.028270162 | HIP-55\|src homology 3 domain-containing protein HIP-55 |
| 1635364 | 3.42E−06 | LSM2\|U6 snRNA-associated Sm-like protein |
| 1551030 | 0.031216746 | CDA\|cytidine deaminase |
| 293328 | 0.000795582 | C2orf2\|chromosome 2 open reading frame 2 |
| 1404396 | 0.000304786 | PLCB3\|phospholipase C, beta 3 (phosphatidylinositol-specific) |
| 292388 | 0.004770631 | LOC55977\|hypothetical protein 24636 |
| 840493 | 0.006951821 | RNASE1\|ribonuclease, RNase A family, 1 (pancreatic) |
| 268188 | 0.000728707 | PRRG1\|proline-rich Gla (G-carboxyglutamic acid) polypeptide 1 |
| 503671 | 2.23E−08 | Homo sapiens cDNA FLJ14368 fis, clone HEMBA1001122 |
| 645495 | 0.014557615 | BICD1\|Bicaudal D (Drosophila) homolog 1 |
| 80946 | 0.005577147 | ABCE1\|ATP-binding cassette, sub-family E (OABP), member 1 |
| 1883327 | 0.015005389 | Homo sapiens cDNA FLJ30999 fis, clone HLUNG1000110, weakly similar to PISTIL-SPECIFIC |
| | | EXTENSIN-LIKE PROTEIN PRECURSOR |
| 897731 | 0.014470496 | KIAA0786\|latrophilin |
| 897732 | 5.30E−05 | SAD1\|SnRNP assembly defective 1 homolog |
| 1950274 | 0.025668592 | ESTs |
| 753236 | 0.000420604 | ESTs, Weakly similar to S71512 hypothetical protein T2 - mouse [M. musculus] |
| 1055581 | 0.038751321 | DKFZP564K192\|likely ortholog of mouse dysbindin |
| 344243 | 0.00041118 | UMPK\|uridine monophosphate kinase |
| 788264 | 0.023178776 | DPAGT1\|dolichyl-phosphate (UDP-N-acetylglucosamine) N-acetylglucosaminephosphotransferase 1 |
| | | (GlcNAc-1-P transferase) |
| 774502 | 0.031310371 | PTPN12\|protein tyrosine phosphatase, non-receptor type 12 |
| 49532 | 8.13E−08 | VIM\|vimentin |
| 1558675 | 2.00E−07 | SOX10\|SRY (sex determining region Y)-box 10 |
| 308682 | 0.000155359 | ABCF2\|ATP-binding cassette, sub-family F (GCN20), member 2 |
| 773564 | 0.000739744 | NIN283 nerve injury gene 283 |
| 2719303 | 0.015247808 | SLC18A1\|solute carrier family 18 (vesicular monoamine), member 1 |
| 125722 | 0.003691973 | DGUOK\|deoxyguanosine kinase |
| 509570 | 0.015704565 | Homo sapiens cDNA: FLJ22656 fis, clone HSI07655 |
| 231574 | 0.048699543 | FBI1\|HIV-1 inducer of short transcripts binding protein; lymphoma related factor |
| 301061 | 0.000176121 | COL18A1\|collagen, type XVIII, alpha 1 |
| 79629 | 0.011251927 | CXCR4\|chemokine (C—X—C motif), receptor 4 (fusin) |
| 344251 | 0.012443564 | FLJ10783\|hypothetical protein FLJ10783 |
| 795499 | 0.010004815 | NDRG4\|NDRG family, member 4 |
| 772912 | 0.02555164 | AGS3\|likely ortholog of rat activator of G-protein signaling 3 |
| 156473 | 0.001838545 | EPHX2\|epoxide hydrolase 2, cytoplasmic |
| 772913 | 1.23E−11 | Homo sapiens cDNA FLJ31951 fis, clone NT2RP7007177, weakly similar to Homo sapiens multiple |
| | | membrane spanning receptor TRC8 mRNA |
| 773571 | 2.09E−06 | GTF3C3\|general transcription factor IIIC, polypeptide 3 (102 kD) |
| 630013 | 0.000293426 | MSH2\|mutS (E. coli) homolog 2 (colon cancer, nonpolyposis type 1) |
| 856167 | 8.78E−05 | TARS\|threonyl-tRNA synthetase |
| 2322079 | 0.020096884 | EST |
| 1472754 | 0.019822944 | COX6B\|cytochrome c oxidase subunit VIb |
| 756595 | 6.16E−05 | S100A10\|S100 calcium-binding protein A10 (annexin II ligand, calpactin I, light polypeptide (p11)) |
| 50884 | 0.002936718 | DYRK2\|dual-specificity tyrosine-(Y)-phosphorylation regulated kinase 2 |
| 509589 | 0.00458557 | LOC51122\|HSPC042 protein |
| 884657 | 0.036349025 | TIMM8B\|translocase of inner mitochondrial membrane 8 (yeast) homolog B |
| 121454 | 0.004821899 | ALOX12\|arachidonate 12-lipoxygenase |
| 112636 | 1.36E−05 | IL1RAP\|interleukin 1 receptor accessory protein |
| 2062415 | 1.01E−05 | GMDS\|GDP-mannose 4,6-dehydratase |
| 135503 | 0.022046067 | BRD4\|bromodomain-containing 4 |
| 2062418 | 0.034945258 | AIM1\|absent in melanoma 1 |
| 843070 | 4.81E−05 | NUP88\|nucleoporin 88 kD |
| 1759582 | 0.000276452 | FN14\|type I transmembrane protein Fn14 |
| 50892 | 0.044429786 | Homo sapiens, clone MGC: 9913 IMAGE: 3870821, mRNA, complete cds |
| 566421 | 0.023116508 | PIMT\|PRIP-interacting protein with methyltransferase domain |
| 841478 | 0.000244327 | VIT1\|vitiligo-associated protein VIT-1 |
| 1860497 | 0.000102553 | Homo sapiens, clone MGC: 5352 IMAGE: 3048106, mRNA, complete cds |
| 418049 | 1.52E−06 | Homo sapiens cDNA: FLJ21909 fis, clone HEP03834 |
| 855521 | 0.040241164 | KRT18\|keratin 18 |
| 767312 | 0.040844999 | PLD2\|phospholipase D2 |
| 235882 | 0.001352198 | KIAA0709\|endocytic receptor (macrophage mannose receptor family) |
| 306318 | 3.49E−06 | ORC6L\|origin recognition complex, subunit 6 (yeast homolog)-like |
| 71087 | 0.022618851 | MAFF\|v-maf musculoaponeurotic fibrosarcoma (avian) oncogene family, protein F |
| 840821 | 0.044848397 | SSR4\|signal sequence receptor, delta (translocon-associated protein delta) |
| 525926 | 6.55E−07 | SDC1\|syndecan 1 |
| 1686766 | 9.44E−06 | RAGD\|Rag D protein |
| 613056 | 0.005068207 | RCN1\|reticulocalbin 1, EF-hand calcium binding domain |
| 79655 | 0.003417271 | MGC2722\|hypothetical protein MGC2722 |
| 489729 | 1.40E−07 | ETS1\|v-ets avian erythroblastosis virus E26 oncogene homolog 1 |
| 2610539 | 0.005368978 | KRT6B\|keratin 6B |
| 795805 | 0.001440423 | KIAA0332\|KIAA0332 protein |
| 841485 | 0.01354434 | Homo sapiens cDNA FLJ31058 fis, clone HSYRA2000828 |
| 74713 | 0.00017506 | FLJ20059\|hypothetical protein FLJ20059 |
| 753278 | 1.39E−05 | CXCL16\|chemokine (C—X—C motif) ligand 16 |
| 796469 | 0.016508823 | HSPC150\|HSPC150 protein similar to ubiquitin-conjugating enzyme |
| 1613496 | 0.001928041 | EST |
| 430894 | 0.012480222 | TLN1\|talin 1 |
| 1947381 | 0.000752302 | FLJ22329\|hypothetical protein FLJ22329 |
| 2499237 | 0.000760968 | SERPINB5\|serine (or cysteine) proteinase inhibitor, clade B (ovalbumin), member 5 |
| 321708 | 0.01621547 | TFDP1\|transcription factor Dp-1 |
| 843091 | 0.000367291 | MGC20533\|similar to RIKEN cDNA 2410004L22 gene (M. musculus) |
| 588368 | 0.01394813 | KIAA0947\|KIAA0947 protein |
| 590640 | 7.53E−05 | PDXK\|pyridoxal (pyridoxine, vitamin B6) kinase |
| 1519013 | 1.16E−06 | Homo sapiens, clone IMAGE: 3537447, mRNA, partial cds |
| 823850 | 0.001603821 | RAI14\|retinoic acid induced 14 |
| 357396 | 0.038563775 | Homo sapiens cDNA: FLJ23137 fis, clone LNG08842 |
| 345234 | 0.002720745 | ESTs, Weakly similar to T00705 N-chimerin homolog F25965_3 [H. sapiens] |
| 753285 | 0.047573891 | GYG\|glycogenin |
| 566443 | 6.48E−06 | Homo sapiens cDNA FLJ12793 fis, clone NT2RP2002033 |
| 796475 | 2.49E−07 | FHL3\|four and a half LIM domains 3 |
| 83549 | 0.000116498 | C1R\|complement component 1, r subcomponent |
| 1637311 | 0.003072498 | KIAA0036\|KIAA0036 gene product |
| 810753 | 0.000438842 | MRPS10\|mitochondrial ribosomal protein S10 |
| 41300 | 8.52E−05 | Homo sapiens clones 24714 and 24715 mRNA sequence |
| 1554727 | 0.005587928 | HSF2\|heat shock transcription factor 2 |
| 743810 | 0.012831119 | MGC2577\|hypothetical protein MGC2577 |
| 140071 | 0.011056053 | FRZB\|frizzled-related protein |
| 726830 | 0.022786019 | FLJ10748\|hypothetical protein FLJ10748 |
| 282977 | 0.039374425 | ADCY2\|adenylate cyclase 2 (brain) |
| 884690 | 0.001462177 | CCT8\|chaperonin containing TCP1, subunit 8 (theta) |
| 282978 | 0.000356685 | TGFB2\|transforming growth factor, beta 2 |
| 823864 | 0.005015434 | TCN2\|transcobalamin II; macrocytic anemia |
| 415814 | 0.000792587 | LOC56901\|NADH: ubiquinone oxidoreductase MLRQ subunit homolog |
| 511107 | 0.005854823 | CCT4\|chaperonin containing TCP1, subunit 4 (delta) |
| 741139 | 0.000808096 | EYA2\|eyes absent (Drosophila) homolog 2 |
| 323322 | 0.008351255 | ESTs |
| 810762 | 0.018133375 | YKT6\|SNARE protein |
| 49595 | 0.010562779 | DKFZP434F091\|DKFZP434F091 protein |
| 1637328 | 0.000165971 | AOX1\|aldehyde oxidase 1 |
| 2062453 | 0.001470365 | DKFZP727G051\|DKFZP727G051 protein |
| 703972 | 0.002002869 | KIAA0699\|KIAA0699 protein |
| 669375 | 6.11E−06 | DKK1\|dickkopf (Xenopus laevis) homolog 1 |
| 489755 | 0.001048773 | ADAM12\|a disintegrin and metalloproteinase domain 12 (meltrin alpha) |
| 611481 | 1.40E−06 | TCF-3\|HMG-box transcription factor TCF-3 |
| 487071 | 0.000245994 | GNRH1\|gonadotropin-releasing hormone 1 (leutinizing-releasing hormone) |
| 417424 | 0.000622484 | ABP/ZF\|Alu-binding protein with zinc finger domain |
| 788617 | 9.28E−09 | LOC91689\|hypothetical gene supported by AL449243 |
| 825478 | 0.010722629 | ZNF146\|zinc finger protein 146 |
| 431869 | 0.017680318 | Homo sapiens, clone IMAGE: 3506202, mRNA, partial cds |
| 897011 | 0.00140216 | KIAA0842\|KIAA0842 protein |
| 1910785 | 0.001443649 | ATP11A\|ATPase, Class VI, type 11A |
| 813609-2 | 5.33E−06 | MP1\|metalloprotease 1 (pitrilysin family) |
| 284592 | 5.11E−11 | DKFZp762A227\|hypothetical protein DKFZp762A227 |
| 788620 | 4.20E−05 | Homo sapiens, clone MGC: 17225 IMAGE: 4151716, mRNA, complete cds |
| 1837280 | 0.002741302 | EST |
| 504940-2 | 6.84E−05 | ESTs |
| 795841 | 0.000205324 | FLJ14466\|hypothetical protein FLJ14466 |
| 345262 | 0.032131824 | PCDHB2\|protocadherin beta 2 |
| 162308 | 0.000536545 | CYYR1\|cysteine and tyrosine-rich protein 1 |
| 853977 | 0.000294844 | ESTs |
| 1534430 | 0.012397017 | GUCY1B2\|guanylate cyclase 1, soluble, beta 2 |
| 450464 | 0.008351255 | ACVR2\|activin A receptor, type II |
| 142788 | 4.92E−08 | SERPINH2\|serine (or cysteine) proteinase inhibitor, clade H (heat shock protein 47), member 2 |
| 486436 | 0.002295943 | UGP2\|UDP-glucose pyrophosphorylase 2 |
| 629839 | 0.034919482 | dJ908M14.2\|hypothetical protein dJ908M14.2 |
| 781014 | 2.92E−05 | ST5\|suppression of tumorigenicity 5 |
| 241705 | 0.002362784 | PYGL\|phosphorylase, glycogen; liver (Hers disease, glycogen storage disease type VI) |
| 782756 | 1.92E−05 | TBPL1\|TBP-like 1 |
| 293715 | 0.047831695 | RAB1\|RAB1, member RAS oncogene family |
| 2345302 | 0.027994066 | KIAA0675\|KIAA0675 gene product |
| 509943 | 0.000400309 | DKC1\|dyskeratosis congenita 1, dyskerin |
| 1534440 | 0.033905314 | EST |
| 743851 | 0.024878213 | ESTs, Weakly similar to S55916 ribosomal protein S5, cytosolic [H. sapiens] |
| 1456799 | 0.019816713 | Homo sapiens cDNA FLJ32537 fis, clone SMINT2000400, highly similar to Homo sapiens FRG1 mRNA |
| 415851 | 6.35E−08 | ARHE\|ras homolog gene family, member E |
| 248256 | 2.71E−07 | KIAA1691\|KIAA1691 protein |
| 810002 | 3.90E−05 | Homo sapiens, clone MGC: 19762 IMAGE: 3636045, mRNA, complete cds |
| 626502 | 4.10E−08 | ARPC1B\|actin related protein 2/3 complex, subunit 1B (41 kD) |
| 167268 | 0.047573891 | ESTs |
| 781026 | 0.016470796 | Homo sapiens cDNA FLJ11801 fis, clone HEMBA1006253, weakly similar to DNA-DAMAGE- |
| | | REPAIR/TOLERATION PROTEIN DRT111 PRECURSOR |
| 462926 | 0.01888892 | NEK2\|NIMA (never in mitosis gene a)-related kinase 2 |
| 156270-2 | 0.004049154 | ESTs |
| 44692 | 0.000993361 | CRHR1\|corticotropin releasing hormone receptor 1 |
| 753625 | 0.00994019 | Homo sapiens cDNA FLJ14844 fis, clone PLACE1000133, highly similar to TRANSCRIPTION FACTOR |
| | | BTF3 |
| 683059 | 2.83E−06 | CEP3\|Cdc42 effector protein 3 |
| 756968 | 0.015062537 | EFNB1\|ephrin-B1 |
| 1898426 | 0.02483948 | MGC4268\|hypothetical protein MGC4268 |
| 739983 | 0.0041899 | KIAA0042\|KIAA0042 gene product |
| 1473788 | 0.016370991 | TRADD\|TNFRSF1A-associated via death domain |
| 66902 | 0.000352001 | Homo sapiens cDNA FLJ32121 fis, clone PEBLM1000083 |
| 41358 | 0.037052367 | MEIS2\|Meis (mouse) homolog 2 |
| 743866 | 2.59E−07 | FLJ10201\|hypothetical protein FLJ10201 |
| 725143 | 0.040000458 | FLJ22418\|hypothetical protein FLJ22418 |
| 49920 | 0.035411663 | PTDSS1\|phosphatidylserine synthase 1 |
| 260303 | 4.78E−05 | ETS2\|v-ets avian erythroblastosis virus E26 oncogene homolog 2 |
| 343695 | 0.002155839 | Homo sapiens cDNA FLJ31668 fis, clone NT2RI2004916 |
| 713782 | 0.000328235 | ADAM15\|a disintegrin and metalloproteinase domain 15 (metargidin) |
| 240780 | 0.000237546 | NINJ2\|ninjurin 2 |
| 136534 | 0.003770645 | MGC10753\|hypothetical protein MGC10753 |
| 462939 | 0.036642651 | Homo sapiens cDNA FLJ31303 fis, clone LIVER1000082 |
| 291057 | 0.024002401 | CDKN2C\|cyclin-dependent kinase inhibitor 2C (p18, inhibits CDK4) |
| 417473 | 0.010898808 | GNAS1\|guanine nucleotide binding protein (G protein), alpha stimulating activity polypeptide 1 |
| 725152 | 3.29E−10 | DKFZp762A227\|hypothetical protein DKFZp762A227 |
| 814309 | 0.031933058 | PIST\|PDZ/coiled-coil domain binding partner for the rho-family GTPase TC10 |
| 858153 | 2.72E−07 | NFIL3\|nuclear factor, interleukin 3 regulated |
| 772220 | 7.74E−06 | PDIR\|for protein disulfide isomerase-related |
| 2057973 | 0.043575209 | CCND3\|cyclin D3 |
| 781047 | 0.000900648 | BUB1\|budding uninhibited by benzimidazoles 1 (yeast homolog) |
| 37491 | 0.000305556 | TRIP10\|thyroid hormone receptor interactor 10 |
| 134942 | 0.011506861 | FBXL11\|f-box and leucine-rich repeat protein 11 |
| 1553054 | 0.013614879 | PRSS12\|protease, serine, 12 (neurotrypsin, motopsin) |
| 208718 | 1.94E−09 | ANXA1\|annexin A1 |
| 230235 | 0.018133375 | CRFG\|G protein-binding protein CRFG |
| 214990 | 1.04E−07 | GSN\|gelsolin (amyloidosis, Finnish type) |
| 418422 | 9.66E−07 | PPBP\|pro-platelet basic protein (includes platelet basic protein, beta-thromboglobulin, connective tissue- |
| | | activating peptide III, neutrophil-activating peptide-2) |
| 123441 | 0.02798739 | GALNT2\|UDP-N-acetyl-alpha-D-galactosamine: polypeptide N-acetylgalactosaminyltransferase 2 |
| | | (GaINAc-T2) |
| 742143 | 0.03760007 | CD5\|CD5 antigen (p56-62) |
| 69184 | 0.000101368 | SUDD\|sudD (suppressor of bimD6, Aspergillus nidulans) homolog |
| 795893 | 0.010292994 | PPP1R15A\|protein phosphatase 1, regulatory (inhibitor) subunit 15A |
| 120106 | 0.018649252 | CASP1\|caspase 1, apoptosis-related cysteine protease (interleukin 1, beta, convertase) |
| 2919651 | 0.000222692 | PGLYRP\|peptidoglycan recognition protein |
| 898010 | 0.000371143 | EST |
| 858167 | 8.01E−05 | FACL4\|fatty-acid-Coenzyme A ligase, long-chain 4 |
| 782794 | 0.001204679 | SLC25A19\|solute carrier family 25 (mitochondrial deoxynucleotide carrier), member 19 |
| 33500 | 2.81E−08 | Homo sapiens clone 23556 mRNA sequence |
| 133213 | 0.001335593 | FUT4\|fucosyltransferase 4 (alpha (1,3) fucosyltransferase, myeloid-specific) |
| 1553065 | 0.010881395 | FLJ14993\|hypothetical protein FLJ14993 |
| 416833 | 0.016095957 | FKBP5\|FK506-binding protein 5 |
| 68534 | 0.006753838 | Homo sapiens cDNA: FLJ22290 fis, clone HRC04405 |
| 1574330 | 7.62E−05 | GROS1\|growth suppressor 1 |
| 502367 | 0.027654372 | FBLN1\|fibulin 1 |
| 502369 | 3.35E−06 | PDCD5\|programmed cell death 5 |
| 856575 | 6.13E−05 | APP\|amyloid beta (A4) precursor protein (protease nexin-II, Alzheimer disease) |
| 366591 | 3.53E−05 | TIAM2\|T-cell lymphoma invasion and metastasis 2 |
| 290422 | 2.17E−06 | ZNF216\|zinc finger protein 216 |
| 1534493 | 1.17E−07 | ESTs |
| 280666 | 0.011314426 | SEMA4D\|sema domain, immunoglobulin domain (Ig), transmembrane domain (TM) and short |
| | | cytoplasmic domain, (semaphorin) 4D |
| 344672 | 0.017131408 | DKFZp761J139\|hypothetical protein DKFZp761J139 |
| 23758 | 0.005068225 | Homo sapiens clone 23758 mRNA sequence |
| 858183 | 0.001057603 | Homo sapiens mRNA full length insert cDNA clone EUROIMAGE 684990 |
| 629896 | 0.000370779 | MAP1B\|microtubule-associated protein 1B |
| 725188 | 0.027824136 | MDH1\|malate dehydrogenase 1, NAD (soluble) |
| 858188 | 0.042931319 | Homo sapiens cDNA FLJ32144 fis, clone PLACE5000105, highly similar to Mus musculus mRNA for |
| | | heparan sulfate 6-sulfotransferase 2 |
| 810057 | 6.03E−08 | CSDA\|cold shock domain protein A |
| 2062825 | 0.001409555 | KIAA0964\|KIAA0964 protein |
| 50506 | 0.000975654 | MAPK6\|mitogen-activated protein kinase 6 |
| 739235 | 0.000209372 | STK25\|serine/threonine kinase 25 (Ste20, yeast homolog) |
| 345621 | 0.002744036 | CXX1\|CAAX box 1 |
| 743113 | 1.29E−05 | KLK13\|kallikrein 13 |
| 133236 | 4.10E−05 | RBMX\|RNA binding motif protein, X chromosome |
| 814341 | 0.004173726 | DAPP1\|dual adaptor of phosphotyrosine and 3-phosphoinositides |
| 2391494 | 0.000271971 | EFNA4\|ephrin-A4 |
| 772261 | 0.028380323 | MAPK14\|mitogen-activated protein kinase 14 |
| 704216 | 2.82E−05 | MAP4K2\|mitogen-activating protein kinase kinase kinase kinase 2 |
| 293785 | 0.000141314 | ESTs, Weakly similar to A46010 X-linked retinopathy protein [H. sapiens] |
| 770665 | 0.000828447 | FLJ12750\|hypothetical protein FLJ12750 |
| 264640 | 0.010224986 | CLTB\|clathrin, light polypeptide (Lcb) |
| 824117 | 0.01125807 | VRK2\|vaccinia related kinase 2 |
| 1711877 | 0.01333655 | ESTs |
| 503335 | 0.002578724 | FLJ11196\|hypothetical protein FLJ11196 |
| 811013 | 0.001613372 | AMPD2\|adenosine monophosphate deaminase 2 (isoform L) |
| 1475730 | 0.015717591 | CCT6A\|chaperonin containing TCP1, subunit 6A (zeta 1) |
| 52120 | 4.80E−07 | DTX1\|deltex (Drosophila) homolog 1 |
| 2013094 | 0.003571882 | KIF1C\|kinesin family member 1C |
| 768393 | 0.001209009 | FLJ10829\|hypothetical protein FLJ10829 |
| 128711 | 7.44E−05 | ANLN\|anillin (Drosophila Scraps homolog), actin binding protein |
| 1635032 | 0.002192122 | NOLA1\|nucleolar protein family A, member 1 (H/ACA small nucleolar RNPs) |
| 137535 | 0.003910158 | TIF1\|transcriptional intermediary factor 1 |
| 1870684 | 0.020116819 | PCYT1A\|phosphate cytidylyltransferase 1, choline, alpha isoform |
| 825861 | 0.047376087 | NUP153\|nucleoporin 153 kD |
| 809824 | 0.000141145 | TMEPAI\|transmembrane, prostate androgen induced RNA |
| 815303 | 0.002677229 | DARS\|aspartyl-tRNA synthetase |
| 842849 | 0.002998058 | POLA2\|polymerase (DNA-directed), alpha (70 kD) |
| 22040 | 0.004853144 | MMP9\|matrix metalloproteinase 9 (gelatinase B, 92 kD gelatinase, 92 kD type IV collagenase) |
| 24729 | 4.25E−06 | CHRM1\|cholinergic receptor, muscarinic 1 |
| 159462 | 9.93E−07 | MSE55\|serum constituent protein |
| 811020 | 1.65E−11 | Homo sapiens, clone IMAGE: 4278205, mRNA, partial cds |
| 2307420 | 0.040870882 | TUBB\|tubulin, beta polypeptide |
| 627521 | 0.014104273 | ESTs, Weakly similar to KIAA1140 protein [H. sapiens] |
| 361688 | 0.023373299 | DPP6\|dipeptidylpeptidase VI |
| 1603358 | 0.019799335 | FAT\|FAT tumor suppressor (Drosophila) homolog |
| 1946069 | 6.72E−08 | SPHK1\|sphingosine kinase 1 |
| 268946 | 0.000809644 | Homo sapiens cDNA FLJ31861 fis, clone NT2RP7001319 |
| 824132 | 0.00066839 | Homo sapiens, Similar to cofactor required for Sp1 transcriptional activation, subunit 8 (34 kD), clone |
| | | MGC: 11274 IMAGE: 3944264, mRNA, complete cds |
| 266263 | 0.012980026 | ESTs |
| 1710152 | 0.041184366 | SOAT1\|sterol O-acyltransferase (acyl-Coenzyme A: cholesterol acyltransferase) 1 |
| 813714 | 0.000195754 | CFLAR\|CASP8 and FADD-like apoptosis regulator |
| 813719 | 0.000693345 | ESTs, Weakly similar to DAP1_HUMAN DEATH-ASSOCIATED PROTEIN 1 [H. sapiens] |
| 712401 | 0.005587928 | PIK3CD\|phosphoinositide-3-kinase, catalytic, delta polypeptide |
| 785392 | 0.036628745 | DKFZP434F2021\|DKFZP434F2021 protein |
| 1883008 | 0.008613382 | EST, Weakly similar to T24581 hypothetical protein T06D8.8 - Caenorhabditis elegans [C. elegans] |
| 1637732 | 5.51E−05 | PPAN\|peter pan (Drosophila) homolog |
| 1493390 | 4.71E−06 | ILF2\|interleukin enhancer binding factor 2, 45 kD |
| 595213 | 0.027525584 | BM-009\|hypothetical protein |
| 842861 | 0.018460389 | HNRPR\|heterogeneous nuclear ribonucleoprotein R |
| 878833 | 1.99E−05 | UCHL1\|ubiquitin carboxyl-terminal esterase L1 (ubiquitin thiolesterase) |
| 1631853 | 0.000227064 | Homo sapiens, Similar to RIKEN cDNA 2410003K15 gene, clone MGC: 20424 IMAGE: 4646294, mRNA, |
| | | complete cds |
| 132619 | 0.003085822 | ESTs |
| 502701 | 1.67E−08 | FREQ\|frequenin (Drosophila) homolog |
| 504308 | 3.20E−05 | FLJ10540\|hypothetical protein FLJ10540 |
| 950600 | 2.03E−06 | Homo sapiens mRNA; cDNA DKFZp586C1019 (from clone DKFZp586C1019) |
| 80633 | 0.001826411 | TLR2\|toll-like receptor 2 |
| 784744 | 0.001112368 | MPHOSPH6\|M-phase phosphoprotein 6 |
| 811044 | 1.63E−08 | EST |
| 845419 | 5.05E−05 | FANCA\|Fanconi anemia, complementation group A |
| 897427 | 0.018369144 | Homo sapiens, clone IMAGE: 3638994, mRNA, partial cds |
| 878846 | 9.96E−12 | BRI3\|brain protein I3 |
| 345670 | 2.09E−06 | NEYC\|nuclear transcription factor Y, gamma |
| 1631868 | 0.038386276 | MGC13061\|hypothetical protein MGC13061 |
| 811054 | 0.019642843 | GNA12\|guanine nucleotide binding protein (G protein) alpha 12 |
| 839941 | 1.31E−05 | FLJ14590\|hypothetical protein FLJ14590 |
| 589751 | 0.000104128 | HTATIP2\|HIV-1 Tat interactive protein 2, 30 kDa |
| 208001 | 0.000566004 | CD59\|CD59 antigen p18-20 (antigen identified by monoclonal antibodies 16.3A5, EJ16, EJ30, EL32 and |
| | | G344) |
| 1637756 | 0.000324573 | ENO1\|enolase 1, (alpha) |
| 755612 | 9.22E−06 | GPR39\|G protein-coupled receptor 39 |
| 685045 | 0.002900139 | ESTs, Weakly similar to A47582 B-cell growth factor precursor [H. sapiens] |
| 306771 | 1.68E−06 | TMSNB\|thymosin, beta, identified in neuroblastoma cells |
| 377368 | 0.001270033 | LOC57099\|cell death regulator aven |
| 898092 | 2.20E−05 | CTGF\|connective tissue growth factor |
| 1631874 | 0.020656474 | LOC51068\|CGI-07 protein |
| 841149 | 0.004225448 | TGFBR2\|transforming growth factor, beta receptor II (70-80 kD) |
| 813742 | 6.19E−07 | PTK7\|PTK7 protein tyrosine kinase 7 |
| 785707 | 0.001862647 | PRC1\|protein regulator of cytokinesis 1 |

TABLE 3
Genes that are overexpressed in ER positive cells, and underexpressed in ER negative cells

| GeneID | P-value | Description |
|---|---|---|
| AW473119 | p < 1e−07 | ESR1\|estrogen receptor 1 |
| AF088867 | p < 1e−07 | AGR2\|anterior gradient 2 homolog (Xenepus laevis) |
| AW015443 | p < 1e−07 | ESTs, Weakly similar to JE0350 Anterior gradient-2 [H. sapiens] |
| AW137092 | p < 1e−07 | Homo sapiens cDNA FLJ10561 fis, clone NT2RP2002672 |
| BC017859 | p < 1e−07 | TFF3\|trefoil factor 3 (intestinal) |
| AL137566 | p < 1e−07 | Homo sapiens mRNA; cDNA DKFZp586G0321 (from clone DKFZp586G0321) |
| NM_004496 | p < 1e−07 | HNF3A\|hepatocyte nuclear factor 3, alpha |
| AW451986 | p < 1e−07 | ESTs |
| AW207206 | p < 1e−07 | ESTs |
| AA412108 | p < 1e−07 | ESTs |
| NM_001218 | p < 1e−07 | CA12\|carbonic anhydrase XII |
| AI128582 | p < 1e−07 | ESTs |
| R41823 | p < 1e−07 | ESTs |
| AF356492 | p < 1e−07 | DACH\|dachshund homolog (Drosophila) |
| BC014962 | p < 1e−07 | GFRA1\|GDNF family receptor alpha 1 |
| AA688021 | p < 1e−07 | ESTs |
| AI200852 | p < 1e−07 | ESTs |
| AW086163 | p < 1e−07 | ESTs |
| AF007153 | p < 1e−07 | Homo sapiens clone 23736 mRNA sequence |
| AI050855 | p < 1e−07 | ESTs |
| AA569340 | p < 1e−07 | CYP2B7\|cytochrome P450, subfamily IIB (phenobarbital-inducible), polypeptide 7 |
| AI125908 | p < 1e−07 | Homo sapiens cDNA FLJ30555 fis, clone BRAWH2003818 |
| BC013732 | p < 1e−07 | NAT1\|N-acetyltransferase 1 (arylamine N-acetyltransferase) |
| AA542898 | p < 1e−07 | P28\|dynein, axonemal, light intermediate polypeptide |
| AI820662 | p < 1e−07 | ESTs |
| AA846824 | p < 1e−07 | ESTs |
| NM_020974 | p < 1e−07 | CEGP1\|CEGP1 protein |
| AI373462 | p < 1e−07 | ESTs |
| AJ009985 | p < 1e−07 | ANXA9\|annexin A9 |
| BF433570 | p < 1e−07 | ESTs |
| AI377320 | p < 1e−07 | ESTs |
| NM_000767 | p < 1e−07 | CYP2B6\|cytochrome P450, subfamily IIB (phenobarbital-inducible), polypeptide 6 |
| AF070632 | p < 1e−07 | Homo sapiens clone 24405 mRNA sequence |
| AF131785 | p < 1e−07 | KIAA0882\|KIAA0882 protein |
| AW300348 | p < 1e−07 | Homo sapiens ovarian cancer-related protein 2 (OCR2) mRNA, complete cds |
| AL512749 | p < 1e−07 | Homo sapiens mRNA; cDNA DKFZp667D095 (from clone DKFZp667D095) |
| AI733682 | p < 1e−07 | ESTs |
| AW780011 | p < 1e−07 | ESTs |
| AW135648 | p < 1e−07 | ESTs |
| AI263695 | p < 1e−07 | NME5\|non-metastatic cells 5, protein expressed in (nucleoside-diphosphate kinase) |
| NM_000044 | p < 1e−07 | AR\|androgen receptor (dihydrotestosterone receptor; testicular feminization; spinal and bulbar muscular atrophy; |
| | | Kennedy disease) |
| AK000158 | p < 1e−07 | FLJ20151\|hypothetical protein FLJ20151 |
| AF176012 | p < 1e−07 | JDP1\|J domain containing protein 1 |
| BC018035 | p < 1e−07 | Homo sapiens, clone IMAGE: 4819956, mRNA, partial cds |
| BC008429 | p < 1e−07 | C20orf114\|chromosome 20 open reading frame 114 |
| BF436400 | p < 1e−07 | EST |
| BC006496 | p < 1e−07 | PDZK1\|PDZ domain containing 1 |
| BC014659 | p < 1e−07 | NG22\|NG22 protein |
| AA182477 | p < 1e−07 | ESTs |
| AI272823 | p < 1e−07 | ESTs |
| BC010951 | p < 1e−07 | PIP\|prolactin-induced protein |
| BC014948 | p < 1e−07 | MLPH\|melanophilin |
| AI026825 | p < 1e−07 | DNLC2B\|dynein light chain 2B |
| BC015338 | p < 1e−07 | CPB1\|carboxypeptidase B1 (tissue) |
| AI355477 | p < 1e−07 | ESTs |
| R60583 | p < 1e−07 | NOVA1\|neuro-oncological ventral antigen 1 |
| AK058158 | p < 1e−07 | Homo sapiens cDNA FLJ25429 fis, clone TST05630 |
| BC014601 | p < 1e−07 | Homo sapiens, Similar to RIKEN cDNA 1700017I11 gene, clone MGC: 26847 IMAGE: 4821517, mRNA, complete |
| | | cds |
| AK024204 | p < 1e−07 | Homo sapiens cDNA FLJ14142 fis, clone MAMMA1002880 |
| AI874215 | p < 1e−07 | KIAA0575\|KIAA0575 gene product |
| AK027176 | p < 1e−07 | CAPN13\|calpain 13 |
| BC008915 | p < 1e−07 | SERPINA5\|serine (or cysteine) proteinase inhibitor, clade A (alpha-1 antiproteinase, antitrypsin), member 5 |
| BC017758 | p < 1e−07 | CYP4B1\|cytochrome P450, subfamily IVB, polypeptide 1 |
| AI871614 | p < 1e−07 | HSA250839\|gene for serine/threonine protein kinase |
| AA903339 | p < 1e−07 | RET\|ret proto-oncogene (multiple endocrine neoplasia and medullary thyroid carcinoma 1, Hirschsprung disease) |
| AA553710 | p < 1e−07 | EST |
| BC016861 | p < 1e−07 | Homo sapiens, clone MGC: 17393 IMAGE: 3914851, mRNA, complete cds |
| AY009106 | p < 1e−07 | DKFZP434I092\|DKFZP434I092 protein |
| BC013438 | p < 1e−07 | Homo sapiens, clone IMAGE: 3899073, mRNA, partial cds |
| NM_005518 | p < 1e−07 | HMGCS2\|3-hydroxy-3-methylglutaryl-Coenzyme A synthase 2 (mitochondrial) |
| AW024203 | p < 1e−07 | Homo sapiens, clone MGC: 5522 IMAGE: 3454199, mRNA, complete cds |
| AL133619 | p < 1e−07 | Homo sapiens mRNA; cDNA DKFZp434E2321 (from clone DKFZp434E2321); partial cds |
| NM_000507 | p < 1e−07 | FBP1\|fructose-1,6-bisphosphatase 1 |
| AF237813 | p < 1e−07 | NPD009\|NPD009 protein |
| AI828071 | p < 1e−07 | BCAS1\|breast carcinoma amplified sequence 1 |
| AA557177 | p < 1e−07 | EST |
| Z11162 | p < 1e−07 | AGTR1\|angiotensin receptor 1 |
| NM_007050 | p < 1e−07 | PTPRT\|protein tyrosine phosphatase, receptor type, T |
| NM_007293 | p < 1e−07 | C4A\|complement component 4A |
| AK021931 | p < 1e−07 | Homo sapiens cDNA FLJ11869 fis, clone HEMBA1007002 |
| AA130089 | p < 1e−07 | ESTs |
| BC007997 | p < 1e−07 | RERG\|RAS-like, estrogen-regulated, growth-inhibitor |
| AK055698 | p < 1e−07 | Homo sapiens cDNA FLJ31136 fis, clone IMR322001029 |
| AI668602 | p < 1e−07 | ESTs, Weakly similar to JX0331 laurate omega-hydroxylase [H. sapiens] |
| AW261844 | p < 1e−07 | ESTs |
| AI337740 | p < 1e−07 | FLJ12650\|hypothetical protein FLJ12650 |
| AW073282 | p < 1e−07 | ESTs |
| BC000558 | p < 1e−07 | MAPT\|microtubule-associated protein tau |
| AI498298 | p < 1e−07 | FACVL1\|fatty-acid-Coenzyme A ligase, very long-chain 1 |
| AI570494 | p < 1e−07 | ESTs |
| AF209930 | p < 1e−07 | CHRD\|chordin |
| AK026756 | p < 1e−07 | KIAA1603\|KIAA1603 protein |
| AW070592 | p < 1e−07 | ESTs |
| BC014189 | p < 1e−07 | MGC20702\|hypothetical protein MGC20702 |
| AL117406 | p < 1e−07 | ABCC11\|ATP-binding cassette, sub-family C (CFTR/MRP), member 11 |
| AI190693 | p < 1e−07 | ESTs, Weakly similar to I78885 serine/threonine-specific protein kinase [H. sapiens] |
| NM_003561 | p < 1e−07 | PLA2G10\|phospholipase A2, group X |
| AA513718 | p < 1e−07 | ESTs |
| BF591743 | p < 1e−07 | LPHB\|lipophilin B (uteroglobin family member), prostatein-like |
| AK026958 | p < 1e−07 | FLJ23305\|hypothetical protein FLJ23305 |
| AW016378 | p < 1e−07 | ESTs |
| BC017701 | p < 1e−07 | AD036\|AD036 protein |
| AW294316 | p < 1e−07 | ESTs |
| BC006793 | p < 1e−07 | GATA3\|GATA binding protein 3 |
| AK026427 | p < 1e−07 | FLJ22774\|hypothetical protein FLJ22774 |
| NM_004310 | p < 1e−07 | ARHH\|ras homolog gene family, member H |
| AK000181 | p < 1e−07 | FLJ20174\|hypothetical protein FLJ20174 |
| NM_016613 | p < 1e−07 | LOC51313\|AD021 protein |
| BC000088 | p < 1e−07 | GSTM3\|glutathione S-transferase M3 (brain) |
| BC011721 | p < 1e−07 | TUBA2\|tubulin, alpha 2 |
| AI631708 | p < 1e−07 | ESTs, Weakly similar to 1312318A cystatin SA [H. sapiens] |
| NM_001756 | p < 1e−07 | SERPINA6\|serine (or cysteine) proteinase inhibitor, clade A (alpha-1 antiproteinase, antitrypsin), member 6 |
| NM_004525 | p < 1e−07 | LRP2\|low density lipoprotein-related protein 2 |
| BC012613 | p < 1e−07 | CPA3\|carboxypeptidase A3 (mast cell) |
| BC011348 | p < 1e−07 | FLJ12910\|hypothetical protein FLJ12910 |
| NM_001502 | p < 1e−07 | GP2\|glycoprotein 2 (zymogen granule membrane) |
| AA807457 | p < 1e−07 | MYB\|v-myb myeloblastosis viral oncogene homolog (avian) |
| AL157505 | p < 1e−07 | Homo sapiens mRNA; cDNA DKFZp586P1124 (from clone DKFZp586P1124) |
| NM_005080 | p < 1e−07 | XBP1\|X-box binding protein 1 |
| AI277016 | p < 1e−07 | ESTs |
| AA648777 | p < 1e−07 | MS4A7\|membrane-spanning 4-domains, subfamily A, member 7 |
| AL360204 | p < 1e−07 | Homo sapiens mRNA full length insert cDNA clone EUROIMAGE 980547 |
| BE967259 | p < 1e−07 | BCL2\|B-cell CLL/lymphoma 2 |
| BC012372 | p < 1e−07 | C4B\|complement component 4B |
| AI797041 | p < 1e−07 | TRH\|thyrotropin-releasing hormone |
| AI804588 | p < 1e−07 | BLu\|BLu protein |
| AI309080 | p < 1e−07 | ESTs |
| BF725007 | p < 1e−07 | ADRA2A\|adrenergic, alpha-2A-, receptor |
| AI253198 | p < 1e−07 | Homo sapiens cDNA FLJ13945 fis, clone Y79AA1000969 |
| AK000373 | p < 1e−07 | FLJ20366\|hypothetical protein FLJ20366 |
| AI028773 | p < 1e−07 | ACOX2\|acyl-Coenzyme A oxidase 2, branched chain |
| AW664923 | 0.0000002 | GRIA2\|glutamate receptor, ionotropic, AMPA 2 |
| BC004518 | p < 1e−07 | LOC51760\|B/K protein |
| NM_002145 | p < 1e−07 | HOXB2\|homeo box B2 |
| NM_000792 | p < 1e−07 | DIO1\|deiodinase, iodothyronine, type I |
| AK024872 | p < 1e−07 | SYTL2\|synaptotagmin-like 2 |
| BC015628 | p < 1e−07 | ABAT\|4-aminobutyrate aminotransferase |
| BC000658 | p < 1e−07 | STC2\|stanniocalcin 2 |
| AL050227 | p < 1e−07 | Homo sapiens mRNA; cDNA DKFZp586M0723 (from clone DKFZp586M0723) |
| AK027006 | p < 1e−07 | TNRC9\|trinucleotide repeat containing 9 |
| AA659816 | p < 1e−07 | ESTs |
| AA845338 | p < 1e−07 | FMO5\|flavin containing monooxygenase 5 |
| BC012625 | p < 1e−07 | PPP1R3C\|protein phosphatase 1, regulatory (inhibitor) subunit 3C |
| AW015140 | p < 1e−07 | ESTs |
| AI992108 | p < 1e−07 | Homo sapiens cDNA FLJ25333 fis, clone TST00656 |
| R39691 | p < 1e−07 | SYT\|synaptotagmin I |
| BC001665 | p < 1e−07 | Homo sapiens, Similar to KIAA0843 protein, clone MGC: 1835 IMAGE: 2988043, mRNA, complete cds |
| AF403428 | p < 1e−07 | LRG\|leucine-rich alpha-2-glycoprotein |
| AK057315 | p < 1e−07 | TSGA2\|testes specific A2 homolog (mouse) |
| R40824 | p < 1e−07 | FBXL7\|F-box and leucine-rich repeat protein 7 |
| AI732534 | p < 1e−07 | MGB1\|mammaglobin 1 |
| BC012375 | p < 1e−07 | KIAA1001\|KIAA1001 protein |
| AI587351 | 0.0000012 | CEACAM6\|carcinoembryonic antigen-related cell adhesion molecule 6 (non-specific cross reacting antigen) |
| BC007681 | p < 1e−07 | RAB26\|RAB26, member RAS oncogene family |
| AI694966 | p < 1e−07 | RAI2\|retinoic acid induced 2 |
| AL136926 | p < 1e−07 | DKFZP586M1120\|hypothetical protein DKFZp586M1120 |
| AW070746 | p < 1e−07 | NCAM2\|neural cell adhesion molecule 2 |
| AI432856 | p < 1e−07 | EST |
| AF384856 | p < 1e−07 | PGLYRP\|peptidoglycan recognition protein L precursor |
| AI823785 | p < 1e−07 | BC008967\|hypothetical gene BC008967 |
| BC014250 | p < 1e−07 | CHST8\|carbohydrate (N-acetylgalactosamine 4-0) sulfotransferase 8 |
| R49089 | p < 1e−07 | ESTs, Moderately similar to T12539 hypothetical protein DKFZp434J154.1 [H. sapiens] |
| AW134740 | p < 1e−07 | DCAMKL1\|doublecortin and CaM kinase-like 1 |
| AL390084 | p < 1e−07 | LOC56964\|hypothetical protein from EUROIMAGE 384293 |
| AI056882 | p < 1e−07 | KIAA1493\|KIAA1493 protein |
| AK000532 | p < 1e−07 | Homo sapiens cDNA FLJ20525 fis, clone KAT10610 |
| N22687 | p < 1e−07 | ESTs |
| NM_003770 | p < 1e−07 | KRTHA7\|keratin, hair, acidic, 7 |
| AK000563 | p < 1e−07 | HRASLS2\|HRAS-like suppressor 2 |
| AW082090 | 0.0000001 | Homo sapiens cDNA FLJ33105 fis, clone TRACH2000926, weakly similar to PROBABLE OXIDOREDUCTASE |
| | | EPHD (EC 1.—.—.—) |
| AA069179 | p < 1e−07 | INPP4B\|inositol polyphosphate-4-phosphatase, type II, 105 kD |
| NM_007210 | p < 1e−07 | GALNT6\|UDP-N-acetyl-alpha-D-galactosamine: polypeptide N-acetylgalactosaminyltransferase 6 (GalNAc-T6) |
| AI826254 | p < 1e−07 | HPN\|hepsin (transmembrane protease, serine 1) |
| AL080222 | p < 1e−07 | FLJ13110\|hypothetical protein FLJ13110 |
| AI082272 | p < 1e−07 | KIAA1467\|KIAA1467 protein |
| AF257081 | p < 1e−07 | KCNK15\|potassium channel, subfamily K, member 15 (TASK-5) |
| AI439798 | p < 1e−07 | FGD3\|FGD1 family, member 3 |
| AI471793 | p < 1e−07 | Homo sapiens cDNA FLJ13598 fis, clone PLACE1009921 |
| AI741657 | p < 1e−07 | ITPR1\|inositol 1,4,5-triphosphate receptor, type 1 |
| AK023655 | p < 1e−07 | FLJ13593\|hypothetical protein FLJ13593 |
| NM_006763 | p < 1e−07 | BTG2\|BTG family, member 2 |
| AK001783 | p < 1e−07 | FLJ10921\|hypothetical protein FLJ10921 |
| BC008814 | p < 1e−07 | SLC7A4\|solute carrier family 7 (cationic amino acid transporter, y+ system), member 4 |
| BC006526 | p < 1e−07 | SCNN1A\|sodium channel, nonvoltage-gated 1 alpha |
| AJ224171 | p < 1e−07 | LPHA\|lipophilin A (uteroglobin family member) |
| AI955511 | 0.0000003 | ESTs |
| AA255953 | p < 1e−07 | ACADSB\|acyl-Coenzyme A dehydrogenase, short/branched chain |
| AJ249248 | p < 1e−07 | GPCR150\|putative G protein-coupled receptor |
| AI276310 | p < 1e−07 | EST, Moderately similar to G02666 cysteine-rich protein 1 [H. sapiens] |
| R60224 | 0.0000001 | ESTs, Weakly similar to B28096 line-1 protein ORF2 [H. sapiens] |
| AF224266 | p < 1e−07 | IL20\|interleukin 20 |
| AJ420562 | p < 1e−07 | PKIB\|protein kinase (cAMP-dependent, catalytic) inhibitor beta |
| AF052504 | p < 1e−07 | RNB6\|RNB6 |
| AI420473 | p < 1e−07 | EST |
| NM_014599 | p < 1e−07 | EHD4\|EH-domain containing 4 |
| AL080151 | p < 1e−07 | Homo sapiens mRNA; cDNA DKFZp434D024 (from clone DKFZp434D024) |
| BC012727 | p < 1e−07 | FLJ22593\|hypothetical protein FLJ22593 |
| AA574174 | p < 1e−07 | CYP2A7\|cytochrome P450, subfamily IIA (phenobarbital-inducible), polypeptide 7 |
| AI193043 | p < 1e−07 | ESTs, Weakly similar to T17226 hypothetical protein DKFZp566G223.1 [H. sapiens] |
| AF048700 | p < 1e−07 | SPINK4\|serine protease inhibitor, Kazal type 4 |
| AA860090 | p < 1e−07 | ESTs |
| AA904027 | p < 1e−07 | Homo sapiens cDNA FLJ30147 fis, clone BRACE2000266 |
| NM_012244 | p < 1e−07 | SLC7A8\|solute carrier family 7 (cationic amino acid transporter, y+ system), member 8 |
| AI151349 | p < 1e−07 | ENPP1\|ectonucleotide pyrophosphatase/phosphodiesterase 1 |
| BC005395 | p < 1e−07 | HPX\|hemopexin |
| AI247880 | p < 1e−07 | F7\|coagulation factor VII (serum prothrombin conversion accelerator) |
| AW207804 | p < 1e−07 | ESTs, Weakly similar to A36036 cytochrome P450 2F1 [H. sapiens] |
| AK025256 | p < 1e−07 | FLJ21603\|hypothetical protein FLJ21603 |
| BF434467 | p < 1e−07 | ESTs |
| AI799887 | p < 1e−07 | EST |
| AA804407 | p < 1e−07 | FLJ10980\|hypothetical protein FLJ10980 |
| AW138427 | p < 1e−07 | ESTs |
| BC010626 | p < 1e−07 | Homo sapiens, clone MGC: 17687 IMAGE: 3865868, mRNA, complete cds |
| BC009722 | p < 1e−07 | Homo sapiens, clone MGC: 9575 IMAGE: 3879368, mRNA, complete cds |
| BF129497 | p < 1e−07 | EST |
| BC014851 | p < 1e−07 | LFNG\|lunatic fringe homolog (Drosophila) |
| BF510534 | p < 1e−07 | UMOD\|uromodulin (uromucoid, Tamm-Horsfall glycoprotein) |
| AA627358 | p < 1e−07 | ESTs |
| AA846278 | p < 1e−07 | ESTs |
| AA933076 | p < 1e−07 | ESTs, Weakly similar to ALU1_HUMAN ALU SUBFAMILY J SEQUENCE CONTAMINATION WARNING ENTRY |
| | | [H. sapiens] |
| BC004143 | p < 1e−07 | BF\|B-factor, properdin |
| BC014607 | p < 1e−07 | ASP\|AKAP-associated sperm protein |
| AA810085 | p < 1e−07 | CXorf10\|chromosome X open reading frame 10 |
| AF070604 | p < 1e−07 | CACNA1H\|calcium channel, voltage-dependent, alpha 1H subunit |
| BC001278 | p < 1e−07 | SPUVE\|protease, serine, 23 |
| AF038191 | p < 1e−07 | BAIAP3\|BAI1-associated protein 3 |
| BC013430 | p < 1e−07 | UGDH\|UDP-glucose dehydrogenase |
| BC008962 | p < 1e−07 | PLAB\|prostate differentiation factor |
| U45975 | p < 1e−07 | PIB5PA\|phosphatidylinositol (4,5) bisphosphate 5-phosphatase, A |
| BF476394 | p < 1e−07 | ESTs, Weakly similar to I38022 hypothetical protein [H. sapiens] |
| BC006428 | p < 1e−07 | HSPC195\|hypothetical protein |
| NM_004419 | p < 1e−07 | DUSP5\|dual specificity phosphatase 5 |
| AL512743 | 0.0000054 | SYT13\|synaptotagmin XIII |
| BC014213 | p < 1e−07 | RAB30\|RAB30, member RAS oncogene family |
| NM_000352 | p < 1e−07 | ABCC8\|ATP-binding cassette, sub-family C (CFTR/MRP), member 8 |
| X83301 | p < 1e−07 | SMA5\|SMA5 |
| AL049265 | p < 1e−07 | Homo sapiens mRNA; cDNA DKFZp564F053 (from clone DKFZp564F053) |
| AF237905 | p < 1e−07 | MS4A8B\|membrane-spanning 4-domains, subfamily A, member 8B |
| BC002738 | p < 1e−07 | CRIP1\|cysteine-rich protein 1 (intestinal) |
| BF476123 | p < 1e−07 | ESTs, Weakly similar to S65824 reverse transcriptase homolog [H. sapiens] |
| AK026912 | 0.0000001 | FLJ23259\|hypothetical protein FLJ23259 |
| AI254131 | p < 1e−07 | ESTs |
| AK027159 | p < 1e−07 | FLJ23506\|hypothetical protein FLJ23506 |
| BC004488 | p < 1e−07 | GRP\|gastrin-releasing peptide |
| AK021798 | p < 1e−07 | FLJ11736\|hypothetical protein FLJ11736 |
| AF007170 | p < 1e−07 | KIAA0452\|DEME-6 protein |
| BC010607 | p < 1e−07 | Homo sapiens, clone MGC: 18216 IMAGE: 4156235, mRNA, complete cds |
| AK027311 | p < 1e−07 | KIAA1276\|KIAA1276 protein |
| NM_057158 | p < 1e−07 | DUSP4\|dual specificity phosphatase 4 |
| AI198522 | p < 1e−07 | SLC11A3\|solute carrier family 11 (proton-coupled divalent metal ion transporters), member 3 |
| BC003513 | 0.0000003 | SCYB14\|small inducible cytokine subfamily B (Cys-X-Cys), member 14 (BRAK) |
| AI352080 | p < 1e−07 | FLJ14299\|hypothetical protein FLJ14299 |
| NM_002381 | p < 1e−07 | MATN3\|matrilin 3 |
| L07738 | p < 1e−07 | CACNG1\|calcium channel, voltage-dependent, gamma subunit 1 |
| AI636974 | p < 1e−07 | LOC51097\|CGI-49 protein |
| AF191017 | 0.0000007 | H11\|protein kinase H11 |
| AI620482 | p < 1e−07 | EST |
| AW300891 | p < 1e−07 | FLJ20753\|hypothetical protein FLJ20753 |
| BC005273 | p < 1e−07 | Homo sapiens, clone MGC: 12318 IMAGE: 3685423, mRNA, complete cds |
| AF367473 | p < 1e−07 | NYD-SP21\|testes development-related NYD-SP21 |
| AL137761 | p < 1e−07 | Homo sapiens mRNA; cDNA DKFZp586L2424 (from clone DKFZp586L2424) |
| AI468574 | p < 1e−07 | ESTs |
| AW292427 | p < 1e−07 | Homo sapiens cDNA FLJ13596 fis, clone PLACE1009637 |
| AJ420423 | p < 1e−07 | Homo sapiens mRNA full length insert cDNA clone EUROIMAGE 1287006 |
| AI432615 | p < 1e−07 | DXS1283E\|GS2 gene |
| AW207201 | p < 1e−07 | CHAD\|chondroadherin |
| BC012900 | p < 1e−07 | Homo sapiens, clone IMAGE: 3881549, mRNA |
| AI559487 | p < 1e−07 | SALL2\|sal-like 2 (Drosophila) |
| AW194426 | p < 1e−07 | ESTs |
| NM_003225 | p < 1e−07 | TFF1\|trefoil factor 1 (breast cancer, estrogen-inducible sequence expressed in) |
| AW270334 | p < 1e−07 | ESTs |
| BC013591 | p < 1e−07 | NTN4\|netrin 4 |
| NM_004923 | p < 1e−07 | MTL5\|metallothionein-like 5, testis-specific (tesmin) |
| AI685841 | p < 1e−07 | ESTs |
| AW613732 | p < 1e−07 | Homo sapiens cDNA FLJ31137 fis, clone IMR322001049 |
| AL133644 | p < 1e−07 | Homo sapiens mRNA; cDNA DKFZp434P1514 (from clone DKFZp434P1514); partial cds |
| AY048757 | p < 1e−07 | ABCG1\|ATP-binding cassette, sub-family G (WHITE), member 1 |
| AW292522 | p < 1e−07 | ESTs, Highly similar to A55713 inositol 1,4,5-triphosphate receptor type 1 [H. sapiens] |
| BC001809 | p < 1e−07 | MGC3771\|hypothetical protein MGC3771 |
| AB038463 | p < 1e−07 | CAPN9\|calpain 9 (nCL-4) |
| NM_033380 | p < 1e−07 | COL4A5\|collagen, type IV, alpha 5 (Alport syndrome) |
| BC004240 | p < 1e−07 | CITED1\|Cbp/p300-interacting transactivator, with Glu/Asp-rich carboxy-terminal domain, 1 |
| AF435925 | p < 1e−07 | VLGR1\|very large G protein-coupled receptor 1 |
| AI378647 | p < 1e−07 | ESTs |
| AF035408 | p < 1e−07 | CILP\|cartilage intermediate layer protein, nucleotide pyrophosphohydrolase |
| U11058 | p < 1e−07 | KCNMA1\|potassium large conductance calcium-activated channel, subfamily M, alpha member 1 |
| AI814187 | p < 1e−07 | KIAA1061\|KIAA1061 protein |
| AI399653 | p < 1e−07 | ESTs |
| AL050090 | p < 1e−07 | DKFZP586F1018\|DKFZP586F1018 protein |
| AY043361 | p < 1e−07 | WWP1\|WW domain-containing protein 1 |
| AK027241 | p < 1e−07 | FLJ23588\|hypothetical protein FLJ23588 |
| AK024892 | p < 1e−07 | SH3BGRL\|SH3 domain binding glutamic acid-rich protein like |
| AI928365 | 0.0000003 | SLC1A1\|solute carrier family 1 (neuronal/epithelial high affinity glutamate transporter, system Xag), member 1 |
| BE675157 | p < 1e−07 | ESTs |
| AI669410 | p < 1e−07 | ESTs |
| AA826324 | p < 1e−07 | Homo sapiens cDNA FLJ32320 fis, clone PROST2003537 |
| BC009203 | p < 1e−07 | LOC90355\|hypothetical gene supported by AF038182; BC009203 |
| AI828600 | p < 1e−07 | ESTs |
| AK026141 | p < 1e−07 | Homo sapiens cDNA: FLJ22488 fis, clone HRC10948, highly similar to HSU79298 Human clone 23803 mRNA |
| AI208471 | 0.0000001 | HRLP5\|H-rev107-like protein 5 |
| AK000978 | p < 1e−07 | FLJ10116\|hypothetical protein FLJ10116 |
| AL136579 | p < 1e−07 | KIAA1415\|KIAA1415 protein |
| AI479704 | p < 1e−07 | CYP21A2\|cytochrome P450, subfamily XXIA (steroid 21-hydroxylase, congenital adrenal hyperplasia), |
| | | polypeptide 2 |
| AW148827 | p < 1e−07 | SEZ6L\|seizure related 6 homolog (mouse)-like |
| AF220189 | 0.0000329 | BEX1\|brain expressed, X-linked 1 |
| AA557341 | p < 1e−07 | EST |
| BC004504 | p < 1e−07 | MGC11138\|hypothetical protein MGC11138 |
| AI922204 | 0.0000002 | Homo sapiens cDNA FLJ30298 fis, clone BRACE2003172 |
| AI969835 | 0.0000003 | NELL2\|NEL-like 2 (chicken) |
| NM_000841 | 0.0000012 | GRM4\|glutamate receptor, metabotropic 4 |
| AW205768 | 0.0000269 | THRSP\|thyroid hormone responsive (SPOT14 homolog, rat) |
| AF100759 | p < 1e−07 | NET-6\|tetraspan NET-6 protein |
| AF121129 | p < 1e−07 | NR2E3\|nuclear receptor subfamily 2, group E, member 3 |
| AI126271 | p < 1e−07 | Homo sapiens cDNA FLJ31235 fis, clone KIDNE2004681, moderately similar to Mus musculus peroxisomal long |
| | | chain acyl-CoA thioesterase lb (Pte1b) gene |
| AI457338 | p < 1e−07 | Homo sapiens cDNA FLJ33115 fis, clone TRACH2001314 |
| BM126815 | p < 1e−07 | Homo sapiens cDNA FLJ11375 fis, clone HEMBA1000411, weakly similar to ANKYRIN |
| BF002534 | p < 1e−07 | FLJ10244\|hypothetical protein FLJ10244 |
| AA700070 | 0.0000066 | SLC6A4\|solute carrier family 6 (neurotransmitter transporter, serotonin), member 4 |
| BC014913 | p < 1e−07 | Homo sapiens, Similar to synaptotagmin-like 4, clone MGC: 17313 IMAGE: 3908307, mRNA, complete cds |
| AF026477 | p < 1e−07 | GALNAC4S-6ST\|B cell RAG associated protein |
| R16838 | p < 1e−07 | ESTs |
| AF116660 | 0.0000006 | C8orf4\|chromosome 8 open reading frame 4 |
| AI568799 | p < 1e−07 | ENPP5\|ectonucleotide pyrophosphatase/phosphodiesterase 5 (putative function) |
| AA633845 | p < 1e−07 | ESTs |
| AW265341 | p < 1e−07 | ESTs |
| AA738043 | p < 1e−07 | SCAP1\|src family associated phosphoprotein 1 |
| AK024999 | p < 1e−07 | Homo sapiens cDNA: FLJ21346 fis, clone COL02705 |
| M76558 | p < 1e−07 | CACNA1D\|calcium channel, voltage-dependent, L type, alpha 1D subunit |
| AW016396 | p < 1e−07 | PACE4\|paired basic amino acid cleaving system 4 |
| H04392 | p < 1e−07 | ESTs |
| BC000314 | 0.0000001 | RTN1\|reticulon 1 |
| AI823800 | 0.0000002 | GPR54\|G protein-coupled receptor 54 |
| AW135596 | p < 1e−07 | FLJ10058\|hypothetical protein FLJ10058 |
| AI364725 | p < 1e−07 | KIAA0239\|KIAA0239 protein |
| AI200759 | p < 1e−07 | Homo sapiens cDNA FLJ32802 fis, clone TESTI2002580 |
| NM_005544 | p < 1e−07 | IRS1\|insulin receptor substrate 1 |
| AA580157 | p < 1e−07 | EIF4B\|eukaryotic translation initiation factor 4B |
| AI926614 | p < 1e−07 | EST |
| NM_006113 | p < 1e−07 | VAV3\|vav 3 oncogene |
| AW007851 | p < 1e−07 | RAB5EP\|rabaptin-5 |
| BC009373 | p < 1e−07 | Homo sapiens, clone MGC: 16638 IMAGE: 4121964, mRNA, complete cds |
| AL157499 | p < 1e−07 | Homo sapiens mRNA; cDNA DKFZp434N2412 (from clone DKFZp434N2412) |
| BC015520 | p < 1e−07 | RNASE4\|ribonuclease, RNase A family, 4 |
| AB050468 | p < 1e−07 | LIG1\|ortholog of mouse integral membrane glycoprotein LIG-1 |
| AI446183 | p < 1e−07 | ESTs, Highly similar to CYA5_HUMAN ADENYLATE CYCLASE, TYPE V [H. sapiens] |
| AI806109 | 0.0000003 | KIAA1580\|KIAA1580 protein |
| AI479419 | p < 1e−07 | KIAA1511\|KIAA1511 protein |
| AL136858 | p < 1e−07 | DKFZp434N2435\|hypothetical protein DKFZp434N2435 |
| BI491333 | p < 1e−07 | Homo sapiens cDNA FLJ13289 fis, clone OVARC1001170 |
| AF035282 | p < 1e−07 | C1orf21\|chromosome 1 open reading frame 21 |
| R36615 | p < 1e−07 | ESTs |
| AI673455 | p < 1e−07 | ESTs |
| BC000809 | p < 1e−07 | TCEAL1\|transcription elongation factor A (SII)-like 1 |
| AW450693 | p < 1e−07 | Homo sapiens mRNA, exon 1, 2, 3, 4, clone: RES4-24A |
| AV646597 | p < 1e−07 | ESTs, Weakly similar to ALU7_HUMAN ALU SUBFAMILY SQ SEQUENCE CONTAMINATION WARNING ENTRY |
| | | [H. sapiens] |
| AA749341 | p < 1e−07 | KIAA0040\|KIAA0040 gene product |
| BC000576 | p < 1e−07 | QDPR\|quinoid dihydropteridine reductase |
| BC003559 | p < 1e−07 | SERPINA3\|serine (or cysteine) proteinase inhibitor, clade A (alpha-1 antiproteinase, antitrypsin), member 3 |
| BC008940 | 0.0000051 | HOXB5\|homeo box B5 |
| AF057164 | p < 1e−07 | SLC22A5\|solute carrier family 22 (organic cation transporter), member 5 |
| L02785 | 0.0000373 | SLC26A3\|solute carrier family 26, member 3 |
| AF054910 | p < 1e−07 | TEKT2\|tektin 2 (testicular) |
| NM_002847 | p < 1e−07 | PTPRN2\|protein tyrosine phosphatase, receptor type, N polypeptide 2 |
| AA775255 | p < 1e−07 | ANKHZN\|ANKHZN protein |
| AL080192 | p < 1e−07 | Homo sapiens mRNA; cDNA DKFZp434B102 (from clone DKFZp434B102) |
| AK027206 | p < 1e−07 | MGC16309\|hypothetical gene MGC16309 |
| BE671061 | p < 1e−07 | C20orf103\|chromosome 20 open reading frame 103 |
| X95097 | 0.0000007 | VIPR2\|vasoactive intestinal peptide receptor 2 |
| M57609 | p < 1e−07 | GLI3\|GLI-Kruppel family member GLI3 (Greig cephalopolysyndactyly syndrome) |
| AF248053 | p < 1e−07 | SLC2A10\|solute carrier family 2 (facilitated glucose transporter), member 10 |
| AK022441 | p < 1e−07 | Homo sapiens cDNA FLJ12379 fis, clone MAMMA1002554 |
| AB026707 | p < 1e−07 | NMES1\|normal mucosa of esophagus specific 1 |
| BC005153 | p < 1e−07 | RPH3AL\|rabphilin 3A-like (without C2 domains) |
| AW449824 | p < 1e−07 | ESTs |
| AW080831 | p < 1e−07 | SEC14L2\|SEC14-like 2 (S. cerevisiae) |
| AI674226 | p < 1e−07 | Homo sapiens, Similar to RIKEN cDNA 1700008D07 gene, clone MGC: 9830 IMAGE: 3863323, mRNA, complete |
| | | cds |
| AI382972 | p < 1e−07 | TPBG\|trophoblast glycoprotein |
| AK025198 | 0.0000003 | Homo sapiens cDNA: FLJ21545 fis, clone COL06195 |
| BC000662 | p < 1e−07 | MGC2601\|hypothetical protein MGC2601 |
| AL133622 | p < 1e−07 | KIAA0876\|KIAA0876 protein |
| R42185 | p < 1e−07 | Homo sapiens cDNA FLJ32064 fis, clone OCBBF1000080 |
| U62325 | p < 1e−07 | APBB2\|amyloid beta (A4) precursor protein-binding, family B, member 2 (Fe65-like) |
| BC008317 | p < 1e−07 | LIV-1\|LIV-1 protein, estrogen regulated |
| AI825621 | 0.0000001 | KIAA1130\|KIAA1130 protein |
| M93119 | 0.0006433 | INSM1\|insulinoma-associated 1 |
| BC016018 | p < 1e−07 | Homo sapiens, clone MGC: 27478 IMAGE: 4714682, mRNA, complete cds |
| D89675 | p < 1e−07 | BMPR1B\|bone morphogenetic protein receptor, type IB |
| AI093769 | p < 1e−07 | ESTs |
| NM_006006 | 0.0000002 | ZNF145\|zinc finger protein 145 (Kruppel-like, expressed in promyelocytic leukemia) |
| AW294857 | p < 1e−07 | LOC51161\|g20 protein |
| AK000269 | p < 1e−07 | FLJ20262\|hypothetical protein FLJ20262 |
| AI674190 | p < 1e−07 | FLJ13941\|hypothetical protein FLJ13941 |
| AW207696 | 0.0000453 | MGB2\|mammaglobin 2 |
| R41429 | p < 1e−07 | ATP7B\|ATPase, Cu++ transporting, beta polypeptide (Wilson disease) |
| AF131784 | p < 1e−07 | Homo sapiens clone 25194 mRNA sequence |
| BC010963 | 0.0000002 | TNNT1\|troponin T1, skeletal, slow |
| BC009799 | 0.0000392 | AREG\|amphiregulin (schwannoma-derived growth factor) |
| AJ278018 | p < 1e−07 | CS2\|calsyntenin-2 |
| BI494440 | p < 1e−07 | KIAA0222\|KIAA0222 gene product |
| AF140240 | p < 1e−07 | TBX3\|T-box 3 (ulnar mammary syndrome) |
| D50840 | p < 1e−07 | UGCG\|UDP-glucose ceramide glucosyltransferase |
| AW157331 | p < 1e−07 | KIAA1246\|KIAA1246 protein |
| AW074266 | p < 1e−07 | ESTs |
| BF110928 | p < 1e−07 | ESTs, Weakly similar to I38022 hypothetical protein [H. sapiens] |
| AI961444 | p < 1e−07 | P37NB\|37 kDa leucine-rich repeat (LRR) protein |
| AK027251 | p < 1e−07 | FLJ23598\|hypothetical protein FLJ23598 |
| AF131832 | p < 1e−07 | DKFZp761F2014\|hypothetical protein DKFZp761F2014 |
| AI866858 | 0.0000002 | MUC1\|mucin 1, transmembrane |
| AI688494 | 0.0000005 | FLJ13189\|hypothetical protein FLJ13189 |
| AW150267 | 0.0000037 | C21orf9\|chromosome 21 open reading frame 9 |
| | | Human DNA sequence from clone 34B21 on chromosome 6p12.1-21.1. Contains part of a gene for a novel protein |
| | | with ZU5 domain similar to part of Tight Junction Protein ZO1 (TJP1) and UNC5 Homologs, the gene for a novel |
| | | BZRP (peripheral benzodiazapine receptor (MBR, PBR, PBKS, IBP, Isoquinoline-binding protein)) LIKE protein, the |
| | | gene for a novel protein similar to part of APOBEC1 (Phorbolin 1, Apolipoprotein B mRNA editing protein), and the |
| | | NFYA gene for nuclear transcription factor Y, alpha (CCAAT-Binding transcription factor subunit B, CBF-B, CAAT- |
| | | Box DNA binding protein subunit A). Contains ESTs, STSs, GSSs, two putative CpG islands and a ca repeat |
| AI038082 | p < 1e−07 | polymorphism |
| AL110139 | p < 1e−07 | Homo sapiens mRNA; cDNA DKFZp564O1763 (from clone DKFZp564O1763) |
| BC002471 | p < 1e−07 | CPLX1\|complexin 1 |
| AB031549 | p < 1e−07 | PDEF\|prostate epithelium-specific Ets transcription factor |
| U63008 | 0.0000033 | HGD\|homogentisate 1,2-dioxygenase (homogentisate oxidase) |
| AF153330 | p < 1e−07 | SLC19A2\|solute carrier family 19 (thiamine transporter), member 2 |
| AL136693 | p < 1e−07 | FLJ23462\|duodenal cytochrome b |
| BC007339 | 0.0000001 | HEP27\|short-chain alcohol dehydrogenase family member |
| AF146192 | p < 1e−07 | MRPS30\|mitochondrial ribosomal protein S30 |
| AL137748 | p < 1e−07 | NBEA\|neurobeachin |
| AI989600 | p < 1e−07 | ESTs |
| AW469017 | p < 1e−07 | ESTs |
| U56725 | 0.0000001 | HSPA2\|heat shock 70 kD protein 2 |
| AK057601 | p < 1e−07 | KIAA0303\|KIAA0303 protein |
| AI147370 | p < 1e−07 | ESTs |
| AA497018 | p < 1e−07 | ADCY1\|adenylate cyclase 1 (brain) |
| BF510257 | 0.0000001 | PLAC1\|placenta-specific 1 |
| AB028967 | 0.0000032 | KCND2\|potassium voltage-gated channel, Shal-related subfamily, member 2 |
| NM_003221 | 0.0004633 | TFAP2B\|transcription factor AP-2 beta (activating enhancer binding protein 2 beta) |
| AI349306 | 0.0000004 | ESTs |
| AY029191 | 0.0000011 | ASPN\|asporin (LRR class 1) |
| BG272534 | p < 1e−07 | ESTs, Weakly similar to 2004399A chromosomal protein [H. sapiens] |
| AI288717 | p < 1e−07 | RFX2\|regulatory factor X, 2 (influences HLA class II expression) |
| AI080649 | p < 1e−07 | KIAA0938\|KIAA0938 protein |
| AA177001 | p < 1e−07 | DKFZP586J0619\|DKFZP586J0619 protein |
| AW340797 | 0.0000006 | ESTs |
| N95437 | 0.000001 | ESTs |
| BC016153 | 0.0000058 | Homo sapiens, Similar to hypothetical protein FLJ10134, clone MGC: 13208 IMAGE: 3841102, mRNA, complete |
| | | cds |
| AL136592 | p < 1e−07 | DKFZP761I172\|hypothetical protein DKFZp761I172 |
| AI217517 | p < 1e−07 | KIAA0557\|KIAA0557 protein |
| AI128331 | p < 1e−07 | ENDOFIN\|endosome-associated FYVE-domain protein |
| BF446355 | p < 1e−07 | ESTs |
| AK000634 | p < 1e−07 | FLJ20627\|hypothetical protein FLJ20627 |
| AI655104 | p < 1e−07 | ESTs |
| AL136901 | p < 1e−07 | FLJ22944\|hypothetical protein FLJ22944 |
| NM_000909 | 0.0000001 | NPY1R\|neuropeptide Y receptor Y1 |
| AI379600 | p < 1e−07 | FLJ11715\|hypothetical protein FLJ11715 |
| AF098277 | p < 1e−07 | SLC23A2\|solute carrier family 23 (nucleobase transporters), member 2 |
| AI631667 | p < 1e−07 | Homo sapiens cDNA FLJ31763 fis, clone NT2RI2007827 |
| AL157484 | p < 1e−07 | Homo sapiens mRNA; cDNA DKFZp762M127 (from clone DKFZp762M127) |
| AY040546 | 0.0001123 | Homo sapiens RPE-spondin (RPESP) mRNA, partial cds |
| AI373939 | 0.00001 | GJA1\|gap junction protein, alpha 1, 43 kD (connexin 43) |
| NM_001322 | p < 1e−07 | CST2\|cystatin SA |
| U96136 | 0.0000023 | CTNND2\|catenin (cadherin-associated protein), delta 2 (neural plakophilin-related arm-repeat protein) |
| AB038518 | p < 1e−07 | COLEC12\|collectin sub-family member 12 |
| AI332972 | p < 1e−07 | Homo sapiens, clone MGC: 9889 IMAGE: 3868330, mRNA, complete cds |
| AL161952 | p < 1e−07 | GLUL\|glutamate-ammonia ligase (glutamine synthase) |
| BE464993 | p < 1e−07 | Homo sapiens PAC clone RP4-751H13 from 7q35-qter |
| AI394310 | p < 1e−07 | GALNT7\|UDP-N-acetyl-alpha-D-galactosamine:polypeptide N-acetylgalactosaminyltransferase 7 (GalNAc-T7) |
| AW149773 | 0.0000001 | p25\|brain-specific protein p25 alpha |
| Y17978 | p < 1e−07 | FUT8\|fucosyltransferase 8 (alpha (1,6) fucosyltransferase) |
| AF183421 | p < 1e−07 | RAB31\|RAB31, member RAS oncogene family |
| BE676675 | p < 1e−07 | RIPK3\|receptor-interacting serine-threonine kinase 3 |
| AL096739 | p < 1e−07 | DKFZp586H0623\|putative UDP-GalNAc:polypeptide N-acetylgalactosaminyltransferase T9 |
| R37016 | 0.0000002 | KIAA0534\|KIAA0534 protein |
| AB018286 | 0.0000069 | NRXN3\|neurexin 3 |
| AK026842 | p < 1e−07 | FLJ23189 hypothetical protein FLJ23189 |
| NM_003489 | p < 1e−07 | NRIP1\|nuclear receptor interacting protein 1 |
| D86322 | 0.0000004 | CLGN\|calmegin |
| AI951144 | p < 1e−07 | ESTs, Weakly similar to 2109260A B cell growth factor [H. sapiens] |
| AA706017 | p < 1e−07 | ESTs |
| AA631782 | 0.0000004 | EST |
| AL117550 | p < 1e−07 | LOC51643\|CGI-119 protein |
| AI000682 | p < 1e−07 | ESTs, Weakly similar to ALU1_HUMAN ALU SUBFAMILY J SEQUENCE CONTAMINATION WARNING ENTRY |
| | | [H. sapiens] |
| AJ420490 | 0.0000818 | IL20RA\|interleukin 20 receptor, alpha |
| AY007114 | p < 1e−07 | Homo sapiens clone TCCCTA00151 mRNA sequence |
| AF035299 | p < 1e−07 | DOK1\|docking protein 1, 62 kD (downstream of tyrosine kinase 1) |
| AI039854 | p < 1e−07 | SYNCOILIN\|intermediate filament protein syncoilin |
| AW139266 | p < 1e−07 | FLJ12057\|hypothetical protein FLJ12057 |
| NM_003961 | p < 1e−07 | RHBDL\|rhomboid, veinlet-like 1 (Drosophila) |
| AI277269 | p < 1e−07 | KIAA0944\|KIAA0944 protein |
| AF153605 | p < 1e−07 | AIG-1\|androgen induced protein |
| M60445 | 0.0000066 | HDC\|histidine decarboxylase |
| BC002668 | p < 1e−07 | PECI\|peroxisomal D3,D2-enoyl-CoA isomerase |
| AW296675 | p < 1e−07 | NUCB2\|nucleobindin 2 |
| BE464662 | p < 1e−07 | ESTs |
| AW205080 | p < 1e−07 | NEURL\|neuralized-like (Drosophila) |
| Y10183 | p < 1e−07 | ALCAM\|activated leucocyte cell adhesion molecule |
| AL137449 | 0.0000081 | HOXB4\|homeo box B4 |
| AF004222 | p < 1e−07 | RTN2\|reticulon 2 |
| AK022482 | p < 1e−07 | Homo sapiens cDNA FLJ12420 fis, clone MAMMA1003049 |
| NM_007069 | p < 1e−07 | HRASLS3\|HRAS-like suppressor 3 |
| NM_001231 | p < 1e−07 | CASQ1\|calsequestrin 1 (fast-twitch, skeletal muscle) |
| AL136760 | p < 1e−07 | DKFZP434H0115\|hypothetical protein DKFZp434H0115 |
| AI244129 | 0.0000001 | RAMP3\|receptor (calcitonin) activity modifying protein 3 |
| BC000548 | 0.0000002 | RAMP1\|receptor (calcitonin) activity modifying protein 1 |
| BC005257 | 0.0000724 | MSMB\|microseminoprotein, beta- |
| AF116574 | p < 1e−07 | ASTN2\|astrotactin 2 |
| AA782483 | p < 1e−07 | REPS2\|RALBP1 associated Eps domain containing 2 |
| AI912086 | p < 1e−07 | Homo sapiens cDNA FLJ30744 fis, clone FEBRA2000378 |
| U78168 | p < 1e−07 | EPAC\|Rap1 guanine-nucleotide-exchange factor directly activated by cAMP |
| AA016043 | p < 1e−07 | SAG\|S-antigen; retina and pineal gland (arrestin) |
| NM_003155 | 0.0000041 | STC1\|stanniocalcin 1 |
| BE672557 | p < 1e−07 | ESTs |
| AK026275 | p < 1e−07 | FLJ22622\|hypothetical protein FLJ22622 |
| AA993573 | p < 1e−07 | TGFB3\|transforming growth factor, beta 3 |
| BC018043 | 0.0000003 | SERPINI1\|serine (or cysteine) proteinase inhibitor, clade I (neuroserpin), member 1 |
| BC006168 | p < 1e−07 | KIAA0089\|KIAA0089 protein |
| AW206445 | p < 1e−07 | ESTs, Weakly similar to UOG1_HUMAN UOG-1 PROTEIN [H. sapiens] |
| AA513133 | p < 1e−07 | ESTs, Weakly similar to 1209280A tropomyosin [H. sapiens] |
| AL080094 | 0.0000673 | Homo sapiens mRNA; cDNA DKFZp564O1262 (from clone DKFZp564O1262) |
| BC009211 | p < 1e−07 | ELP2\|elongator protein 2 |
| BC004161 | p < 1e−07 | NET-7\|transmembrane 4 superfamily member (tetraspan NET-7) |
| NM_001094 | 0.0000052 | ACCN1\|amiloride-sensitive cation channel 1, neuronal (degenerin) |
| AL136539 | p < 1e−07 | DKFZP761E2110\|hypothetical protein DKFZp761E2110 |
| AA846880 | p < 1e−07 | MEIS3\|Meis1, myeloid ecotropic viral integration site 1 homolog 3 (mouse) |
| AF144094 | p < 1e−07 | MYO15A\|myosin XVA |
| AF159570 | 0.0000001 | RGS5\|regulator of G-protein signalling 5 |
| AW015327 | p < 1e−07 | IGF1R\|insulin-like growth factor 1 receptor |
| AK021540 | p < 1e−07 | Homo sapiens cDNA FLJ11478 fis, clone HEMBA1001781 |
| BC007618 | 0.0000001 | MGC15730\|hypothetical protein MGC15730 |
| NM_001899 | p < 1e−07 | CST4\|cystatin S |
| R74353 | p < 1e−07 | HNMT\|histamine N-methyltransferase |
| AI262988 | p < 1e−07 | KIAA1548\|KIAA1548 protein |
| AA505938 | p < 1e−07 | EST |
| AI866828 | 0.0000073 | COL10A1\|collagen, type X, alpha 1(Schmid metaphyseal chondrodysplasia) |
| AI076506 | 0.0000001 | RORC\|RAR-related orphan receptor C |
| AJ245503 | 0.0000001 | PXR2b\|PXR2b protein |
| AF217970 | p < 1e−07 | Homo sapiens clone PP1195 unknown mRNA |
| AI963850 | p < 1e−07 | KIAA1324\|KIAA1324 protein |
| U80811 | p < 1e−07 | EDG2\|endothelial differentiation, lysophosphatidic acid G-protein-coupled receptor, 2 |
| AA745593 | p < 1e−07 | BATF\|basic leucine zipper transcription factor, ATF-like |
| AW328488 | 0.0000002 | PTK6\|PTK6 protein tyrosine kinase 6 |
| AA258057 | p < 1e−07 | ESTs |
| AW104057 | p < 1e−07 | ESTs |
| AK027148 | p < 1e−07 | FLJ23495\|hypothetical protein FLJ23495 |
| AW070918 | p < 1e−07 | ESTs, Weakly similar to T2D3_HUMAN TRANSCRIPTION INITIATION FACTOR TFIID 135 KDA SUBUNIT |
| | | [H. sapiens] |
| AI369214 | p < 1e−07 | ESTs |
| BC002591 | 0.0001249 | MMP10\|matrix metalloproteinase 10 (stromelysin 2) |
| AA431407 | 0.0000011 | MACS1\|medium-chain acyl-CoA synthetase |
| BC016955 | p < 1e−07 | Homo sapiens cDNA: FLJ22182 fis, clone HRC00953 |
| AI033912 | 0.0000003 | RLN2\|relaxin 2 (H2) |
| AA600175 | 0.0000428 | ESTs |
| AI436323 | 0.0000487 | ROBO2\|roundabout, axon guidance receptor, homolog 2 (Drosophila) |
| AL136939 | p < 1e−07 | HELO1\|homolog of yeast long chain polyunsaturated fatty acid elongation enzyme 2 |
| AI471839 | p < 1e−07 | THBD\|thrombomodulin |
| S61953 | p < 1e−07 | ERBB3\|v-erb-b2 erythroblastic leukemia viral oncogene homolog 3 (avian) |
| AA595802 | p < 1e−07 | ESTs, Weakly similar to T17279 hypothetical protein DKFZp434F1935.1 [H. sapiens] |
| AA865325 | p < 1e−07 | RAMP2\|receptor (calcitonin) activity modifying protein 2 |
| AI866867 | 0.0000002 | SEMA3C\|sema domain, immunoglobulin domain (Ig), short basic domain, secreted, (semaphorin) 3C |
| AI768204 | 0.000014 | CDSN\|corneodesmosin |
| AI200391 | 0.0000004 | ESTs, Weakly similar to S26689 hypothetical protein hc1 - mouse [M. musculus] |
| AI670779 | p < 1e−07 | ESTs, Weakly similar to ALUC_HUMAN !!!! ALU CLASS C WARNING ENTRY !!! [H. sapiens] |
| BC010924 | 0.0000001 | PRO1073\|PRO1073 protein |
| NM_006103 | 0.0000086 | WFDC2\|WAP four-disulfide core domain 2 |
| AI955538 | p < 1e−07 | Homo sapiens cDNA FLJ33142 fis, clone UTERU1000192 |
| BC010522 | p < 1e−07 | Homo sapiens, clone MGC: 18084 IMAGE: 4150952, mRNA, complete cds |
| N89991 | p < 1e−07 | Homo sapiens cDNA FLJ31149 fis, clone IMR322001491, moderately similar to Rattus norvegicus tricarboxylate |
| | | carrier-like protein mRNA |
| AK026813 | p < 1e−07 | PCANAP1\|prostate cancer associated protein 1 |
| BC003098 | p < 1e−07 | HSD17B4\|hydroxysteroid (17-beta) dehydrogenase 4 |
| AK027056 | 0.0000001 | FLJ23403\|hypothetical protein FLJ23403 |
| AA582929 | 0.0000095 | SERHL\|kraken-like |
| AV656862 | p < 1e−07 | Homo sapiens cDNA FLJ13603 fis, clone PLACE1010270 |
| AI686160 | p < 1e−07 | ESTs |
| R42732 | p < 1e−07 | ESTs |
| AK025000 | p < 1e−07 | FLJ21347\|hypothetical protein FLJ21347 |
| AW247958 | p < 1e−07 | SDCCAG28\|serologically defined colon cancer antigen 28 |
| AI804711 | p < 1e−07 | EST, Moderately similar to ALU5_HUMAN ALU SUBFAMILY SC SEQUENCE CONTAMINATION WARNING |
| | | ENTRY [H. sapiens] |
| BC010553 | 0.0000179 | TIMP4\|tissue inhibitor of metalloproteinase 4 |
| AK027024 | p < 1e−07 | Homo sapiens cDNA: FLJ23371 fis, clone HEP16068, highly similar to HSTFIISH Homo sapiens mRNA for |
| | | transcription elongation factor TFIIS |
| AL133591 | 0.0000241 | Homo sapiens mRNA; cDNA DKFZp434N079 (from clone DKFZp434N079) |
| AW513195 | p < 1e−07 | Homo sapiens cDNA FLJ13524 fis, clone PLACE1006002 |
| AI866286 | p < 1e−07 | ESTs, Weakly similar to CA13 MOUSE COLLAGEN ALPHA 1(III) CHAIN PRECURSOR [M. musculus] |
| AL583912 | p < 1e−07 | KIF13B\|kinesin family member 13B |
| NM_018653 | 0.0000004 | GPRC5C\|G protein-coupled receptor, family C, group 5, member C |
| AI218954 | p < 1e−07 | KIAA0717\|KIAA0717 protein |
| AJ420566 | p < 1e−07 | Homo sapiens mRNA full length insert cDNA clone EUROIMAGE 1572223 |
| BF447150 | 0.0000017 | ESTs |
| AK025296 | p < 1e−07 | FLJ20273\|hypothetical protein |
| AK025732 | p < 1e−07 | ASAH\|N-acylsphingosine amidohydrolase (acid ceramidase) |
| AK001648 | p < 1e−07 | FLJ10786\|hypothetical protein FLJ10786 |
| BC015182 | p < 1e−07 | CYB5\|cytochrome b-5 |
| R56053 | 0.0000002 | ME3\|malic enzyme 3, NADP(+)-dependent, mitochondrial |
| AW450080 | 0.0000018 | Human clone 137308 mRNA, partial cds |
| NM_033014 | 0.0000032 | OGN\|osteoglycin (osteoinductive factor, mimecan) |
| AK026817 | p < 1e−07 | FLJ23577\|hypothetical protein FLJ23577 |
| R61469 | 0.0000454 | Human (clone CTG-A4) mRNA sequence |
| BC002619 | 0.000176 | LOC51617\|HMP19 protein |
| AI150439 | p < 1e−07 | KIAA1407\|KIAA1407 protein |
| BC017338 | p < 1e−07 | FUCA1\|fucosidase, alpha-L-1, tissue |
| AF117708 | p < 1e−07 | KCNK6\|potassium channel, subfamily K, member 6 (TWIK-2) |
| AW139414 | p < 1e−07 | PRLR\|prolactin receptor |
| AI559968 | p < 1e−07 | RGL3\|RalGEF-like protein 3, mouse homolog |
| AW195539 | p < 1e−07 | DDO\|D-aspartate oxidase |
| AK024964 | p < 1e−07 | NFIA\|nuclear factor I/A |
| BC004925 | 0.0000004 | Homo sapiens, Similar to G protein-coupled receptor, family C, group 5, member C, clone MGC: 10304 |
| | | IMAGE: 3622005, mRNA, complete cds |
| AJ420521 | 0.0000005 | SMOC2\|secreted modular calcium-binding protein 2 |
| AF271783 | p < 1e−07 | FLJ21174\|hypothetical protein FLJ21174 |
| AA883246 | 0.0000032 | Homo sapiens cDNA FLJ30428 fis, clone BRACE2008941 |
| AW050970 | 0.0000062 | DF\|D component of complement (adipsin) |
| BC016615 | p < 1e−07 | Homo sapiens, Similar to RAB37, member of RAS oncogene family, clone MGC: 21391 IMAGE: 4520191, mRNA, |
| | | complete cds |
| AI972866 | p < 1e−07 | ESTs |
| AA554481 | 0.0000003 | ESTs |
| NM_014279 | 0.0000094 | OLFM1\|olfactomedin 1 |
| AI628869 | p < 1e−07 | FLJ12953\|hypothetical protein FLJ12953 similar to Mus musculus D3Mm3e |
| AL133636 | p < 1e−07 | CIRBP\|cold inducible RNA binding protein |
| AW148329 | p < 1e−07 | ESTs |
| AL043469 | 0.0000007 | F13A1\|coagulation factor XIII, A1 polypeptide |
| BC005989 | 0.0001701 | PNLIPRP2\|pancreatic lipase-related protein 2 |
| AL133033 | p < 1e−07 | KIAA1025\|KIAA1025 protein |
| AI536750 | 0.0000003 | ROR2\|receptor tyrosine kinase-like orphan receptor 2 |
| BC001638 | 0.000447 | ASCL1\|achaete-scute complex-like 1 (Drosophila) |
| BC008185 | p < 1e−07 | FABGL\|FabG (beta-ketoacyl-[acyl-carrier-protein] reductase, E. coli) like (E. coli) |
| AA399226 | p < 1e−07 | TJP3\|tight junction protein 3 (zona occludens 3) |
| NM_003979 | p < 1e−07 | RAI3\|retinoic acid induced 3 |
| AI367233 | p < 1e−07 | EST |
| BC018636 | p < 1e−07 | PLAT\|plasminogen activator, tissue |
| AI565489 | p < 1e−07 | PDE4A\|phosphodiesterase 4A, cAMP-specific (phosphodiesterase E2 dunce homolog, Drosophila) |
| AW136191 | p < 1e−07 | ESTs |
| AI421708 | p < 1e−07 | Homo sapiens cDNA FLJ13137 fis, clone NT2RP3003150 |
| AW057705 | 0.0000003 | FLT3\|fms-related tyrosine kinase 3 |
| AA843666 | 0.0000031 | ESTs |
| BC000232 | p < 1e−07 | D5S346\|DNA segment, single copy probe LNS-CAI/LNS-CAII (deleted in polyposis |
| AB023416 | p < 1e−07 | ASC\|apoptosis-associated speck-like protein containing a CARD |
| AL049987 | p < 1e−07 | Homo sapiens mRNA; cDNA DKFZp564F112 (from clone DKFZp564F112) |
| AF261655 | p < 1e−07 | HMIC\|1,2-alpha-mannosidase IC |
| AK025683 | 0.0000001 | Homo sapiens cDNA: FLJ22030 fis, clone HEP08669 |
| AA401311 | 0.0000002 | KIAA1377\|KIAA1377 protein |
| BF446757 | p < 1e−07 | ESTs |
| AW076080 | p < 1e−07 | Homo sapiens, clone IMAGE: 3463399, mRNA, partial cds |
| BC009778 | 0.0000007 | LOC51207\|protein phosphatase |
| X13897 | 0.0000008 | CYP2A6\|cytochrome P450, subfamily IIA (phenobarbital-inducible), polypeptide 6 |
| AW450142 | 0.0000002 | SLC22A4\|solute carrier family 22 (organic cation transporter), member 4 |
| AA086248 | 0.0000009 | ESTs |
| AW150018 | 0.0000001 | FMOD\|fibromodulin |
| AA831831 | p < 1e−07 | MORF\|histone acetyltransferase |
| AL050143 | p < 1e−07 | DKFZP586B2420\|DKFZP586B2420 protein |
| AL110252 | 0.0000155 | Homo sapiens mRNA; cDNA DKFZp566A1046 (from clone DKFZp566A1046) |
| M92432 | 0.0000243 | GUCY2D\|guanylate cyclase 2D, membrane (retina-specific) |
| AI185187 | p < 1e−07 | LOC51133\|NY-REN-45 antigen |
| AF094758 | p < 1e−07 | NESG1\|nasopharyngeal epithelium specific protein 1 |
| BC012541 | 0.0000005 | ALEX2\|armadillo repeat protein ALEX2 |
| AW140108 | 0.0000121 | HOXD8\|homeo box D8 |
| AF212239 | 0.0000254 | RBP5\|retinol binding protein 5, cellular |
| AK000207 | p < 1e−07 | FLJ20200\|hypothetical protein FLJ20200 |
| BC002842 | 0.0000004 | H2BFB\|H2B histone family, member B |
| AI263595 | 0.0000118 | ESTs |
| AL136911 | p < 1e−07 | EPLIN\|epithelial protein lost in neoplasm beta |
| NM_004694 | p < 1e−07 | SLC16A6\|solute carrier family 16 (monocarboxylic acid transporters), member 6 |
| BC007323 | 0.0000001 | NDUFC2\|NADH dehydrogenase (ubiquinone) 1, subcomplex unknown, 2 (14.5 kD, B14.5b) |
| AI474354 | 0.0000155 | TSLRP\|testis specific leucine rich repeat protein |
| NM_004430 | 0.0000026 | EGR3\|early growth response 3 |
| BM128958 | p < 1e−07 | KIAA1643\|KIAA1643 protein |
| AL360141 | p < 1e−07 | PEX11A\|peroxisomal biogenesis factor 11A |
| BC000465 | p < 1e−07 | GADD45G\|growth arrest and DNA-damage-inducible, gamma |
| AI629046 | p < 1e−07 | EMX1\|empty spiracles homolog 1 (Drosophila) |
| AF267859 | 0.0000002 | FLJ13153\|hypothetical protein FLJ13153 |
| AA654227 | p < 1e−07 | BCAS4\|breast carcinoma amplified sequence 4 |
| U38276 | p < 1e−07 | SEMA3F\|sema domain, immunoglobulin domain (Ig), short basic domain, secreted, (semaphorin) 3F |
| NM_053056 | p < 1e−07 | CCND1\|cyclin D1 (PRAD1: parathyroid adenomatosis 1) |
| AL117393 | p < 1e−07 | KIF5C\|kinesin family member 5C |
| AI921122 | 0.000001 | ESTs, Highly similar to dJ25J6.2 [H. sapiens] |
| BC009381 | 0.0000055 | STMN3\|stathmin-like 3 |
| AW207140 | 0.0000101 | ARNT2\|aryl-hydrocarbon receptor nuclear translocator 2 |
| AW083384 | p < 1e−07 | ESTs, Moderately similar to T46395 hypothetical protein DKFZp434I1120.1 [H. sapiens] |
| AJ420536 | p < 1e−07 | Homo sapiens mRNA full length insert cDNA clone EUROIMAGE 994183 |
| AI561078 | p < 1e−07 | TSPAN-1\|tetraspan 1 |
| AK000209 | p < 1e−07 | FLJ20202\|hypothetical protein FLJ20202 |
| AI674472 | p < 1e−07 | EST |
| R40176 | 0.0000412 | Homo sapiens cDNA FLJ32051 fis, clone NTONG2001428 |
| NM_003248 | 0.0000968 | THBS4\|thrombospondin 4 |
| AK023243 | p < 1e−07 | FLJ13181\|hypothetical protein FLJ13181 |
| AB000509 | p < 1e−07 | TRAF5\|TNF receptor-associated factor 5 |
| AA400069 | 0.0000004 | FLJ13031\|hypothetical protein FLJ13031 |
| AA846684 | p < 1e−07 | ESTs |
| AI744756 | p < 1e−07 | ESTs |
| AK001889 | 0.0000002 | Homo sapiens cDNA FLJ11027 fis, clone PLACE1004114 |
| AA557345 | 0.0000009 | EST, Weakly similar to 2109260A B cell growth factor [H. sapiens] |
| NM_012168 | 0.0000109 | FBXO2\|F-box only protein 2 |
| BF475464 | p < 1e−07 | ESTs, Weakly similar to I38022 hypothetical protein [H. sapiens] |
| AL050116 | p < 1e−07 | Homo sapiens mRNA; cDNA DKFZp586A131 (from clone DKFZp586A131) |
| NM_001089 | p < 1e−07 | ABCA3\|ATP-binding cassette, sub-family A (ABC1), member 3 |
| BC017586 | 0.0000002 | Homo sapiens, Similar to RIKEN cDNA 1700028N11 gene, clone MGC: 26610 IMAGE: 4837506, mRNA, complete |
| | | cds |
| AW006452 | 0.0000006 | ESTs, Weakly similar to ALU5_HUMAN ALU SUBFAMILY SC SEQUENCE CONTAMINATION WARNING ENTRY |
| | | [H. sapiens] |
| AI097387 | p < 1e−07 | PRO1489\|hypothetical protein PRO1489 |
| AA601573 | p < 1e−07 | ESTs |
| AW080826 | p < 1e−07 | KIAA0984\|KIAA0984 protein |
| AI809547 | 0.0000005 | ESTs |
| AJ420459 | p < 1e−07 | LOC51184\|protein x 0004 |
| BE856073 | p < 1e−07 | STK39\|serine threonine kinase 39 (STE20/SPS1 homolog, yeast) |
| AK000416 | 0.0000062 | SLC16A5\|solute carrier family 16 (monocarboxylic acid transporters), member 5 |
| AW291846 | p < 1e−07 | C21orf15\|chromosome 21 open reading frame 15 |
| AI971681 | 0.000003 | PLCE\|phospholipase C, epsilon |
| AL157455 | 0.000004 | Homo sapiens mRNA; cDNA DKFZp761I1912 (from clone DKFZp761I1912) |
| AI823660 | 0.0000048 | Homo sapiens cDNA: FLJ22133 fis, clone HEP20529 |
| AF137027 | 0.0000933 | TCL1B\|T-cell leukemia/lymphoma 1B |
| AF161423 | p < 1e−07 | PTD002\|PTD002 protein |
| NM_006186 | 0.0000034 | NR4A2\|nuclear receptor subfamily 4, group A, member 2 |
| BC009522 | 0.0000046 | SSTR2\|somatostatin receptor 2 |
| AI783781 | 0.0002071 | EST |
| AL157492 | p < 1e−07 | DKFZP434P0111\|hypothetical protein DKFZp434P0111 |
| AK000691 | p < 1e−07 | FLJ22174\|hypothetical protein FLJ22174 |
| BC004312 | 0.0000013 | IGFBP2\|insulin-like growth factor binding protein 2 (36 kD) |
| AA973836 | 0.0001602 | ESTs |
| AL049223 | p < 1e−07 | SCAMP1\|secretory carrier membrane protein 1 |
| AI650407 | p < 1e−07 | ESTs |
| AA054983 | p < 1e−07 | EST |
| AA643697 | 0.000009 | ESTs |
| BC000059 | 0.0000092 | CELSR1\|cadherin, EGF LAG seven-pass G-type receptor 1 (flamingo homolog, Drosophila) |
| BC008703 | p < 1e−07 | MGC15737\|hypothetical protein MGC15737 |
| AK054785 | p < 1e−07 | Homo sapiens cDNA FLJ30223 fis, clone BRACE2001785 |
| AK025209 | p < 1e−07 | Homo sapiens cDNA: FLJ21556 fis, clone COL06353 |
| AY033290 | p < 1e−07 | LABH1\|lung alpha/beta hydrolase 1 |
| AW074143 | 0.0000002 | ESTs |
| AL137419 | 0.0001168 | C12orf3\|chromosome 12 open reading frame 3 |
| AI031941 | p < 1e−07 | ESTs, Weakly similar to YEX0_YEAST HYPOTHETICAL 64.8 KDA PROTEIN IN GDI1-COX15 INTERGENIC |
| | | REGION [S. cerevisiae] |
| BF511644 | p < 1e−07 | ESTs, Weakly similar to Z195_HUMAN ZINC FINGER PROTEIN 195 [H. sapiens] |
| BF436282 | p < 1e−07 | RBSK\|ribokinase |
| AA854788 | p < 1e−07 | KAL1\|Kallmann syndrome 1 sequence |
| H08145 | 0.0000011 | ESTs |
| NM_000139 | 0.0000023 | MS4A2\|membrane-spanning 4-domains, subfamily A, member 1 |
| NM_000142 | 0.000013 | FGFR3\|fibroblast growth factor receptor 3 (achondroplasia, thanatophoric dwarfism) |
| AI204186 | 0.0001851 | ESTs |
| AI341790 | p < 1e−07 | Homo sapiens, clone MGC: 2492 IMAGE: 3353520, mRNA, complete cds |
| AI499814 | p < 1e−07 | KIAA1372\|KIAA1372 protein |
| BC014000 | p < 1e−07 | Homo sapiens, clone MGC: 20208 IMAGE: 3936339, mRNA, complete cds |
| AI221721 | 0.0000001 | CBFA2T3\|core-binding factor, runt domain, alpha subunit 2; translocated to, 3 |
| AW139237 | 0.000015 | ESTs |
| AA910336 | 0.0000163 | ESTs |
| AK022443 | 0.0000003 | Homo sapiens cDNA FLJ12381 fis, clone MAMMA1002566 |
| AL133030 | 0.0000029 | KIAA1666\|KIAA1666 protein |
| AK025532 | p < 1e−07 | FLJ21213\|hypothetical protein FLJ21213 |
| AL119854 | 0.0000017 | KIAA1789\|KIAA1789 protein |
| AA613425 | 0.0000152 | COMP\|cartilage oligomeric matrix protein (pseudoachondroplasia, epiphyseal dysplasia 1, multiple) |
| AF245044 | p < 1e−07 | HT023\|hypothetical protein HT023 |
| AI951472 | p < 1e−07 | FLJ10724\|melanoma antigen recognized by T cells 2 |
| AK026216 | p < 1e−07 | Homo sapiens cDNA: FLJ22563 fis, clone HSI01928 |
| BC017772 | 0.0000182 | HT021\|HT021 |
| AI810347 | p < 1e−07 | ESTs, Weakly similar to ABP2_HUMAN ENDOTHELIAL ACTIN-BINDING PROTEIN [H. sapiens] |
| X86163 | 0.0000009 | BDKRB2\|bradykinin receptor B2 |
| U67784 | 0.0000164 | RDC1\|G protein-coupled receptor |
| BC013083 | p < 1e−07 | CST3\|cystatin C (amyloid angiopathy and cerebral hemorrhage) |
| AK025047 | 0.0000001 | FLJ21394\|hypothetical protein FLJ21394 |
| AF288571 | 0.0000002 | LEF1\|lymphoid enhancer-binding factor 1 |
| BC002865 | 0.0000004 | MGC11242\|hypothetical protein MGC11242 |
| BF061965 | 0.0000011 | KIAA1257\|KIAA1257 protein |
| H05388 | 0.0000808 | KIAA0844\|KIAA0844 protein |
| AF243505 | 0.0002923 | OTOR\|otoraplin |
| AL136721 | p < 1e−07 | DKFZP566K1946\|hypothetical protein DKFZp566K1946 |
| AK024715 | p < 1e−07 | FLJ21062\|hypothetical protein FLJ21062 |
| W52485 | p < 1e−07 | MST1\|macrophage stimulating 1 (hepatocyte growth factor-like) |
| AA398015 | 0.0006358 | CLECSF1\|C-type (calcium dependent, carbohydrate-recognition domain) lectin, superfamily member 1 (cartilage- |
| | | derived) |
| NM_001771 | p < 1e−07 | CD22\|CD22 antigen |
| AW451965 | p < 1e−07 | ESTs |
| NM_004425 | 0.0000213 | ECM1\|extracellular matrix protein 1 |
| AA962285 | p < 1e−07 | KIAA1010\|KIAA1010 protein |
| BC011806 | p < 1e−07 | FLJ13187\|phafin 2 |
| AI304875 | p < 1e−07 | DGKD\|diacylglycerol kinase, delta (130 kD) |
| AA406206 | 0.0000026 | EPB41L4\|erythrocyte protein band 4.1-like 4 |
| BC004540 | 0.0000031 | MGC11287\|hypothetical protein MGC11287 similar to ribosomal protein S6 kinase, |
| L13773 | p < 1e−07 | MLLT2\|myeloid/lymphoid or mixed-lineage leukemia (trithorax homolog, Drosophila); translocated to, 2 |
| AK026698 | p < 1e−07 | FLJ23045\|hypothetical protein FLJ23045 |
| BC007617 | p < 1e−07 | G6PT1\|glucose-6-phosphatase, transport (glucose-6-phosphate) protein 1 |
| BF447022 | 0.0000008 | EST |
| AL080136 | 0.0005363 | SPAG6\|sperm associated antigen 6 |
| BC000691 | p < 1e−07 | LOC51673\|brain specific protein |
| BF939249 | p < 1e−07 | ESTs |
| AF113212 | 0.0000001 | MSTP032\|MSTP032 protein |
| AI620139 | p < 1e−07 | ESTs |
| BC014336 | 0.0000002 | HHEX\|hematopoietically expressed homeobox |
| AI241077 | 0.0001517 | PAH\|phenylalanine hydroxylase |
| BC015571 | p < 1e−07 | SLC22A1L\|solute carrier family 22 (organic cation transporter), member 1-like |
| T23696 | p < 1e−07 | FLJ20015\|hypothetical protein |
| AW196636 | p < 1e−07 | ESTs |
| R38689 | 0.0000006 | PCLO\|piccolo (presynaptic cytomatrix protein) |
| AJ251053 | 0.0000017 | STHM\|sialyltransferase |
| BC002757 | 0.0000033 | COX7A1\|cytochrome c oxidase subunit VIIa polypeptide 1 (muscle) |
| X59766 | 0.0000194 | AZGP1\|alpha-2-glycoprotein 1, zinc |
| AK000772 | p < 1e−07 | BA526D8.4\|KRAB box containing C2H2 type zinc finger bA526D8.4 |
| AW134884 | 0.0000014 | GHRH\|growth hormone releasing hormone |
| AF263545 | 0.0000217 | Homo sapiens HUT11 protein mRNA, partial 3′ UTR |
| AF034996 | 0.0000879 | AMPH\|amphiphysin (Stiff-Mann syndrome with breast cancer 128 kD autoantigen) |
| NM_004029 | p < 1e−07 | IRF7\|interferon regulatory factor 7 |
| AI216751 | 0.0005965 | ESTs |
| AI350518 | p < 1e−07 | ESTs |
| AL049329 | p < 1e−07 | DKFZP564G196\|DKFZP564G196 protein |
| AK023757 | p < 1e−07 | Homo sapiens cDNA FLJ13695 fis, clone PLACE2000124 |
| AI338252 | p < 1e−07 | LARGE\|like-glycosyltransferase |
| NM_005410 | 0.0001146 | SEPP1\|selenoprotein P, plasma, 1 |
| AK024898 | p < 1e−07 | Homo sapiens cDNA: FLJ21245 fis, clone COL01184 |
| AW207060 | p < 1e−07 | DNAJL1\|hypothetical protein similar to mouse Dnajl1 |
| AL136790 | p < 1e−07 | DKFZp434F1819\|hypothetical protein DKFZp434F1819 |
| AI973194 | p < 1e−07 | OVGP1\|oviductal glycoprotein 1, 120 kD (mucin 9, oviductin) |
| U01824 | 0.0000003 | SLC1A2\|solute carrier family 1 (glial high affinity glutamate transporter), member 2 |
| AL136789 | 0.0000006 | DKFZp434F1719\|hypothetical protein DKFZp434F1719 |
| BC014277 | 0.0000017 | TIMP3\|tissue inhibitor of metalloproteinase 3 (Sorsby fundus dystrophy, pseudoinflammatory) |
| AK024750 | 0.0000299 | C11orf14\|chromosome 11 open reading frame 14 |
| BC000187 | 0.0000002 | COX6C\|cytochrome c oxidase subunit VIc |
| AB025904 | p < 1e−07 | CA14\|carbonic anhydrase XIV |
| AW004703 | 0.0000002 | FLJ10916\|hypothetical protein FLJ10916 |
| AK001865 | 0.0000065 | Homo sapiens cDNA FLJ11003 fis, clone PLACE1002851 |
| AK026690 | p < 1e−07 | Homo sapiens cDNA: FLJ23037 fis, clone LNG02036, highly similar to HSU68019 Homo sapiens mad protein |
| | | homolog (hMAD-3) mRNA |
| BC001526 | p < 1e−07 | Homo sapiens, clone MGC: 3245 IMAGE: 3505639, mRNA, complete cds |
| BC014651 | 0.0000068 | HOXB6\|homeo box B6 |
| AF085233 | 0.0000138 | SGKL\|serum/glucocorticoid regulated kinase-like |
| AF000546 | 0.0000001 | P2Y5\|purinergic receptor (family A group 5) |
| AL109959 | p < 1e−07 | Homo sapiens mRNA full length insert cDNA clone EUROIMAGE 1019273 |
| R15800 | p < 1e−07 | ESTs |
| AW069800 | 0.0000002 | Homo sapiens cDNA FLJ11685 fis, clone HEMBA1004934 |
| AA906471 | 0.0000003 | ESTs, Weakly similar to A28996 proline-rich protein M14 precursor - mouse [M. musculus] |
| AA233862 | 0.0000017 | ESTs |
| AK056155 | p < 1e−07 | Homo sapiens cDNA FLJ31593 fis, clone NT2RI2002481 |
| AI214383 | p < 1e−07 | SYNGR1\|synaptogyrin 1 |
| AW070489 | p < 1e−07 | HOXC6\|homeo box C6 |
| AI401095 | p < 1e−07 | EST |
| AK027245 | 0.0000001 | LOC55815\|hypothetical protein |
| AW732240 | 0.0000024 | ESTs |
| AF130068 | 0.0000027 | SERPINA1\|serine (or cysteine) proteinase inhibitor, clade A (alpha-1 antiproteinase, antitrypsin), member 1 |
| AA766862 | p < 1e−07 | KIAA0298\|KIAA0298 gene product |
| AA765256 | 0.0000004 | ESTs, Weakly similar to unnamed protein product [H. sapiens] |
| AA805317 | 0.0000038 | HOXC4\|homeo box C4 |
| AF119815 | 0.0000147 | GPR74\|G protein-coupled receptor 74 |
| X97881 | p < 1e−07 | GPRK2L\|G protein-coupled receptor kinase 2-like (Drosophila) |
| BF431989 | 0.0000001 | ESTs |
| AI939560 | p < 1e−07 | ESTs, Weakly similar to CIKF_HUMAN VOLTAGE-GATED POTASSIUM CHANNEL PROTEIN KV3.3 [H. sapiens] |
| AK024106 | 0.0000006 | Homo sapiens cDNA FLJ14044 fis, clone HEMBA1006124 |
| X95715 | 0.0000013 | ABCC6\|ATP-binding cassette, sub-family C (CFTR/MRP), member 6 |
| AJ409065 | p < 1e−07 | LEAP-2\|liver-expressed antimicrobial peptide 2 |
| AW590215 | p < 1e−07 | SLC4A8\|solute carrier family 4, sodium bicarbonate cotransporter, member 8 |
| BF508608 | 0.000066 | Homo sapiens cDNA FLJ12993 fis, clone NT2RP3000197 |
| BF508719 | 0.0006111 | CGI-96\|CGI-96 protein |
| AK023905 | p < 1e−07 | Homo sapiens cDNA FLJ13843 fis, clone THYRO1000796 |
| AA631898 | p < 1e−07 | EST |
| AF030186 | 0.0001916 | GPC4\|glypican 4 |
| AI655416 | p < 1e−07 | FLJ11756\|hypothetical protein FLJ11756 |
| BI492432 | 0.0000001 | PMP22\|peripheral myelin protein 22 |
| AI273078 | 0.0000001 | ELL2\|ELL-RELATED RNA POLYMERASE II, ELONGATION FACTOR |
| BC011690 | p < 1e−07 | CORO2A\|coronin, actin binding protein, 2A |
| AI695809 | p < 1e−07 | A1BG\|alpha-1-B glycoprotein |
| AI753700 | p < 1e−07 | ESTs |
| AI567961 | 0.0000001 | Homo sapiens cDNA FLJ13557 fis, clone PLACE1007737 |
| AA053400 | 0.0000015 | ESTs |
| AF182316 | p < 1e−07 | FER1L3\|fer-1-like 3, myoferlin (C. elegans) |
| AI096474 | 0.0000001 | APXL\|apical protein-like (Xenopus laevis) |
| BF475375 | 0.0000002 | EST |
| AI125473 | 0.0000075 | ESTs |
| NM_001900 | 0.0000093 | CST5\|cystatin D |
| AF173081 | 0.0006871 | VELI1\|Vertebrate LIN7 homolog 1, Tax interaction protein 33 |
| AK024733 | 0.0000009 | FLJ21080\|hypothetical protein FLJ21080 |
| AI341378 | 0.0000025 | ESTs, Moderately similar to KIAA0940 protein [H. sapiens] |
| AF334710 | 0.0000246 | Homo sapiens pyruvate dehydrogenase kinase 4 mRNA, 3′ untranslated region, partial sequence |
| AW073096 | 0.0001789 | ESTs |
| AI373912 | 0.0005046 | HABP2\|hyaluronan binding protein 2 |
| BC014949 | p < 1e−07 | LGP2\|likely ortholog of mouse D11Igp2 |
| AA160784 | p < 1e−07 | ESTs |
| AL359558 | 0.0000013 | Homo sapiens mRNA; cDNA DKFZp762O1615 (from clone DKFZp762O1615) |
| AI335013 | 0.0000015 | Homo sapiens cDNA FLJ12052 fis, clone HEMBB1002042, moderately similar to CYTOCHROME P450 4C1 (EC |
| | | 1.14.14.1) |
| AW138839 | 0.0000113 | ESTs |
| R54824 | 0.0001665 | PCDH19\|protocadherin 19 |
| N52765 | 0.0002372 | CACNA1I\|calcium channel, voltage-dependent, alpha 1I subunit |
| AI571426 | 0.0006325 | SFRP4\|secreted frizzled-related protein 4 |
| AI675370 | 0.0000005 | Homo sapiens cDNA FLJ30279 fis, clone BRACE2002772, moderately similar to HYDROXYACYLGLUTATHIONE |
| | | HYDROLASE (EC 3.1.2.6) |
| AW452172 | 0.000098 | ESTs |
| BC001356 | p < 1e−07 | IFI35\|interferon-induced protein 35 |
| AK026642 | 0.0000001 | C20orf35\|chromosome 20 open reading frame 35 |
| AL133102 | 0.0000012 | CGI-142\|CGI-142 |
| AI950985 | 0.0000045 | ESTs |
| BC002709 | p < 1e−07 | TP53TG1\|TP53 target gene 1 |
| BE465899 | p < 1e−07 | ESTs |
| AA905217 | 0.0000049 | Homo sapiens cDNA FLJ25460 fis, clone TST09046 |
| BC007659 | 0.0000091 | NQO1\|NAD(P)H dehydrogenase, quinone 1 |
| M73481 | p < 1e−07 | GRPR\|gastrin-releasing peptide receptor |
| AI912080 | p < 1e−07 | ESTs |
| AF039698 | p < 1e−07 | SDCCAG33\|serologically defined colon cancer antigen 33 |
| AI668861 | p < 1e−07 | C18orf1\|chromosome 18 open reading frame 1 |
| AW139766 | p < 1e−07 | ESTs |
| AK000322 | 0.0000112 | FLJ20315\|hypothetical protein FLJ20315 |
| R52665 | 0.0000001 | KIAA0323\|KIAA0323 protein |
| AY014282 | 0.0000008 | FLJ12476\|hypothetical protein FLJ12476 |
| AF264784 | p < 1e−07 | TRPS1\|trichorhinophalangeal syndrome I |
| AF073770 | 0.0000002 | CROT\|carnitine O-octanoyltransferase |
| X08020 | 0.0000003 | GSTM1\|glutathione 5-transferase M1 |
| L03785 | p < 1e−07 | MYL5\|myosin, light polypeptide 5, regulatory |
| AI651570 | p < 1e−07 | PCM1\|pericentriolar material 1 |
| AL049949 | p < 1e−07 | Homo sapiens mRNA; cDNA DKFZp564L0822 (from clone DKFZp564L0822) |
| AK027191 | 0.0000008 | Homo sapiens cDNA: FLJ23538 fis, clone LNG08010, highly similar to BETA2 Human MEN1 region clone |
| | | epsilon/beta mRNA |
| AW450777 | 0.0000011 | FLJ14009\|hypothetical protein FLJ14009 |
| AI027237 | 0.0000012 | CACNA2D2\|calcium channel, voltage-dependent, alpha 2/delta subunit 2 |
| AA668884 | 0.0000034 | ESTs |
| AL360254 | 0.0000007 | SLI\|neuronal Shc adaptor homolog |
| BC014123 | p < 1e−07 | FLJ20920\|hypothetical protein FLJ20920 |
| AF334676 | 0.0001389 | TEKT3\|tektin 3 |
| AI123176 | p < 1e−07 | ESTs |
| AK026782 | p < 1e−07 | FLJ23129\|hypothetical protein FLJ23129 |
| AF047033 | 0.0000008 | SLC4A7\|solute carrier family 4, sodium bicarbonate cotransporter, member 7 |
| BF509554 | 0.0000021 | ESTs |
| NM_001337 | 0.0003886 | CX3CR1\|chemokine (C-X3-C) receptor 1 |
| AL353936 | 0.0004393 | DKFZp761K1423\|hypothetical protein DKFZp761K1423 |
| AF285836 | 0.0000001 | NPDC1\|neural proliferation, differentiation and control, 1 |
| AF038633 | 0.0000003 | UCN\|urocortin |
| AW301079 | 0.0006045 | ESTs, Moderately similar to ALU7_HUMAN ALU SUBFAMILY SQ SEQUENCE CONTAMINATION WARNING |
| | | ENTRY [H. sapiens] |
| BF432086 | 0.0006733 | LOC63923\|hypothetical protein similar to tenascin-R |
| T52873 | 0.0000001 | ESTs, Moderately similar to G02075 transcription repressor zinc finger protein 85 [H. sapiens] |
| AB018303 | 0.0000147 | OAZ\|OLF-1/EBF associated zinc finger gene |
| AA233494 | 0.000036 | ZNF132\|zinc finger protein 132 (clone pHZ-12) |
| AK057894 | 0.0000335 | Homo sapiens cDNA FLJ25165 fis, clone CBR08421 |
| AF132197 | p < 1e−07 | PRO1331\|hypothetical protein PRO1331 |
| AL122071 | 0.0004254 | Homo sapiens mRNA; cDNA DKFZp434H1235 (from clone DKFZp434H1235); partial cds |
| AK054617 | 0.0006328 | Homo sapiens cDNA FLJ30055 fis, clone ADRGL1000165, weakly similar to IMIDAZOLONEPROPIONASE (EC |
| | | 3.5.2.7) |
| AK024986 | 0.0000001 | Homo sapiens cDNA: FLJ21333 fis, clone COL02535 |
| L11672 | 0.0000009 | ZNF91\|zinc finger protein 91 (HPF7, HTF10) |
| AK025583 | 0.0000089 | Homo sapiens cDNA: FLJ21930 fis, clone HEP04301, highly similar to HSU90916 Human clone 23815 mRNA |
| | | sequence |
| R92436 | 0.0000707 | ESTs |
| AF062595 | 0.0002667 | AK5\|adenylate kinase 5 |
| AW575162 | 0.0000014 | ACP5\|acid phosphatase 5, tartrate resistant |
| AB067468 | 0.0000552 | KIAA1881\|KIAA1881 protein |
| AF192522 | 0.0001271 | NPC1L1\|NPC1 (Niemann-Pick disease, type C1, gene)-like 1 |
| AI668626 | 0.0000009 | Homo sapiens cDNA FLJ11648 fis, clone HEMBA1004405 |
| NM_017458 | p < 1e−07 | MVP\|major vault protein |
| AI393709 | 0.0000001 | DFFA\|DNA fragmentation factor, 45 kD, alpha polypeptide |
| BC003667 | 0.0000003 | RPS27L\|ribosomal protein S27-like |
| BE857676 | 0.0004456 | ESTs |
| AI910755 | 0.0000002 | ESTs |
| Z48633 | 0.0000007 | H. sapiens mRNA for retrotransposon |
| AK024192 | 0.000001 | Homo sapiens cDNA FLJ14130 fis, clone MAMMA1002618 |
| L10386 | 0.0000027 | TGM3\|transglutaminase 3 (E polypeptide, protein-glutamine-gamma-glutamyltransferase) |
| BC014105 | 0.0000005 | LOC51015\|CGI-111 protein |
| H10298 | 0.000003 | ESTs |
| AI620965 | 0.0000143 | ESTs |
| BF056618 | 0.0002275 | C20orf39\|chromosome 20 open reading frame 39 |
| AI984359 | 0.0000002 | MAWBP\|MAWD binding protein |
| BC018001 | 0.0000004 | MB\|myoglobin |
| AK026839 | 0.0000347 | FLJ23186\|hypothetical protein FLJ23186 |
| BC012069 | p < 1e−07 | NUDT4\|nudix (nucleoside diphosphate linked moiety X)-type motif 4 |
| H66199 | 0.0000002 | OR2W3P\|olfactory receptor, family 2, subfamily W, member 3 pseudogene |
| AL080213 | 0.0000003 | Homo sapiens mRNA; cDNA DKFZp586I1823 (from clone DKFZp586I1823) |
| NM_030761 | 0.0000204 | WNT4\|wingless-type MMTV integration site family, member 4 |
| AF052224 | 0.0000265 | BSN\|bassoon (presynaptic cytomatrix protein) |
| NM_002625 | 0.0000553 | PFKFB1\|6-phosphofructo-2-kinase/fructose-2,6-biphosphatase 1 |
| AI741469 | 0.0000946 | ESTs |
| AW293219 | 0.0004089 | Homo sapiens cDNA FLJ12280 fis, clone MAMMA1001744 |
| BC002752 | 0.0000008 | PTD015\|PTD015 protein |
| BC010082 | 0.0000028 | Homo sapiens, Similar to hypothetical protein FLJ10761, clone MGC: 19512 IMAGE: 4329734, mRNA, complete |
| | | cds |
| AI954686 | 0.0001698 | BFSP2\|beaded filament structural protein 2, phakinin |
| AI620480 | 0.0003718 | EST |
| AA758546 | 0.0004796 | LOC51086\|putative protein-tyrosine kinase |
| NM_001873 | 0.000995 | CPE\|carboxypeptidase E |
| BC009789 | 0.0000001 | NPHP1\|nephronophthisis 1 (juvenile) |
| AF218006 | 0.0000018 | FLJ13612\|hypothetical protein FLJ13612 |
| AA580186 | 0.0000046 | EST |
| AI247211 | 0.0000002 | ESTs |
| BC010117 | 0.0000006 | MGC13038\|hypothetical protein MGC13038 |
| X55720 | 0.0000006 | CSF3R\|colony stimulating factor 3 receptor (granulocyte) |
| X79676 | 0.0000117 | PTPN13\|protein tyrosine phosphatase, non-receptor type 13 (APO-1/CD95 (Fas)-associated phosphatase) |
| BC002415 | 0.0002872 | GSTT2\|glutathione S-transferase theta 2 |
| AI703322 | 0.0006439 | PLIN\|perilipin |
| NM_004370 | 0.0000819 | COL12A1\|collagen, type XII, alpha 1 |
| AW129056 | 0.0002021 | KIAA0485\|KIAA0485 protein |
| AL512723 | 0.0000013 | DKFZP547L112\|hypothetical protein DKFZp547L112 |
| AW613521 | 0.0000207 | CLST11240\|CLST 11240 protein |
| AI280246 | 0.0000795 | ESTs |
| AA827649 | 0.0000004 | ESTs |
| AL161983 | 0.0000075 | Homo sapiens mRNA; cDNA DKFZp761K2024 (from clone DKFZp761K2024) |
| AW453001 | 0.0006444 | CEBPD\|CCAAT/enhancer binding protein (C/EBP), delta |
| AK025062 | 0.0000513 | Homo sapiens cDNA: FLJ21409 fis, clone COL03924 |
| H19223 | 0.0000011 | ESTs, Weakly similar to JC5238 galactosylceramide-like protein, GCP [H. sapiens] |
| BC011628 | 0.0000027 | EPHX2\|epoxide hydrolase 2, cytoplasmic |
| NM_007023 | 0.0000149 | CAMP-GEFII\|cAMP-regulated guanine nucleotide exchange factor II |
| AI888490 | 0.0002268 | ESTs, Weakly similar to I38022 hypothetical protein [H. sapiens] |
| AL133606 | 0.0000006 | FLJ11142\|hypothetical protein FLJ11142 |
| AL096842 | 0.0000008 | ATIP1\|AT2 receptor-interacting protein 1 |
| AW301074 | 0.0000056 | ESTs, Weakly similar to GTXH_HUMAN GLUTATHIONE-S-TRANSFERASE HOMOLO [H. sapiens] |
| AF131794 | 0.0004664 | KIAA0878\|KIAA0878 protein |
| AI499501 | 0.0001437 | ESTs, Weakly similar to FMOD_HUMAN FIBROMODULIN PRECURSOR [H. sapiens] |
| AF002672 | 0.0002138 | LOH11CR2A\|loss of heterozygosity, 11, chromosomal region 2, gene A |
| AK000785 | 0.0000103 | FLJ20778\|epsin 3 |
| AW183685 | 0.0000151 | ESTs |
| BC017422 | 0.0000003 | Homo sapiens, clone MGC: 27375 IMAGE: 4688423, mRNA, complete cds |
| BE501103 | 0.0000003 | Homo sapiens cDNA FLJ32173 fis, clone PLACE6000953 |
| BC005066 | 0.0000003 | Homo sapiens, clone IMAGE: 3347954, mRNA, partial cds |
| BC006534 | 0.0000028 | SARCOSIN\|sarcomeric muscle protein |
| AI800042 | 0.000004 | ESTs |
| AB062787 | 0.0000042 | TREM2\|triggering receptor expressed on myeloid cells 2 |
| NM_001197 | 0.0000451 | BIK\|BCL2-interacting killer (apoptosis-inducing) |
| BC013151 | 0.0000006 | Homo sapiens, Similar to RIKEN cDNA 4933433C09 gene, clone MGC: 21628 IMAGE: 4385421, mRNA, complete |
| | | cds |
| BC001314 | 0.0000018 | Homo sapiens, Similar to clone FLB3816, clone IMAGE: 3454380, mRNA |
| BC000006 | 0.0000506 | ATP1B1\|ATPase, Na+/K+ transporting, beta 1 polypeptide |
| R63503 | 0.0000015 | ESTs |
| AI733491 | 0.0000215 | ESTs |
| BC009753 | 0.0000598 | Homo sapiens, clone IMAGE: 3833472, mRNA |
| BC008857 | 0.0000022 | NFATC4\|nuclear factor of activated T-cells, cytoplasmic, calcineurin-dependent 4 |
| S77873 | 0.0000023 | CYP2E\|cytochrome P450, subfamily IIE (ethanol-inducible) |
| AF131833 | 0.0003466 | LOC57795\|hypothetical protein from clone 24828 |
| AF101477 | 0.0004668 | GNMT\|glycine N-methyltransferase |
| AA708035 | 0.0005873 | ESTs |
| BC005997 | 0.0000003 | MGC14801\|hypothetical protein MGC14801 |
| BE501474 | 0.000001 | Homo sapiens mRNA; cDNA DKFZp434P182 (from clone DKFZp434P182) |
| AI499593 | 0.0000015 | IRX5\|iroquois homeobox protein 5 |
| AW136984 | 0.0000906 | FLJ11235\|hypothetical protein |
| BC007808 | 0.000002 | MYH7B\|myosin, heavy polypeptide 7B, cardiac muscle, beta |
| AI311127 | 0.0000065 | ESTs |
| AF094754 | 0.0000257 | GLRB\|glycine receptor, beta |
| AW118445 | 0.0000387 | CELSR2\|cadherin, EGF LAG seven-pass G-type receptor 2 (flamingo homolog, Drosophila) |
| AL136678 | 0.0000276 | FLJ12428\|hypothetical protein FLJ12428 |
| AI733703 | 0.0004539 | ESTs |
| AK025915 | 0.0000003 | Homo sapiens cDNA FLJ10728 fis, clone NT2RP3001236 |
| AW205835 | 0.0000026 | ESTs |
| AI205008 | 0.0000041 | SLC1A4\|solute carrier family 1 (glutamate/neutral amino acid transporter), member 4 |
| AL161993 | 0.0000451 | LOC57168\|similar to aspartate beta hydroxylase (ASPH) |
| NM_013267 | 0.00005 | GA\|breast cell glutaminase |
| AA758934 | 0.000002 | Homo sapiens cDNA FLJ13362 fis, clone PLACE1000236 |
| BC015947 | 0.0001039 | KCNS3\|potassium voltage-gated channel, delayed-rectifier, subfamily S, member 3 |
| AF226730 | 0.0003918 | Cyt19\|Cyt19 protein |
| AW139590 | 0.0002061 | KIAA1443\|KIAA1443 protein |
| NM_005906 | 0.0003675 | MAK\|male germ cell-associated kinase |
| BC005124 | 0.0008792 | HOXD3\|homeo box D3 |
| AI922007 | 0.0000011 | ESTs |
| AW082270 | 0.0000015 | ESTs, Highly similar to AC004836 1 similar to cadherin and Drosophila Fat protein [H. sapiens] |
| AY014284 | 0.0000449 | TSGA10\|testis specific, 10 |
| AJ406933 | 0.0001677 | KAP3.3\|keratin associated protein 3.3 |
| AW470748 | 0.0000153 | BCAR3\|breast cancer anti-estrogen resistance 3 |
| U06641 | 0.0000758 | UGT2B15\|UDP glycosyltransferase 2 family, polypeptide B15 |
| AI658924 | 0.0006314 | EST |
| BC002689 | 0.0000016 | GLA\|galactosidase, alpha |
| AF023676 | 0.000007 | TM7SF2\|transmembrane 7 superfamily member 2 |
| BC001109 | 0.0001481 | CRABP2\|cellular retinoic acid binding protein 2 |
| BC000897 | 0.0005356 | IFITM1\|interferon induced transmembrane protein 1 (9-27) |
| BC017703 | 0.0002482 | LRRFIP2\|leucine rich repeat (in FLII) interacting protein 2 |
| BF436849 | 0.0000019 | ESTs |
| AV661191 | 0.0000777 | PDCD4\|programmed cell death 4 (neoplastic transformation inhibitor) |
| BC015657 | 0.0004846 | OA1\|ocular albinism 1 (Nettleship-Falls) |
| N94443 | 0.0006496 | KIAA0802\|KIAA0802 protein |
| AK025431 | 0.0000008 | Homo sapiens cDNA: FLJ21778 fis, clone HEP00201 |
| AF016267 | 0.0000016 | TNFRSF10C\|tumor necrosis factor receptor superfamily, member 10c, decoy without an intracellular domain |
| AF297872 | 0.000004 | TReP-132\|transcriptional regulating protein 132 |
| BC003180 | 0.0000047 | SNRPN\|small nuclear ribonucleoprotein polypeptide N |
| AI804733 | 0.0000051 | Homo sapiens cDNA FLJ32322 fis, clone PROST2003577 |
| AI026838 | 0.0000127 | ESTs, Weakly similar to NUCL_HUMAN NUCLEOLIN [H. sapiens] |
| AI097422 | 0.0000658 | LOC57100\|organic cation transporter |
| AA912183 | 0.0005194 | ESTs |
| AI399698 | 0.0000126 | EST |
| AI272951 | 0.0000286 | SERPINC1\|serine (or cysteine) proteinase inhibitor, clade C (antithrombin), member 1 |
| AW448954 | 0.0000899 | FLJ14871\|hypothetical protein FLJ14871 |
| AK027207 | 0.0007043 | FLJ23554\|hypothetical protein FLJ23554 |
| BC010691 | 0.0000064 | MGC9564\|similar to RIKEN cDNA 1110002C08 gene |
| BC011973 | 0.0000109 | FLJ20605\|hypothetical protein FLJ20605 |
| AW341854 | 0.000012 | SPAG8\|sperm associated antigen 8 |
| BC014456 | 0.0006392 | CHRNA6\|cholinergic receptor, nicotinic, alpha polypeptide 6 |
| AF037070 | 0.0000154 | KIAA0464\|ligand of neuronal nitric oxide synthase with carboxyl-terminal PDZ domain |
| BC007373 | 0.000041 | MGC16179\|hypothetical protein MGC16179 |
| BC010690 | 0.0003139 | FLJ14529\|hypothetical protein FLJ14529 |
| AI740671 | 0.0003413 | Homo sapiens cDNA FLJ32430 fis, clone SKMUS2001129, weakly similar to NAD-DEPENDENT METHANOL |
| | | DEHYDROGENASE (EC 1.1.1.244) |
| AI199647 | 0.0000028 | SCNN1D\|sodium channel, nonvoltage-gated 1, delta |
| AI659491 | 0.0000053 | EST |
| AA854341 | 0.0000092 | Homo sapiens cDNA FLJ12924 fis, clone NT2RP2004709 |
| NM_005677 | 0.0000028 | COLQ\|collagen-like tail subunit (single strand of homotrimer) of asymmetric acetylcholinesterase |
| AK057782 | 0.000412 | Homo sapiens cDNA FLJ25053 fis, clone CBL04266 |
| AL080078 | 0.0000036 | Homo sapiens mRNA; cDNA DKFZp564D1462 (from clone DKFZp564D1462) |
| AI434326 | 0.0004414 | ATP2A3\|ATPase, Ca++ transporting, ubiquitous |
| AF331643 | 0.000005 | Homo sapiens chromosome 17 open reading frame 26 (C17orf26) mRNA, complete cds |
| BC007438 | 0.0000058 | MGC4251\|hypothetical protein MGC4251 |
| AA555036 | 0.0000147 | SORD\|sorbitol dehydrogenase |
| AK021467 | 0.0000544 | Homo sapiens cDNA FLJ11405 fis, clone HEMBA1000769 |
| BF001174 | 0.0000709 | CAP2\|adenylyl cyclase-associated protein 2 |
| AL136680 | 0.0004515 | Homo sapiens mRNA; cDNA DKFZp564C2478 (from clone DKFZp564C2478); complete cds |
| AL110174 | 0.0005604 | STMN2\|stathmin-like 2 |
| AK026288 | 0.0004417 | FLJ22635\|hypothetical protein FLJ22635 |
| NM_003512 | 0.0005828 | H2AFL\|H2A histone family, member L |
| AW070686 | 0.000002 | ESTs |
| BC000866 | 0.0000034 | TIMP1\|tissue inhibitor of metalloproteinase 1 (erythroid potentiating activity, collagenase inhibitor) |
| AF070555 | 0.0000131 | PRSS11\|protease, serine, 11 (IGF binding) |
| AI962213 | 0.0001497 | KIAA0705\|atrophin-1 interacting protein 1; activin receptor interacting protein 1 |
| AW419319 | 0.0008904 | ODZ2\|odd Oz/ten-m homolog 2 (Drosophila, mouse) |
| N70848 | 0.0000023 | ESTs, Moderately similar to I38022 hypothetical protein [H. sapiens] |
| NM_001773 | 0.0009366 | CD34\|CD34 antigen |
| AK026301 | 0.0009911 | Homo sapiens cDNA: FLJ22648 fis, clone HSI07329 |
| F10094 | 0.0000158 | ESTs |
| AK055747 | 0.0007753 | FLJ13236\|hypothetical protein FLJ13236 |
| AI797308 | 0.0000092 | ESTs, Weakly similar to alternatively spliced product using exon 13A [H. sapiens] |
| AA746504 | 0.0000207 | Homo sapiens cDNA FLJ30188 fis, clone BRACE2001267 |
| AK025307 | 0.0000623 | CPT1A\|carnitine palmitoyltransferase I, liver |
| BC005357 | 0.0000049 | MGC12458\|hypothetical protein MGC12458 |
| U31214 | 0.0000475 | FXYD3\|FXYD domain-containing ion transport regulator 3 |
| AW243677 | 0.00001 | FLJ22457\|hypothetical protein FLJ22457 |
| AI868636 | 0.0001498 | Homo sapiens cDNA FLJ30403 fis, clone BRACE2008480 |
| AI659877 | 0.0008135 | ESTs |
| AK025627 | 0.0000049 | Homo sapiens, clone IMAGE: 3937015, mRNA, partial cds |
| AI811067 | 0.0000149 | Homo sapiens cDNA FLJ31422 fis, clone NT2NE2000384, weakly similar to MICRONUCLEAR LINKER HISTONE |
| | | POLYPROTEIN |
| AW139902 | 0.0002142 | ESTs |
| AB020711 | 0.0008427 | KIAA0904\|KIAA0904 protein |
| AW339037 | 0.0000243 | ESTs |
| AV647264 | 0.0000324 | ESTs |
| AI002941 | 0.0000063 | Human glucose transporter pseudogene |
| BC017073 | 0.0007021 | Homo sapiens, Similar to RIKEN cDNA 1810054O13 gene, clone IMAGE: 3845933, mRNA, partial cds |
| AW102641 | 0.0000112 | ESTs |
| AK000341 | 0.0000133 | ELOVL2\|elongation of very long chain fatty acids (FEN1/Elo2, SUR4/Elo3, yeast)-like 2 |
| NM_030878 | 0.0007446 | CYP2C8\|cytochrome P450, subfamily IIC (mephenytoin 4-hydroxylase), polypeptide 8 |
| AF288399 | 0.0000055 | C1orf25\|chromosome 1 open reading frame 25 |
| BC005956 | 0.0000248 | RLN1\|relaxin 1 (H1) |
| D50419 | 0.0000877 | ZNF175\|zinc finger protein 175 |
| AK025905 | 0.0002948 | SOX17\|SRY-related HMG-box transcription factor SOX17 |
| AK026760 | 0.0000265 | C20orf119\|chromosome 20 open reading frame 119 |
| AA999861 | 0.0001219 | ESTs |
| AA883836 | 0.0003305 | ESTs, Weakly similar to CGHU7L collagen alpha 1(III) chain precursor [H. sapiens] |
| AJ420485 | 0.0000088 | MRPL33\|mitochondrial ribosomal protein L33 |
| BF511682 | 0.0004053 | HSH2\|hematopoietic SH2 protein |
| M59303 | 0.0000685 | PROZ\|protein Z, vitamin K-dependent plasma glycoprotein |
| BM127823 | 0.0001431 | KIAA1917\|KIAA1917 protein |
| U60975 | 0.0002497 | SORL1\|sortilin-related receptor, L(DLR class) A repeats-containing |
| AA659779 | 0.0000288 | ESTs |
| BC006289 | 0.0000416 | FLJ11151\|hypothetical protein FLJ11151 |
| AF059321 | 0.0000839 | SIAT4A\|sialyltransferase 4A (beta-galactosidase alpha-2,3-sialytransferase) |
| N59238 | 0.0000132 | DKFZp761O0113\|hypothetical protein DKFZp761O0113 |
| AB032417 | 0.0003999 | FZD4\|frizzled homolog 4 (Drosophila) |
| L13220 | 0.0009771 | CALB3\|calbindin 3, (vitamin D-dependent calcium binding protein) |
| AI085836 | 0.0000535 | ESTs, Weakly similar to R5HU22 ribosomal protein L17, cytosolic [H. sapiens] |
| AI492117 | 0.0001096 | Homo sapiens putative ion channel protein CATSPER2 (CATSPER2), mRNA |
| AI311775 | 0.0001113 | ESTs |
| AW295401 | 0.0005407 | FLJ10647\|hypothetical protein FLJ10647 |
| BC013107 | 0.0000099 | H326\|H326 |
| BC000566 | 0.0000109 | RAYL\|putative GTP-binding protein similar to RAY/RAB1C |
| AA149305 | 0.0005606 | ITGBL1\|integrin, beta-like 1 (with EGF-like repeat domains) |
| AK025757 | 0.0000282 | FLJ22104\|hypothetical protein FLJ22104 |
| AW070798 | 0.0000334 | EST |
| BE549691 | 0.0000361 | Homo sapiens cDNA FLJ30927 fis, clone FEBRA2006736 |
| AI475953 | 0.0000816 | ESTs |
| AK026438 | 0.0000114 | Homo sapiens cDNA: FLJ22785 fis, clone KAIA2081 |
| AL161961 | 0.0000246 | KIAA1554\|KIAA1554 protein |
| AF242557 | 0.0001086 | TSCOT\|thymic stromal co-transporter |
| BC016628 | 0.0000127 | DKFZP434D146\|DKFZP434D146 protein |
| AA761787 | 0.0000158 | ESTs |
| BE670572 | 0.0009122 | ESTs |
| BC006131 | 0.0000961 | MGC13047\|hypothetical protein MGC13047 |
| AF024690 | 0.0002962 | GPR43\|G protein-coupled receptor 43 |
| AI961887 | 0.0007391 | COL3A1\|collagen, type III, alpha 1 (Ehlers-Danlos syndrome type IV, autosomal dominant) |
| BC004471 | 0.0000513 | APM2\|adipose specific 2 |
| AK000542 | 0.0000616 | FLJ20535\|hypothetical protein FLJ20535 |
| BC010652 | 0.0000856 | MGC9753\|hypothetical gene MGC9753 |
| AI859284 | 0.0001958 | ESTs, Weakly similar to T42709 hypothetical protein DKFZp586I0821.1 [H. sapiens] |
| BC005364 | 0.0000764 | FLJ11218\|hypothetical protein FLJ11218 |
| AI732388 | 0.0003354 | MGAT4A\|mannosyl (alpha-1,3-)-glycoprotein beta-1,4-N-acetylglucosaminyltransferase, isoenzyme A |
| AI248610 | 0.0000466 | ESTs |
| AK026720 | 0.0001708 | Homo sapiens cDNA: FLJ23067 fis, clone LNG04993 |
| NM_000609 | 0.0008601 | SDF1\|stromal cell-derived factor 1 |
| NM_006713 | 0.000127 | PC4\|activated RNA polymerase II transcription cofactor 4 |
| AK054839 | 0.0001666 | Homo sapiens cDNA FLJ30277 fis, clone BRACE2002752 |
| AI087975 | 0.0001916 | ESTs |
| AF220030 | 0.0002753 | TRIM6\|tripartite motif-containing 6 |
| BC000632 | 0.0000354 | S100A13\|S100 calcium binding protein A13 |
| BC015613 | 0.0001785 | GATA2\|GATA binding protein 2 |
| AA603977 | 0.000372 | KLK4\|kallikrein 4 (prostase, enamel matrix, prostate) |
| AW197863 | 0.0000468 | ESTs |
| BM127867 | 0.0000528 | MDM1\|nuclear protein double minute 1 |
| AA830549 | 0.0003809 | VAMP1\|vesicle-associated membrane protein 1 (synaptobrevin 1) |
| BF477905 | 0.0005735 | ESTs |
| R45048 | 0.0001759 | C22orf1\|chromosome 22 open reading frame 1 |
| AB033110 | 0.0001596 | KIAA1284\|KIAA1284 protein |
| AW293849 | 0.0001623 | ESTs, Moderately similar to I54374 gene NF2 protein [H. sapiens] |
| BC014117 | 0.0002492 | TBXAS1\|thromboxane A synthase 1 (platelet, cytochrome P450, subfamily V) |
| AK022060 | 0.0005048 | Homo sapiens cDNA FLJ11998 fis, clone HEMBB1001521 |
| AA806859 | 0.0007266 | FLJ14281\|hypothetical protein FLJ14281 |
| AW291180 | 0.0000866 | ADORA3\|adenosine A3 receptor |
| BF510778 | 0.0001368 | ESTs |
| AA603743 | 0.0002352 | EST |
| BC005305 | 0.0006323 | COL4A6\|collagen, type IV, alpha 6 |
| AF068180 | 0.0001499 | BLNK\|B-cell linker |
| R46824 | 0.0002785 | SRGAP3\|KIAA0411 gene product |
| AI081593 | 0.0001534 | ESTs |
| AF311325 | 0.0001578 | GPD2\|glycerol-3-phosphate dehydrogenase 2 (mitochondrial) |
| AK055280 | 0.0001954 | Homo sapiens cDNA FLJ30718 fis, clone FCBBF2001675 |
| AB018316 | 0.0006996 | KIAA0773\|KIAA0773 gene product |
| AK025168 | 0.0000869 | OSBPL6\|oxysterol binding protein-like 6 |
| AL117616 | 0.0001068 | SRI\|sorcin |
| AF332975 | 0.0005447 | ZAN\|zonadhesin |
| R43793 | 0.0001576 | HSPA1L\|heat shock 70 kD protein 1-like |
| AI307801 | 0.000302 | KIAA1072\|KIAA1072 protein |
| AK027036 | 0.0008803 | RU2\|RU2S |
| BC006378 | 0.0001365 | UBE1L\|ubiquitin-activating enzyme E1-like |
| AL137559 | 0.0004641 | PCANAP7\|prostate cancer associated protein 7 |
| J03607 | 0.0001134 | KRT19\|keratin 19 |
| N93663 | 0.0003213 | ESTs |
| AA767639 | 0.0006013 | KIAA0227\|KIAA0227 protein |
| BF448987 | 0.0002835 | ESTs |
| AL110131 | 0.0006287 | KIAA1223\|KIAA1223 protein |
| NM_006455 | 0.0001119 | SC65\|nucleolar autoantigen (55 kD) similar to rat synaptonemal complex protein |
| R56793 | 0.0009148 | AGXT2L1\|alanine-glyoxylate aminotransferase 2-like 1 |
| AF059617 | 0.0006528 | SNK\|serum-inducible kinase |
| BC005055 | 0.0003206 | FOXP1\|forkhead box P1 |
| AF141332 | 0.0004598 | APOB48R\|apolipoprotein B48 receptor |
| AK025772 | 0.0001313 | Homo sapiens cDNA: FLJ22119 fis, clone HEP18852 |
| AI424409 | 0.0002138 | ESTs, Highly similar to LB4D_HUMAN NADP-DEPENDENT LEUKOTRIENE B4 12- |
| | | HYDROXYDEHYDROGENASE [H. sapiens] |
| AK056154 | 0.0007347 | Homo sapiens cDNA FLJ31592 fis, clone NT2RI2002447 |
| BC014533 | 0.0001558 | MEF-2\|myelin gene expression factor 2 |
| AI089428 | 0.0007681 | ESTs |
| BC017411 | 0.0001941 | Homo sapiens, clone IMAGE: 4618419, mRNA, partial cds |
| AF263462 | 0.0003563 | CGN\|cingulin |
| BC003608 | 0.0005011 | RBPMS\|RNA-binding protein gene with multiple splicing |
| AK027146 | 0.0004053 | RPL5\|ribosomal protein L5 |
| U90914 | 0.0009563 | CPD\|carboxypeptidase D |
| BF508058 | 0.0002228 | DKFZP564D166\|putative ankyrin-repeat containing protein |
| AK026256 | 0.0007805 | MGC4171\|hypothetical protein MGC4171 |
| AB069698 | 0.0005545 | ADAMTS13\|a disintegrin-like and metalloprotease (reprolysin type) with thrombospondin type 1 motif, 13 |
| BC005359 | 0.0002707 | GMFB\|glia maturation factor, beta |
| NM_021194 | 0.0002725 | SLC30A1\|solute carrier family 30 (zinc transporter), member 1 |
| AI311898 | 0.0003307 | ESTs |
| NM_002538 | 0.0005858 | OCLN\|occludin |
| AF222340 | 0.0007234 | ARTS-1\|type 1 tumor necrosis factor receptor shedding aminopeptidase regulator |
| W32171 | 0.0005402 | Homo sapiens cDNA FLJ13613 fis, clone PLACE1010856 |
| BC009869 | 0.0008215 | SERF2\|small EDRK-rich factor 2 |
| AK021674 | 0.0003543 | Homo sapiens cDNA FLJ11612 fis, clone HEMBA1004011 |
| AB039903 | 0.0007986 | TRIM34\|tripartite motif-containing 34 |
| AJ252236 | 0.0004879 | CAMK2B\|calcium/calmodulin-dependent protein kinase (CaM kinase) II beta |
| BC002745 | 0.0006025 | ENO2\|enolase 2, (gamma, neuronal) |
| BE349538 | 0.000829 | TM7SF1\|transmembrane 7 superfamily member 1 (upregulated in kidney) |
| NM_014583 | 0.0009047 | LMCD1\|LIM and cysteine-rich domains 1 |
| AL046192 | 0.0004687 | PVT1\|Pvt1 oncogene homolog, MYC activator (mouse) |
| AK023312 | 0.0008557 | Homo sapiens cDNA FLJ13250 fis, clone OVARC1000724 |
| AF054181 | 0.0007103 | NDUFB1\|NADH dehydrogenase (ubiquinone) 1 beta subcomplex, 1 (7 kD, MNLL) |
| AI268717 | 0.000927 | ESTs, Highly similar to AC004836 1 similar to cadherin and Drosophila Fat protein [H. sapiens] |
| AI733236 | 0.000961 | ESTs, Weakly similar to ALU1_HUMAN ALU SUBFAMILY J SEQUENCE CONTAMINATION WARNING ENTRY |
| | | [H. sapiens] |

TABLE 4
Genes that are overexpressed in ER negative cells, and underexpressed in ER positive cells

| GeneID | P-value | Description |
|---|---|---|
| NM_014211 | p < 1e−07 | GABRP\|gamma-aminobutyric acid (GABA) A receptor, pi |
| BC017913 | p < 1e−07 | ART3\|ADP-ribosyltransferase 3 |
| H29323 | p < 1e−07 | SFRP1\|secreted frizzled-related protein 1 |
| AW192480 | p < 1e−07 | SLC6A14\|solute carrier family 6 (neurotransmitter transporter), member 14 |
| AI332979 | p < 1e−07 | KCNG1\|potassium voltage-gated channel, subfamily G, member 1 |
| AJ404611 | p < 1e−07 | BCL11A\|B-cell CLL/lymphoma 11A (zinc finger protein) |
| AW139831 | p < 1e−07 | Homo sapiens cDNA FLJ11796 fis, clone HEMBA1006158, highly similar to Homo sapiens transcription factor |
| | | forkhead-like 7 (FKHL7) gene |
| BC008484 | p < 1e−07 | DKFZP564D206\|DKFZP564D206 protein |
| BC014152 | p < 1e−07 | KRT6A\|keratin 6A |
| BC000045 | p < 1e−07 | TONDU\|TONDU |
| AB028641 | p < 1e−07 | SOX11\|SRY (sex determining region Y)-box 11 |
| AA452915 | p < 1e−07 | EN1\|engrailed homolog 1 |
| AI367357 | p < 1e−07 | ESTs |
| U95089 | p < 1e−07 | EGFR\|epidermal growth factor receptor (erythroblastic leukemia viral (v-erb-b) oncogene homolog, avian) |
| NM_001453 | p < 1e−07 | FOXC1\|forkhead box C1 |
| AW136298 | p < 1e−07 | E48\|lymphocyte antigen 6 complex, locus D |
| AI358916 | p < 1e−07 | ZIC1\|Zic family member 1 (odd-paired homolog, Drosophila) |
| BC015525 | p < 1e−07 | KLK6\|kallikrein 6 (neurosin, zyme) |
| BC012089 | p < 1e−07 | PROML1\|prominin-like 1 (mouse) |
| AW004032 | p < 1e−07 | LOC56963\|hypothetical protein from EUROIMAGE 363668 |
| AF084830 | p < 1e−07 | KCNK5\|potassium channel, subfamily K, member 5 (TASK-2) |
| AW450675 | p < 1e−07 | ESTs |
| AA452928 | p < 1e−07 | GPR51\|G protein-coupled receptor 51 |
| AF308297 | p < 1e−07 | PPP1R14C\|protein phosphatase 1, regulatory (inhibitor) subunit 14C |
| NM_003508 | p < 1e−07 | FZD9\|frizzled homolog 9 (Drosophila) |
| AW291189 | p < 1e−07 | ESTs |
| BC012299 | p < 1e−07 | FABP7\|fatty acid binding protein 7, brain |
| BC005910 | p < 1e−07 | MIA\|melanoma inhibitory activity |
| AA609183 | p < 1e−07 | ESTs |
| Z73678 | p < 1e−07 | PKP1\|plakophilin 1 (ectodermal dysplasia/skin fragility syndrome) |
| BC007008 | p < 1e−07 | CRYAB\|crystallin, alpha B |
| NM_032047 | p < 1e−07 | B3GNT5\|UDP-GlcNAc: betaGal beta-1,3-N-acetylglucosaminyltransferase 5 |
| AY029179 | p < 1e−07 | JPO1\|c-Myc target JPO1 |
| AF055982 | p < 1e−07 | KLK8\|kallikrein 8 (neuropsin/ovasin) |
| AI160174 | p < 1e−07 | ESTs |
| AF257472 | p < 1e−07 | C21orf68\|chromosome 21 open reading frame 68 |
| R50991 | p < 1e−07 | FLJ11413\|hypothetical protein FLJ11413 |
| AK025066 | p < 1e−07 | FLJ10901\|hypothetical protein FLJ10901 |
| BC003628 | p < 1e−07 | HBP17\|heparin-binding growth factor binding protein |
| AI279525 | p < 1e−07 | ELF5\|E74-like factor 5 (ets domain transcription factor) |
| NM_024423 | p < 1e−07 | DSC3\|desmocollin 3 |
| BC007394 | p < 1e−07 | MGC16291\|hypothetical protein MGC16291 |
| AK026818 | p < 1e−07 | Homo sapiens cDNA: FLJ23165 fis, clone LNG09846 |
| AA521195 | p < 1e−07 | LAMP3\|lysosomal-associated membrane protein 3 |
| AA156097 | p < 1e−07 | ESTs, Weakly similar to LKHU proteoglycan link protein precursor H. sapiens |
| AW137881 | p < 1e−07 | RARRES1\|retinoic acid receptor responder (tazarotene induced) 1 |
| BC014941 | p < 1e−07 | ID4\|inhibitor of DNA binding 4, dominant negative helix-loop-helix protein |
| AA622986 | p < 1e−07 | ESTs |
| AA922144 | p < 1e−07 | AQP5\|aquaporin 5 |
| AI800206 | p < 1e−07 | STAC\|src homology three (SH3) and cysteine rich domain |
| NM_002964 | p < 1e−07 | S100A8\|S100 calcium binding protein A8 (calgranulin A) |
| BC007595 | p < 1e−07 | SOX10\|SRY (sex determining region Y)-box 10 |
| AK000442 | p < 1e−07 | FLJ20435\|hypothetical protein FLJ20435 |
| BC017352 | p < 1e−07 | TRIM29\|tripartite motif-containing 29 |
| AJ000388 | p < 1e−07 | CAPN6\|calpain 6 |
| AL137311 | p < 1e−07 | Homo sapiens mRNA; cDNA DKFZp761G02121 (from clone DKFZp761G02121); partial cds |
| BC012914 | p < 1e−07 | Homo sapiens cDNA FLJ31360 fis, clone MESAN2000572 |
| AA194943 | p < 1e−07 | DEFB1\|defensin, beta 1 |
| AL110178 | p < 1e−07 | TRIM2\|tripartite motif-containing 2 |
| AW204371 | p < 1e−07 | DSC2\|desmocollin 2 |
| AI049783 | p < 1e−07 | ESTs |
| BC000183 | p < 1e−07 | TMSNB\|thymosin, beta, identified in neuroblastoma cells |
| BI495988 | p < 1e−07 | MET\|met proto-oncogene (hepatocyte growth factor receptor) |
| AL110126 | p < 1e−07 | Homo sapiens mRNA; cDNA DKFZp564H1916 (from clone DKFZp564H1916) |
| AW140023 | p < 1e−07 | FLJ13204\|hypothetical protein FLJ13204 |
| BC003635 | p < 1e−07 | MMP7\|matrix metalloproteinase 7 (matrilysin, uterine) |
| BC016004 | p < 1e−07 | MARCO\|macrophage receptor with collagenous structure |
| AW264415 | p < 1e−07 | Homo sapiens, clone MGC: 24011 IMAGE: 4091916, mRNA, complete cds |
| AI375448 | p < 1e−07 | GPM6B\|glycoprotein M6B |
| N64339 | p < 1e−07 | GJB6\|gap junction protein, beta 6 (connexin 30) |
| AK026420 | p < 1e−07 | DMN\|desmuslin |
| BF433019 | p < 1e−07 | ESTs, Weakly similar to TRHY_HUMAN TRICHOHYALI H. sapiens |
| AI885044 | p < 1e−07 | KIT\|v-kit Hardy-Zuckerman 4 feline sarcoma viral oncogene homolog |
| AK021785 | p < 1e−07 | Homo sapiens cDNA FLJ11723 fis, clone HEMBA1005314 |
| AF089854 | p < 1e−07 | TU3A\|TU3A protein |
| AF033021 | p < 1e−07 | KCNN4\|potassium intermediate/small conductance calcium-activated channel, subfamily N, member 4 |
| AI948466 | p < 1e−07 | INDO\|indoleamine-pyrrole 2,3 dioxygenase |
| AW291290 | p < 1e−07 | ESTs |
| AF052117 | p < 1e−07 | Homo sapiens clone 23809 mRNA sequence |
| NM_002965 | p < 1e−07 | S100A9\|S100 calcium binding protein A9 (calgranulin B) |
| NM_006522 | p < 1e−07 | WNT6\|wingless-type MMTV integration site family, member 6 |
| U17077 | p < 1e−07 | BENE\|BENE protein |
| AW449979 | p < 1e−07 | SLC15A1\|solute carrier family 15 (oligopeptide transporter), member 1 |
| NM_001793 | p < 1e−07 | CDH3\|cadherin 3, type 1, P-cadherin (placental) |
| AY035400 | p < 1e−07 | PCOLCE2\|procollagen C-endopeptidase enhancer 2 |
| AF047348 | p < 1e−07 | APBA2\|amyloid beta (A4) precursor protein-binding, family A, member 2 (X11-like) |
| AI732309 | p < 1e−07 | EPHB1\|EphB1 |
| AA584033 | p < 1e−07 | KRT6B\|keratin 6B |
| NM_002639 | p < 1e−07 | SERPINB5\|serine (or cysteine) proteinase inhibitor, clade B (ovalbumin), member 5 |
| BC000332 | p < 1e−07 | UCHL1\|ubiquitin carboxyl-terminal esterase L1 (ubiquitin thiolesterase) |
| BF224381 | p < 1e−07 | ESTs |
| BC017583 | p < 1e−07 | SCRG1\|scrapie responsive protein 1 |
| NM_004961 | p < 1e−07 | GABRE\|gamma-aminobutyric acid (GABA) A receptor, epsilon |
| AI351524 | p < 1e−07 | DMD\|dystrophin (muscular dystrophy, Duchenne and Becker types), includes DXS142, DXS164, DXS206, |
| | | DXS230, DXS239, DXS268, DXS269, DXS270, DXS272 |
| BC001283 | p < 1e−07 | NFIB\|nuclear factor I/B |
| BC004376 | p < 1e−07 | ANXA8\|annexin A8 |
| AB037813 | p < 1e−07 | DKFZp762K222\|hypothetical protein DKFZp762K222 |
| AK024990 | p < 1e−07 | PPARGC1\|peroxisome proliferative activated receptor, gamma, coactivator 1 |
| AF241254 | p < 1e−07 | ACE2\|angiotensin I converting enzyme (peptidyl-dipeptidase A) 2 |
| AI800677 | p < 1e−07 | SLPI\|secretory leukocyte protease inhibitor (antileukoproteinase) |
| BC012908 | p < 1e−07 | NMU\|neuromedin U |
| BC012097 | p < 1e−07 | OCA2\|oculocutaneous albinism II (pink-eye dilution homolog, mouse) |
| R44714 | p < 1e−07 | Homo sapiens cDNA FLJ13136 fis, clone NT2RP3003139 |
| R15881 | p < 1e−07 | CHRM3\|cholinergic receptor, muscarinic 3 |
| AK025909 | p < 1e−07 | Homo sapiens cDNA: FLJ22256 fis, clone HRC02860 |
| AI754805 | p < 1e−07 | SMOC1\|secreted modular calcium-binding protein 1 |
| BC009581 | p < 1e−07 | HSD17B2\|hydroxysteroid (17-beta) dehydrogenase 2 |
| X59770 | p < 1e−07 | IL1R2\|interleukin 1 receptor, type II |
| M76482 | p < 1e−07 | DSG3\|desmoglein 3 (pemphigus vulgaris antigen) |
| AI368024 | p < 1e−07 | ESTs |
| BC015753 | p < 1e−07 | GRO2\|GRO2 oncogene |
| BC014950 | p < 1e−07 | CA9\|carbonic anhydrase IX |
| BC008366 | p < 1e−07 | DDC\|dopa decarboxylase (aromatic L-amino acid decarboxylase) |
| AI970748 | p < 1e−07 | ESTs |
| BC001766 | p < 1e−07 | S100B\|S100 calcium binding protein, beta (neural) |
| AW206460 | p < 1e−07 | KIAA0481\|KIAA0481 gene product |
| AF361486 | p < 1e−07 | MUC16\|mucin 16 |
| BC000992 | p < 1e−07 | CSRP2\|cysteine and glycine-rich protein 2 |
| AI202593 | p < 1e−07 | CYP7B1\|cytochrome P450, subfamily VIIB (oxysterol 7 alpha-hydroxylase), polypeptide 1 |
| NM_006115 | p < 1e−07 | PRAME\|preferentially expressed antigen in melanoma |
| BC011976 | p < 1e−07 | GRO1\|GRO1 oncogene (melanoma growth stimulating activity, alpha) |
| BC006226 | p < 1e−07 | DLX5\|distal-less homeo box 5 |
| AL137289 | p < 1e−07 | ARHGEF4\|Rho guanine nucleotide exchange factor (GEF) 4 |
| BC005083 | p < 1e−07 | MGC13057\|hypothetical protein MGC13057 |
| AI261967 | p < 1e−07 | ADORA2B\|adenosine A2b receptor |
| AI273928 | p < 1e−07 | ALDH1A3\|aldehyde dehydrogenase 1 family, member A3 |
| BC005948 | p < 1e−07 | SMPX\|small muscle protein, X-linked |
| NM_006769 | p < 1e−07 | LMO4\|LIM domain only 4 |
| AL117452 | p < 1e−07 | DKFZP586G1517\|DKFZP586G1517 protein |
| AA587049 | p < 1e−07 | PLA2G4A\|phospholipase A2, group IVA (cytosolic, calcium-dependent) |
| AI804716 | p < 1e−07 | ESTs |
| AI637899 | p < 1e−07 | GAL\|galanin |
| AF269101 | p < 1e−07 | MID1\|midline 1 (Opitz/BBB syndrome) |
| NM_003034 | p < 1e−07 | SIAT8A\|sialyltransferase 8A (alpha-N-acetylneuraminate: alpha-2,8-sialytransferase, GD3 synthase) |
| AF141339 | p < 1e−07 | DKFZP564D0764\|DKFZP564D0764 protein |
| AI095049 | p < 1e−07 | Homo sapiens cDNA FLJ31627 fis, clone NT2RI2003338 |
| AI498405 | p < 1e−07 | ESTs, Weakly similar to B49647 GTP-binding protein rab8 H. sapiens |
| AF130728 | p < 1e−07 | DMRT1\|doublesex and mab-3 related transcription factor 1 |
| AW206700 | p < 1e−07 | KLK7\|kallikrein 7 (chymotryptic, stratum corneum) |
| AB042405 | p < 1e−07 | MLZE\|melanoma-derived leucine zipper, extra-nuclear factor |
| AF104032 | p < 1e−07 | SLC7A5\|solute carrier family 7 (cationic amino acid transporter, y+ system), member 5 |
| AA521486 | p < 1e−07 | CECR7\|cat eye syndrome chromosome region, candidate 7 |
| AI024603 | p < 1e−07 | HAPIP\|huntingtin-associated protein interacting protein (duo) |
| AI830532 | p < 1e−07 | ESTs |
| M57892 | p < 1e−07 | CA6\|carbonic anhydrase VI |
| NM_003914 | p < 1e−07 | CCNA1\|cyclin A1 |
| S72493 | p < 1e−07 | KRT16\|keratin 16 (focal non-epidermolytic palmoplantar keratoderma) |
| NM_004938 | p < 1e−07 | DAPK1\|death-associated protein kinase 1 |
| AW071804 | p < 1e−07 | ESTs |
| NM_002996 | p < 1e−07 | SCYD1\|small inducible cytokine subfamily D (Cys-X3-Cys), member 1 (fractalkine, neurotactin) |
| NM_002299 | p < 1e−07 | LCT\|lactase |
| R55787 | p < 1e−07 | KIAA1069\|KIAA1069 protein |
| AF271781 | p < 1e−07 | ITM3\|integral membrane protein 3 |
| AA716090 | p < 1e−07 | BOC\|brother of CDO |
| AJ420468 | p < 1e−07 | KIAA1209\|KIAA1209 protein |
| H09748 | p < 1e−07 | BCL11B\|B-cell CLL/lymphoma 11B (zinc finger protein) |
| AA806280 | p < 1e−07 | Homo sapiens cDNA FLJ31951 fis, clone NT2RP7007177, weakly similar to Homo sapiens multiple membrane |
| | | spanning receptor TRC8 mRNA |
| BI492630 | p < 1e−07 | SLC9A6\|solute carrier family 9 (sodium/hydrogen exchanger), isoform 6 |
| BC004397 | p < 1e−07 | MGC10981\|hypothetical protein MGC10981 |
| AF095719 | p < 1e−07 | CPA4\|carboxypeptidase A4 |
| AF331525 | p < 1e−07 | SLC26A9\|solute carrier family 26, member 9 |
| R38090 | p < 1e−07 | G2\|G2 protein |
| AF117108 | p < 1e−07 | KOC1\|IGF-II mRNA-binding protein 3 |
| AF127563 | p < 1e−07 | DKK1\|dickkopf homolog 1 (Xenopus laevis) |
| AA633482 | p < 1e−07 | EST |
| BC008588 | p < 1e−07 | PLS3\|plastin 3 (T isoform) |
| NM_002852 | p < 1e−07 | PTX3\|pentaxin-related gene, rapidly induced by IL-1 beta |
| AF123074 | p < 1e−07 | DNCI1\|dynein, cytoplasmic, intermediate polypeptide 1 |
| AK000123 | p < 1e−07 | C20orf42\|chromosome 20 open reading frame 42 |
| AW444437 | 0.0000001 | ESTs |
| AI557322 | p < 1e−07 | ESTs |
| AB053450 | p < 1e−07 | KIAA1776\|fibrillin3 |
| X53578 | p < 1e−07 | FUT3\|fucosyltransferase 3 (galactoside 3(4)-L-fucosyltransferase, Lewis blood group included) |
| AF043473 | p < 1e−07 | KCNS1\|potassium voltage-gated channel, delayed-rectifier, subfamily S, member 1 |
| AF197137 | p < 1e−07 | FN5\|FN5 protein |
| BC017733 | p < 1e−07 | MRAS\|muscle RAS oncogene homolog |
| AW265625 | p < 1e−07 | ESTs |
| AA527072 | p < 1e−07 | EST |
| BC016666 | p < 1e−07 | Homo sapiens, clone IMAGE: 3917693, mRNA |
| AF220183 | p < 1e−07 | HT009\|uncharacterized hypothalamus protein HT009 |
| AI356375 | p < 1e−07 | CDKN2A\|cyclin-dependent kinase inhibitor 2A (melanoma, p16, inhibits CDK4) |
| AW780208 | p < 1e−07 | Homo sapiens, clone MGC: 15887 IMAGE: 3530481, mRNA, complete cds |
| BC006097 | p < 1e−07 | HLA-DOB\|major histocompatibility complex, class II, DO beta |
| AI740531 | p < 1e−07 | MAPK4\|mitogen-activated protein kinase 4 |
| L20433 | p < 1e−07 | POU4F1\|POU domain, class 4, transcription factor 1 |
| NM_003806 | p < 1e−07 | HRK\|harakiri, BCL2 interacting protein (contains only BH3 domain) |
| BC007541 | p < 1e−07 | MGC15437\|hypothetical protein MGC15437 |
| AF091092 | p < 1e−07 | CL683\|weakly similar to glutathione peroxidase 2 |
| AI609709 | p < 1e−07 | CXorf6\|chromosome X open reading frame 6 |
| BG236645 | 0.0000003 | ESTs |
| AF082868 | p < 1e−07 | BBOX1\|butyrobetaine (gamma), 2-oxoglutarate dioxygenase (gamma-butyrobetaine hydroxylase) 1 |
| BE326342 | p < 1e−07 | ESTs, Highly similar to bK989H11.1 H. sapiens |
| AL080079 | p < 1e−07 | DKFZP564D0462\|hypothetical protein DKFZp564D0462 |
| AF181862 | p < 1e−07 | GPRC5B\|G protein-coupled receptor, family C, group 1, member B |
| BC012844 | p < 1e−07 | PRNP\|prion protein (p27-30) (Creutzfeld-Jakob disease, Gerstmann-Strausler-Scheinker syndrome, fatal |
| | | familial insomnia) |
| AA977269 | p < 1e−07 | FOXD1\|forkhead box D1 |
| AA999973 | p < 1e−07 | FLJ14054\|hypothetical protein FLJ14054 |
| AW195038 | p < 1e−07 | KIP2\|DNA-dependent protein kinase catalytic subunit-interacting protein 2 |
| AA433906 | p < 1e−07 | KSP37\|Ksp37 protein |
| AF070614 | p < 1e−07 | SCHIP1\|schwannomin interacting protein 1 |
| BF511352 | p < 1e−07 | LYN\|v-yes-1 Yamaguchi sarcoma viral related oncogene homolog |
| BC011901 | p < 1e−07 | KRT17\|keratin 17 |
| N31940 | p < 1e−07 | ESTs, Weakly similar to 2004399A chromosomal protein H. sapiens |
| BC002829 | p < 1e−07 | S100A2\|S100 calcium binding protein A2 |
| AL137332 | p < 1e−07 | Homo sapiens mRNA; cDNA DKFZp434K2172 (from clone DKFZp434K2172) |
| AB022918 | p < 1e−07 | ST3GALVI\|alpha2,3-sialyltransferase |
| BC008709 | p < 1e−07 | ADD2\|adducin 2 (beta) |
| AK026733 | p < 1e−07 | Homo sapiens cDNA: FLJ23080 fis, clone LNG06052 |
| H10667 | p < 1e−07 | ROR1\|receptor tyrosine kinase-like orphan receptor 1 |
| X70340 | p < 1e−07 | TGFA\|transforming growth factor, alpha |
| Y15723 | p < 1e−07 | GUCY1A3\|guanylate cyclase 1, soluble, alpha 3 |
| NM_005044 | p < 1e−07 | PRKX\|protein kinase, X-linked |
| AB018329 | p < 1e−07 | KIAA0786\|latrophilin |
| BF444945 | p < 1e−07 | HRASLS\|HRAS-like suppressor |
| BF589790 | p < 1e−07 | ADM\|adrenomedullin |
| AF132818 | p < 1e−07 | KLF5\|Kruppel-like factor S (intestinal) |
| D11613 | p < 1e−07 | VSNL1\|visinin-like 1 |
| BC001275 | p < 1e−07 | ANXA1\|annexin A1 |
| M99487 | p < 1e−07 | FOLH1\|folate hydrolase (prostate-specific membrane antigen) 1 |
| AB052906 | p < 1e−07 | ULBP2\|UL16 binding protein 2 |
| BC012107 | p < 1e−07 | SH2D2A\|SH2 domain protein 2A |
| AA586749 | 0.0000001 | KRT5\|keratin 5 (epidermolysis bullosa simplex, Dowling-Meara/Kobner/Weber-Cockayne types) |
| BC017918 | p < 1e−07 | LOC64148\|17 kD fetal brain protein |
| NM_005378 | p < 1e−07 | MYCN\|v-myc myelocytomatosis viral related oncogene, neuroblastoma derived (avian) |
| AA767129 | p < 1e−07 | PRKY\|protein kinase, Y-linked |
| AW139720 | p < 1e−07 | FLJ12649\|hypothetical protein FLJ12649 |
| BC012432 | p < 1e−07 | FAT\|FAT tumor suppressor homolog 1 (Drosophila) |
| BC010003 | p < 1e−07 | Homo sapiens, clone IMAGE: 4134852, mRNA, partial cds |
| AF155651 | p < 1e−07 | PAK3\|p21 (CDKN1A)-activated kinase 3 |
| BC018646 | p < 1e−07 | PLCG2\|phospholipase C, gamma 2 (phosphatidylinositol-specific) |
| BC011705 | p < 1e−07 | COL9A3\|collagen, type IX, alpha 3 |
| AF242518 | p < 1e−07 | LOC57115\|hypothetical protein SBBI67 |
| AL136755 | p < 1e−07 | DKFZP434A1315\|hypothetical protein DKFZp434A1315 |
| NM_001238 | p < 1e−07 | CCNE1\|cyclin E1 |
| AB023430 | p < 1e−07 | BIT\|brain-immunoglobulin-like molecule with tyrosine-based activation motifs |
| AL050374 | p < 1e−07 | Homo sapiens mRNA; cDNA DKFZp586C1619 (from clone DKFZp586C1619) |
| AK056040 | p < 1e−07 | RE2\|G-protein coupled receptor |
| BC007293 | p < 1e−07 | MGC15668\|hypothetical protein MGC15668 |
| AY004253 | p < 1e−07 | PRDM13\|PR domain containing 13 |
| BC012381 | p < 1e−07 | FLJ10430\|hypothetical protein FLJ10430 |
| AW118456 | p < 1e−07 | CRABP1\|cellular retinoic acid binding protein 1 |
| AF131789 | p < 1e−07 | DKFZP566A1524\|hypothetical protein DKFZp566A1524 |
| BC007382 | p < 1e−07 | MGC16212\|hypothetical protein MGC16212 |
| NM_014274 | p < 1e−07 | ABP/ZF\|Alu-binding protein with zinc finger domain |
| AF239820 | p < 1e−07 | C4ST\|chondroitin 4-sulfotransferase |
| AL389942 | p < 1e−07 | Homo sapiens mRNA full length insert cDNA clone EUROIMAGE 2005635 |
| AW574736 | p < 1e−07 | PRKCN\|protein kinase C, nu |
| AL136566 | p < 1e−07 | FLJ12783\|hypothetical protein FLJ12783 |
| BC012142 | p < 1e−07 | P5\|protein disulfide isomerase-related protein |
| AI560801 | p < 1e−07 | PRSS12\|protease, serine, 12 (neurotrypsin, motopsin) |
| BC000765 | p < 1e−07 | MGC2742\|hypothetical protein MGC2742 |
| AK021728 | p < 1e−07 | Homo sapiens cDNA FLJ11666 fis, clone HEMBA1004672 |
| AF051321 | p < 1e−07 | T-STAR\|Sam68-like phosphotyrosine protein, T-STAR |
| BE965331 | p < 1e−07 | CTSL2\|cathepsin L2 |
| R76294 | p < 1e−07 | ESTs |
| AL080235 | p < 1e−07 | RIS1\|Ras-induced senescence 1 |
| NM_002988 | 0.0000575 | SCYA18\|small inducible cytokine subfamily A (Cys—Cys), member 18, pulmonary and activation-regulated |
| BC009701 | p < 1e−07 | PDI2\|peptidyl arginine deiminase, type II |
| AF133059 | p < 1e−07 | NFE2L3\|nuclear factor (erythroid-derived 2)-like 3 |
| AF356193 | p < 1e−07 | CARD6\|caspase recruitment domain family, member 6 |
| BC009744 | p < 1e−07 | CSDA\|cold shock domain protein A |
| BC015663 | p < 1e−07 | TNRC3\|trinucleotide repeat containing 3 |
| BC010039 | p < 1e−07 | CLP\|coactosin-like protein |
| N47717 | p < 1e−07 | FABP5\|fatty acid binding protein 5 (psoriasis-associated) |
| AF280086 | p < 1e−07 | CHST6\|carbohydrate (N-acetylglucosamine 6-O) sulfotransferase 6 |
| AL117633 | p < 1e−07 | Homo sapiens mRNA; cDNA DKFZp434E235 (from clone DKFZp434E235) |
| BC009010 | p < 1e−07 | Homo sapiens, clone MGC: 18257 IMAGE: 4158300, mRNA, complete cds |
| BC008568 | p < 1e−07 | CHI3L1\|chitinase 3-like 1 (cartilage glycoprotein-39) |
| BC005963 | 0.0000357 | MAGEA3\|melanoma antigen, family A, 3 |
| BC010920 | p < 1e−07 | CLDN10\|claudin 10 |
| BC011460 | p < 1e−07 | CHI3L2\|chitinase 3-like 2 |
| AF131235 | p < 1e−07 | CHST4\|carbohydrate (N-acetylglucosamine 6-O) sulfotransferase 4 |
| BC004411 | p < 1e−07 | HCA127\|hepatocellular carcinoma-associated antigen 127 |
| AK025347 | p < 1e−07 | KIAA1357\|KIAA1357 protein |
| BC000563 | p < 1e−07 | SLC6A2\|solute carrier family 6 (neurotransmitter transporter, noradrenalin), member 2 |
| AF057352 | p < 1e−07 | IMP-2\|IGF-II mRNA-binding protein 2 |
| X77197 | p < 1e−07 | CLCN4\|chloride channel 4 |
| AF073515 | p < 1e−07 | CRLF1\|cytokine receptor-like factor 1 |
| AW172994 | 0.0000008 | MMP12\|matrix metalloproteinase 12 (macrophage elastase) |
| AI678454 | p < 1e−07 | MYO10\|myosin X |
| AK025251 | p < 1e−07 | CHST3\|carbohydrate (chondroitin 6) sulfotransferase 3 |
| BC013300 | p < 1e−07 | STK12\|serine/threonine kinase 12 |
| BC010379 | 0.0000002 | CSTA\|cystatin A (stefin A) |
| AI460151 | 0.0000025 | ACTG2\|actin, gamma 2, smooth muscle, enteric |
| BC013153 | p < 1e−07 | LOXL4\|lysyl oxidase-like 4 |
| BC007049 | p < 1e−07 | RGS2\|regulator of G-protein signalling 2, 24 kD |
| AK056666 | p < 1e−07 | Homo sapiens cDNA FLJ32104 fis, clone OCBBF2001307 |
| BC000871 | p < 1e−07 | ANXA3\|annexin A3 |
| AI433792 | p < 1e−07 | ESTs |
| BC008952 | p < 1e−07 | LDHB\|lactate dehydrogenase B |
| AF083066 | p < 1e−07 | CHST2\|carbohydrate (N-acetylglucosamine-6-O) sulfotransferase 2 |
| NM_003878 | p < 1e−07 | GGH\|gamma-glutamyl hydrolase (conjugase, folylpolygammaglutamyl hydrolase) |
| AA631303 | p < 1e−07 | Homo sapiens prostate-specific membrane antigen PSM mRNA, exon 18 alternative splice variant, partial cds |
| AF336127 | p < 1e−07 | SLC4A11\|solute carrier family 4, sodium bicarbonate transporter-like, member 11 |
| AK026946 | p < 1e−07 | FLJ23293\|likely ortholog of mouse ADP-ribosylation-like factor 6 interacting protein 2 |
| BC015359 | p < 1e−07 | Homo sapiens, Similar to RIKEN cDNA 1810037C20 gene, clone MGC: 21481 IMAGE: 3852062, mRNA, |
| | | complete cds |
| AJ420564 | p < 1e−07 | Homo sapiens cDNA: FLJ22314 fis, clone HRC05250 |
| AF052126 | p < 1e−07 | SRD5A1\|steroid-5-alpha-reductase, alpha polypeptide 1 (3-oxo-5 alpha-steroid delta 4-dehydrogenase alpha 1) |
| AK000708 | p < 1e−07 | FLJ20701\|hypothetical protein FLJ20701 |
| NM_003504 | p < 1e−07 | CDC45L\|CDC45 cell division cycle 45-like (S. cerevisiae) |
| BC000881 | p < 1e−07 | CENPA\|centromere protein A (17 kD) |
| AI142639 | p < 1e−07 | ESTs |
| AK026790 | p < 1e−07 | Homo sapiens cDNA: FLJ23137 fis, clone LNG08842 |
| AA761512 | p < 1e−07 | LPIN1\|lipin 1 |
| BC017293 | p < 1e−07 | MSN\|moesin |
| AL049977 | 0.000019 | CLDN8\|claudin 8 |
| AL122077 | p < 1e−07 | DNAH17\|dynein, axonemal, heavy polypeptide 17 |
| BC003554 | p < 1e−07 | UBE2E3\|ubiquitin-conjugating enzyme E2E 3 (UBC4/5 homolog, yeast) |
| AF245436 | p < 1e−07 | FLJ23518\|hypothetical protein FLJ23518 |
| AF333769 | p < 1e−07 | CASPR3\|cell recognition molecule CASPR3 |
| AB051513 | p < 1e−07 | KIAA1726\|KIAA1726 protein |
| AA766903 | p < 1e−07 | ESTs, Highly similar to A46297 beta-1,6-N-acetylglucosaminyltransferase H. sapiens |
| AI823561 | p < 1e−07 | LPL\|lipoprotein lipase |
| NM_002284 | p < 1e−07 | KRTHB6\|keratin, hair, basic, 6 (monilethrix) |
| AA424146 | p < 1e−07 | LOC51312\|mitochondrial solute carrier |
| AI200443 | 0.0000155 | MAGEA5\|melanoma antigen, family A, 5 |
| H18932 | p < 1e−07 | XK\|Kell blood group precursor (McLeod phenotype) |
| BF062000 | p < 1e−07 | ESTs, Highly similar to unnamed protein product H. sapiens |
| AF035594 | p < 1e−07 | Homo sapiens protein kinase C-alpha mRNA, partial 3′ UTR |
| BC002895 | p < 1e−07 | GSTA2\|glutathione S-transferase A2 |
| AA648922 | p < 1e−07 | CDC25A\|cell division cycle 25A |
| BC004199 | p < 1e−07 | RLBP1\|retinaldehyde binding protein 1 |
| BC002947 | p < 1e−07 | FOLR1\|folate receptor 1 (adult) |
| AA716439 | p < 1e−07 | BCE-1\|BCE-1 protein |
| AI275604 | p < 1e−07 | PRRG1\|proline-rich Gla (G-carboxyglutamic acid) polypeptide 1 |
| BC017381 | p < 1e−07 | FLJ12929\|hypothetical protein FLJ12929 |
| AI188827 | p < 1e−07 | PIM1\|pim-1 oncogene |
| AL080059 | 0.0000025 | KIAA1750\|KIAA1750 protein |
| AY037298 | p < 1e−07 | ELOVL4\|elongation of very long chain fatty acids (FEN1/Elo2, SUR4/Elo3, yeast)-like 4 |
| AF245517 | 0.0000145 | ATP6N1B\|ATPase, H+ transporting, lysosomal (vacuolar proton pump) non-catalytic accessory protein 1B |
| BC017730 | p < 1e−07 | TNFRSF21\|tumor necrosis factor receptor superfamily, member 21 |
| BC018652 | p < 1e−07 | FXYD6\|FXYD domain-containing ion transport regulator 6 |
| AI741143 | p < 1e−07 | Homo sapiens cDNA FLJ32401 fis, clone SKMUS2000339 |
| X64318 | p < 1e−07 | NFIL3\|nuclear factor, interleukin 3 regulated |
| AB047783 | p < 1e−07 | ANKRD3\|ankyrin repeat domain 3 |
| AF038185 | p < 1e−07 | Homo sapiens clone 23700 mRNA sequence |
| BC016838 | p < 1e−07 | CPVL\|carboxypeptidase, vitellogenic-like |
| AF193809 | p < 1e−07 | RHCG\|Rh type C glycoprotein |
| AJ420473 | p < 1e−07 | Homo sapiens mRNA full length insert cDNA clone EUROIMAGE 1502032 |
| AF285109 | p < 1e−07 | 3-Sep\|septin 3 |
| NM_001604 | p < 1e−07 | PAX6\|paired box gene 6 (aniridia, keratitis) |
| BC009945 | p < 1e−07 | KIAA0746\|KIAA0746 protein |
| AF026547 | p < 1e−07 | CSPG3\|chondroitin sulfate proteoglycan 3 (neurocan) |
| AF101051 | p < 1e−07 | CLDN1\|claudin 1 |
| BC002710 | p < 1e−07 | KLK10\|kallikrein 10 |
| AA873846 | p < 1e−07 | LOC91689\|hypothetical gene supported by AL449243 |
| AI452634 | p < 1e−07 | GPR64\|G protein-coupled receptor 64 |
| AK027126 | p < 1e−07 | ASS\|argininosuccinate synthetase |
| AJ420542 | p < 1e−07 | Homo sapiens mRNA full length insert cDNA clone EUROIMAGE 1090104 |
| AK024818 | 0.000002 | MMP1\|matrix metalloproteinase 1 (interstitial collagenase) |
| AW265278 | p < 1e−07 | IVL\|involucrin |
| AW445220 | 0.0000115 | HSJ001348\|cDNA for differentially expressed CO16 gene |
| AF108138 | p < 1e−07 | PIFI\|DNA helicase homolog PIF1 |
| BC002660 | p < 1e−07 | TMOD\|tropomodulin |
| AA909006 | p < 1e−07 | LBP-32\|LBP protein 32 |
| AI097663 | p < 1e−07 | P450RAI-2\|cytochrome P450 retinoid metabolizing protein |
| BC009742 | p < 1e−07 | LAD1\|ladinin 1 |
| AB028140 | p < 1e−07 | TMPRSS5\|transmembrane protease, serine 5 (spinesin) |
| AI569989 | p < 1e−07 | KIAA1755\|KIAA1755 protein |
| BF508352 | p < 1e−07 | DSG1\|desmoglein 1 |
| R49390 | p < 1e−07 | KIAA1678\|KIAA1678 |
| AI174254 | p < 1e−07 | Homo sapiens cDNA FLJ31586 fis, clone NT2RI2002211 |
| BE046069 | p < 1e−07 | ESTs |
| AL109775 | p < 1e−07 | SH3GL3\|SH3-domain GRB2-like 3 |
| AI522215 | p < 1e−07 | KIAA1804\|KIAA1804 protein |
| BC008682 | p < 1e−07 | ARP3BETA\|actin-related protein 3-beta |
| L26584 | p < 1e−07 | RASGRF1\|Ras protein-specific guanine nucleotide-releasing factor 1 |
| AL136856 | p < 1e−07 | NTT5\|NTT5 protein |
| AF038173 | p < 1e−07 | ATSV\|axonal transport of synaptic vesicles |
| NM_003360 | p < 1e−07 | UGT8\|UDP glycosyltransferase 8 (UDP-galactose ceramide galactosyltransferase) |
| BC015915 | p < 1e−07 | FZD7\|frizzled homolog 7 (Drosophila) |
| BG939593 | p < 1e−07 | ANGPT1\|angiopoietin 1 |
| BF446789 | p < 1e−07 | CAV2\|caveolin 2 |
| AI538937 | p < 1e−07 | GDF5\|growth differentiation factor 5 (cartilage-derived morphogenetic protein-1) |
| BC011672 | p < 1e−07 | KIAA0514\|KIAA0514 gene product |
| AI494546 | p < 1e−07 | ICB-1\|basement membrane-induced gene |
| BC008764 | p < 1e−07 | KNSL6\|kinesin-like 6 (mitotic centromere-associated kinesin) |
| BC010358 | p < 1e−07 | CYP39A1\|oxysterol 7alpha-hydroxylase |
| M14584 | p < 1e−07 | IL6\|interleukin 6 (interferon, beta 2) |
| R41459 | p < 1e−07 | KIAA1136\|KIAA1136 protein |
| AL157459 | p < 1e−07 | Homo sapiens mRNA; cDNA DKFZp434B0425 (from clone DKFZp434B0425) |
| BC001068 | p < 1e−07 | C20orf129\|chromosome 20 open reading frame 129 |
| AL133624 | p < 1e−07 | DKFZp434B1222\|AKAP-binding sperm protein ropporin |
| AJ243500 | p < 1e−07 | TRPV6\|transient receptor potential cation channel, subfamily V, member 6 |
| AI950130 | 0.0000006 | GZMB\|granzyme B (granzyme 2, cytotoxic T-lymphocyte-associated serine esterase 1) |
| AK023678 | p < 1e−07 | Homo sapiens cDNA FLJ13616 fis, clone PLACE1010916 |
| BC002325 | 0.0000019 | MGC4309\|hypothetical protein MGC4309 |
| AB049586 | p < 1e−07 | B3GNT4\|UDP-GlcNAc: betaGal beta-1,3-N-acetylglucosaminyltransferase 4 |
| AL136840 | p < 1e−07 | MCM10\|MCM10 minichromosome maintenance deficient 10 (S. cerevisiae) |
| BC014039 | p < 1e−07 | KIAA0175\|likely ortholog of maternal embryonic leucine zipper kinase |
| AL122086 | p < 1e−07 | TIAM2\|T-cell lymphoma invasion and metastasis 2 |
| AK055431 | p < 1e−07 | Homo sapiens cDNA FLJ30869 fis, clone FEBRA2004224 |
| AW472810 | p < 1e−07 | IL12A\|interleukin 12A (natural killer cell stimulatory factor 1, cytotoxic lymphocyte maturation factor 1, p35) |
| BC010353 | 0.0000004 | PTPLA\|protein tyrosine phosphatase-like (proline instead of catalytic arginine), member a |
| BI493178 | p < 1e−07 | KIAA0161\|KIAA0161 gene product |
| AA181334 | p < 1e−07 | TNNI2\|troponin I, skeletal, fast |
| AF067223 | p < 1e−07 | PDE9A\|phosphodiesterase 9A |
| BC010954 | 0.0000008 | SCYB10\|small inducible cytokine subfamily B (Cys-X-Cys), member 10 |
| AI218529 | p < 1e−07 | KIAA1754\|KIAA1754 protein |
| AI380218 | p < 1e−07 | FLJ13385\|hypothetical protein FLJ13385 |
| BC013134 | p < 1e−07 | Homo sapiens, clone IMAGE: 4512785, mRNA |
| AJ245600 | p < 1e−07 | Homo sapiens mRNA for hypothetical protein (TR2/D15 gene) |
| BC007609 | p < 1e−07 | Homo sapiens, Similar to hypothetical protein PRO1722, clone MGC: 15692 IMAGE: 3351479, mRNA, complete |
| | | cds |
| AW589741 | p < 1e−07 | FLJ10159\|hypothetical protein FLJ10159 |
| BC016308 | p < 1e−07 | GRO3\|GRO3 oncogene |
| BC013362 | p < 1e−07 | RUNX3\|runt-related transcription factor 3 |
| AI582813 | p < 1e−07 | CHRNA5\|cholinergic receptor, nicotinic, alpha polypeptide 5 |
| AI436812 | p < 1e−07 | ESTs, Weakly similar to hyperpolarization-activated cyclic nucleotide-gated channel hHCN2 H. sapiens |
| AF039843 | p < 1e−07 | SPRY2\|sprouty homolog 2 (Drosophila) |
| AF097021 | 0.0003463 | GW112\|differentially expressed in hematopoietic lineages |
| AF054839 | p < 1e−07 | TSPAN-2\|tetraspan 2 |
| BC005029 | p < 1e−07 | FLJ10718\|hypothetical protein FLJ10718 |
| AL137580 | p < 1e−07 | C20orf130\|chromosome 20 open reading frame 130 |
| BF447279 | 0.0000004 | ESTs |
| AI767898 | 0.0000015 | GBP5\|guanylate binding protein 5 |
| AI140061 | p < 1e−07 | E2F3\|E2F transcription factor 3 |
| BI963794 | p < 1e−07 | PLA2G7\|phospholipase A2, group VII (platelet-activating factor acetylhydrolase, plasma) |
| AI632467 | p < 1e−07 | OKB1\|organic cation transporter OKB1 |
| AI366784 | p < 1e−07 | TAF4B\|TAF4b RNA polymerase II, TATA box binding protein (TBP)-associated factor, 105 kD |
| AW204385 | p < 1e−07 | PDE7A\|phosphodiesterase 7A |
| AW451872 | p < 1e−07 | KIAA0173\|KIAA0173 gene product |
| AL162059 | p < 1e−07 | TLE4\|transducin-like enhancer of split 4 (E(sp1) homolog, Drosophila) |
| AA664218 | p < 1e−07 | ETV5\|ets variant gene 5 (ets-related molecule) |
| AL136836 | p < 1e−07 | LOC51678\|MAGUK protein p55T; Protein Associated with Lins 2 |
| AL133590 | p < 1e−07 | WASF3\|WAS protein family, member 3 |
| AK058009 | 0.0000022 | Homo sapiens cDNA FLJ25280 fis, clone STM06543 |
| AI207256 | p < 1e−07 | Homo sapiens mRNA for FLJ00074 protein, partial cds |
| AL137281 | p < 1e−07 | Homo sapiens mRNA; cDNA DKFZp434C2016 (from clone DKFZp434C2016) |
| BF433296 | p < 1e−07 | GBP1\|guanylate binding protein 1, interferon-inducible, 67 kD |
| AA714213 | p < 1e−07 | ESTs, Highly similar to T47163 hypothetical protein DKFZp762E1312.1 H. sapiens |
| BC013966 | p < 1e−07 | FLJ10156\|hypothetical protein |
| AI082049 | p < 1e−07 | ESTs |
| U74612 | p < 1e−07 | FOXM1\|forkhead box M1 |
| W94858 | p < 1e−07 | GAS1\|growth arrest-specific 1 |
| BI492329 | p < 1e−07 | KIAA1214\|KIAA1214 protein |
| AB041036 | 0.0001467 | KLK11\|kallikrein 11 |
| AA931563 | p < 1e−07 | OR7E27P\|olfactory receptor, family 7, subfamily E, member 27 pseudogene |
| AF118070 | p < 1e−07 | DKFZp762A227\|hypothetical protein DKFZp762A227 |
| AI567843 | p < 1e−07 | ESTs, Weakly similar to JC5314 CDC28/cdc2-like kinase associating arginine-serine cyclophilin H. sapiens |
| BC006443 | p < 1e−07 | LRP8\|low density lipoprotein receptor-related protein 8, apolipoprotein e receptor |
| AF178532 | p < 1e−07 | BACE2\|beta-site APP-cleaving enzyme 2 |
| AA205947 | p < 1e−07 | HHGP\|HHGP protein |
| U85267 | p < 1e−07 | DSCR1\|Down syndrome critical region gene 1 |
| AW290960 | p < 1e−07 | ESTs, Highly similar to T00391 hypothetical protein KIAA0612 H. sapiens |
| AK025747 | p < 1e−07 | FIGN\|fidgetin |
| AB027289 | p < 1e−07 | LOC51191\|cyclin-E binding protein 1 |
| L20470 | p < 1e−07 | VLDLR\|very low density lipoprotein receptor |
| BC015592 | p < 1e−07 | KIAA0937\|KIAA0937 protein |
| AF247820 | p < 1e−07 | CALD1\|caldesmon 1 |
| AK00012 | p < 1e−07 | FLJ20005\|hypothetical protein FLJ20005 |
| AL157483 | p < 1e−07 | FLJ11264\|hypothetical protein FLJ11264 |
| AA810022 | p < 1e−07 | KIAA0846\|KIAA0846 protein |
| AB048286 | p < 1e−07 | DGAT2\|diacylglycerol O-acyltransferase homolog 2 (mouse) |
| BC017575 | p < 1e−07 | CHEK1\|CHK1 checkpoint homolog (S. pombe) |
| AA877761 | p < 1e−07 | ESTs, Weakly similar to SYN1 MOUSE SYNAPSIN I [M. musculus] |
| AJ010277 | p < 1e−07 | TBX19\|T-box 19 |
| AF237763 | 0.0000112 | GPR87\|G protein-coupled receptor 87 |
| AW874308 | p < 1e−07 | OR7E38P\|olfactory receptor, family 7, subfamily E, member 38 pseudogene |
| AF230904 | p < 1e−07 | SH3KBP1\|SH3-domain kinase binding protein 1 |
| Y16645 | 0.0000455 | SCYA8\|small inducible cytokine subfamily A (Cys—Cys), member 8 (monocyte chemotactic protein 2) |
| BC010466 | p < 1e−07 | B3GAT1\|beta-1,3-glucuronyltransferase 1 (glucuronosyltransferase P) |
| AK024298 | p < 1e−07 | Homo sapiens cDNA FLJ14236 fis, clone NT2RP4000515 |
| AK026095 | p < 1e−07 | SNTB1\|syntrophin, beta 1 (dystrophin-associated protein A1, 59 kD, basic component 1) |
| AK022914 | p < 1e−07 | Homo sapiens cDNA FLJ12852 fis, clone NT2RP2003445 |
| AJ276230 | p < 1e−07 | PLD1\|phospholipase D1, phophatidylcholine-specific |
| AK022089 | p < 1e−07 | PAMCI\|peptidylglycine alpha-amidating monooxygenase COOH-terminal interactor |
| AI023133 | p < 1e−07 | ESTs |
| BC002536 | p < 1e−07 | PFKP\|phosphofructokinase, platelet |
| NM_005401 | p < 1e−07 | PTPN14\|protein tyrosine phosphatase, non-receptor type 14 |
| AI350087 | 0.0000002 | ESTs, Weakly similar to MUC2_HUMAN MUCIN 2 PRECURSOR H. sapiens |
| NM_005409 | 0.0000011 | SCYB11\|small inducible cytokine subfamily B (Cys-X-Cys), member 11 |
| AK024865 | p < 1e−07 | FLJ21212\|hypothetical protein FLJ21212 |
| BC015484 | p < 1e−07 | CALB2\|calbindin 2, (29 kD, calretinin) |
| AK025562 | p < 1e−07 | Homo sapiens cDNA: FLJ21909 fis, clone HEP03834 |
| AI538226 | 0.0000006 | GNG4\|guanine nucleotide binding protein 4 |
| AW409700 | 0.0000002 | ESTs, Weakly similar to similar to ankyrin of Chromatium vinosum H. sapiens |
| AI656548 | p < 1e−07 | ESTs |
| AJ223366 | 0.0000019 | Homo sapiens mRNA for OCIM (Oncogene in Multiple Myeloma) protein |
| AI524385 | p < 1e−07 | ANLN\|anillin, actin binding protein (scraps homolog, Drosophila) |
| AF241534 | p < 1e−07 | HYMAI\|hydatidiform mole associated and imprinted |
| BC004379 | 0.0000005 | GJB5\|gap junction protein, beta 5 (connexin 31.1) |
| BC012198 | p < 1e−07 | Homo sapiens, clone MGC: 4408 IMAGE: 2906200, mRNA, complete cds |
| AL136653 | p < 1e−07 | DEPP\|decidual protein induced by progesterone |
| AK026139 | p < 1e−07 | GJB3\|gap junction protein, beta 3, 31 kD (connexin 31) |
| BC012513 | p < 1e−07 | ARHE\|ras homolog gene family, member E |
| BC010044 | p < 1e−07 | CDC20\|CDC20 cell division cycle 20 homolog (S. cerevisiae) |
| AL162079 | p < 1e−07 | SLC16A1\|solute carrier family 16 (monocarboxylic acid transporters), member 1 |
| BC000218 | p < 1e−07 | DLL3\|delta-like 3 (Drosophila) |
| AK000770 | p < 1e−07 | Homo sapiens cDNA FLJ20763 fis, clone COL09911 |
| BC001211 | p < 1e−07 | KIFC3\|kinesin family member C3 |
| BC010612 | p < 1e−07 | Homo sapiens, clone IMAGE: 4157546, mRNA, partial cds |
| BC003610 | p < 1e−07 | MFGE8\|milk fat globule-EGF factor 8 protein |
| AK025511 | p < 1e−07 | ATP11A\|ATPase, Class VI, type 11A |
| AF102848 | 0.0001895 | HAIK1\|type I intermediate filament cytokeratin |
| AL035878 | 0.0003777 | DCX\|doublecortex; lissencephaly, X-linked (doublecortin) |
| AI188749 | 0.0000001 | ESTs |
| AI004681 | p < 1e−07 | ESTs, Weakly similar to T33068 hypothetical protein C35E7.9 - Caenorhabditis elegans [C. elegans] |
| AA988024 | 0.0000093 | FLJ14503\|hypothetical protein FLJ14503 |
| BC013106 | p <1e−07 | RRAS2\|related RAS viral (r-ras) oncogene homolog 2 |
| BC006537 | 0.0001025 | HOXA9\|homeo box A9 |
| BC007964 | 0.0000001 | CD38\|CD38 antigen (p45) |
| AA903532 | 0.0000001 | SV2B\|synaptic vesicle protein 2B homolog |
| BC015839 | p < 1e−07 | Homo sapiens, clone IMAGE: 4296901, mRNA |
| BC009232 | 0.0000005 | Homo sapiens, Similar to G antigen 8, clone MGC: 16513 IMAGE: 3960352, mRNA, complete cds |
| AI015671 | 0.0000001 | FLJ12604\|hypothetical protein FLJ12604 |
| D25538 | p < 1e−07 | ADCY7\|adenylate cyclase 7 |
| BC011000 | p < 1e−07 | MGC16386\|similar to RIKEN cDNA 2610036L13 |
| BM052808 | p < 1e−07 | KIAA0237\|KIAA0237 gene product |
| AK026826 | p < 1e−07 | Homo sapiens cDNA: FLJ23173 fis, clone LNG10019 |
| BC000078 | 0.0000001 | MGC3279\|hypothetical protein MGC3279 similar to collectins |
| AL049933 | p < 1e−07 | GNAI1\|guanine nucleotide binding protein (G protein), alpha inhibiting activity polypeptide 1 |
| AF136631 | p < 1e−07 | LOC51299\|neuritin |
| AW205836 | p < 1e−07 | RAB6B\|RAB6B, member RAS oncogene family |
| BC015640 | 0.0000005 | CBRI\|carbonyl reductase 1 |
| AI654872 | p < 1e−07 | RASSF2\|Ras association (RaIGDS/AF-6) domain family 2 |
| AF053305 | p < 1e−07 | BUB1\|BUB1 budding uninhibited by benzimidazoles 1 homolog (yeast) |
| AF331521 | p < 1e−07 | SLC26A7\|solute carrier family 26, member 7 |
| AA993639 | p < 1e−07 | FLJ10748\|hypothetical protein FLJ10748 |
| AL157443 | p < 1e−07 | FAT2\|FAT tumor suppressor homolog 2 (Drosophila) |
| BC007407 | p < 1e−07 | NMB\|neuromedin B |
| AL080058 | p < 1e−07 | DKFZP564G202\|DKFZP564G202 protein |
| M59305 | p < 1e−07 | NPR3\|natriuretic peptide receptor C/guanylate cyclase C (atrionatriuretic peptide receptor C) |
| BC000703 | p < 1e−07 | FLJ10468\|hypothetical protein FLJ10468 |
| S79869 | p < 1e−07 | HOXA1\|homeo box A1 |
| BC008584 | 0.0000021 | BMP7\|bone morphogenetic protein 7 (osteogenic protein 1) |
| BC008947 | p < 1e−07 | FLJ10540\|hypothetical protein FLJ10540 |
| BC008036 | 0.0000079 | KLK5\|kallikrein 5 |
| BC012204 | p < 1e−07 | Homo sapiens, clone MGC: 10002 IMAGE: 3882800, mRNA, complete cds |
| BC001847 | p < 1e−07 | MGC4504\|hypothetical protein MGC4504 |
| AA088779 | p < 1e−07 | MFHAS1\|malignant fibrous histiocytoma amplified sequence 1 |
| AF177337 | p < 1e−07 | FLJ13391\|hypothetical protein FLJ13391 |
| AF283670 | 0.0000011 | KLK14\|kallikrein 14 |
| BC115310 | p < 1e−07 | FLJ21069\|hypothetical protein FLJ21069 |
| AF201933 | p < 1e−07 | DC11\|DC11 protein |
| AL136820 | p < 1e−07 | KIAA1411\|KIAA1411 protein |
| BC000260 | p < 1e−07 | AKR1B1\|aldo-keto reductase family 1, member B1 (aldose reductase) |
| AI081342 | p < 1e−07 | OR7E37P\|olfactory receptor, family 7, subfamily E, member 37 pseudogene |
| BC007491 | p < 1e−07 | EXO1\|exonuclease 1 |
| AK026061 | p < 1e−07 | FLJ22408\|hypothetical protein FLJ22408 |
| W69074 | p < 1e−07 | S100A4\|S100 calcium binding protein A4 (calcium protein, calvasculin, metastasin, murine placental homolog) |
| BC007707 | 0.0000112 | HEY2\|hairy/enhancer-of-split related with YRPW motif 2 |
| AK057943 | 0.0000001 | Homo sapiens cDNA FLJ25214 fis, clone REC08615 |
| AF222980 | p < 1e−07 | DISC1\|disrupted in schizophrenia 1 |
| AF172852 | p < 1e−07 | CLSP\|calmodulin-like skin protein |
| AI569288 | p < 1e−07 | ESTs |
| AF153609 | p < 1e−07 | SGK\|serum/glucocorticoid regulated kinase |
| BF512938 | p < 1e−07 | RARB\|retinoic acid receptor, beta |
| AI633770 | p < 1e−07 | ESTs |
| BC014645 | p < 1e−07 | PRX2\|paired related homeobox protein |
| BC007951 | 0.0000002 | SOX9\|SRY (sex determining region Y)-box 9 (campomelic dysplasia, autosomal sex-reversal) |
| AI299541 | p < 1e−07 | ESTs |
| BC001940 | p < 1e−07 | DKFZp762E1312\|hypothetical protein DKFZp762E1312 |
| AA857853 | 0.0000006 | LCN2\|lipocalin 2 (oncogene 24p3) |
| N22033 | p < 1e−07 | COL11A2\|collagen, type Xl, alpha 2 |
| AI200706 | 0.0000004 | FLJ10134\|hypothetical protein FLJ10134 |
| BC010100 | p < 1e−07 | TLE1\|transducin-like enhancer of split 1 (E(sp1) homolog, Drosophila) |
| BC010078 | p < 1e−07 | RISC\|likely homolog of rat and mouse retinoid-inducible serine carboxypeptidase |
| AI936089 | 0.0000004 | Homo sapiens cDNA FLJ14181 fis, clone NT2RP2004300 |
| BC001207 | 0.0000153 | MAGE-E1\|MAGE-E1 protein |
| AK056720 | p < 1e−07 | Homo sapiens cDNA FLJ32158 fis, clone PLACE6000231 |
| M86609 | 0.0000002 | AKR1C1\|aldo-keto reductase family 1, member C1 (dihydrodiol dehydrogenase 1; 20-alpha (3-alpha)- |
| | | hydroxysteroid dehydrogenase) |
| AI688729 | p < 1e−07 | ATP11C\|ATPase, Class VI, type 11C |
| J05428 | 0.0000025 | UGT2B7\|UDP glycosyltransferase 2 family, polypeptide B7 |
| AI298332 | p < 1e−07 | ESTs |
| AI097664 | p < 1e−07 | ESTs, Weakly similar to S23650 retrovirus-related hypothetical protein II H. sapiens |
| NM_002465 | 0.0000202 | MYBPC1\|myosin binding protein C, slow type |
| BF939405 | p < 1e−07 | EST |
| AF166350 | p < 1e−07 | ST7\|potential tumor suppressor |
| BC009401 | p < 1e−07 | NK4\|natural killer cell transcript 4 |
| BC009989 | p < 1e−07 | SMOH smoothened homolog (Drosophila) |
| AV655170 | p < 1e−07 | C21orf35\|chromosome 21 open reading frame 35 |
| BC005246 | 0.0001085 | TM4SF3\|transmembrane 4 superfamily member 3 |
| AK024732 | p < 1e−07 | FLJ21079\|hypothetical protein FLJ21079 |
| NM_002281 | p < 1e−07 | KRTHB1\|keratin, hair, basic, 1 |
| AW293632 | 0.0000467 | ESTs, Weakly similar to YK33_YEAST HYPOTHETICAL 46.5 KD PROTEIN IN MRS4-DYN1 INTERGENIC |
| | | REGION [S. cerevisiae] |
| X55362 | p < 1e−07 | ODC1\|ornithine decarboxylase 1 |
| AF060877 | p < 1e−07 | RGS20\|regulator of G-protein signalling 20 |
| BC007022 | 0.0000109 | SAA1\|serum amyloid A1 |
| BC007026 | p < 1e−07 | SAA4\|serum amyloid A4, constitutive |
| AA643687 | 0.000722 | Homo sapiens cDNA FLJ11980 fis, clone HEMBB1001304 |
| AI978664 | p < 1e−07 | Homo sapiens DNA sequence from PAC 127D3 on chromosome 1q23-25. Contains FMO2 and FMO3 genes for |
| | | Flavin-containing Monooxygenase 2 and Flavin-containing Monooxygenase 3 (Dimethylaniline Monooxygenase |
| | | (N-Oxide 3, EC1.14.13.8, Dimethylaniline Oxidase 3, FMO II, FMO 3), and a gene for another, unknown, Flavin- |
| | | containing Monooxygenase family protein. Contains ESTs and GSSs |
| AW015394 | 0.0000083 | OR2I6\|olfactory receptor, family 2, subfamily I, member 6 |
| AW449368 | p < 1e−07 | PDL2\|programmed death ligand 2 |
| AW139532 | 0.000027 | Homo sapiens cDNA FLJ32375 fis, clone SALGL1000065, weakly similar to POLYHOMEOTIC-PROXIMAL |
| | | CHROMATIN PROTEIN |
| AI278360 | 0.0000005 | ESTs |
| BF939156 | 0.0000005 | SCYA5\|small inducible cytokine A5 (RANTES) |
| AL137740 | 0.0001579 | LBP-9\|LBP protein; likely ortholog of mouse CRTR-1 |
| AI924205 | 0.000124 | ADAMDEC1\|ADAM-like, decysin 1 |
| BC002551 | p < 1e−07 | MGC2577\|hypothetical protein MGC2577 |
| AF239668 | p < 1e−07 | CCKBR\|cholecystokinin B receptor |
| BC013353 | 0.0000001 | FLJ14166\|hypothetical protein FLJ14166 |
| AW410306 | 0.0000021 | NXPH4\|neurexophilin 4 |
| BC008832 | p < 1e−07 | HMGIY\|high-mobility group (nonhistone chromosomal) protein isoforms I and Y |
| AL080146 | p < 1e−07 | CCNB2\|cyclin B2 |
| BE047139 | p < 1e−07 | ESTs |
| AL365412 | 0.0000115 | LOC56932\|hypothetical protein from EUROIMAGE 1759349 |
| BC008651 | 0.0002167 | Homo sapiens, clone MGC: 9913 IMAGE: 3870821, mRNA, complete cds |
| AA398573 | p < 1e−07 | EIF5A2\|eukaryotic translation initiation factor 5A2 |
| AI457921 | p < 1e−07 | Homo sapiens cDNA FLJ11436 fis, clone HEMBA1001213 |
| AF280546 | p < 1e−07 | NRP2\|neuropilin 2 |
| BF447302 | p < 1e−07 | ESTs |
| BC010091 | p < 1e−07 | BICD1\|Bicaudal D homolog 1 (Drosophila) |
| AK026015 | p < 1e−07 | FLJ22362\|hypothetical protein FLJ22362 |
| BC014126 | p < 1e−07 | Homo sapiens, Similar to RIKEN cDNA 4932416D09 gene, clone IMAGE: 4578228, mRNA, partial cds |
| U61374 | p < 1e−07 | SRPX\|sushi-repeat-containing protein, X chromosome |
| AI280215 | 0.0000014 | ESTs |
| NM_001559 | p < 1e−07 | IL12RB2\|interleukin 12 receptor, beta 2 |
| BC011795 | p < 1e−07 | NDRG4\|NDRG family member 4 |
| AA903387 | p < 1e−07 | SIL\|TAL1 (SCL) interrupting locus |
| NM_021226 | p < 1e−07 | LOC58504\|hypothetical protein from clones 23549 and 23762 |
| BC009942 | p < 1e−07 | FLJ10628\|hypothetical protein FLJ10628 |
| AA772897 | p < 1e−07 | DNCH1\|dynein, cytoplasmic, heavy polypeptide 1 |
| BC006322 | 0.0000001 | ATF3\|activating transcription factor 3 |
| AI765381 | 0.0000017 | DJ434O14.3\|hypothetical protein dJ434O14.3 |
| BC007631 | p < 1e−07 | MGC15827\|hypothetical protein MGC15827 |
| AA937401 | 0.0006517 | ESTs |
| BC001886 | p < 1e−07 | RRM2\|ribonucleotide reductase M2 polypeptide |
| AK057339 | 0.0000005 | LOC81569\|actin like protein |
| BC007318 | p < 1e−07 | MAPRE2\|microtubule-associated protein, RP/EB family, member 2 |
| AA573096 | p < 1e−07 | FLJ10829\|hypothetical protein FLJ10829 |
| BC007656 | p < 1e−07 | UBE2C\|ubiquitin-conjugating enzyme E2C |
| BM055132 | 0.0000057 | CFTR\|cystic fibrosis transmembrane conductance regulator, ATP-binding cassette (sub-family C, member 7) |
| AB011422 | p < 1e−07 | TRAD\|serine/threonine kinase with DbI- and pleckstrin homology domains |
| BC016040 | p < 1e−07 | STK17B\|serine/threonine kinase 17b (apoptosis-inducing) |
| AK055323 | p < 1e−07 | Homo sapiens cDNA FLJ30761 fis, clone FEBRA2000538 |
| BC005894 | 0.0000001 | FMO2\|flavin containing monooxygenase 2 |
| NM_001078 | 0.0000011 | VCAM1\|vascular cell adhesion molecule 1 |
| AL136819 | p < 1e−07 | DKFZP434G2226\|hypothetical protein DKFZp434G2226 |
| AF054993 | 0.0000008 | SNAP91\|synaptosomal-associated protein, 91 kD homolog (mouse) |
| AY044429 | p < 1e−07 | IL22RA2\|class II cytokine receptor |
| AB033109 | 0.0000196 | KIAA1283\|KIAA1283 protein |
| AI871458 | p < 1e−07 | ESTs |
| AI569068 | 0.0000063 | ESTs |
| AK056817 | p < 1e−07 | Homo sapiens cDNA FLJ32255 fis, clone PROST1000226 |
| BC006267 | 0.0000711 | FLJ22316\|hypothetical protein FLJ22316 |
| AA810180 | p < 1e−07 | FLJ10517\|hypothetical protein FLJ10517 |
| AF017790 | p < 1e−07 | HEC\|highly expressed in cancer, rich in leucine heptad repeats |
| BC010935 | 0.0000241 | CSN10\|casein, kappa |
| AI741771 | p < 1e−07 | RYR3\|ryanodine receptor 3 |
| H06285 | p < 1e−07 | Homo sapiens cDNA FLJ30553 fis, clone BRAWH2003689, highly similar to Mus musculus clone mouse1-9 |
| | | putative protein phosphatase type 2C mRNA |
| AA613572 | p < 1e−07 | ESTs |
| AL512731 | 0.0000001 | Homo sapiens mRNA; cDNA DKFZp667B0711 (from clone DKFZp667B0711) |
| W57704 | 0.0000001 | CNGA1\|cyclic nucleotide gated channel alpha 1 |
| BF224101 | p < 1e−07 | DOCK3\|dedicator of cyto-kinesis 3 |
| AJ420539 | p < 1e−07 | DEK\|DEK oncogene (DNA binding) |
| D10522 | p < 1e−07 | MARCKS\|myristoylated alanine-rich protein kinase C substrate |
| BC008691 | p < 1e−07 | RSU1\|Ras suppressor protein 1 |
| AF207664 | p < 1e−07 | ADAMTS1\|a disintegrin-like and metalloprotease (reprolysin type) with thrombospondin type 1 motif, 1 |
| AF070592 | p < 1e−07 | HSKM-B\|HSKM-B protein |
| AK025290 | p < 1e−07 | ATP10B\|ATPase, Class V, type 10B |
| BC001749 | p < 1e−07 | WNT5B\|wingless-type MMTV integration site family, member 5B |
| AK025425 | p < 1e−07 | MGC21854\|hypothetical protein MGC21854 |
| AA642589 | p < 1e−07 | SEMG2\|semenogelin II |
| BC010116 | 0.0000001 | Homo sapiens, clone MGC: 19762 IMAGE: 3636045, mRNA, complete cds |
| NM_002510 | 0.0000001 | GPNMB\|glycoprotein (transmembrane) nmb |
| BC002744 | p < 1e−07 | MGC4090\|hypothetical protein MGC4090 |
| AA574056 | p < 1e−07 | OR7E119P\|olfactory receptor, family 7, subfamily A, member 119 pseudogene |
| AI500430 | 0.0000052 | KIAA1909\|KIAA1909 protein |
| AI972591 | 0.0000062 | ESTs |
| AA232651 | p < 1e−07 | SUV39H2\|suppressor of variegation 3-9 (Drosophila) homolog 2; hypothetical protein FLJ23414 |
| BC009238 | p < 1e−07 | TUBA1\|tubulin, alpha 1 (testis specific) |
| NM_022977 | p < 1e−07 | FACL4\|fatty-acid-Coenzyme A ligase, long-chain 4 |
| BC001844 | 0.000001 | MEIS2\|Meis1, myeloid ecotropic viral integration site 1 homolog 2 (mouse) |
| N51717 | 0.0000117 | ESTs |
| BC010658 | p < 1e−07 | KIAA0008\|KIAA0008 gene product |
| BC013200 | 0.0000001 | LCK\|lymphocyte-specific protein tyrosine kinase |
| AF027866 | p < 1e−07 | SERPINB7\|serine (or cysteine) proteinase inhibitor, clade B (ovalbumin), member 7 |
| AK026964 | p < 1e−07 | FLJ23311\|hypothetical protein FLJ23311 |
| BC010923 | 0.0000002 | CART1\|cartilage paired-class homeoprotein 1 |
| R38438 | p < 1e−07 | SLC15A2\|solute carrier family 15 (H+/peptide transporter), member 2 |
| AL110202 | p < 1e−07 | PORIMIN\|pro-oncosis receptor inducing membrane injury gene |
| AK025215 | 0.0000001 | FLJ21562\|hypothetical protein FLJ21562 |
| D90239 | 0.0005882 | GLDC\|glycine dehydrogenase (decarboxylating; glycine decarboxylase, glycine cleavage system protein P) |
| AI636660 | p < 1e−07 | C21orf91\|chromosome 21 open reading frame 91 |
| BC000633 | p < 1e−07 | TTK\|TTK protein kinase |
| BF511873 | p < 1e−07 | ESTs |
| AI816719 | 0.0000012 | KIAA1870\|KIAA1870 protein |
| AF053306 | p < 1e−07 | BUB1B\|BUB1 budding uninhibited by benzimidazoles 1 homolog beta (yeast) |
| NM_005257 | 0.0000001 | GATA6\|GATA binding protein 6 |
| BC011920 | p < 1e−07 | FLJ20896\|hypothetical protein FLJ20896 |
| X12892 | 0.000001 | PROS1\|protein S (alpha) |
| M62925 | p < 1e−07 | TGM1\|transglutaminase 1 (K polypeptide epidermal type I, protein-glutamine-gamma-glutamyltransferase) |
| AF239821 | 0.0000003 | GALNAC4ST-2\|GalNAc-4-sulfotransferase 2 |
| BF437448 | p < 1e−07 | EST |
| AF098482 | p < 1e−07 | PSIP1\|PC4 and SFRS1 interacting protein 1 |
| AI096342 | p < 1e−07 | RGC32\|RGC32 protein |
| BC015706 | p < 1e−07 | Homo sapiens, Similar to RIKEN cDNA2810433K01 gene, clone MGC: 10200 IMAGE: 3909951, mRNA, |
| | | complete cds |
| BC011561 | 0.0000001 | HEPH\|hephaestin |
| AA903183 | 0.0000011 | IL2RA\|interleukin 2 receptor, alpha |
| NM_001237 | p < 1e−07 | CCNA2\|cyclin A2 |
| AL137502 | p < 1e−07 | RAGD\|Rag D protein |
| BC006359 | p < 1e−07 | INA\|internexin neuronal intermediate filament protein, alpha |
| BF592799 | p < 1e−07 | PRKCQ\|protein kinase C, theta |
| AI697249 | 0.0000294 | EBI2\|Epstein-Barr virus induced gene 2 (lymphocyte-specific G protein-coupled receptor) |
| BC016719 | 0.000725 | Homo sapiens, clone IMAGE: 4070464, mRNA, partial cds |
| AF014398 | p < 1e−07 | IMPA2\|inositol(myo)-1(or 4)-monophosphatase 2 |
| AL136828 | 0.0000001 | DKFZP434K0427\|hypothetical protein DKFZp434K0427 |
| AI972473 | 0.0000723 | EST |
| AW104435 | 0.0000002 | UBE2E2\|ubiquitin-conjugating enzyme E2E 2 (UBC4/5 homolog, yeast) |
| M74447 | p < 1e−07 | TAP2\|transporter 2, ATP-binding cassette, sub-family B (MDR/TAP) |
| NM_005048 | 0.000801 | PTHR2\|parathyroid hormone receptor 2 |
| AK024854 | p < 1e−07 | DJ742C19.2\|phorbolin (similar to apolipoprotein B mRNA editing protein) |
| AK027274 | p < 1e−07 | Homo sapiens cDNA FLJ14368 fis, clone HEMBA1001122 |
| AF147204 | 0.0000002 | CXCR4\|chemokine (C-X-C motif), receptor 4 (fusin) |
| BC005232 | p < 1e−07 | RPP38\|ribonuclease P (38 kD) |
| BC004897 | 0.0000011 | CA3\|carbonic anhydrase III, muscle specific |
| NM_002963 | 0.0000573 | S100A7\|S100 calcium binding protein A7 (psoriasin 1) |
| AF154845 | 0.0000003 | MARK1\|MAP/microtubule affinity-regulating kinase 1 |
| AK000659 | p < 1e−07 | MT-ACT48\|Mitochondrial Acyl-CoA Thioesterase |
| AJ420567 | p < 1e−07 | AMD1\|S-adenosylmethionine decarboxylase 1 |
| AF030942 | p < 1e−07 | PPP1R14BP1\|protein phosphatase 1, regulatory (inhibitor) subunit 14B pseudogene 1 |
| AL359561 | p < 1e−07 | DKFZP762N2316\|hypothetical protein DKFZp762N2316 |
| NM_002417 | p < 1e−07 | MKI67\|antigen identified by monoclonal antibody Ki-67 |
| NM_003503 | p < 1e−07 | CDC7L1\|CDC7 cell division cycle 7-like 1 (S. cerevisiae) |
| AA084672 | 0.0000034 | MSI1\|musashi homolog 1 (Drosophila) |
| AL136919 | p < 1e−07 | DKFZp586J1119\|hypothetical protein DKFZp586J1119 |
| BC001051 | p < 1e−07 | ARL7\|ADP-ribosylation factor-like 7 |
| AF136715 | 0.0000176 | TRAG3\|taxol resistance associated gene 3 |
| X97868 | 0.0000001 | ARSF\|arylsulfatase F |
| AI568024 | 0.0000032 | LAMB3\|laminin, beta 3 (nicein (125 kD), kalinin (140 kD), BM600 (125 kD)) |
| AW191026 | 0.0000001 | PLSCR1\|phospholipid scramblase 1 |
| BC017705 | p < 1e−07 | KNSL5\|kinesin-like 5 (mitotic kinesin-like protein 1) |
| AB032698 | 0.0000007 | BIN2\|bridging integrator 2 |
| BC001692 | 0.0000005 | SLC2A5\|solute carrier family 2 (facilitated glucose/fructose transporter), member 5 |
| AB043585 | 0.0000001 | REPRIMO\|candidate mediator of the p53-dependent G2 arrest |
| AA262504 | p < 1e−07 | EYA3\|eyes absent homolog 3 (Drosophila) |
| BC017656 | p < 1e−07 | MONDOA\|Mlx interactor |
| AK055596 | p < 1e−07 | Homo sapiens cDNA FLJ31034 fis, clone HSYRA1000178 |
| NM_000057 | p < 1e−07 | BLM\|Bloom syndrome |
| BC012527 | 0.0000001 | HIF1A\|hypoxia-inducible factor 1, alpha subunit (basic helix-loop-helix transcription factor) |
| BF196234 | 0.0000026 | ESTs |
| AA805970 | 0.0000034 | KIAA0275\|KIAA0275 gene product |
| AL050066 | p < 1e−07 | NTT73\|homolog of rat orphan transporter v7-3 |
| AF339763 | p < 1e−07 | ABCC4\|ATP-binding cassette, sub-family C (CFTR/MRP), member 4 |
| AW189851 | 0.0000008 | CUGBP2\|CUG triplet repeat, RNA binding protein 2 |
| NM_003122 | 0.0000169 | SPINK1\|serine protease inhibitor, Kazal type 1 |
| AA826489 | 0.0000001 | MY040\|hypothetical brain protein my040 |
| AK000054 | p < 1e−07 | FLJ20047\|hypothetical protein FLJ20047 |
| AI653207 | p < 1e−07 | ESTs |
| AL137735 | p < 1e−07 | ASB2\|ankyrin repeat and SOCS box-containing 2 |
| AI273114 | p < 1e−07 | ESTs, Weakly similar to I78885 serine/threonine-specific protein kinase H. sapiens |
| BI492625 | p < 1e−07 | Homo sapiens cDNA FLJ30075 fis, clone BGGI11000285 |
| NM_006235 | 0.0002076 | POU2AF1\|POU domain, class 2, associating factor 1 |
| AK000208 | p < 1e−07 | Homo sapiens cDNA FLJ20201 fis, clone COLF1210 |
| AI669751 | 0.0000664 | ZIP2\|zinc transporter |
| BF513300 | p < 1e−07 | KIAA0074\|KIAA0074 protein |
| M15990 | p < 1e−07 | YES1\|v-yes-1 Yamaguchi sarcoma viral oncogene homolog 1 |
| BE671269 | p < 1e−07 | HAAO\|3-hydroxyanthranilate 3,4-dioxygenase |
| BC000404 | p < 1e−07 | TRIP13\|thyroid hormone receptor interactor 13 |
| AL050388 | 0.0000001 | Homo sapiens mRNA; cDNA DKFZp564M2422 (from clone DKFZp564M2422); partial cds |
| BC014526 | 0.0000024 | ANKH\|ankylosis, progressive homolog (mouse) |
| BC007585 | p < 1e−07 | MYBL2\|v-myb myeloblastosis viral oncogene homolog (avian)-like 2 |
| AK025926 | 0.0000002 | AK3\|adenylate kinase 3 |
| BC005127 | p < 1e−07 | ADFP\|adipose differentiation-related protein |
| AK054683 | p < 1e−07 | Homo sapiens cDNA FLJ30121 fis, clone BRACE1000084 |
| BE206076 | p < 1e−07 | ALK\|anaplastic lymphoma kinase (Ki-1) |
| R05446 | p < 1e−07 | KIAA1424\|KIAA1424 protein |
| BC003684 | 0.000076 | CXADR\|coxsackie virus and adenovirus receptor |
| BC016451 | p < 1e−07 | ADAMTS3\|a disintegrin-like and metalloprotease (reprolysin type) with thrombospondin type 1 motif, 3 |
| AF070609 | p < 1e−07 | SLC1A3\|solute carrier family 1 (glial high affinity glutamate transporter), member 3 |
| BF724533 | p < 1e−07 | MGC3169\|hypothetical protein MGC3169 |
| AB053172 | p < 1e−07 | CDT1\|DNA replication factor |
| AF124735 | 0.0005833 | LHX2\|LIM homeobox protein 2 |
| AL157471 | 0.0000189 | KIAA1500\|KIAA1500 protein |
| AF326731 | p < 1e−07 | NUF2R\|hypothetical protein NUF2R |
| BC013767 | p < 1e−07 | Homo sapiens, Similar to RIKEN cDNA 2610528G05 gene, clone IMAGE: 3875837, mRNA, partial cds |
| AA827777 | p < 1e−07 | ESTs |
| NM_006329 | p < 1e−07 | FBLN5\|fibulin 5 |
| BC014018 | p < 1e−07 | FLJ11196\|hypothetical protein FLJ11196 |
| BE047394 | 0.0000013 | ESTs, Weakly similar to S71512 hypothetical protein T2 - mouse [M. musculus] |
| AW079735 | 0.0000016 | SPRR2B\|small proline-rich protein 2B |
| AK023080 | p < 1e−07 | HSNOV1\|novel protein |
| AL134596 | 0.0002539 | ATP2B2\|ATPase, Ca++ transporting, plasma membrane 2 |
| AF331796 | p < 1e−07 | HCAP-G\|chromosome condensation protein G |
| AF329840 | p < 1e−07 | ZSIG37\|G protein coupled receptor interacting protein, complement-c1q tumor necrosis factor-related |
| R40565 | p < 1e−07 | FYN\|FYN oncogene related to SRC, FGR, YES |
| BC002700 | 0.0000032 | KRT7\|keratin 7 |
| AA877996 | 0.0002536 | ESTs, Weakly similar to JC5314 CDC28/cdc2-like kinase associating arginine-serine cyclophilin H. sapiens |
| R48461 | 0.0000037 | ESTs |
| AA730123 | 0.0000001 | PRDM1\|PR domain containing 1, with ZNF domain |
| AF230495 | 0.00002 | DUOX1\|dual oxidase 1 |
| AL039118 | 0.0000661 | FOXG1B\|forkhead box G1B |
| AF146761 | 0.0001401 | BLAME\|BCM-like membrane protein precursor |
| BC012885 | 0.0000035 | TEM7R\|tumor endothelial marker 7-related precursor |
| AK026949 | 0.0000964 | ZNF300\|kruppel-like zinc finger protein |
| AF130988 | p < 1e−07 | EDAR\|ectodysplasin 1, anhidrotic receptor |
| AA489383 | p < 1e−07 | BMP2\|bone morphogenetic protein 2 |
| AF245356 | p < 1e−07 | TES\|testis derived transcript (3 LIM domains) |
| AI184027 | 0.0000001 | ESTs, Weakly similar to FATH_HUMAN CADHERIN-RELATED TUMOR SUPPRESSOR HOMOLOG |
| | | PRECURSOR H. sapiens |
| AI687303 | 0.0000032 | GPR49\|G protein-coupled receptor 49 |
| BC000715 | 0.0000108 | CLECSF9\|C-type (calcium dependent, carbohydrate-recognition domain) lectin, superfamily member 9 |
| H05918 | 0.0000019 | KIAA1867\|KIAA1867 protein |
| BF437096 | 0.0000015 | EST |
| AI193122 | 0.0000124 | CYYR1\|cysteine and tyrosine-rich 1 |
| AK027009 | 0.0002318 | FLJ23356\|hypothetical protein FLJ23356 |
| AI274576 | p < 1e−07 | CTSC\|cathepsin C |
| M90366 | 0.000042 | ZP2\|zona pellucida glycoprotein 2 (sperm receptor) |
| AY026763 | p < 1e−07 | GOA\|gene overexpressed in astrocytoma |
| AI049877 | p < 1e−07 | KIAA0186\|KIAA0186 gene product |
| R85380 | p < 1e−07 | ESTs |
| AA812178 | 0.0000001 | TLR1\|toll-like receptor 1 |
| U18548 | p < 1e−07 | GPR12\|G protein-coupled receptor 12 |
| AK000381 | p < 1e−07 | FLJ20374\|hypothetical protein FLJ20374 |
| BC001346 | 0.0000005 | LOC51700\|cytochrome b5 reductase b5R.2 |
| U87460 | 0.0000026 | GPR37\|G protein-coupled receptor 37 (endothelin receptor type B-like) |
| AW137459 | 0.0000029 | TMOD2\|tropomodulin 2 (neuronal) |
| AA485276 | 0.0005783 | CRTAC1\|cartilage acidic protein 1 |
| AI283489 | 0.0000008 | FACL2\|fatty-acid-Coenzyme A ligase, long-chain 2 |
| AI433440 | p < 1e−07 | TNFAIP3\|tumor necrosis factor, alpha-induced protein 3 |
| BC010940 | p < 1e−07 | AIM2\|absent in melanoma 2 |
| AF202636 | 0.0000004 | ANGPTL4\|angiopoietin-like 4 |
| BC012126 | 0.0000002 | CLDN14\|claudin 14 |
| BC000748 | 0.0000003 | TUBB4\|tubulin, beta, 4 |
| AK025320 | p < 1e−07 | SCEL\|sciellin |
| NM_003662 | 0.0000016 | PIR\|Pirin |
| AL133105 | 0.0000055 | DKFZp434F2322\|hypothetical protein DKFZp434F2322 |
| AK001643 | 0.000527 | FLJ10781\|hypothetical protein FLJ10781 |
| AW511572 | 0.0000029 | FZD6\|frizzled homolog 6 (Drosophila) |
| AI796505 | p < 1e−07 | HOXA2\|homeo box A2 |
| BF513683 | p < 1e−07 | GAJ\|GAJ protein |
| BC008283 | 0.0000169 | CCK\|cholecystokinin |
| NM_001784 | p < 1e−07 | CD97\|CD97 antigen |
| BC007633 | p < 1e−07 | EIF2C2\|eukaryotic translation initiation factor 2C, 2 |
| BC011403 | p < 1e−07 | GABRA5\|gamma-aminobutyric acid (GABA) A receptor, alpha 5 |
| BC002812 | p < 1e−07 | CBR3\|carbonyl reductase 3 |
| BC003630 | 0.0000048 | KRT4\|keratin 4 |
| AI362949 | 0.0000242 | ESTs |
| AW138010 | 0.0001317 | SAA2\|serum amyloid A2 |
| NM_005985 | p < 1e−07 | SNAI1\|snail 1 homolog, zinc finger protein (Drosophila) |
| BC012609 | p < 1e−07 | SERPINB2\|serine (or cysteine) proteinase inhibitor, clade B (ovalbumin), member 2 |
| BC010128 | 0.0000005 | FLJ10970\|hypothetical protein FLJ10970 |
| AK025309 | 0.0000032 | C1S\|complement component 1, s subcomponent |
| NM_025245 | 0.0000001 | PBX4\|pre-B-cell leukemia transcription factor 4 |
| BF507686 | p < 1e−07 | CERD4\|cer-d4 (mouse) homolog |
| AA731869 | 0.0002721 | CD1A\|CD1A antigen, a polypeptide |
| AK000193 | p < 1e−07 | FLJ20186\|hypothetical protein FLJ20186 |
| BC015859 | p < 1e−07 | Homo sapiens, clone MGC: 27381 IMAGE: 4689539, mRNA, complete cds |
| AL136855 | p < 1e−07 | DKFZp434K2435\|hypothetical protein DKFZp434K2435 |
| AF089868 | p < 1e−07 | MCAM\|melanoma cell adhesion molecule |
| AL360258 | 0.0000009 | FLJ11175\|hypothetical protein FLJ11175 |
| AK026248 | p < 1e−07 | FLJ22595\|hypothetical protein FLJ22595 |
| AI669793 | p < 1e−07 | ESTs |
| BC017840 | 0.0003003 | Homo sapiens, clone IMAGE: 4689305, mRNA, partial cds |
| BF112017 | p < 1e−07 | KCNE1L\|potassium voltage-gated channel, lsk-related family, member 1-like |
| AI375142 | 0.0000095 | LOC57822\|hypothetical protein LOC57822 |
| BC002377 | p < 1e−07 | PTK7\|PTK7 protein tyrosine kinase 7 |
| AB040057 | 0.0000006 | MST4\|serine/threonine protein kinase MASK |
| AF027299 | p < 1e−07 | EPB41L2\|erythrocyte membrane protein band 4.1-like 2 |
| AK026659 | 0.0000003 | Homo sapiens cDNA: FLJ23006 fis, clone LNG00414 |
| AF078749 | 0.0000014 | SLC22A3\|solute carrier family 22 (extraneuronal monoamine transporter), member 3 |
| AF277375 | p < 1e−07 | KIF4A\|kinesin family member 4A |
| AA747541 | 0.000033 | MYBL1\|v-myb myeloblastosis viral oncogene homolog (avian)-like 1 |
| BC007921 | 0.0000042 | SPIB\|Spi-B transcription factor (Spi-1/PU.1 related) |
| AI823489 | 0.0005112 | BARX2\|BarH-like homeobox 2 |
| NM_004233 | p < 1e−07 | CD83\|CD83 antigen (activated B lymphocytes, immunoglobulin superfamily) |
| AI886575 | p < 1e−07 | ESTs |
| AI439152 | 0.0000001 | KIAA1145\|KIAA1145 protein |
| NM_019035 | 0.0000022 | PCDH18\|protocadherin 18 |
| BF109847 | 0.0000025 | ESTs |
| AL137558 | 0.0000353 | VANGL2\|vang, van gogh-like 2 (Drosophila) |
| AI553863 | p < 1e−07 | KIAA1204\|AA1204 protein |
| AA866043 | p < 1e−07 | CBS\|cystathionine-beta-synthase |
| AW341053 | p < 1e−07 | ESTs |
| AK025912 | 0.0000004 | COL4A2\|collagen, type IV, alpha 2 |
| AA634391 | p < 1e−07 | MAGEB4\|melanoma antigen, family B, 4 |
| AI475447 | 0.0000043 | FLJ12567\|hypothetical protein FLJ12567 |
| NM_005430 | p < 1e−07 | WNT1\|wingless-type MMTV integration site family, member 1 |
| NM_001998 | p < 1e−07 | FBLN2\|fibulin 2 |
| AI803166 | 0.0000007 | ESTs |
| AL040372 | p < 1e−07 | ESTs, Weakly similar to A54854 Ras GTPase activating protein-related protein H. sapiens |
| BC012385 | p < 1e−07 | FBG4\|F-box protein FBG4 |
| AF045888 | 0.0000001 | DNMT2\|DNA (cytosine-5-)-methyltransferase 2 |
| AA588167 | 0.0000004 | KIAA1729\|KIAA1729 protein |
| AF051152 | 0.0000006 | TLR2\|toll-like receptor 2 |
| AL136550 | 0.0000077 | BCMP1\|brain cell membrane protein 1 |
| AI969883 | p < 1e−07 | EST |
| AK026181 | 0.000001 | Homo sapiens cDNA: FLJ22528 fis, clone HRC12825 |
| AI469557 | 0.0000026 | EPHB3\|EphB3 |
| AB037670 | 0.0000007 | SLC7A10\|solute carrier family 7, (cationic amino acid transporter, y+ system) member 10 |
| AK027157 | p < 1e−07 | DKFZp547N157\|hypothetical protein DKFZp547N157 |
| AB000520 | p < 1e−07 | APS\|adaptor protein with pleckstrin homology and src homology 2 domains |
| AW204447 | 0.0000005 | OATPRP4\|organic anion transporter polypeptide-related protein 4 |
| AJ420520 | 0.0000813 | MGC12335\|hypothetical protein MGC12335 |
| AF146796 | p < 1e−07 | SLC34A2\|solute carrier family 34 (sodium phosphate), member 2 |
| BC010659 | 0.0008625 | FLJ12691\|hypothetical protein FLJ12691 |
| AA732224 | p < 1e−07 | ESTs |
| AL136889 | p < 1e−07 | DKFZp434B227\|hypothetical protein DKFZp434B227 |
| AW193227 | p < 1e−07 | ESTs |
| AK024680 | 0.0000032 | PRO2714\|hypothetical protein PRO2714 |
| AW069753 | 0.000012 | CTGF\|connective tissue growth factor |
| NM_004532 | 0.0000654 | MUC4\|mucin 4, tracheobronchial |
| AF008203 | 0.0000116 | ALX3\|aristaless-like homeobox 3 |
| BF513361 | p < 1e−07 | MAP2\|microtubule-associated protein 2 |
| BF507741 | p < 1e−07 | AF15Q14\|AF15q14 protein |
| BC011587 | 0.0000108 | Homo sapiens, Similar to RIKEN cDNA 1700018O18 gene, clone IMAGE: 4121436, mRNA, partial cds |
| NM_002514 | 0.0000778 | NOV\|nephroblastoma overexpressed gene |
| AA573170 | 0.0000001 | EST |
| AK000520 | 0.0002189 | FLJ20513\|hypothetical protein FLJ20513 |
| AI459117 | p < 1e−07 | C6orf34\|chromosome 6 open reading frame 34 |
| AF011468 | p < 1e−07 | STK15\|serine/threonine kinase 15 |
| AI269345 | 0.0001152 | FLJ10851\|hypothetical protein FLJ10851 |
| AL050151 | p < 1e−07 | Homo sapiens mRNA; cDNA DKFZp586J0720 (from clone DKFZp586J0720) |
| AI042183 | 0.0000001 | TP53BP2\|tumor protein p53 binding protein, 2 |
| AI307682 | 0.000746 | IL18RAP\|interleukin 18 receptor accessory protein |
| AL136712 | 0.0000795 | KIAA0820\|KIAA0820 protein |
| BC000209 | p < 1e−07 | MGC2603\|hypothetical protein MGC2603 |
| AK026777 | 0.0000001 | KIAA1573\|KIAA1573 protein |
| AJ002367 | p < 1e−07 | SHOX2\|short stature homeobox 2 |
| AK025624 | 0.0000144 | MGC5350\|hypothetical protein MGC5350 |
| BC016047 | 0.0000063 | Homo sapiens, Similar to RIKEN cDNA 2310014B08 gene, clone IMAGE: 4639904, mRNA, partial cds |
| BC010904 | p < 1e−07 | FLJ11252\|hypothetical protein FLJ11252 |
| NM_004609 | 0.0000003 | TCF15\|transcription factor 15 (basic helix-loop-helix) |
| BC002654 | p < 1e−07 | TUBB-5\|tubulin beta-5 |
| NM_052813 | 0.0000001 | CARD9\|caspase recruitment domain family, member 9 |
| AA864938 | 0.0000005 | LALBA\|lactalbumin, alpha- |
| BC012136 | 0.0000022 | FLJ10160\|hypothetical protein FLJ10160 similar to insulin related protein 2 |
| BC001969 | 0.0000153 | ROBO1\|roundabout, axon guidance receptor, homolog 1 (Drosophila) |
| AL137736 | p < 1e−07 | Homo sapiens mRNA; cDNA DKFZp586P2321 (from clone DKFZp586P2321) |
| U64197 | 0.0000001 | SCYA20\|small inducible cytokine subfamily A (Cys—Cys), member 20 |
| BE465918 | 0.0000005 | VNN1\|vanin 1 |
| X78202 | 0.0001038 | FOXG1A\|forkhead box G1A |
| AF329454 | 0.0004222 | TMPRSS2\|transmembrane protease, serine 2 |
| BC011268 | 0.0000001 | KIAA1796\|KIAA1796 protein |
| BC013919 | 0.0000001 | TYMS\|thymidylate synthetase |
| AB058769 | 0.0003505 | KIAA1866\|KIAA1866 protein |
| BC005300 | 0.0004476 | DTNA\|dystrobrevin, alpha |
| AI199268 | 0.0000006 | Homo sapiens, Similar to RIKEN cDNA 2010317E24 gene, clone IMAGE: 3502019, mRNA, partial cds |
| X53587 | 0.000001 | ITGB4\|integrin, beta 4 |
| BF111883 | 0.0000093 | bA430M15.1\|novel protein (ortholog of rat four repeat ion channel) |
| AF035279 | 0.0000001 | IL15RA\|interleukin 15 receptor, alpha |
| NM_004936 | 0.0000147 | CDKN2B\|cyclin-dependent kinase inhibitor 2B (p15, inhibits CDK4) |
| AK000364 | 0.0000001 | KIAA1416\|KIAA1416 protein |
| AF281255 | 0.0000014 | BCLG\|apoptosis regulator BCL-G |
| AK026265 | 0.0000121 | FLJ22612\|hypothetical protein FLJ22612 |
| AA515100 | 0.0001378 | ESTs |
| AA461301 | 0.000014 | Homo sapiens nanos mRNA, partial cds |
| AF007133 | 0.000076 | SIAT1\|sialyltransferase 1 (beta-galactoside alpha-2,6-sialytransferase) |
| AF186780 | 0.0000009 | RGL\|RalGDS-like gene |
| AK026054 | 0.0000025 | Homo sapiens cDNA: FLJ22401 fis, clone HRC08032, highly similar to AB027466 Homo sapiens SPON2 mRNA |
| | | for spondin 2 |
| BC008191 | p < 1e−07 | PSCD3\|pleckstrin homology, Sec7 and coiled/coil domains 3 |
| BC002707 | 0.0000011 | SPON2\|spondin 2, extracellular matrix protein |
| BC001386 | 0.0000015 | TM4SF4\|transmembrane 4 superfamily member 4 |
| BC010120 | p < 1e−07 | RCN1\|reticulocalbin 1, EF-hand calcium binding domain |
| NM_001792 | 0.000069 | CDH2\|cadherin 2, type 1, N-cadherin (neuronal) |
| AL133061 | 0.0000001 | ATP11B\|ATPase, Class VI, type 11B |
| AF290004 | p < 1e−07 | TSA1902\|eosinophil chemotactic cytokine |
| AF019386 | 0.0000011 | HS3ST1\|heparan sulfate (glucosamine) 3-O-sulfotransferase 1 |
| BC017409 | 0.0002052 | Homo sapiens, clone IMAGE: 4332938, mRNA |
| AA747738 | 0.0000004 | STAG3\|stromal antigen 3 |
| AK000337 | 0.0000003 | FLJ20330\|hypothetical protein |
| AF081513 | p < 1e−07 | EBAF\|endometrial bleeding associated factor (left-right determination, factor A; transforming growth factor beta |
| | | superfamily) |
| AF131762 | 0.0000001 | Homo sapiens clone 25218 mRNA sequence |
| U97105 | 0.0000002 | DPYSL2\|dihydropyrimidinase-like 2 |
| AL137308 | 0.000041 | DKFZp761C07121\|hypothetical protein DKFZp761C07121 |
| AW139156 | 0.0000001 | CRMP5\|collapsin response mediator protein-5; CRMP3-associated molecule |
| M74297 | p < 1e−07 | HOXA4\|homeo box A4 |
| AK055386 | 0.0000751 | Homo sapiens cDNA FLJ30824 fis, clone FEBRA2001698 |
| AL096737 | p < 1e−07 | Homo sapiens mRNA; cDNA DKFZp434F152 (from clone DKFZp434F152) |
| AF267981 | 0.0000001 | DUOX2\|dual oxidase 2 |
| AF277289 | 0.0000005 | FLJ21162\|hypothetical protein FLJ21162 |
| BG655508 | 0.000001 | SATB1\|special AT-rich sequence binding protein 1 (binds to nuclear matrix/scaffold-associating DNA's) |
| BC001400 | p < 1e−07 | LOC57228\|hypothetical protein from clone 643 |
| BF195312 | 0.000023 | ESTs |
| BC018127 | 0.0000141 | PAM\|peptidylglycine alpha-amidating monooxygenase |
| N56968 | p < 1e−07 | C21orf37\|chromosome 21 open reading frame 37 |
| AI203899 | 0.0000001 | EPIM\|epimorphin |
| BC000117 | p < 1e−07 | GMDS\|GDP-mannose 4,6-dehydratase |
| AJ276678 | 0.0000001 | C20orf32\|chromosome 20 open reading frame 32 |
| AK056230 | 0.0000105 | Homo sapiens cDNA FLJ31668 fis, clone NT2RI2004916 |
| AL137461 | p < 1e−07 | Homo sapiens mRNA; cDNA DKFZp434K0621 (from clone DKFZp434K0621); partial cds |
| AF140242 | 0.0000048 | OPN3\|opsin 3 (encephalopsin, panopsin) |
| AF161508 | p < 1e−07 | HSPC159\|HSPC159 protein |
| AJ251830 | 0.0000059 | PIGPC1\|p53-induced protein PIGPC1 |
| BC002641 | 0.0000064 | KRT15\|keratin 15 |
| AF161505 | 0.000013 | HSPC156\|HSPC156 protein |
| AA923424 | 0.0001192 | ESTs, Weakly similar to I54374 gene NF2 protein H. sapiens |
| AL157432 | p < 1e−07 | TERA\|TERA protein |
| AK025494 | 0.0000003 | FLJ21841\|hypothetical protein FLJ21841 |
| AK022869 | 0.0000001 | Homo sapiens cDNA FLJ12807 fis, clone NT2RP2002316 |
| BC008765 | p < 1e−07 | SDC1\|syndecan 1 |
| AI383975 | 0.0000034 | SPRR2A\|small proline-rich protein 2A |
| BE328136 | 0.0000034 | DPYSL3\|dihydropyrimidinase-like 3 |
| AF106912 | 0.0000003 | WSX1\|class I cytokine receptor |
| BG054505 | p < 1e−07 | ESTs |
| BC008442 | p < 1e−07 | TM4SF1\|transmembrane 4 superfamily member 1 |
| BE045473 | p < 1e−07 | EST |
| R41498 | 0.0000002 | KIAA0231\|KIAA0231 protein |
| BC004277 | p < 1e−07 | FLJ10719\|hypothetical protein FLJ10719 |
| BF509349 | p < 1e−07 | Homo sapiens cDNA: FLJ23020 fis, clone LNG00943 |
| AA910884 | 0.0000202 | ESTs |
| AA810953 | 0.0000001 | BIC\|Homo sapiens BIC noncoding mRNA, complete sequence |
| M84757 | 0.0000668 | SPRR1B\|small proline-rich protein 1B (cornifin) |
| AL359605 | 0.0000752 | Homo sapiens mRNA; cDNA DKFZp547G036 (from clone DKFZp547G036) |
| L40377 | 0.0000001 | SERPINB8\|serine (or cysteine) proteinase inhibitor, clade B (ovalbumin), member 8 |
| AL136932 | 0.0000001 | KIAA0922\|KIAA0922 protein |
| BE348529 | 0.0001864 | ESTs, Weakly similar to SYN1 MOUSE SYNAPSIN I [M. musculus] |
| AI269387 | 0.0000001 | BAG2\|BCL2-associated athanogene 2 |
| BF434466 | 0.0000572 | ITGA6\|integrin, alpha 6 |
| AI251449 | 0.0000037 | ESTs |
| AF063020 | 0.0000071 | PSIP2\|PC4 and SFRS1 interacting protein 2 |
| AF285592 | p < 1e−07 | FTHL17\|ferritin, heavy polypeptide-like 17 |
| AL110124 | 0.0000017 | DKFZP564A2416\|DKFZP564A2416 protein |
| AA521246 | 0.0000054 | ESTs, Weakly similar to I38022 hypothetical protein H. sapiens |
| AA844695 | 0.0000014 | ESTs |
| AI566899 | 0.0000195 | NFIX\|nuclear factor I/X (CCAAT-binding transcription factor) |
| BE512918 | 0.0001109 | AGC1\|aggrecan 1 (chondroitin sulfate proteoglycan 1, large aggregating proteoglycan, antigen identified by |
| | | monoclonal antibody A0122) |
| L20971 | 0.0005197 | PDE4B\|phosphodiesterase 4B, cAMP-specific (phosphodiesterase E4 dunce homolog, Drosophila) |
| AL136778 | 0.0000001 | DKFZp434J0617\|hypothetical protein DKFZp434J0617 |
| D84212 | p < 1e−07 | STK6\|serine/threonine kinase 6 |
| S48196 | 0.0000007 | KAI1\|kangai 1 (suppression of tumorigenicity 6, prostate; CD82 antigen (R2 leukocyte antigen, antigen |
| | | detected by monoclonal and antibody IA4)) |
| AI743249 | p < 1e−07 | H_GS165L15.1\|cAMP response element-binding protein CRE-BPa |
| BC006376 | 0.0000001 | NMT2\|N-myristoyltransferase 2 |
| AK000140 | 0.0000629 | LOC51316\|hypothetical protein |
| AL117410 | 0.0000003 | SVIL\|supervillin |
| BC005313 | 0.0000218 | KLK1\|kallikrein 1, renal/pancreas/salivary |
| R42342 | 0.0000771 | EFNB2\|ephrin-B2 |
| BC017785 | 0.0000015 | PTPN22\|protein tyrosine phosphatase, non-receptor type 22 (lymphoid) |
| AF068918 | p < 1e−07 | BIN1\|bridging integrator 1 |
| BI792323 | 0.0000475 | LOC51186\|pp21 homolog |
| NM_004038 | 0.0000731 | AMY1A\|amylase, alpha 1A; salivary |
| AL035295 | 0.0000001 | H. sapiens gene from PAC 106H8, similar to Dynamin |
| NM_000935 | 0.0000159 | PLOD2\|procollagen-lysine, 2-oxoglutarate 5-dioxygenase (lysine hydroxylase) 2 |
| AB030000 | 0.0000805 | IL23A\|interleukin 23, alpha subunit p19 |
| AL049332 | p < 1e−07 | BTG3\|BTG family, member 3 |
| AI827546 | 0.0000001 | ESTs, Highly similar to I39451 amyloid-beta protein H. sapiens |
| BC007836 | 0.0000005 | MDFI\|MyoD family inhibitor |
| AI591051 | 0.0000002 | Homo sapiens, clone IMAGE: 4100953, mRNA |
| NM_001262 | 0.0000005 | CDKN2C\|cyclin-dependent kinase inhibitor 2C (p18, inhibits CDK4) |
| AA804433 | 0.0000182 | MX2\|myxovirus (influenza virus) resistance 2 (mouse) |
| BC017901 | 0.0000307 | Homo sapiens, Similar to phospholipid scramblase 1, clone MGC: 23806 IMAGE: 4253596, mRNA, complete cds |
| BC001459 | 0.0000001 | RAD51\|RAD51 homolog (RecA homolog, E. coli) (S. cerevisiae) |
| AF191771 | 0.0000084 | CED-6\|CED-6 protein |
| AW300933 | 0.0002531 | AQP9\|aquaporin 9 |
| AA731110 | 0.0000001 | LOC51185\|protein × 0001 |
| AA736984 | 0.0000004 | ADARB1\|adenosine deaminase, RNA-specific, B1 (RED1 homolog rat) |
| AI656385 | 0.000015 | SETBP1\|SET binding protein 1 |
| BC000612 | 0.0000021 | MGC2599\|hypothetical protein MGC2599 similar to katanin p60 subunit A 1 2599 |
| BC015940 | 0.0000202 | NT5\|5′ nucleotidase (CD73) |
| AK026589 | 0.0000043 | 2-Sep\|septin 6 |
| AL117485 | 0.0000865 | DKFZp434K191\|hypothetical protein DKFZp434K191 |
| AF161398 | 0.0000012 | PRO2013\|hypothetical protein PRO2013 |
| BC017498 | 0.0000018 | LOC65243\|hypothetical protein |
| BC008981 | 0.0000002 | MGC9341\|similar to RIKEN cDNA 4930500C14 gene |
| AI970276 | 0.0000007 | KIAA1676\|KIAA1676 |
| AA732062 | 0.0000008 | SIAT8D\|sialyltransferase 8D (alpha-2, 8-polysialytransferase) |
| BC014564 | 0.0000347 | MEST\|mesoderm specific transcript homolog (mouse) |
| BC003536 | 0.0006849 | MGC10796\|hypothetical protein MGC10796 |
| AA861036 | 0.0000009 | ESTs, Weakly similar to T33110 hypothetical protein C18H7.3 - Caenorhabditis elegans [C. elegans] |
| AF065857 | 0.0000272 | OR7E2P\|olfactory receptor, family 7, subfamily E, member 2 pseudogene |
| AF304051 | 0.0000001 | NDRG2\|N-myc downstream-regulated gene 2 |
| AL110298 | 0.0000045 | SLC2A3\|solute carrier family 2 (facilitated glucose transporter), member 3 |
| AI424406 | 0.0000004 | FLJ14033\|hypothetical protein FLJ14033 similar to hypoxia inducible factor 3, alpha subunit |
| BC009791 | 0.0000042 | HTN3\|histatin 3 |
| AL137686 | 0.0003622 | KIAA0855\|golgin-67 |
| NM_002451 | 0.0000014 | MTAP\|methylthioadenosine phosphorylase |
| BE673992 | 0.0000187 | ESTs |
| AL136612 | 0.0000417 | NCALD\|neurocalcin delta |
| BC001268 | 0.0000001 | NMI\|N-myc (and STAT) interactor |
| AF305083 | 0.0000048 | FUT4\|fucosyltransferase 4 (alpha (1,3) fucosyltransferase, myeloid-specific) |
| AJ420516 | 0.0000001 | Homo sapiens mRNA full length insert cDNA clone EUROIMAGE 966164 |
| AI805080 | 0.0000002 | EST |
| AA669114 | 0.0000008 | KIAA1842\|KIAA1842 protein |
| AI022925 | 0.0000167 | EMP1\|epithelial membrane protein 1 |
| NM_006271 | 0.0000516 | S100A1\|S100 calcium binding protein A1 |
| AK055576 | 0.0000001 | MPN\|marapsin |
| W46451 | 0.0000028 | LIF\|leukemia inhibitory factor (cholinergic differentiation factor) |
| AF217989 | 0.0000028 | PP1665\|hypothetical protein PP1665 |
| U66065 | 0.000005 | GRB10\|growth factor receptor-bound protein 10 |
| AI245337 | 0.0001557 | HSPB2\|heat shock 27 kD protein 2 |
| AI969251 | 0.0000054 | RAB7L1\|RAB7, member RAS oncogene family-like 1 |
| AW016075 | 0.0000125 | ESTs, Weakly similar to ALUA_HUMAN !!! ALU CLASS A WARNING ENTRY !!! H. sapiens |
| W60891 | 0.000027 | LGALS7\|lectin, galactoside-binding, soluble, 7 (galectin 7) |
| AF15382 | 0.0001128 | KCNJ2\|potassium inwardly-rectifying channel, subfamily J, member 2 |
| NM_006089 | 0.0001405 | SCML2\|sex comb on midleg-like 2 (Drosophila) |
| AI492884 | 0.0000558 | IRF4\|interferon regulatory factor 4 |
| NM_006308 | 0.0000187 | HSPB3\|heat shock 27 kD protein 3 |
| AI632869 | 0.00004 | UPK1B\|uroplakin 1B |
| AI821911 | 0.0000007 | ESTs |
| H06481 | 0.0000921 | KCNK9\|potassium channel, subfamily K, member 9 (TASK-3) |
| NM_001254 | 0.0000022 | CDC6\|CDC6 cell division cycle 6 homolog (S. cerevisiae) |
| BF446419 | 0.0000022 | LOC85414\|prostein protein |
| AL136570 | 0.000012 | LHX6\|LIM homeobox protein 6 |
| M62626 | 0.0000301 | HOX11\|homeo box 11 (T-cell lymphoma 3-associated breakpoint) |
| AK025796 | 0.0002692 | LOC55862\|uncharacterized hypothalamus protein HCDASE |
| BF513331 | 0.0001054 | ESTs, Weakly similar to S26689 hypothetical protein hc1 - mouse [M. musculus] |
| AL117592 | 0.0000001 | EMILIN-2\|extracellular glycoprotein EMILIN-2 precursor |
| BC007348 | 0.0000024 | UP\|uridine phosphorylase |
| AA761284 | 0.0000027 | FMR2\|fragile X mental retardation 2 |
| X70297 | 0.0000226 | CHRNA7\|cholinergic receptor, nicotinic, alpha polypeptide 7 |
| D87119 | 0.0000128 | GS3955\|GS3955 protein |
| AI128198 | 0.0000516 | ESTs |
| BC016330 | 0.0000018 | PIR51\|RAD51-interacting protein |
| BF510979 | 0.0000355 | DHDH\|dihydrodiol dehydrogenase (dimeric) |
| AI758745 | 0.0000001 | ESTs, Weakly similar to I38022 hypothetical protein H. sapiens |
| AI67849 | 0.0000008 | TBC1D1\|TBC1 (tre-2/USP6, BUB2, cdc16) domain family, member 1 |
| BC008729 | 0.0005722 | KIAA1309\|KIAA1309 protein |
| BC012328 | 0.0000048 | RPL39L\|ribosomal protein L39-like |
| AA588742 | 0.0000561 | XDH\|xanthene dehydrogenase |
| M99061 | 0.0001016 | KRT2A\|keratin 2A (epidermal ichthyosis bullosa of Siemens) |
| AK024822 | 0.0000003 | ELAC1\|elaC homolog 1 (E. coli) |
| BC011393 | 0.0000597 | CHN1\|chimerin (chimaerin) 1 |
| AI950844 | 0.0000171 | AIM1L\|absent in melanoma 1-like |
| BC007636 | 0.00001 94 | C20orf100\|chromosome 20 open reading frame 100 |
| BC017401 | 0.0000342 | SERPINB4\|serine (or cysteine) proteinase inhibitor, clade B (ovalbumin), member 4 |
| AF414120 | 0.0001959 | CTLA4\|cytotoxic T-lymphocyte-associated protein 4 |
| BC002776 | 0.000003 | SEMA5B\|sema domain, seven thrombospondin repeats (type 1 and type 1-like), transmembrane domain (TM) |
| | | and short cytoplasmic domain, (semaphorin) 5B |
| AW014507 | 0.0000143 | KIAA1858\|KIAA1858 protein |
| AI740744 | 0.0001645 | ESTs |
| X97859 | 0.0000085 | S100A12\|S100 calcium binding protein A12 (calgranulin C) |
| BC013982 | 0.0000365 | KIAA1921\|KIAA1921 protein |
| AK024896 | 0.0000017 | Homo sapiens cDNA: FLJ21243 fis, clone COL01164 |
| BC007079 | 0.0000158 | AF1Q\|ALL1-fused gene from chromosome 1q |
| BC007556 | 0.0000321 | Homo sapiens, Similar to TEA domain family member 2, clone MGC: 15481 IMAGE: 2967735, mRNA, complete |
| | | cds |
| AF011333 | 0.0000364 | LY75\|lymphocyte antigen 75 |
| M26683 | 0.0003079 | SCYA2\|small inducible cytokine A2 (monocyte chemotactic protein 1) |
| AW151360 | 0.0000384 | TNFSF13B\|tumor necrosis factor (ligand) superfamily, member 13b |
| AW131780 | 0.0005158 | NCAM1\|neural cell adhesion molecule 1 |
| BM126079 | 0.000573 | ESTs |
| AF316113 | 0.0000034 | GAL3ST-4\|beta-galactose-3-O-sulfotransferase, 4 |
| AF064254 | 0.0000111 | VLCS-H1\|VLCS-H1 protein |
| BC001492 | 0.0001032 | CNTFR\|ciliary neurotrophic factor receptor |
| AI200660 | 0.0000004 | ESTs, Weakly similar to T26845 hypothetical protein Y43F4B.7 - Caenorhabditis elegans [C. elegans] |
| NM_004137 | 0.0000019 | KCNMB1\|potassium large conductance calcium-activated channel, subfamily M, beta member 1 |
| AL110257 | 0.0000839 | Homo sapiens mRNA; cDNA DKFZp566P2346 (from clone DKFZp566P2346) |
| AI096369 | 0.0004741 | ESTs |
| AL133035 | 0.0000002 | DKFZp434G171\|hypothetical protein DKFZp434G171 |
| BM129291 | 0.0001921 | LMO1\|LIM domain only 1 (rhombotin 1) |
| AA731866 | 0.00026 | ESTs, Weakly similar to A45841 T-complex-associated-testes-expressed-1 protein - mouse [M. musculus] |
| AL039716 | 0.0000003 | CDC2L2\|cell division cycle 2-like 2 |
| AW137630 | 0.000012 | ESTs |
| AI283926 | 0.0000099 | CSPG4\|chondroitin sulfate proteoglycan 4 (melanoma-associated) |
| AI445650 | 0.000064 | PRF1\|perforin 1 (pore forming protein) |
| BC005861 | 0.0000175 | ITGB2\|integrin, beta 2 (antigen CD18 (p95), lymphocyte function-associated antigen 1; macrophage antigen 1 |
| | | (mac-1) beta subunit) |
| AI927378 | 0.0000334 | H. sapiens cDNA FLJ31267 fis, clone KIDNE2006053, moderately similar to M. musculus mRNA for |
| | | acetylglucosaminyltransferase-like protein |
| AI144219 | 0.0005353 | Human I-myc gene exon 3 and 3′-flanking region |
| AI268609 | 0.0000005 | ESPL1\|extra spindle poles like 1 (S. cerevisiae) |
| AF257182 | 0.0007686 | GPR48\|G protein-coupled receptor 48 |
| H17636 | 0.0000004 | GNAL\|guanine nucleotide binding protein (G protein), alpha activating activity polypeptide, olfactory type |
| BC015050 | 0.0000026 | OIP5\|Opa-interacting protein 5 |
| AF208043 | 0.0000872 | IFI16\|interferon, gamma-inducible protein 16 |
| AB042624 | 0.0001852 | SIRPB2\|signal-regulatory protein beta 2 |
| AW014836 | 0.0000065 | ESTs |
| BC013011 | 0.000118 | MGC2555\|hypothetical protein MGC2555 |
| R42108 | 0.0003179 | KIAA0378\|KIAA0378 protein |
| AI371090 | 0.0000082 | KIF3C\|kinesin family member 3C |
| BC004295 | 0.0000105 | PTRF\|polymerase\|and transcript release factor |
| AB017800 | 0.0004322 | NOL4\|nucleolar protein 4 |
| BC006458 | 0.000002 | CKS2\|CDC28 protein kinase 2 |
| AA883715 | 0.0000007 | ESTs |
| AF118088 | 0.0000086 | PRO2000\|PRO2000 protein |
| NM_005864 | 0.0000268 | EFS2\|signal transduction protein (SH3 containing) |
| BC009055 | 0.0001277 | FLJ13942\|hypothetical protein FLJ13942 |
| AF257238 | 0.0007241 | KIAA1634\|KIAA1634 protein |
| AW451039 | 0.0000006 | FLJ11808\|hypothetical protein FLJ11808 |
| AI910678 | 0.0000136 | WASF1\|WAS protein family, member 1 |
| AF286095 | 0.0004553 | IL22R\|interleukin 22 receptor |
| NM_002497 | 0.0000048 | NEK2\|NIMA (never in mitosis gene a)-related kinase 2 |
| NM_006142 | 0.000009 | SFN\|stratifin |
| AL136842 | 0.0002278 | CEP3\|Cdc42 effector protein 3 |
| AL049443 | 0.0000451 | Homo sapiens mRNA; cDNA DKFZp586N2020 (from clone DKFZp586N2020) |
| BC006000 | 0.0000675 | MGC12536\|hypothetical protein MGC12536 |
| AL133706 | 0.0001957 | ESTs |
| BF433809 | 0.0000769 | SCYC1\|small inducible cytokine subfamily C, member 1 (lymphotactin) |
| AK025953 | 0.0001653 | Homo sapiens cDNA: FLJ22300 fis, clone HRC04759 |
| AK025428 | 0.0000283 | KIAA0172\|KIAA0172 protein |
| BE858970 | 0.000055 | EST |
| AA827154 | 0.0003051 | PAG\|phosphoprotein associated with glycosphingolipid-enriched microdomains |
| AW512550 | 0.0004485 | ESTs |
| AA946776 | 0.0000011 | FGF9\|fibroblast growth factor 9 (glia-activating factor) |
| AF018253 | 0.0000509 | TNFRSF11A\|tumor necrosis factor receptor superfamily, member 11a, activator of NFKB |
| N32241 | 0.0003411 | Homo sapiens cDNA FLJ32214 fis, clone PLACE6003705 |
| AW293306 | 0.0001228 | K6HF\|cytokeratin type II |
| BC000141 | 0.0001528 | MYC\|v-myc myelocytomatosis viral oncogene homolog (avian) |
| AF009227 | 0.0007033 | NRG1\|neuregulin 1 |
| AI597863 | 0.0007186 | ESTs |
| AI659900 | 0.0003695 | EST |
| AW194680 | 0.0004206 | HOXD11\|homeo box D11 |
| AA868889 | 0.0000187 | RFPL3S\|ret finger protein-like 3 antisense |
| BC009352 | 0.0001185 | DKFZp762L0311\|hypothetical protein DKFZp762L0311 |
| AW291447 | 0.0001196 | CCR1\|chemokine (C—C motif) receptor 1 |
| AK002052 | 0.0005596 | C20orf46\|chromosome 20 open reading frame 46 |
| AI446519 | 0.0000451 | ESTs |
| NM_000734 | 0.0000771 | CD3Z\|CD3Z antigen, zeta polypeptide (TiT3 complex) |
| NM_032968 | 0.0000017 | PCDH11\|protocadherin 11 |
| BF509016 | 0.0000177 | Homo sapiens cDNA FLJ30899 fis, clone FEBRA2005726 |
| M93426 | 0.0000025 | PTPRZ1\|protein tyrosine phosphatase, receptor-type, Z polypeptide 1 |
| AK026663 | 0.0000204 | FLJ21034\|hypothetical protein FLJ21034 |
| BF433635 | 0.0000216 | ESTs |
| AF104313 | 0.0002268 | ESDN\|endothelial and smooth muscle cell-derived neuropilin-like protein |
| BC000356 | 0.0000032 | MAD2L1\|MAD2 mitotic arrest deficient-like 1 (yeast) |
| AI625086 | 0.0000526 | FLJ11585\|hypothetical protein FLJ11585 |
| AI560377 | 0.0003146 | ESTs |
| BF112041 | 0.0000027 | KIAA0401\|KIAA0401 protein |
| AI831078 | 0.0003873 | ESTs, Weakly similar to CA18 MOUSE COLLAGEN ALPHA 1(VIII) CHAIN PRECURSOR [M. musculus] |
| AF007194 | 0.0000116 | MUC3A\|mucin 3A, intestinal |
| NM_000436 | 0.0000291 | OXCT\|3-oxoacid CoA transferase |
| BC007828 | 0.0000485 | MGC14128\|hypothetical protein MGC14128 |
| AF396928 | 0.000446 | EPSTI1\|epithelial stromal interaction 1 (breast) |
| U43203 | 0.0004703 | TITF1\|thyroid transcription factor 1 |
| BC000021 | 0.0002624 | GFPT2\|glutamine-fructose-6-phosphate transaminase 2 |
| AF047711 | 0.0000029 | RASAL2\|RAS protein activator like 2 |
| AF053453 | 0.0001192 | TM4SF6\|transmembrane 4 superfamily member 6 |
| BC017054 | 0.0000027 | MTHFD2\|methylene tetrahydrofolate dehydrogenase (NAD+ dependent), methenyltetrahydrofolate |
| | | cyclohydrolase |
| AI769930 | 0.0000068 | Homo sapiens (clone B3B3E13) Huntington's disease candidate region mRNA fragment |
| BC002914 | 0.0000453 | WASPIP\|Wiskott-Aldrich syndrome protein interacting protein |
| BE671403 | 0.0004777 | ESTs |
| AI249782 | 0.0001099 | PPP4R2\|protein phosphatase 4 regulatory subunit 2 |
| AJ420537 | 0.0000104 | dJ55C23.6\|dJ55C23.6 gene |
| AI239743 | 0.0001411 | LTA\|lymphotoxin alpha (TNF superfamily, member 1) |
| NM_001890 | 0.0003456 | CSN1\|casein, alpha |
| AF303058 | 0.0000457 | NP25\|neuronal protein |
| BC004336 | 0.0000035 | MGC10946\|hypothetical protein MGC10946 |
| AI590055 | 0.0000213 | ESTs |
| AW301017 | 0.0001513 | ESTs |
| BC015497 | 0.0000283 | TEAD4\|TEA domain family member 4 |
| BC008483 | 0.0000566 | SERPINB3 serine (or cysteine) proteinase inhibitor, clade B (ovalbumin), member 3 |
| BC007072 | 0.0000858 | MGC12538\|hypothetical protein MGC12538 |
| AI384087 | 0.0001257 | KIAA0603\|KIAA0603 gene product |
| AB023135 | 0.000755 | ICOS\|inducible T-cell co-stimulator |
| AB023967 | 0.0000858 | ROD1\|ROD1 regulator of differentiation 1 (S. pombe) |
| AI560576 | 0.0006927 | PTPRK\|protein tyrosine phosphatase, receptor type, K |
| AK024951 | 0.0000497 | C1R\|complement component 1, r subcomponent |
| AW452231 | 0.0004972 | CD244\|natural killer cell receptor 2B4 |
| AK027515 | 0.0000623 | Homo sapiens cDNA FLJ14609 fis, clone NT2RP1000944 |
| AL043170 | 0.0001136 | Homo sapiens colon cancer antigen NY-CO-45 mRNA, partial cds |
| AK025636 | 0.0001079 | Homo sapiens cDNA: FLJ21983 fis, clone HEP06219 |
| NM_006278 | 0.0000121 | SIAT4C\|sialyltransferase 4C (beta-galactosidase alpha-2,3-sialytransferase) |
| BC003388 | 0.0001059 | TANK\|TRAF family member-associated NFKB activator |
| AA777592 | 0.0005802 | ESTs, Weakly similar to ubiquitous TPR motif, Y isoform H. sapiens |
| BC016623 | 0.0000111 | ETV4\|ets variant gene 4 (E1A enhancer binding protein, E1AF) |
| BC005408 | 0.0000236 | FLJ23119\|hypothetical protein FLJ23119 |
| AF233588 | 0.0001101 | LOC51285\|Ris |
| BC018644 | 0.0001366 | NUDT8\|nudix (nucleoside diphosphate linked moiety X)-type motif 8 |
| AW057670 | 0.0000078 | ESTs, Weakly similar to S65824 reverse transcriptase homolog H. sapiens |
| AF414088 | 0.000153 | COL21A1\|collagen, type XXI, alpha 1 |
| X96757 | 0.0001749 | MAP2K6\|mitogen-activated protein kinase kinase 6 |
| NM_005965 | 0.0002044 | MYLK\|myosin, light polypeptide kinase |
| R40207 | 0.00041 | PIP5K1B\|phosphatidylinositol-4-phosphate 5-kinase, type I, beta |
| AK026315 | 0.0005265 | FLJ22662\|hypothetical protein FLJ22662 |
| AK025130 | 0.0000064 | FLJ21477\|hypothetical protein FLJ21477 |
| BC014444 | 0.0000298 | ASML3B\|acid sphingomyelinase-like phosphodiesterase |
| AJ012214 | 0.0000751 | 11-Oct\|POU transcription factor |
| AL137343 | 0.0000089 | Homo sapiens mRNA for putative NSE1 protein |
| AK024855 | 0.0000346 | CTSS\|cathepsin S |
| BC002727 | 0.0001627 | SDCBP2\|syndecan binding protein (syntenin) 2 |
| AK026337 | 0.0000268 | FLJ22684\|hypothetical protein FLJ22684 |
| AK025346 | 0.0009842 | Homo sapiens cDNA: FLJ21693 fis, clone COL09609 |
| AI536895 | 0.0001916 | LOX\|lysyl oxidase |
| AF339783 | 0.0004293 | FLJ20159\|hypothetical protein FLJ20159 |
| AB038781 | 0.0000178 | MUC3B\|mucin 3B |
| BC011969 | 0.0001809 | Homo sapiens, clone IMAGE: 3880654, mRNA |
| AF326964 | 0.0001063 | BCL2L10\|BCL2-like 10 (apoptosis facilitator) |
| AF033347 | 0.0004868 | KCNQ3\|potassium voltage-gated channel, KQT-like subfamily, member 3 |
| AF070631 | 0.0000349 | TMPO\|thymopoietin |
| BC015507 | 0.000679 | CST7\|cystatin F (leukocystatin) |
| AI811072 | 0.000041 | VGF\|VGF nerve growth factor inducible |
| BC006346 | 0.000113 | APEG1\|nuclear protein, marker for differentiated aortic smooth muscle and down-regulated with vascular injury |
| AF247167 | 0.0001179 | AD031\|AD031 protein |
| L27711 | 0.0001896 | CDKN3\|cyclin-dependent kinase inhibitor 3 (CDK2-associated dual specificity phosphatase) |
| AA810577 | 0.0000119 | ARSB\|arylsulfatase B |
| AF328296 | 0.0000516 | FLJ14437\|myopalladin |
| AI147926 | 0.0001123 | CSF2RB\|colony stimulating factor 2 receptor, beta, low-affinity (granulocyte-macrophage) |
| AF017988 | 0.00017 | SFRP5\|secreted frizzled-related protein 5 |
| AI151185 | 0.0000495 | LHPP\|phospholysine phosphohistidine inorganic pyrophosphate phosphatase |
| AW271272 | 0.0000564 | FOXC2\|forkhead box C2 (MFH-1, mesenchyme forkhead 1) |
| BI324932 | 0.0000637 | PTGER4\|prostaglandin E receptor 4 (subtype EP4) |
| AF105378 | 0.00025 | HS3ST4\|heparan sulfate (glucosamine) 3-O-sulfotransferase 4 |
| BC009642 | 0.0000406 | FXYD5\|FXYD domain-containing ion transport regulator 5 |
| BF002341 | 0.0000493 | ESTs, Moderately similar to AF174605 1 F-box protein Fbx2S H. sapiens |
| H06160 | 0.0001828 | C1orf17\|chromosome 1 open reading frame 17 |
| NM_001839 | 0.0000549 | CNN3\|calponin 3, acidic |
| BC015397 | 0.0001164 | Homo sapiens, Similar to RIKEN cDNA 4921507P07 gene, clone MGC: 21710 IMAGE: 4448480, mRNA, |
| | | complete cds |
| AK026144 | 0.0006923 | TEL2\|transcription factor ets |
| BC008749 | 0.0000146 | MBP\|myelin basic protein |
| BC007678 | 0.0000353 | ADA\|adenosine deaminase |
| AA814043 | 0.0004392 | ESTs |
| AW291196 | 0.0001248 | ESTs |
| AI090561 | 0.0005695 | M160\|scavenger receptor cysteine-rich type 1 protein M160 precursor |
| AF098066 | 0.0000203 | SART-2\|squamous cell carcinoma antigen recognized by T cell |
| AK025615 | 0.0001353 | Homo sapiens cDNA: FLJ21962 fis, clone HEP05564 |
| AU152414 | 0.0004928 | DNAH11\|dynein, axonemal, heavy polypeptide 11 |
| U49395 | 0.0006226 | P2RX5\|purinergic receptor P2X, ligand-gated ion channel, 5 |
| AI611312 | 0.0002789 | KIAA0942\|KIAA0942 protein |
| AI689069 | 0.0001012 | CORO1A\|coronin, actin binding protein, 1A |
| AF117386 | 0.0000326 | USP1\|ubiquitin specific protease 1 |
| NM_001452 | 0.0001101 | FOXF2\|forkhead box F2 |
| AI025919 | 0.0000271 | ESTs |
| AF225416 | 0.0000275 | AD024\|AD024 protein |
| AA910331 | 0.0006572 | COL4A1\|collagen, type IV, alpha 1 |
| AA743321 | 0.0000364 | ESTs |
| AI290556 | 0.0000413 | ESTs |
| BE676183 | 0.0002021 | KIAA1798\|KIAA1798 protein |
| AA831508 | 0.0008458 | ESTs |
| BC012945 | 0.0000572 | MGC11271\|hypothetical protein MGC11271 |
| BC004278 | 0.0000617 | FLJ23239\|hypothetical protein FLJ23239 |
| BC004267 | 0.0001122 | MGC10818\|hypothetical protein MGC10818 |
| AW449550 | 0.0001866 | GAP1IP4BP\|RAS p21 protein activator (GTPase activating protein) 3 (lns(1,3,4,5)P4-binding protein) |
| BC000049 | 0.0002093 | FLJ10493\|hypothetical protein FLJ10493 |
| R56080 | 0.0003624 | RELN\|reelin |
| AF039655 | 0.0000292 | BFSP1\|beaded filament structural protein 1, filensin |
| AI656630 | 0.0002032 | TMEM7\|transmembrane protein 7 |
| AW411170 | 0.0003073 | EAF1\|EAF1 protein |
| AI280476 | 0.0004985 | APG-1\|heat shock protein (hsp110 family) |
| BF510271 | 0.0000551 | LOC51063\|hypothetical protein |
| BC007533 | 0.0001578 | NRP1\|neuropilin 1 |
| AI671448 | 0.00021 | ESTs |
| U62824 | 0.0009236 | H. sapiens mRNA for HLA-C alpha chain (Cw*1701) |
| AF025887 | 0.0000808 | GSTA4\|glutathione 5-transferase A4 |
| BF513346 | 0.0004176 | ESTs |
| AW452418 | 0.0006057 | RYR2\|ryanodine receptor 2 (cardiac) |
| AL117401 | 0.0006587 | DKFZP434P211\|DKFZp434P211 protein |
| AF324830 | 0.0007875 | ILT11\|leukocyte immunoglobulin-like receptor, subfamily B (with TM and ITIM domains), member 7 |
| BC004291 | 0.0000444 | KIAA0080\|KIAA0080 protein |
| AF223943 | 0.0000452 | GLS\|glutaminase |
| AK000626 | 0.000305 | FLJ20619\|hypothetical protein FLJ20619 |
| AI766890 | 0.0008599 | ESTs, Weakly similar to NAH6_HUMAN SODIUM/HYDROGEN EXCHANGER 6 H. sapiens |
| BM141806 | 0.0002858 | Homo sapiens cDNA FLJ30865 fis, clone FEBRA2004099 |
| Y13786 | 0.0001617 | ADAM19\|a disintegrin and metalloproteinase domain 19 (meltrin beta) |
| BF508503 | 0.0000643 | Homo sapiens cDNA FLJ32102 fis, clone OCBBF2001196 |
| AW073971 | 0.0000986 | ESTs, Weakly similar to T00705 N-chimerin homolog F25965_3 H. sapiens |
| M87507 | 0.0002201 | CASP1\|caspase 1, apoptosis-related cysteine protease (interleukin 1, beta, convertase) |
| AJ315514 | 0.000686 | NR3C2\|nuclear receptor subfamily 3, group C, member 2 |
| AI240521 | 0.0002527 | CHML\|choroideremia-like (Rab escort protein 2) |
| NM_057749 | 0.0003057 | CCNE2\|cyclin E2 |
| BC012113 | 0.000344 | HOMER-3\|Homer, neuronal immediate early gene, 3 |
| BE551149 | 0.0005021 | ESTs |
| AW081673 | 0.0000873 | SAH\|SA hypertension-associated homolog (rat) |
| AB016901 | 0.0001215 | HGC6.2\|HGC6.2 protein |
| BC006093 | 0.0001312 | MMP9\|matrix metalloproteinase 9 (gelatinase B, 92 kD gelatinase, 92 kD type IV collagenase) |
| NM_000795 | 0.000318 | DRD2\|dopamine receptor D2 |
| AA630788 | 0.000387 | TOP2A\|topoisomerase (DNA) II alpha (170 kD) |
| AL137274 | 0.000862 | MIG-6\|Gene 33/Mig-6 |
| U89995 | 0.0001892 | FOXE1\|forkhead box E1 (thyroid transcription factor 2) |
| M85294 | 0.0002409 | PRKR\|protein kinase, interferon-inducible double stranded RNA dependent |
| BC003097 | 0.0000678 | ICAM2\|intercellular adhesion molecule 2 |
| AL137346 | 0.0000816 | Homo sapiens mRNA; cDNA DKFZp761M0111 (from clone DKFZp761M0111) |
| AK027138 | 0.0000856 | KIAA0914\|KIAA0914 gene product |
| BC009628 | 0.0000828 | Homo sapiens, clone IMAGE: 3898148, mRNA, partial cds |
| AB037715 | 0.0002083 | FLJ10210\|hypothetical protein FLJ10210 |
| AW084130 | 0.0008664 | NOS3\|nitric oxide synthase 3 (endothelial cell) |
| AA807955 | 0.0002328 | EST |
| AK024697 | 0.0007384 | FLJ21044\|hypothetical protein FLJ21044 similar to Rbig1 |
| AL137662 | 0.0002524 | Homo sapiens mRNA; cDNA DKFZp434P086 (from clone DKFZp434P086); partial cds |
| AI732321 | 0.0001257 | ESTs |
| AL137506 | 0.0007917 | FLJ23563\|hypothetical protein FLJ23563 |
| R43523 | 0.0005701 | FLJ22202 hypothetical protein FLJ22202 |
| AI869243 | 0.0003403 | ESTs |
| AW291368 | 0.0002584 | ESTs, Weakly similar to KIAA0738 protein H. sapiens |
| BC001350 | 0.0009786 | ARG2\|arginase, type II |
| AK022735 | 0.0001058 | FLJ12673\|hypothetical protein FLJ12673 |
| BF510331 | 0.0001294 | MCM3AP\|MCM3 minichromosome maintenance deficient 3 (S. cerevisiae) associated protein |
| AL136703 | 0.0004855 | DKFZP566J091\|hypothetical protein DKFZp566J091 |
| AI953054 | 0.0001233 | TKT\|transketolase (Wernicke-Korsakoff syndrome) |
| BC014317 | 0.0002587 | NX-17\|kidney-specific membrane protein |
| BC014890 | 0.0004285 | SLUG\|slug homolog, zinc finger protein (chicken) |
| BC001645 | 0.000697 | SYK\|spleen tyrosine kinase |
| AI188578 | 0.0002173 | FLJ11036\|hypothetical protein FLJ11036 |
| AI733497 | 0.0005164 | ESTs |
| AW293496 | 0.00068 | ESTs |
| AF269223 | 0.0005681 | TCP11\|t-complex 11 (mouse) |
| AB012643 | 0.0006384 | ALPL\|alkaline phosphatase, liver/bone/kidney |
| U75309 | 0.0006677 | TAF5\|TAF5 RNA polymerase II, TATA box binding protein (TBP)-associated factor, 100 kD |
| NM_015901 | 0.0001783 | LOC51055\|unknown |
| AK021801 | 0.0002326 | Homo sapiens cDNA FLJ11739 fis, clone HEMBA1005497 |
| AW295061 | 0.0002448 | WSX1\|class I cytokine receptor |
| AI559983 | 0.0004987 | CDC2\|cell division cycle 2, G1 to S and G2 to M |
| BC015496 | 0.000254 | INPP1\|inositol polyphosphate-1-phosphatase |
| X93595 | 0.0002125 | KIR3DL2\|killer cell immunoglobulin-like receptor, three domains, long cytoplasmic tail, 2 |
| AI765908 | 0.0005868 | Homo sapiens cDNA FLJ30999 fis, clone HLUNG1000110, weakly similar to PISTIL-SPECIFIC EXTENSIN-LIKE |
| | | PROTEIN PRECURSOR |
| NM_005923 | 0.00086 | MAP3K5\|mitogen-activated protein kinase kinase kinase 5 |
| BC016757 | 0.0002739 | SAP30\|sin3-associated polypeptide, 30 kD |
| AK027121 | 0.0002624 | FLJ23468\|hypothetical protein FLJ23468 |
| BE644686 | 0.0005227 | KIAA0509\|KIAA0509 protein |
| AI418207 | 0.0007707 | ESTs |
| BC009198 | 0.0004215 | Homo sapiens, Similar to RNA polymerase I transcription factor RRN3, clone MGC: 15321 IMAGE: 3678732, |
| | | mRNA, complete cds |
| AA761502 | 0.0005644 | KIAA0009\|KIAA0009 gene product |
| BC018112 | 0.0005867 | PROL2\|proline rich 2 |
| BC002718 | 0.000673 | FN14\|type I transmembrane protein Fn14 |
| AL353948 | 0.0009563 | Homo sapiens mRNA; cDNA DKFZp761P0114 (from clone DKFZp761P0114) |
| AK023806 | 0.0009698 | FLJ13744\|hypothetical protein FLJ13744 |
| AI264615 | 0.0007686 | SIRT4\|sirtuin silent mating type information regulation 2 homolog 4 (S. cerevisiae) |
| BC002715 | 0.0007069 | PPARD\|peroxisome proliferative activated receptor, delta |
| M96843 | 0.0007716 | ID2B\|striated muscle contraction regulatory protein |
| BC007289 | 0.0008457 | ARPM1\|actin related protein M1 |
| AI338498 | 0.0008464 | ZNF295\|zinc finger protein 295 |
| W46976 | 0.0009251 | EST |

| GenBank | | |
|---|---|---|
| accession | CloneID | UnigeneID |
| AW473119 | 725321 | Hs.1657 |
| AW015443 | 841621 | Hs.100686 |
| NM_001218 | 594633 | Hs.5338 |
| AF131785 | 2284924 | Hs.90419 |
| AJ009985 | 239568 | Hs.279928 |
| AF088867 | 2321113 | Hs.91011 |
| AF007153 | 726703 | Hs.7888 |
| BC014948 | 1558642 | Hs.321130 |
| AA542898 | 782688 | Hs.33846 |
| AA569340 | 1631209 | Hs.330780 |
| AW137092 | 1554430 | Hs.94694 |
| NM_005080 | 725968 | Hs.149923 |
| BC006793 | 214068 | Hs.169946 |
| NM_004496 | 1610546 | Hs.299867 |
| U95089 | 137017 | Hs.77432 |
| U95089 | 183468 | Hs.77432 |
| AJ404611 | 1584563 | Hs.130881 |
| AI128582 | 1691237 | Hs.190642 |
| NM_020974 | 242778 | Hs.222399 |
| AI332979 | 2321529 | Hs.118695 |
| AA688021 | 111389 | Hs.179808 |
| AI820662 | 1607198 | Hs.129598 |
| AI125908 | 51185 | Hs.125019 |
| AK026818 | 69378 | Hs.279898 |
| BC014962 | 1700429 | Hs.105445 |
| AW024203 | 530197 | Hs.349344 |
| AL137566 | 504959 | Hs.32405 |
| AI874215 | 299332 | Hs.193914 |
| BC013732 | 66599 | Hs.155956 |
| R41823 | 1558233 | Hs.7413 |
| AK000442 | 1562618 | Hs.11408 |
| AK025256 | 1632235 | Hs.129691 |
| NM_006763 | 213136 | Hs.75462 |
| NM_006763 | 71428 | Hs.75462 |
| NM_001238 | 68950 | Hs.9700 |
| BC011348 | 785795 | Hs.15929 |
| N31940 | 866488 | Hs.14434 |
| BC014601 | 839382 | Hs.88417 |
| AF356492 | 431003 | Hs.63931 |
| BC012375 | 179212 | Hs.279937 |
| BC012375 | 767289 | Hs.279937 |
| NM_000044 | 2250839 | Hs.99915 |
| AI823785 | 782497 | Hs.6349 |
| BC012914 | 592832 | Hs.180059 |
| AF084830 | 1901562 | Hs.127007 |
| AI637899 | 2237353 | Hs.1907 |
| AL110126 | 813265 | Hs.326416 |
| AF033021 | 756708 | Hs.10082 |
| AA749341 | 814054 | Hs.158282 |
| BC000558 | 647397 | Hs.101174 |
| AI364725 | 809779 | Hs.9729 |
| X70340 | 1553998 | Hs.170009 |
| AI471793 | 415550 | Hs.60257 |
| BC017701 | 823655 | Hs.21941 |
| NM_014599 | 730288 | Hs.55058 |
| AA659816 | 795851 | Hs.23830 |
| AA622986 | 731047 | Hs.104830 |
| AI871614 | 344959 | Hs.58241 |
| AK026946 | 220395 | Hs.27099 |
| AA761512 | 783698 | Hs.81412 |
| AL136579 | 219929 | Hs.109315 |
| AW574736 | 261541 | Hs.143460 |
| AI337740 | 41569 | Hs.4243 |
| AI382972 | 773170 | Hs.82128 |
| AJ249248 | 685516 | Hs.97101 |
| BC013300 | 531319 | Hs.180655 |
| AA587049 | 2494805 | Hs.211587 |
| NM_005044 | 1869486 | Hs.147996 |
| NM_005044 | 310034 | Hs.147996 |
| NM_001502 | 1412344 | Hs.53985 |
| AW204371 | 293689 | Hs.239727 |
| AB023430 | 2019426 | Hs.156114 |
| AW136298 | 2449841 | Hs.3185 |
| AL117452 | 767068 | Hs.44155 |
| NM_014211 | 563598 | Hs.70725 |
| AA845338 | 197525 | Hs.14286 |
| AI741657 | 683569 | Hs.198443 |
| AW195038 | 2017930 | Hs.129867 |
| AK000012 | 1517212 | Hs.184634 |
| BF434467 | 82869 | Hs.11805 |
| BC005395 | 1854648 | Hs.346935 |
| BC005395 | 85488 | Hs.346935 |
| AA767129 | 310034 | Hs.56336 |
| AB022918 | 272706 | Hs.34578 |
| AA648922 | 127510 | Hs.1634 |
| AF026477 | 1536215 | Hs.6079 |
| BC001283 | 416959 | Hs.33287 |
| NM_005401 | 2390593 | Hs.159238 |
| NM_007050 | 41647 | Hs.225952 |
| BC003610 | 785744 | Hs.3745 |
| AI631667 | 344073 | Hs.351344 |
| S72493 | 1486533 | Hs.115947 |
| AF193809 | 1456468 | Hs.279682 |
| BC012107 | 391949 | Hs.103527 |
| X83301 | 470261 | Hs.324728 |
| BC002738 | 1323448 | Hs.17409 |
| BE464993 | 2062533 | Hs.112158 |
| BG272534 | 289505 | Hs.44829 |
| R50991 | 1586124 | Hs.12247 |
| AF117108 | 429494 | Hs.79440 |
| AL157505 | 32050 | Hs.21380 |
| N22687 | 266500 | Hs.8236 |
| AJ278018 | 1637767 | Hs.12079 |
| AW294857 | 25274 | Hs.21050 |
| AI804716 | 544639 | Hs.95612 |
| NM_004923 | 1518890 | Hs.121378 |
| AA177001 | 809674 | Hs.112184 |
| BC009722 | 1637893 | Hs.105887 |
| BC000703 | 292936 | Hs.48855 |
| Z73678 | 1662279 | Hs.198382 |
| BC007595 | 1558675 | Hs.44317 |
| BC009203 | 212456 | Hs.25925 |
| AF132818 | 132711 | Hs.84728 |
| AF132818 | 2062507 | Hs.84728 |
| BF110928 | 1556859 | Hs.126932 |
| BM126815 | 1753085 | Hs.316433 |
| AY048757 | 23819 | Hs.10237 |
| AK026420 | 1161564 | Hs.10587 |
| AI567843 | 51078 | Hs.349090 |
| AK025562 | 418049 | Hs.18612 |
| AL080079 | 786695 | Hs.44197 |
| BC002660 | 723986 | Hs.170453 |
| BC008764 | 742798 | Hs.69360 |
| AL137311 | 281392 | Hs.234074 |
| BI493178 | 415191 | Hs.78894 |
| AF181862 | 2016775 | Hs.242407 |
| BC010039 | 144849 | Hs.289092 |
| BE326342 | 155532 | Hs.129043 |
| NM_004419 | 342378 | Hs.2128 |
| BC000881 | 2017415 | Hs.1594 |
| AI498405 | 754346 | Hs.34145 |
| AI636974 | 811774 | Hs.238126 |
| AI494546 | 307255 | Hs.10649 |
| AJ420562 | 26883 | Hs.106106 |
| BC015338 | 2063284 | Hs.180884 |
| AL050227 | 784178 | Hs.27860 |
| BF433019 | 950355 | Hs.110080 |
| AK025296 | 204790 | Hs.95549 |
| AA088779 | 1635970 | Hs.24724 |
| AA846278 | 51700 | Hs.5740 |
| BI495988 | 626841 | Hs.285754 |
| BI495988 | 754509 | Hs.285754 |
| AI150439 | 208387 | Hs.15370 |
| AI732309 | 153760 | Hs.78436 |
| BC006526 | 810873 | Hs.2794 |
| AF035282 | 293569 | Hs.12532 |
| AF089854 | 1947911 | Hs.8022 |
| AA714213 | 66406 | Hs.352002 |
| AY007114 | 296679 | Hs.44898 |
| N89991 | 772962 | Hs.44070 |
| BC000576 | 23776 | Hs.75438 |
| BE672557 | 282980 | Hs.42640 |
| BC015525 | 809784 | Hs.79361 |
| AL136539 | 768068 | Hs.169333 |
| BC009742 | 121551 | Hs.18141 |
| BC017338 | 308437 | Hs.576 |
| D50840 | 812196 | Hs.152601 |
| BC017575 | 246524 | Hs.20295 |
| BC012089 | 27544 | Hs.112360 |
| AJ420473 | 797038 | Hs.7869 |
| M57892 | 823718 | Hs.100322 |
| AI436812 | 1553560 | Hs.143640 |
| M57609 | 767447 | Hs.72916 |
| M57609 | 767495 | Hs.72916 |
| AI797041 | 382787 | Hs.182231 |
| AI741143 | 767164 | Hs.235857 |
| BC001766 | 759948 | Hs.83384 |
| BC009945 | 813697 | Hs.49500 |
| BC014851 | 40887 | Hs.159142 |
| AF039843 | 813698 | Hs.18676 |
| AK025000 | 782787 | Hs.103147 |
| AL133033 | 785710 | Hs.4084 |
| AF053305 | 781047 | Hs.98658 |
| AI207256 | 752837 | Hs.13766 |
| AW780208 | 825343 | Hs.38516 |
| D25538 | 2115545 | Hs.172199 |
| BG939593 | 415769 | Hs.2463 |
| AI557322 | 186965 | Hs.27342 |
| AK000532 | 1457205 | Hs.98491 |
| BC011976 | 323238 | Hs.789 |
| AA648777 | 79576 | Hs.11090 |
| BC004199 | 1618987 | Hs.1933 |
| AW450693 | 782199 | Hs.104258 |
| BC002668 | 951125 | Hs.15250 |
| AI740531 | 741429 | Hs.269222 |
| AA181334 | 611532 | Hs.83760 |
| BC010607 | 428184 | Hs.104679 |
| NM_001604 | 230882 | Hs.89506 |
| BC017730 | 1534888 | Hs.159651 |
| AW070918 | 358936 | Hs.323445 |
| BC004161 | 591157 | Hs.95583 |
| AF141339 | 796767 | Hs.26799 |
| BC007318 | 133518 | Hs.78335 |
| AW083384 | 79726 | Hs.11067 |
| BC010078 | 1519143 | Hs.106747 |
| BC012432 | 1603358 | Hs.166994 |
| BC016615 | 1492587 | Hs.147066 |
| N47717 | 2012523 | Hs.153179 |
| BC008962 | 788832 | Hs.296638 |
| AL136939 | 839081 | Hs.250175 |
| BF446757 | 470348 | Hs.31841 |
| AI885044 | 265060 | Hs.81665 |
| AF230904 | 1604005 | Hs.153260 |
| NM_002639 | 2499237 | Hs.55279 |
| M59305 | 1762111 | Hs.123655 |
| AJ010277 | 2273365 | Hs.50403 |
| AJ276230 | 745007 | Hs.82587 |
| AL080222 | 782306 | Hs.7358 |
| AA262504 | 685912 | Hs.46925 |
| BC008947 | 504308 | Hs.14559 |
| BC014039 | 1517595 | Hs.184339 |
| BC001068 | 200402 | Hs.70704 |
| BC011705 | 361204 | Hs.53563 |
| BC011705 | 897646 | Hs.53563 |
| AF131784 | 155227 | Hs.25318 |
| AF131784 | 25194 | Hs.25318 |
| AF136631 | 140197 | Hs.103291 |
| NM_001237 | 814270 | Hs.85137 |
| NM_001237 | 950690 | Hs.85137 |
| AW070746 | 181877 | Hs.177691 |
| R74353 | 202577 | Hs.81182 |
| AI538937 | 1708335 | Hs.1573 |
| NM_022977 | 858167 | Hs.81452 |
| BC016308 | 1556433 | Hs.89690 |
| AK055431 | 768246 | Hs.349611 |
| AW007851 | 2009779 | Hs.326056 |
| BC007407 | 2306697 | Hs.83321 |
| BC013591 | 143661 | Hs.102541 |
| AL122077 | 2291557 | Hs.144672 |
| AF123074 | 43865 | Hs.65248 |
| AI031941 | 136954 | Hs.25119 |
| BC016040 | 1897302 | Hs.120996 |
| X59770 | 233583 | Hs.25333 |
| BC008703 | 1898619 | Hs.39122 |
| BC009581 | 1576468 | Hs.155109 |
| AI275604 | 268188 | Hs.40637 |
| R05446 | 241308 | Hs.11611 |
| AA258057 | 703383 | Hs.175511 |
| AF055982 | 2243051 | Hs.104570 |
| AA993573 | 486208 | Hs.2025 |
| AF248053 | 758347 | Hs.305971 |
| AA873846 | 788617 | Hs.306083 |
| BF589790 | 774446 | Hs.394 |
| U38276 | 809526 | Hs.32981 |
| AW290960 | 289760 | Hs.6382 |
| BC003554 | 795809 | Hs.4890 |
| AI004681 | 1035796 | Hs.339665 |
| AI142639 | 155857 | Hs.146662 |
| BI494440 | 306924 | Hs.48450 |
| NM_001559 | 1587390 | Hs.73165 |
| AF280546 | 271215 | Hs.17778 |
| AI128331 | 132381 | Hs.83790 |
| NM_003489 | 809627 | Hs.155017 |
| BC001886 | 1422338 | Hs.75319 |
| BC001886 | 624627 | Hs.75319 |
| W94858 | 725365 | Hs.65029 |
| AI356375 | 1161155 | Hs.1174 |
| Z11162 | 210687 | Hs.89472 |
| AA633845 | 1637647 | Hs.269760 |
| BC004376 | 666879 | Hs.87268 |
| AF027866 | 1509935 | Hs.138202 |
| AW206445 | 35147 | Hs.118338 |
| AK027024 | 2017917 | Hs.94952 |
| BC016838 | 39833 | Hs.95594 |
| AI457921 | 768271 | Hs.29692 |
| R36615 | 137344 | Hs.268779 |
| AF117708 | 2063982 | Hs.240395 |
| AW015327 | 148379 | Hs.239176 |
| BC002829 | 810813 | Hs.38991 |
| BC018636 | 25154 | Hs.274404 |
| BC018636 | 813841 | Hs.274404 |
| AF161423 | 431286 | Hs.41767 |
| AW139414 | 138788 | Hs.1906 |
| AI656548 | 1569658 | Hs.110168 |
| AA933076 | 1553996 | Hs.252938 |
| NM_000792 | 296702 | Hs.251415 |
| AB038518 | 284100 | Hs.29423 |
| BC012513 | 415851 | Hs.6838 |
| BF508352 | 1837155 | Hs.2633 |
| AA831831 | 381166 | Hs.27590 |
| AA877761 | 1257131 | Hs.184727 |
| BC015571 | 742862 | Hs.50868 |
| AK024865 | 502625 | Hs.47099 |
| AI332972 | 726699 | Hs.23650 |
| AA766903 | 1550894 | Hs.116346 |
| NM_053056 | 526757 | Hs.82932 |
| NM_053056 | 841641 | Hs.82932 |
| AI569288 | 787860 | Hs.45057 |
| NM_005985 | 810119 | Hs.48029 |
| AW074266 | 280602 | Hs.23071 |
| NM_001784 | 2309141 | Hs.3107 |
| AL133590 | 884355 | Hs.82318 |
| NM_004694 | 266389 | Hs.114924 |
| BC017583 | 796148 | Hs.7122 |
| NM_004525 | 143846 | Hs.153595 |
| NM_004525 | 1899878 | Hs.153595 |
| NM_002964 | 562729 | Hs.100000 |
| AF101051 | 664975 | Hs.7327 |
| AK024715 | 364865 | Hs.151258 |
| NM_003878 | 809588 | Hs.78619 |
| BC003635 | 470393 | Hs.2256 |
| NM_002281 | 1903066 | Hs.32952 |
| AI655104 | 1850224 | Hs.22047 |
| AK026790 | 357396 | Hs.24139 |
| AI553863 | 773329 | Hs.300670 |
| AI553863 | 813396 | Hs.300670 |
| AF089868 | 897531 | Hs.211579 |
| AA642589 | 2313898 | Hs.180016 |
| BC000218 | 1469966 | Hs.127792 |
| BC011806 | 150003 | Hs.29724 |
| BF433296 | 841008 | Hs.62661 |
| AF026547 | 2163910 | Hs.169047 |
| AI350518 | 36491 | Hs.129692 |
| AI433440 | 141806 | Hs.211600 |
| AF038173 | 221828 | Hs.259873 |
| NM_001771 | 1847497 | Hs.171763 |
| BC010003 | 767982 | Hs.20953 |
| AA866043 | 769857 | Hs.84152 |
| L07738 | 344091 | Hs.147989 |
| AL117393 | 278430 | Hs.6641 |
| D10522 | 840865 | Hs.75607 |
| BF511873 | 1648516 | Hs.170278 |
| AA398573 | 1642846 | Hs.104660 |
| BC011690 | 1580874 | Hs.44396 |
| AL109775 | 179283 | Hs.80315 |
| AI810347 | 366039 | Hs.349764 |
| M14584 | 310406 | Hs.93913 |
| AW195539 | 1870049 | Hs.174441 |
| BC005029 | 1472797 | Hs.42824 |
| BC009401 | 810859 | Hs.943 |
| BC003559 | 109316 | Hs.234726 |
| AK000193 | 624867 | Hs.65021 |
| AF027299 | 785967 | Hs.7857 |
| AL157432 | 815015 | Hs.180780 |
| AL050151 | 121898 | Hs.324504 |
| AK026275 | 79960 | Hs.324841 |
| AF014398 | 32299 | Hs.5753 |
| AW207060 | 785571 | Hs.13015 |
| NM_005518 | 757222 | Hs.59889 |
| BE464662 | 140635 | Hs.95893 |
| AJ243500 | 417424 | Hs.302740 |
| AI097387 | 503602 | Hs.197922 |
| AI421708 | 2012854 | Hs.288905 |
| AW080831 | 2019750 | Hs.277728 |
| R39691 | 971399 | Hs.154679 |
| NM_007069 | 785293 | Hs.37189 |
| BC002709 | 277426 | Hs.274329 |
| T23696 | 767171 | Hs.80618 |
| AF011468 | 209066 | Hs.250822 |
| BC010522 | 40100 | Hs.343666 |
| R15800 | 53203 | Hs.94834 |
| AK000691 | 38213 | Hs.7734 |
| AL049223 | 2284803 | Hs.31218 |
| BC010466 | 47460 | Hs.3353 |
| AI299541 | 1897953 | Hs.150124 |
| AW205836 | 731360 | Hs.350251 |
| BC007026 | 1917449 | Hs.1955 |
| AA054983 | 377081 | Hs.63158 |
| AF146796 | 810911 | Hs.105039 |
| BF436282 | 82322 | Hs.11916 |
| AW341053 | 366848 | Hs.173638 |
| NM_001998 | 789012 | Hs.198862 |
| AW291846 | 1473914 | Hs.351321 |
| AB027289 | 2009491 | Hs.26663 |
| AA584033 | 2610539 | Hs.335952 |
| AA584033 | 366481 | Hs.335952 |
| AI097664 | 897509 | Hs.173030 |
| AI668861 | 277463 | Hs.153498 |
| BC001356 | 1422723 | Hs.50842 |
| AW265278 | 2114195 | Hs.157091 |
| AF264784 | 1709786 | Hs.26102 |
| R49390 | 1881774 | Hs.254129 |
| BC012609 | 70692 | Hs.75716 |
| AB026707 | 1056172 | Hs.112242 |
| AW148329 | 1584889 | Hs.175208 |
| AK026813 | 244796 | Hs.118258 |
| R40565 | 271683 | Hs.169370 |
| AI561078 | 2320932 | Hs.38972 |
| AW292522 | 812954 | Hs.88253 |
| AI973194 | 1456937 | Hs.1154 |
| BC009238 | 612274 | Hs.75318 |
| Y10183 | 26617 | Hs.10247 |
| BC008568 | 770212 | Hs.75184 |
| AL049332 | 246304 | Hs.77311 |
| NM_004029 | 1159963 | Hs.166120 |
| AJ002367 | 773322 | Hs.55967 |
| AA601573 | 430623 | Hs.272056 |
| BC009989 | 530958 | Hs.197366 |
| AL137502 | 1686766 | Hs.238679 |
| AL162079 | 486175 | Hs.75231 |
| NM_017458 | 591281 | Hs.80680 |
| AA854788 | 50182 | Hs.89591 |
| AW205080 | 179548 | Hs.172700 |
| BC008442 | 840567 | Hs.351316 |
| AI123176 | 1910895 | Hs.98838 |
| AI939560 | 2017773 | Hs.345183 |
| BF512938 | 46180 | Hs.171495 |
| BC010940 | 814410 | Hs.105115 |
| NM_002847 | 812968 | Hs.74624 |
| AI218954 | 1521361 | Hs.62113 |
| BC010120 | 613056 | Hs.167791 |
| BC013919 | 278483 | Hs.82962 |
| BC013919 | 853368 | Hs.82962 |
| BC000078 | 812187 | Hs.32603 |
| BC006322 | 51448 | Hs.460 |
| AA586749 | 592540 | Hs.195850 |
| AK027056 | 743146 | Hs.293907 |
| AW139156 | 41720 | Hs.299315 |
| AF035279 | 488019 | Hs.12503 |
| AA731110 | 731236 | Hs.18925 |
| AA730123 | 2507971 | Hs.158303 |
| AW082090 | 1708791 | Hs.170673 |
| AI208471 | 23454 | Hs.120869 |
| AW191026 | 489320 | Hs.198282 |
| AF285836 | 2017597 | Hs.105547 |
| AI273078 | 1468220 | Hs.173334 |
| AI188749 | 1723400 | Hs.271594 |
| AA903532 | 40893 | Hs.8071 |
| U64197 | 1874367 | Hs.75498 |
| R52665 | 240505 | Hs.7911 |
| BC010116 | 810002 | Hs.343214 |
| AA810953 | 713158 | Hs.89104 |
| AW150018 | 811162 | Hs.230 |
| BC001268 | 704532 | Hs.54483 |
| AI042183 | 212198 | Hs.44585 |
| BC013200 | 730410 | Hs.1765 |
| AL512731 | 239724 | Hs.235709 |
| BC012527 | 897806 | Hs.197540 |
| AI244129 | 345663 | Hs.25691 |
| BC001459 | 1476053 | Hs.343807 |
| AW149773 | 2016022 | Hs.29353 |
| BC006376 | 855657 | Hs.122647 |
| BC010379 | 345957 | Hs.2621 |
| AW450142 | 257135 | Hs.77239 |
| BC010923 | 767475 | Hs.41683 |
| AF147204 | 79629 | Hs.89414 |
| BC007951 | 753184 | Hs.2316 |
| BC010963 | 1409509 | Hs.73980 |
| AI247211 | 1517749 | Hs.24133 |
| AF073770 | 487697 | Hs.12743 |
| NM_006006 | 2499829 | Hs.37096 |
| U97105 | 841620 | Hs.173381 |
| AI922204 | 301740 | Hs.351546 |
| AI866858 | 840687 | Hs.89603 |
| AI866867 | 486591 | Hs.171921 |
| AW664923 | 282958 | Hs.89582 |
| AB040057 | 254029 | Hs.23643 |
| AI538226 | 1911864 | Hs.32976 |
| AW340797 | 773152 | Hs.98434 |
| AW006452 | 366526 | Hs.58006 |
| AF116660 | 50562 | Hs.283683 |
| AI199268 | 511096 | Hs.19322 |
| AF051152 | 80633 | Hs.63668 |
| AA857853 | 302127 | Hs.204238 |
| R38689 | 35812 | Hs.12376 |
| AA883715 | 1461550 | Hs.125494 |
| AL043469 | 785975 | Hs.80424 |
| AL360254 | 46843 | Hs.30965 |
| AF191017 | 205049 | Hs.111676 |
| S48196 | 1474284 | Hs.323949 |
| S48196 | 1536168 | Hs.323949 |
| AB032698 | 1722980 | Hs.14770 |
| AW172994 | 196612 | Hs.1695 |
| AA732062 | 1896556 | Hs.170180 |
| AW189851 | 488956 | Hs.211610 |
| AL096842 | 744395 | Hs.7946 |
| BC010954 | 1493160 | Hs.2248 |
| AK027191 | 510273 | Hs.240443 |
| AI283489 | 2014138 | Hs.154890 |
| L11672 | 2455403 | Hs.8597 |
| AF186780 | 505864 | Hs.79219 |
| AA086248 | 1910516 | Hs.114889 |
| BG655508 | 345764 | Hs.74592 |
| N95437 | 1663716 | Hs.134479 |
| X53587 | 811096 | Hs.85266 |
| AK026181 | 2012257 | Hs.288850 |
| AK026181 | 753071 | Hs.288850 |
| BC001844 | 41358 | Hs.104105 |
| AI921122 | 1504268 | Hs.148218 |
| AA946776 | 1589786 | Hs.111 |
| H08145 | 48033 | Hs.23606 |
| NM_001078 | 44477 | Hs.109225 |
| AY029191 | 78921 | Hs.10760 |
| AA903183 | 1517171 | Hs.1724 |
| AF019386 | 220372 | Hs.40968 |
| NM_005409 | 1030351 | Hs.103982 |
| AF161398 | 701115 | Hs.238205 |
| AI587351 | 509823 | Hs.73848 |
| AI027237 | 284160 | Hs.127436 |
| AL133102 | 251870 | Hs.127842 |
| BE047394 | 753236 | Hs.8208 |
| BC017054 | 2014034 | Hs.154672 |
| BC006534 | 767284 | Hs.50550 |
| W46451 | 153025 | Hs.2250 |
| AI199647 | 2021882 | Hs.112842 |
| AF217989 | 782501 | Hs.6748 |
| AF047711 | 486304 | Hs.227806 |
| AA843666 | 490946 | Hs.172510 |
| BC002700 | 843321 | Hs.23881 |
| AB028967 | 770427 | Hs.202687 |
| BC000356 | 814701 | Hs.79078 |
| NM_033014 | 629906 | Hs.109439 |
| AA883246 | 782537 | Hs.131740 |
| AK025309 | 85634 | Hs.169756 |
| U63008 | 1649374 | Hs.15113 |
| BC002757 | 1475803 | Hs.114346 |
| AA084672 | 182750 | Hs.158311 |
| AA805970 | 726678 | Hs.74583 |
| AI383975 | 811955 | Hs.11261 |
| AI383975 | 868168 | Hs.11261 |
| AA668884 | 124128 | Hs.19155 |
| NM_006186 | 898221 | Hs.82120 |
| AL080078 | 322511 | Hs.85335 |
| AI251449 | 323322 | Hs.171939 |
| AL157455 | 31299 | Hs.22543 |
| AI205008 | 1709233 | Hs.323878 |
| NM_003155 | 565075 | Hs.25590 |
| BC007921 | 295093 | Hs.192861 |
| AK026589 | 143966 | Hs.90998 |
| AL110298 | 202201 | Hs.7594 |
| BC009522 | 2171573 | Hs.184841 |
| BC003180 | 74119 | Hs.48375 |
| AF140242 | 504461 | Hs.279926 |
| NM_002497 | 462926 | Hs.153704 |
| BC003630 | 1035889 | Hs.3235 |
| AF305083 | 133213 | Hs.2173 |
| AF305083 | 366778 | Hs.2173 |
| AA905217 | 2450261 | Hs.121269 |
| AF331643 | 1900362 | Hs.3402 |
| U66065 | 565379 | Hs.81875 |
| NM_001094 | 2122306 | Hs.6517 |
| AL512743 | 49883 | Hs.12365 |
| AI969251 | 1942497 | Hs.115325 |
| AA521246 | 1715608 | Hs.210792 |
| AW301074 | 591422 | Hs.293533 |
| BM055132 | 2498575 | Hs.663 |
| AW069753 | 898092 | Hs.75511 |
| NM_006278 | 813751 | Hs.75268 |
| AW140108 | 347726 | Hs.301963 |
| AI193122 | 162308 | Hs.132275 |
| AW016075 | 160609 | Hs.284192 |
| BC016628 | 1668067 | Hs.240845 |
| BC016628 | 361659 | Hs.240845 |
| AI026838 | 280763 | Hs.30120 |
| D87119 | 360155 | Hs.155418 |
| NM_000142 | 752631 | Hs.1420 |
| AF161505 | 788524 | Hs.99291 |
| AK000341 | 44387 | Hs.246107 |
| AI910678 | 284734 | Hs.75850 |
| AF085233 | 261609 | Hs.279696 |
| AF085233 | 3316104 | Hs.279696 |
| BC018127 | 140806 | Hs.83920 |
| AW014507 | 703541 | Hs.54925 |
| AK025624 | 1637791 | Hs.71331 |
| AK025624 | 587992 | Hs.71331 |
| AF119815 | 788698 | Hs.99231 |
| AW139237 | 796480 | Hs.30875 |
| AI656385 | 1562231 | Hs.151717 |
| AA613425 | 2062432 | Hs.1584 |
| BC001969 | 595162 | Hs.301198 |
| BC001207 | 841057 | Hs.7457 |
| AL110252 | 49204 | Hs.168950 |
| AA761787 | 49482 | Hs.315482 |
| BC007079 | 812105 | Hs.75823 |
| F10094 | 22428 | Hs.333319 |
| NM_000935 | 263013 | Hs.41270 |
| AA910336 | 1520559 | Hs.135186 |
| U67784 | 246786 | Hs.23016 |
| AI022925 | 1589468 | Hs.79368 |
| BC008283 | 258790 | Hs.80247 |
| NM_003122 | 1412481 | Hs.181286 |
| AA804433 | 701481 | Hs.926 |
| BC017772 | 289057 | Hs.47166 |
| NM_006308 | 2088978 | Hs.41707 |
| BE673992 | 1584638 | Hs.128211 |
| AA868889 | 1638827 | Hs.274285 |
| AL157471 | 33837 | Hs.15420 |
| X59766 | 1456160 | Hs.71 |
| BC007636 | 41332 | Hs.26608 |
| AI566899 | 753034 | Hs.35841 |
| AA910884 | 1520895 | Hs.130217 |
| R40176 | 27769 | Hs.167406 |
| AL136612 | 768520 | Hs.90063 |
| AL136612 | 838478 | Hs.90063 |
| AA600175 | 230560 | Hs.39720 |
| BC004291 | 780937 | Hs.74554 |
| NM_001197 | 1916575 | Hs.155419 |
| AW207696 | 2250736 | Hs.97644 |
| BC002914 | 700299 | Hs.24143 |
| R61469 | 53122 | Hs.281587 |
| Y16645 | 1911099 | Hs.271387 |
| AF303058 | 325160 | Hs.169330 |
| AI248610 | 110578 | Hs.15060 |
| U31214 | 588460 | Hs.301350 |
| AK024951 | 83549 | Hs.1279 |
| NM_013267 | 1911706 | Hs.325443 |
| BC000006 | 897950 | Hs.78629 |
| BC004471 | 740941 | Hs.74120 |
| AK025062 | 564801 | Hs.172129 |
| NM_006271 | 179046 | Hs.292707 |
| NM_001839 | 486787 | Hs.194662 |
| BF510271 | 2049813 | Hs.241545 |
| AB067468 | 85660 | Hs.11006 |
| AI492884 | 1358229 | Hs.82132 |
| AA588742 | 127709 | Hs.250 |
| AW271272 | 2769084 | Hs.239571 |
| BC008483 | 727147 | Hs.227948 |
| BF434466 | 32493 | Hs.227730 |
| NM_002963 | 1088345 | Hs.112408 |
| NM_002988 | 768497 | Hs.16530 |
| BC011393 | 898258 | Hs.169965 |
| BC009753 | 1635307 | Hs.234898 |
| BC004278 | 1417886 | Hs.34516 |
| AK025307 | 1554646 | Hs.259785 |
| AK000140 | 491644 | Hs.107139 |
| BI324932 | 363569 | Hs.199248 |
| AI445650 | 1670188 | Hs.2200 |
| AL039118 | 33051 | Hs.2714 |
| M84757 | 813614 | Hs.1076 |
| BC003097 | 130201 | Hs.347326 |
| NM_001792 | 325182 | Hs.161 |
| R92436 | 196305 | Hs.131837 |
| BF001174 | 487297 | Hs.296341 |
| BC006267 | 1524001 | Hs.7331 |
| BC005257 | 2251833 | Hs.183752 |
| NI1004038 | 809998 | Hs.274376 |
| AI953054 | 530875 | Hs.89643 |
| BC002591 | 1384851 | Hs.2258 |
| AI384087 | 753162 | Hs.173802 |
| AI732321 | 154795 | Hs.348819 |
| BF510331 | 203896 | Hs.168481 |
| BC006093 | 22040 | Hs.151738 |
| BC006378 | 250883 | Hs.16695 |
| BF510778 | 898058 | Hs.53997 |
| AF146761 | 288807 | Hs.20450 |
| AI239743 | 345232 | Hs.36 |
| BM127823 | 1584449 | Hs.294151 |
| BM127823 | 786525 | Hs.294151 |
| AB041036 | 740780 | Hs.57771 |
| AI962213 | 1467799 | Hs.22599 |
| AF068180 | 1565079 | Hs.167746 |
| AW301017 | 1712825 | Hs.146492 |
| AI241077 | 461727 | Hs.1870 |
| BC000141 | 812965 | Hs.79070 |
| AI245337 | 1870305 | Hs.78846 |
| BC014533 | 1908758 | Hs.44268 |
| R43793 | 50615 | Hs.80288 |
| BC007533 | 489535 | Hs.69285 |
| AA973836 | 1586043 | Hs.128739 |
| Y13786 | 1557714 | Hs.278679 |
| BC002727 | 1555478 | Hs.64179 |
| AI740744 | 22773 | Hs.10475 |
| AK025953 | 126341 | Hs.288965 |
| BC005989 | 1874603 | Hs.143113 |
| X96757 | 45578 | Hs.118825 |
| R45048 | 29954 | Hs.159538 |
| BC015613 | 135688 | Hs.334695 |
| BC011969 | 771004 | Hs.347534 |
| H06160 | 1519985 | Hs.106794 |
| AI204186 | 1754094 | Hs.146076 |
| AF102848 | 454970 | Hs.9029 |
| L27711 | 700792 | Hs.84113 |
| AF030186 | 358217 | Hs.58367 |
| AI536895 | 248829 | Hs.102267 |
| BM129291 | 2017950 | Hs.1149 |
| AL133706 | 283748 | Hs.4257 |
| BE676183 | 1670836 | Hs.103483 |
| NM_005965 | 841308 | Hs.211582 |
| BC017409 | 1696831 | Hs.88594 |
| BC017409 | 486626 | Hs.88594 |
| AW139590 | 1571882 | Hs.156051 |
| AA603977 | 2321042 | HS.218366 |
| AL035878 | 896968 | Hs.34780 |
| AA830549 | 859192 | Hs.20021 |
| AA630788 | 825470 | Hs.156346 |
| AI831078 | 1620905 | Hs.14587 |
| NM_001337 | 283023 | Hs.78913 |
| AB032417 | 1891563 | Hs.19545 |
| AK027146 | 469235 | Hs.180946 |
| AW293219 | 785694 | Hs.288467 |
| BF513346 | 841480 | Hs.88780 |
| BC009198 | 746373 | Hs.348989 |
| BC014890 | 293339 | Hs.93005 |
| AF339783 | 72811 | Hs.288809 |
| AB017800 | 781091 | Hs.6414 |
| AI434326 | 1435103 | Hs.5541 |
| BC001638 | 774082 | Hs.1619 |
| AI733703 | 1742850 | Hs.143735 |
| AF141332 | 3117993 | Hs.200333 |
| NM_003221 | 363144 | Hs.33102 |
| AL137559 | 767475 | Hs.27495 |
| AF131794 | 843250 | Hs.10432 |
| AF131794 | 950450 | Hs.10432 |
| AF101477 | 2345693 | Hs.144914 |
| AL046192 | 2165360 | Hs.8854 |
| AL046192 | 295410 | Hs.8854 |
| AL046192 | 563985 | Hs.8854 |
| U43203 | 81427 | Hs.197764 |
| AI096369 | 668007 | Hs.348862 |
| AL136703 | 854678 | Hs.57209 |
| AF033347 | 45636 | Hs.40866 |
| AI280476 | 786605 | Hs.71992 |
| AI559983 | 712505 | Hs.334562 |
| BE551149 | 1620366 | Hs.129319 |
| AI373912 | 611407 | Hs.241363 |
| AI823489 | 1609665 | Hs.167218 |
| AW131780 | 1883065 | Hs.167988 |
| AW131780 | 2164744 | Hs.167988 |
| AA912183 | 284383 | Hs.47447 |
| L20971 | 788136 | Hs.188 |
| AK026315 | 79565 | Hs.178470 |
| AK001643 | 1585650 | Hs.8395 |
| AI144219 | 138917 | Hs.92137 |
| BC000897 | 755599 | Hs.146360 |
| AL110174 | 2306752 | Hs.90005 |
| R43523 | 32777 | Hs.217754 |
| NM_014583 | 786550 | Hs.279943 |
| BE670572 | 430237 | Hs.181297 |
| W46976 | 325070 | Hs.94667 |
| AI268717 | 1910959 | Hs.128401 |
| NM_001773 | 213635 | Hs.85289 |
| AL353948 | 343079 | Hs.6272 |
| AI200852 | 1836699 | Hs.127780 |
| AA130089 | 490965 | Hs.23136 |
| AA255953 | 243100 | Hs.81934 |
| NM_012244 | 415670 | Hs.22891 |
| NM_003508 | 2149968 | Hs.158335 |
| BC017859 | 298417 | Hs.82961 |
| NM_000352 | 1558108 | Hs.54470 |
| AA804407 | 325515 | Hs.29716 |
| AF176012 | 120138 | Hs.260720 |
| AF052504 | 26294 | Hs.241471 |
| AI263695 | 502173 | Hs.72050 |
| AW139831 | 258167 | Hs.284186 |
| AA156097 | 969843 | Hs.29283 |
| BC006428 | 139354 | Hs.15093 |
| AK024872 | 487932 | Hs.92254 |
| AK024872 | 826194 | Hs.92254 |
| BC016861 | 590310 | Hs.351461 |
| AI272823 | 83358 | Hs.76704 |
| AI674226 | 279720 | Hs.125201 |
| AI373462 | 155072 | Hs.29190 |
| AK000978 | 505575 | Hs.79741 |
| AA807457 | 243549 | Hs.1334 |
| AL133622 | 1571106 | Hs.301011 |
| R49089 | 179212 | Hs.24811 |
| AJ420423 | 593431 | Hs.23703 |
| BC017352 | 377275 | Hs.82237 |
| U62325 | 1470333 | Hs.324125 |
| AI804588 | 1733262 | Hs.167380 |
| AI733682 | 1637829 | Hs.130239 |
| BC012372 | 868652 | Hs.278625 |
| AF007170 | 1492468 | Hs.125783 |
| BC007008 | 839736 | Hs.1940 |
| BC013438 | 1640821 | Hs.42792 |
| BC017733 | 1909775 | Hs.349227 |
| BC017733 | 877772 | Hs.349227 |
| AY029179 | 753198 | Hs.333893 |
| AF271781 | 471196 | Hs.111577 |
| BC007681 | 1911343 | Hs.3797 |
| H29323 | 270917 | Hs.7306 |
| AY043361 | 813281 | Hs.324275 |
| AF070632 | 669359 | Hs.23729 |
| NM_004310 | 302591 | Hs.109918 |
| BE967259 | 342181 | Hs.79241 |
| AJ420468 | 1901310 | Hs.51965 |
| AA775255 | 2299609 | Hs.284163 |
| BC018646 | 204897 | Hs.75648 |
| BC010626 | 214205 | Hs.28149 |
| AL161952 | 1609836 | Hs.170171 |
| AI095049 | 1686314 | Hs.6786 |
| AK055698 | 1860909 | Hs.349189 |
| AI261967 | 377252 | Hs.45743 |
| H10667 | 80727 | Hs.274243 |
| AK025347 | 415766 | Hs.170162 |
| BC000088 | 137940 | Hs.2006 |
| BC003628 | 1486082 | Hs.1690 |
| BE965331 | 2309234 | Hs.87417 |
| AI432615 | 785745 | Hs.264 |
| AF100759 | 416374 | Hs.284243 |
| R15881 | 2118505 | Hs.7138 |
| NM_004938 | 2043415 | Hs.153924 |
| AW206460 | 454632 | Hs.6360 |
| BC018035 | 1557994 | Hs.127043 |
| BC014189 | 854763 | Hs.10260 |
| BF433570 | 1557637 | Hs.144479 |
| BC008915 | 1323591 | Hs.76353 |
| AA806280 | 772913 | Hs.349306 |
| AI140061 | 304908 | Hs.1189 |
| AI828071 | 1616253 | Hs.129057 |
| AF269101 | 796539 | Hs.27695 |
| AW294316 | 1847093 | Hs.125608 |
| AF104032 | 1702742 | Hs.184601 |
| AF104032 | 755578 | Hs.184601 |
| BC013430 | 139835 | Hs.28309 |
| BC008317 | 52933 | Hs.79136 |
| BC007541 | 1570910 | Hs.55067 |
| BC014941 | 788234 | Hs.34853 |
| AA069179 | 1661732 | Hs.153687 |
| BC009211 | 125788 | Hs.8739 |
| AI351524 | 796197 | Hs.169470 |
| AF153605 | 1554009 | Hs.107528 |
| BC017758 | 724888 | Hs.687 |
| NM_007210 | 470964 | Hs.151678 |
| AF131789 | 32489 | Hs.4863 |
| BI492630 | 20115 | Hs.62185 |
| BC017918 | 868396 | Hs.302766 |
| AL080192 | 2312454 | Hs.101282 |
| AF091092 | 768007 | Hs.43728 |
| AW207201 | 1507713 | Hs.97220 |
| AL137332 | 754157 | Hs.23869 |
| AI367357 | 255285 | Hs.112083 |
| AB048286 | 161484 | Hs.334305 |
| AL110139 | 1844765 | Hs.27373 |
| BC014913 | 786663 | Hs.247525 |
| AB018329 | 897731 | Hs.24212 |
| AF038191 | 51097 | Hs.134846 |
| AF043473 | 249687 | Hs.117780 |
| AK057601 | 752903 | Hs.54985 |
| AK057601 | 768031 | Hs.54985 |
| BC012844 | 812048 | Hs.74621 |
| AL512749 | 752625 | Hs.93961 |
| BC011721 | 757489 | Hs.349695 |
| NM_001793 | 773301 | Hs.2877 |
| AI273928 | 814798 | Hs.75746 |
| Y17978 | 1968246 | Hs.118722 |
| BC004143 | 741977 | Hs.69771 |
| AI694966 | 490178 | Hs.49597 |
| AI694966 | 501868 | Hs.49597 |
| U74612 | 564803 | Hs.239 |
| BC007609 | 2112387 | Hs.231444 |
| AL117633 | 190325 | Hs.348724 |
| AF222980 | 767471 | Hs.26985 |
| AI678454 | 2052032 | Hs.61638 |
| BC000809 | 786607 | Hs.95243 |
| BC014213 | 746373 | Hs.159505 |
| BC006168 | 840333 | Hs.82432 |
| BC005910 | 346688 | Hs.279651 |
| BC008682 | 726582 | Hs.12887 |
| BF446789 | 1946448 | Hs.139851 |
| BF446789 | 840984 | Hs.139851 |
| AB050468 | 1533710 | Hs.4193 |
| AB050468 | 592594 | Hs.4193 |
| AI126271 | 488202 | Hs.49433 |
| NM_003806 | 1671299 | Hs.87247 |
| BC001211 | 753038 | Hs.23131 |
| AW194426 | 1518845 | Hs.20726 |
| BC001940 | 66406 | Hs.104859 |
| R42732 | 214996 | Hs.283705 |
| AA716439 | 1898758 | Hs.99824 |
| AI202593 | 2119039 | Hs.144877 |
| BF511352 | 193913 | Hs.80887 |
| BC009373 | 1635059 | Hs.326391 |
| NM_006769 | 1706027 | Hs.3844 |
| X55362 | 796646 | Hs.75212 |
| AL136653 | 135811 | Hs.93675 |
| AI866286 | 740788 | Hs.71962 |
| BF476123 | 1583198 | Hs.321176 |
| X64318 | 858153 | Hs.79334 |
| BC008185 | 526945 | Hs.288354 |
| AA903387 | 154999 | Hs.323032 |
| BC003098 | 840606 | Hs.75441 |
| BC012142 | 123627 | Hs.182429 |
| R41429 | 266312 | Hs.84999 |
| AL049933 | 753215 | Hs.203862 |
| BC000232 | 263727 | Hs.178112 |
| U85267 | 884462 | Hs.184222 |
| BC010658 | 686172 | Hs.77695 |
| BE676675 | 1609372 | Hs.268551 |
| AI039854 | 112525 | Hs.348415 |
| BC005153 | 1086070 | Hs.198551 |
| AF280086 | 2405856 | Hs.157439 |
| NM_004961 | 209137 | Hs.22785 |
| BC000332 | 878833 | Hs.76118 |
| M76558 | 49630 | Hs.23838 |
| BC001278 | 1909457 | Hs.25338 |
| BC001278 | 782547 | Hs.25338 |
| AI147370 | 1839935 | Hs.143653 |
| AI288717 | 505334 | Hs.100007 |
| R42185 | 41826 | Hs.102720 |
| BC000183 | 306771 | Hs.56145 |
| BC015182 | 196189 | Hs.83834 |
| BC012727 | 1035359 | Hs.101265 |
| S61953 | 855563 | Hs.199067 |
| NM_002996 | 140574 | Hs.80420 |
| BC013362 | 291478 | Hs.170019 |
| AI200759 | 767322 | Hs.44737 |
| BC008588 | 1568391 | Hs.4114 |
| AK025732 | 855487 | Hs.75811 |
| AK024892 | 1603583 | Hs.14368 |
| BC008832 | 771224 | Hs.139800 |
| BC008832 | 782811 | Hs.139800 |
| AK000708 | 191882 | Hs.169764 |
| AI631708 | 1048592 | Hs.121554 |
| NM_002852 | 1691868 | Hs.2050 |
| BC016004 | 840466 | Hs.67726 |
| AL137281 | 24958 | Hs.17110 |
| BC015663 | 199367 | Hs.21858 |
| AI524385 | 128711 | Hs.62180 |
| BC002551 | 743810 | Hs.30114 |
| AK025511 | 1910785 | Hs.29189 |
| NM_002145 | 1526826 | Hs.2733 |
| AA573096 | 768393 | Hs.57655 |
| AA664218 | 796542 | Hs.43697 |
| AA424146 | 70201 | Hs.300496 |
| AI262988 | 897593 | Hs.6728 |
| AL122086 | 366591 | Hs.12598 |
| BC001051 | 712139 | Hs.111554 |
| BC009744 | 810057 | Hs.198726 |
| AA706017 | 550141 | Hs.119944 |
| AI399653 | 33817 | Hs.22917 |
| BF513300 | 244767 | Hs.1192 |
| AL136911 | 841415 | Hs.10706 |
| AW472810 | 1912458 | Hs.673 |
| AF051321 | 844703 | Hs.13565 |
| AF247820 | 758088 | Hs.325474 |
| AK026139 | 1858230 | Hs.98485 |
| BC015839 | 155768 | Hs.350038 |
| BC012625 | 502198 | Hs.303090 |
| AI279525 | 1864302 | Hs.11713 |
| AI961444 | 2010012 | Hs.155545 |
| AF239668 | 844601 | Hs.203 |
| NM_006113 | 415229 | Hs.267659 |
| AI800677 | 378813 | Hs.251754 |
| AK027126 | 882461 | Hs.160786 |
| AI000682 | 1610158 | Hs.144312 |
| BC017656 | 713238 | Hs.52081 |
| AF133059 | 345069 | Hs.22900 |
| NM_002965 | 1459376 | Hs.112405 |
| BC015753 | 153340 | Hs.75765 |
| AA846684 | 321945 | Hs.25545 |
| BC008366 | 2028959 | Hs.150403 |
| AB047783 | 344854 | Hs.55565 |
| AA922144 | 755881 | Hs.298023 |
| AI366784 | 838408 | Hs.48820 |
| AW270334 | 1877697 | Hs.50831 |
| BF475464 | 33852 | Hs.22883 |
| AV656862 | 1877206 | Hs.25252 |
| BC001275 | 208718 | Hs.78225 |
| X77197 | 2063419 | Hs.199250 |
| AI217517 | 130781 | Hs.101414 |
| BC000633 | 2062329 | Hs.169840 |
| AW296675 | 815794 | Hs.3164 |
| AI028773 | 85450 | Hs.9795 |
| NM_033380 | 1592039 | Hs.169825 |
| AL133636 | 1493383 | Hs.119475 |
| AL133636 | 69124 | Hs.119475 |
| AW247958 | 144740 | Hs.84700 |
| L20470 | 358527 | Hs.73729 |
| BC013106 | 773674 | Hs.206097 |
| BC012900 | 143169 | Hs.346735 |
| AF004222 | 44287 | Hs.3803 |
| AF004222 | 685185 | Hs.3803 |
| NM_057158 | 756596 | Hs.2359 |
| AI668602 | 160532 | Hs.176588 |
| AI468574 | 147826 | Hs.171965 |
| AA205947 | 1588924 | Hs.22835 |
| AA627358 | 1683411 | Hs.142156 |
| BC013083 | 949938 | Hs.135084 |
| AF153609 | 2013515 | Hs.296323 |
| BC012381 | 815556 | Hs.6823 |
| BF591743 | 2709505 | Hs.204096 |
| AJ000388 | 838676 | Hs.169172 |
| BC002536 | 950682 | Hs.99910 |
| L13773 | 302190 | Hs.114765 |
| L13773 | 381021 | Hs.114765 |
| AI560801 | 1553054 | Hs.22404 |
| AF053306 | 842968 | Hs.36708 |
| AF038185 | 31818 | Hs.66187 |
| AA865325 | 838278 | Hs.155106 |
| BC007656 | 769921 | Hs.93002 |
| AW076080 | 669379 | Hs.18987 |
| AW076080 | 73659 | Hs.18987 |
| BC015706 | 564981 | Hs.134726 |
| BC000404 | 856427 | Hs.6566 |
| AI948466 | 740925 | Hs.840 |
| NM_003914 | 2091001 | Hs.79378 |
| AF070592 | 1925681 | Hs.66170 |
| AF178532 | 810454 | Hs.271411 |
| BC000465 | 85195 | Hs.9701 |
| BC015915 | 298122 | Hs.173859 |
| AK026698 | 52103 | Hs.101774 |
| NM_003503 | 287749 | Hs.28853 |
| AA399226 | 741919 | Hs.25527 |
| AA962285 | 795379 | Hs.23860 |
| AF140240 | 70500 | Hs.267182 |
| AL157483 | 1759290 | Hs.11260 |
| AJ420567 | 1542749 | Hs.262476 |
| U78168 | 795382 | Hs.8578 |
| AI732534 | 1609005 | Hs.46452 |
| AI198522 | 2054122 | Hs.5944 |
| AI198522 | 767706 | Hs.5944 |
| NM_014274 | 417424 | Hs.289104 |
| BC012613 | 80221 | Hs.646 |
| AI304875 | 705274 | Hs.115907 |
| AI963850 | 1609538 | Hs.104696 |
| NM_002417 | 769513 | Hs.80976 |
| AK027311 | 1693189 | Hs.182585 |
| AK027311 | 417637 | Hs.182585 |
| BC001526 | 687289 | Hs.173179 |
| AA574174 | 85561 | Hs.250615 |
| AB000509 | 563621 | Hs.29736 |
| AA489383 | 843398 | Hs.73853 |
| AW265625 | 325247 | Hs.56105 |
| AF183421 | 784150 | Hs.223025 |
| AF183421 | 785701 | Hs.223025 |
| AF146192 | 1573946 | Hs.28555 |
| AJ420539 | 1571910 | Hs.110713 |
| AJ420539 | 46213 | Hs.110713 |
| BC002710 | 809616 | Hs.69423 |
| BC001749 | 490023 | Hs.306051 |
| NM_005378 | 41565 | Hs.25960 |
| U80811 | 1703455 | Hs.75794 |
| BC007633 | 2115155 | Hs.193053 |
| BF511644 | 42389 | Hs.271920 |
| BC002377 | 813742 | Hs.90572 |
| AK024298 | 357264 | Hs.183114 |
| AK000381 | 138059 | Hs.8562 |
| AI459117 | 83156 | Hs.111029 |
| AK026015 | 233679 | Hs.27836 |
| BC015484 | 2430219 | Hs.106857 |
| AY044429 | 1570670 | Hs.126891 |
| AW589741 | 376844 | Hs.22505 |
| BC006359 | 784876 | Hs.76888 |
| AF017790 | 345787 | Hs.58169 |
| AA993639 | 726830 | Hs.10414 |
| AW016396 | 415962 | Hs.170414 |
| BE856073 | 2018084 | Hs.199263 |
| BC002947 | 131839 | Hs.73769 |
| BC010100 | 79898 | Hs.28935 |
| NM_003979 | 595037 | Hs.194691 |
| N22033 | 255182 | Hs.121509 |
| AF245356 | 1466237 | Hs.165986 |
| AB011422 | 40011 | Hs.162189 |
| AK024898 | 823615 | Hs.99785 |
| AW206700 | 1710172 | Hs.151254 |
| AK025532 | 1572710 | Hs.128003 |
| BF444945 | 950667 | Hs.36761 |
| AF094758 | 1870550 | Hs.158450 |
| M15990 | 133178 | Hs.194148 |
| AB025904 | 251938 | Hs.235168 |
| U61374 | 785933 | Hs.15154 |
| AW104057 | 757489 | Hs.19193 |
| AF035408 | 2114193 | Hs.151407 |
| AK000770 | 1690262 | Hs.299329 |
| AI378647 | 266323 | Hs.42502 |
| AW118456 | 739193 | Hs.346950 |
| X53578 | 2509539 | Hs.169238 |
| AA613572 | 271356 | Hs.40369 |
| BC004277 | 122821 | Hs.334828 |
| BC004277 | 626206 | Hs.334828 |
| AI565489 | 1593849 | Hs.89901 |
| H06285 | 44096 | Hs.28068 |
| AI912080 | 1882481 | Hs.128443 |
| AI338252 | 470092 | Hs.25220 |
| AA810022 | 754702 | Hs.24024 |
| AF070609 | 1669248 | Hs.75379 |
| AI214383 | 418159 | Hs.6139 |
| BC011795 | 795499 | Hs.322430 |
| AF098277 | 2054945 | Hs.136529 |
| NM_006115 | 897956 | Hs.30743 |
| BC008765 | 525926 | Hs.82109 |
| AK026095 | 731373 | Hs.95011 |
| AF127563 | 669375 | Hs.40499 |
| AI471839 | 205185 | Hs.2030 |
| BC007049 | 2321596 | Hs.78944 |
| AA745593 | 1558151 | Hs.41691 |
| AI744756 | 1947144 | Hs.157117 |
| AA827777 | 156270 | Hs.153084 |
| AL096737 | 625623 | Hs.5167 |
| AW148827 | 2244016 | Hs.194766 |
| AK024964 | 813154 | Hs.173933 |
| AK024964 | 898228 | Hs.173933 |
| AL136693 | 159380 | Hs.31297 |
| BF509349 | 66694 | Hs.6127 |
| AF290004 | 1588326 | Hs.128814 |
| AI193043 | 854831 | Hs.128685 |
| BC000117 | 2062415 | Hs.105435 |
| AK026216 | 415610 | Hs.314314 |
| BC014000 | 415564 | Hs.58461 |
| AI274576 | 845355 | Hs.10029 |
| AL050143 | 306005 | Hs.7949 |
| AK027274 | 503671 | Hs.330716 |
| BC011460 | 47043 | Hs.154138 |
| BC011460 | 815214 | Hs.154138 |
| BC007617 | 839980 | Hs.132760 |
| AL110202 | 2159983 | Hs.172089 |
| BC013134 | 1883756 | Hs.127039 |
| BC004488 | 469306 | Hs.1473 |
| AI753700 | 754474 | Hs.98073 |
| AF261655 | 770785 | Hs.8910 |
| AW451965 | 155197 | Hs.131831 |
| BC001400 | 509458 | Hs.206501 |
| NM_002284 | 1509761 | Hs.278658 |
| BC000691 | 487327 | Hs.279772 |
| BC002812 | 1948085 | Hs.154510 |
| AL049949 | 489509 | Hs.28264 |
| BC004504 | 39929 | Hs.90207 |
| AI823561 | 868169 | Hs.180878 |
| AF207664 | 62263 | Hs.8230 |
| NM_006329 | 50483 | Hs.11494 |
| AF435925 | 1493596 | Hs.153692 |
| BC014018 | 503335 | Hs.6166 |
| AF132197 | 46896 | Hs.301824 |
| BC002895 | 82710 | Hs.89552 |
| AI076506 | 1675273 | Hs.133314 |
| NM_005257 | 234736 | Hs.50924 |
| AF304051 | 75859 | Hs.243960 |
| AK026642 | 898276 | Hs.179666 |
| AI015671 | 588960 | Hs.126485 |
| AI269387 | 768008 | Hs.55220 |
| BC000314 | 28364 | Hs.99947 |
| L40377 | 341978 | Hs.41726 |
| AI567961 | 127970 | Hs.14480 |
| AI221721 | 712600 | Hs.110099 |
| BI492432 | 133273 | Hs.103724 |
| AI439152 | 156962 | Hs.173392 |
| BC005894 | 811061 | Hs.132821 |
| AJ420516 | 41987 | Hs.21177 |
| AF000546 | 303109 | Hs.189999 |
| AK024986 | 366966 | Hs.27865 |
| NM_000909 | 143332 | Hs.169266 |
| AI393709 | 841357 | Hs.105658 |
| NM_002510 | 773330 | Hs.82226 |
| AF159570 | 853809 | Hs.24950 |
| AI984359 | 39178 | Hs.16341 |
| BC000187 | 278531 | Hs.351875 |
| M86609 | 196992 | Hs.306098 |
| AW069800 | 796152 | Hs.250705 |
| AW004703 | 1574914 | Hs.9547 |
| AI350087 | 1859319 | Hs.214410 |
| AW104435 | 795197 | Hs.108323 |
| BC017586 | 1950691 | Hs.55150 |
| AA401311 | 1894931 | Hs.188790 |
| AK025494 | 1470278 | Hs.29076 |
| X08020 | 180561 | Hs.301961 |
| AL080213 | 49385 | Hs.52792 |
| BG236645 | 2032251 | Hs.102793 |
| AW057705 | 2542486 | Hs.385 |
| AI969835 | 176606 | Hs.79389 |
| AA906471 | 310832 | Hs.100748 |
| AF038633 | 2271343 | Hs.134932 |
| AL117410 | 754018 | Hs.154567 |
| BC018043 | 564621 | Hs.78589 |
| BC003667 | 590338 | Hs.108957 |
| BC003513 | 345034 | Hs.24395 |
| AK024822 | 244654 | Hs.47572 |
| AF106912 | 1634832 | Hs.132781 |
| AI936089 | 1632248 | Hs.130874 |
| BC018001 | 611443 | Hs.118836 |
| AI200391 | 1585713 | Hs.98641 |
| BC002842 | 1500000 | Hs.180779 |
| BC004925 | 1635601 | Hs.330425 |
| AA747738 | 788355 | Hs.20132 |
| AI424406 | 43764 | Hs.284256 |
| AF202636 | 69002 | Hs.9613 |
| AK025912 | 769959 | Hs.75617 |
| AA765256 | 1915080 | Hs.135191 |
| H17636 | 50492 | Hs.288642 |
| AA736984 | 842939 | Hs.85302 |
| BC001692 | 704085 | Hs.33084 |
| BC015640 | 711552 | Hs.88778 |
| BF939156 | 840753 | Hs.241392 |
| BC001346 | 49794 | Hs.22142 |
| BE465918 | 1553696 | Hs.12114 |
| BC014105 | 86035 | Hs.11085 |
| AI268609 | 1416055 | Hs.153479 |
| NM_001262 | 291057 | Hs.4854 |
| AJ420521 | 1572196 | Hs.22209 |
| AI278360 | 1655480 | Hs.31409 |
| NM_002451 | 2436784 | Hs.152817 |
| AW575162 | 127821 | Hs.1211 |
| AW134884 | 2273445 | Hs.37023 |
| AI335013 | 417884 | Hs.237642 |
| BC017785 | 1638481 | Hs.87860 |
| AI767898 | 841492 | Hs.237809 |
| AW082270 | 1895781 | Hs.12496 |
| AW082270 | 52071 | Hs.12496 |
| AI499593 | 152453 | Hs.25351 |
| AA053400 | 127063 | Hs.203213 |
| AF016267 | 110226 | Hs.119684 |
| AL119854 | 361587 | Hs.296317 |
| BC014277 | 489519 | Hs.245188 |
| AL110124 | 502446 | Hs.5297 |
| BF447150 | 1556897 | Hs.119383 |
| AF218006 | 2297394 | Hs.24391 |
| AW450080 | 241043 | Hs.322149 |
| BC001314 | 130835 | Hs.96125 |
| AJ223366 | 1915759 | Hs.116051 |
| BC002325 | 1570427 | Hs.32417 |
| BF436849 | 1499900 | Hs.8917 |
| AK024818 | 589115 | Hs.83169 |
| BC006458 | 725454 | Hs.83758 |
| BC008584 | 344430 | Hs.170195 |
| BF509554 | 1842331 | Hs.144921 |
| NM_001254 | 204214 | Hs.69563 |
| BC008857 | 725649 | Hs.77810 |
| U96136 | 51083 | Hs.80220 |
| NM_000139 | 1591736 | Hs.30 |
| S77873 | 179403 | Hs.75183 |
| BC014526 | 25763 | Hs.168640 |
| BC014526 | 429065 | Hs.168640 |
| BC007348 | 489677 | Hs.77573 |
| AW732240 | 22500 | Hs.16365 |
| M93426 | 785148 | Hs.78867 |
| AI460151 | 81289 | Hs.78045 |
| AL080059 | 745011 | Hs.173094 |
| BF196234 | 1884217 | Hs.147491 |
| NM_004430 | 26568 | Hs.74088 |
| U87460 | 41658 | Hs.27747 |
| AI469557 | 813520 | Hs.2913 |
| AW205835 | 1867357 | Hs.110418 |
| AF130068 | 207735 | Hs.297681 |
| BC011628 | 156473 | Hs.113 |
| L10386 | 301735 | Hs.2022 |
| BC007438 | 753252 | Hs.74266 |
| AW050970 | 666128 | Hs.155597 |
| AI569068 | 812294 | Hs.22247 |
| AI002941 | 1698784 | Hs.347970 |
| BC002641 | 1474900 | Hs.80342 |
| BC010691 | 321455 | Hs.343553 |
| AI311127 | 246194 | Hs.125522 |
| AW014836 | 415326 | Hs.18844 |
| BC014651 | 773296 | Hs.98428 |
| AF023676 | 2020772 | Hs.31130 |
| AF063020 | 745118 | Hs.82110 |
| AI866828 | 2421971 | Hs.179729 |
| AL161983 | 502096 | Hs.21415 |
| AI125473 | 2029503 | Hs.157281 |
| BG008036 | 344588 | Hs.50915 |
| AI371090 | 784257 | Hs.21611 |
| X97859 | 342458 | Hs.19413 |
| AF118088 | 280375 | Hs.46677 |
| NM_006103 | 786675 | Hs.2719 |
| AJ420485 | 325520 | Hs.14454 |
| AL137343 | 1688965 | Hs.260855 |
| AL137343 | 177665 | Hs.260855 |
| AK025583 | 767069 | Hs.82845 |
| NM_006142 | 2027515 | Hs.184510 |
| BC007659 | 813387 | Hs.80706 |
| AA854341 | 186301 | Hs.38034 |
| AI797308 | 194638 | Hs.193651 |
| AA988024 | 34720 | Hs.127951 |
| NM_014279 | 52076 | Hs.74376 |
| BC013107 | 327304 | Hs.110707 |
| AI283926 | 40056 | Hs.9004 |
| AI373939 | 839101 | Hs.74471 |
| AW207140 | 363590 | Hs.6111 |
| BC004295 | 767993 | Hs.29759 |
| AK056230 | 343695 | Hs.349319 |
| NM_012168 | 179269 | Hs.132753 |
| BC000566 | 431632 | Hs.50267 |
| BC007022 | 161456 | Hs.332053 |
| BC016623 | 414999 | Hs.77711 |
| AW138839 | 1537001 | Hs.24210 |
| AL365412 | 144880 | Hs.107203 |
| AW445220 | 782575 | Hs.69517 |
| N51717 | 156962 | Hs.29410 |
| X79676 | 866702 | Hs.211595 |
| AI263595 | 344091 | Hs.333199 |
| BC015940 | 42070 | Hs.153952 |
| NM_030761 | 1909317 | Hs.302428 |
| AI590055 | 1523411 | Hs.124110 |
| NM_004425 | 301122 | Hs.81071 |
| AI733491 | 1608923 | Hs.129230 |
| BF433635 | 1911471 | Hs.145711 |
| BC005313 | 1705861 | Hs.123107 |
| AL133591 | 2016924 | Hs.141480 |
| AI362949 | 2019009 | Hs.75169 |
| AF334710 | 78946 | Hs.8364 |
| AL161961 | 1500766 | Hs.17767 |
| AF094754 | 155038 | Hs.32973 |
| AF094754 | 1685363 | Hs.32973 |
| AK026760 | 75059 | Hs.251946 |
| AF052224 | 2432360 | Hs.194684 |
| NM_005864 | 795730 | Hs.24587 |
| AW205768 | 2300372 | Hs.91877 |
| AW139532 | 814154 | Hs.105069 |
| AI025919 | 1757930 | Hs.131674 |
| AF065857 | 2809853 | Hs.248185 |
| AL136678 | 1455603 | Hs.87729 |
| AK025428 | 812975 | Hs.77546 |
| BC015497 | 346696 | Hs.94865 |
| AI272951 | 85643 | Hs.75599 |
| NM_000436 | 28469 | Hs.177584 |
| AI697249 | 1566793 | Hs.784 |
| AK024750 | 726860 | Hs.32017 |
| M62626 | 429368 | Hs.89583 |
| BC017901 | 489320 | Hs.292358 |
| AF117386 | 1581685 | Hs.35086 |
| AA747541 | 1526789 | Hs.300592 |
| AK024855 | 687875 | Hs.181301 |
| BC014564 | 898219 | Hs.79284 |
| AF070631 | 1500821 | Hs.11355 |
| AF070631 | 454083 | Hs.11355 |
| AF070631 | 79761 | Hs.11355 |
| BC007678 | 505881 | Hs.1217 |
| BC000632 | 530035 | Hs.14331 |
| BC005963 | 1631546 | Hs.36978 |
| AA233494 | 2284619 | Hs.159468 |
| BE549691 | 1860836 | Hs.244391 |
| L02785 | 1873533 | Hs.1650 |
| AW151360 | 593690 | Hs.270737 |
| AW118445 | 175103 | Hs.57652 |
| BC009799 | 1410444 | Hs.270833 |
| AK055386 | 365991 | Hs.285050 |
| AJ012214 | 2191807 | Hs.227115 |
| U06641 | 78294 | Hs.150207 |
| BF433809 | 1896981 | Hs.3195 |
| NM_000734 | 1572298 | Hs.97087 |
| R42342 | 796198 | Hs.30942 |
| NM_002514 | 1505534 | Hs.235935 |
| AI280246 | 1893765 | Hs.149504 |
| AF025887 | 504791 | Hs.169907 |
| AL137346 | 22917 | Hs.13299 |
| NM_004370 | 786609 | Hs.101302 |
| BC009628 | 1569815 | Hs.127771 |
| AF059321 | 1610146 | Hs.301698 |
| BC010652 | 415589 | Hs.91668 |
| AW291180 | 1455566 | Hs.258 |
| AF208043 | 824602 | Hs.155530 |
| AW081673 | 296529 | Hs.181345 |
| D50419 | 1404841 | Hs.119014 |
| AF034996 | 44164 | Hs.173034 |
| AW448954 | 490784 | Hs.143046 |
| AI741469 | 1732247 | Hs.57787 |
| NM_003248 | 758266 | Hs.75774 |
| AW073971 | 345234 | Hs.238954 |
| AI689069 | 487988 | Hs.109606 |
| BC015947 | 2138030 | Hs.47584 |
| BC003388 | 502486 | Hs.146847 |
| AL117616 | 280950 | Hs.300741 |
| BC005246 | 509731 | Hs.84072 |
| AI492117 | 1641837 | Hs.131905 |
| AF233588 | 1614140 | Hs.27018 |
| NM_001452 | 310138 | Hs.44481 |
| BF512918 | 2363954 | Hs.2159 |
| AI311775 | 2046679 | Hs.192819 |
| NM_006455 | 347434 | Hs.207251 |
| BC004267 | 950451 | Hs.135200 |
| AI147926 | 141115 | Hs.285401 |
| J03607 | 810131 | Hs.182265 |
| AL043170 | 758284 | Hs.160881 |
| NM_005410 | 530814 | Hs.3314 |
| BC015397 | 1908705 | Hs.122055 |
| BC009352 | 842896 | Hs.351623 |
| AF053453 | 252382 | Hs.121068 |
| AA923424 | 289025 | Hs.135567 |
| AW291447 | 472008 | Hs.301921 |
| AA999861 | 1636369 | Hs.126892 |
| NM_006235 | 1541958 | Hs.2407 |
| AB037715 | 32122 | Hs.183639 |
| AI424409 | 1493107 | Hs.348909 |
| AF002672 | 2502722 | Hs.152944 |
| BC008651 | 50892 | Hs.23133 |
| M87507 | 120106 | Hs.2490 |
| BF508058 | 744374 | Hs.4996 |
| AI888490 | 666707 | Hs.55902 |
| AF104313 | 782460 | Hs.173374 |
| BF056618 | 2019526 | Hs.233634 |
| AL136842 | 683059 | Hs.260024 |
| M85294 | 323185 | Hs.274382 |
| BC017703 | 884789 | Hs.57672 |
| BC014117 | 143443 | Hs.2001 |
| U60975 | 2413337 | Hs.278571 |
| AF105378 | 1569187 | Hs.8040 |
| AL137662 | 85193 | Hs.274401 |
| AL134596 | 2098508 | Hs.89512 |
| BC015496 | 180803 | Hs.32309 |
| AW291368 | 196435 | Hs.34455 |
| AK027121 | 795746 | Hs.38178 |
| AF062595 | 39600 | Hs.18268 |
| AK025796 | 595420 | Hs.239218 |
| BC005359 | 2016194 | Hs.151413 |
| AA731869 | 1847618 | Hs.1309 |
| BC016757 | 502142 | Hs.20985 |
| BM141806 | 772373 | Hs.47094 |
| BC002415 | 823928 | Hs.1581 |
| NM_057749 | 773345 | Hs.30464 |
| M26683 | 768561 | Hs.303649 |
| BC010690 | 1554167 | Hs.334703 |
| NM_000795 | 2017689 | Hs.73893 |
| BC005055 | 1049346 | Hs.274344 |
| N93663 | 307019 | Hs.118240 |
| AI311898 | 2047361 | Hs.140197 |
| AI732388 | 159362 | Hs.177576 |
| AI869243 | 898249 | Hs.17882 |
| AI740671 | 299274 | Hs.351568 |
| BC012113 | 1592715 | Hs.166146 |
| AF097021 | 2312470 | Hs.273321 |
| AB058769 | 754126 | Hs.334838 |
| AF263462 | 296444 | Hs.18376 |
| AL137686 | 712166 | Hs.182982 |
| R56080 | 34888 | Hs.12246 |
| NM_005906 | 1841007 | Hs.148496 |
| BM126079 | 1894405 | Hs.124130 |
| BF477905 | 366209 | Hs.58104 |
| AA777592 | 71116 | Hs.288582 |
| NM_003512 | 283919 | Hs.28777 |
| NM_002538 | 1881469 | Hs.171952 |
| BC018112 | 857002 | Hs.75969 |
| AI765908 | 1883327 | Hs.129166 |
| AI216751 | 1884505 | Hs.143977 |
| AA767639 | 34149 | Hs.79170 |
| BC002745 | 789147 | Hs.146580 |
| U49395 | 486678 | Hs.77807 |
| AL110131 | 726599 | Hs.28783 |
| BC005305 | 2322038 | Hs.408 |
| AI571426 | 841282 | Hs.105700 |
| AA398015 | 726645 | Hs.287364 |
| AB012643 | 1475595 | Hs.250769 |
| M93119 | 22895 | Hs.89584 |
| AI703322 | 108330 | Hs.103253 |
| AW453001 | 487820 | Hs.76722 |
| AA937401 | 1569876 | Hs.13234 |
| AF059617 | 795877 | Hs.3838 |
| AA910331 | 491692 | Hs.119129 |
| BC002718 | 1759582 | Hs.10086 |
| AJ315514 | 784296 | Hs.1790 |
| AI560576 | 146123 | Hs.79005 |
| BC001645 | 897822 | Hs.74101 |
| BC017073 | 128695 | Hs.22015 |
| AF009227 | 155716 | Hs.172816 |
| AF054181 | 809455 | Hs.183435 |
| AF222340 | 725126 | Hs.247043 |
| BC016719 | 626348 | Hs.17558 |
| AA806859 | 147651 | Hs.266957 |
| AK024697 | 300632 | Hs.25477 |
| AI961887 | 418262 | Hs.119571 |
| AI089428 | 278404 | Hs.21964 |
| AF257182 | 430717 | Hs.160271 |
| AK026256 | 796079 | Hs.289015 |
| BE349538 | 826256 | Hs.15791 |
| AA831508 | 366830 | Hs.32553 |
| AI766890 | 428163 | Hs.30022 |
| NM_005923 | 1880757 | Hs.151988 |
| NM_000609 | 2239290 | Hs.237356 |
| AL137274 | 742642 | Hs.11169 |
| AW084130 | 1468820 | Hs.166373 |
| AK027036 | 490329 | Hs.61345 |
| U90914 | 1896838 | Hs.5057 |
| AI733236 | 1909535 | Hs.128312 |
| BC001350 | 50480 | Hs.172851 |
| AK025346 | 812256 | Hs.48965 |
| NM_001873 | 40558 | Hs.75360 |

Having now generally described the invention, the same will be more readily understood through reference to the following examples which are provided by way of illustration, and are not intended to be limiting of the present invention, unless specified.

### EXAMPLES

### Example I

Clinical specimen collection and clinicopathological parameters. 86 patients were expression profiled, 57 of these had clinical follow-up, specifically overall survival. Biomarker status is shown below in Table 3 for all 86 patients

TABLE 3
Age and biomarker status for the 86 patients
subsequently gene expression profiled

| | | No. of Cases | Percentage |
|---|---|---|---|
| Age | <45 | 12 | 14% |
| | 45-55 | 24 | 28% |
| | >55 | 50 | 58% |
| Estrogen-receptor status | positive | 41 | 48% |
| | negative | 45 | 52% |
| Progesterone-receptor status | positive | 32 | 37% |
| | negative | 54 | 63% |
| Her2/Neu status | positive | 16 | 19% |
| | intermediate | 23 | 27% |
| | negative | 45 | 54% |

The estrogen receptor (ER) status profile was discovered by the following steps
(i) patient biopsy set was divided up into two groups based on prior knowledge of their respective ER status (ER-positive and ER-negative);
(ii) a standard t-test was completed on each gene to identify genes whose expression was different between ER−/+; and
(iii) the P values were adjusted via the Benjamini-Hochberg false discovery rate method procedure for the inclusion of all genes which had p <0.05.

This example was used for a hierarchical clustering of 86 patients with an overall total of 3,105 genes that correspond to an ER signature. The results are shown in Tables 1 and 2.

### Example II

Frozen biological breast cancer samples from 247 patients were used for expression profiling to identify genes that correlate with ER+ and ER− states in a manner analogous to the previous example. The analysis identified 2608 genes that correlated with one of two ER states. The results are shown in Tables 3 and 4.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. An array comprising polynucleotide probes capable of hybridizing to nucleic acid molecules of one or more of the genes listed in Table 1, 2, 3, or 4 and comprising sequences within 350 nucleotides of the polyadenylation site of said genes, said probes hybridized to nucleic acids derived from a cell of a subject afflicted with, or suspected of having, breast cancer

2. The array of claim 1 comprising 11 or more of the genes in Table 1, 2, 3, or 4.

3. The array of claim 2 comprising all the genes in Table 1, 2, 3, or 4.

4. The array of claim 1 wherein said cell is from a human subject or such a subject afflicted with breast cancer.

5. The array of claim 1 wherein said nucleic acids derived from one or more breast cancer cells are prepared by mRNA amplification.

6. The array of claim 1 wherein said nucleic acids derived from one or more breast cancer cells are cDNA.

7. The array of claim 1 wherein said cell is in a section of tissue from a subject or is microdissected from said section.

8. A method to determine the ER status of breast cancer cells in a sample from a subject comprising assaying said sample for expression of one or more genes in Table 1 or 3 and/or one or more genes in Table 2 or 4.

9. The method of claim 8 wherein said assaying comprises preparing RNA, optionally labeled, from said sample and optionally converting said RNA into cDNA, optionally labeled.

10. The method of claim 9 wherein said RNA is not labeled and used for quantitative PCR.

11. The method of claim 9 wherein said assaying comprises using an array.

12. The method of claim 8 wherein said sample is a ductal lavage or fine needle aspiration or FFPE breast tissue sample.

13. The method of claim 12 wherein said sample is microdissected to isolate one or more cells that are breast cancer cells or suspected of being breast cancer cells.

14. The method of claim 10 further comprising determination of the ratio of the expression of a gene in Table 1 or 3 to the expression of a gene in Table 2 or 4 as an indicator of ER status.

15. A method to determine therapeutic treatment for a patient having breast cancer comprising
identifying said patient as being ER positive or ER negative after assaying breast cancer cells of said patient for expression of one or more genes listed in Table 1, 2, 3, and/or 4 and
selecting the appropriate treatment for a patient having cells of such ER status.

16. The method of claim 15 wherein said assaying comprises preparing RNA, optionally labeled, from said sample and optionally converting said RNA into cDNA, optionally labeled.

17. The method of claim 16 wherein said RNA is not labeled and used for quantitative PCR.

18. The method of claim 16 wherein said assaying comprises using an array.

19. The method of claim 16 wherein said sample is a ductal lavage or fine needle aspiration or FFPE breast tissue sample.

20. The method of claim 19 wherein said sample is microdissected to isolate one or more cells that are breast cancer cells or suspected of being breast cancer cells.

21. The method of claim 16 wherein said assaying comprises determination of the ratio of the expression of a gene in Table 1 or 3 to the expression of a gene in Table 2 or 4 as an indicator of ER status.
